(19) United States
(12) Patent Application Publication
(10) Pub. No.: US 2011/0301155 A1
(43) Pub. Date: Dec. 8, 2011

(54) INDAZOLE COMPOUNDS FOR ACTIVATING GLUCOKINASE

(76) Inventors: Tsuneo Yasuma, Osaka-shi (JP); Shigekazu Sasaki, Osaka-shi (JP); Osamu Ujikawa, Osaka-shi (JP); Yasufumi Miyamoto, Osaka-shi (JP); Stephen L. Gwaltney, San Diego, CA (US); Sheldon Cao, San Diego, CA (US); Andy Jennings, San Diego, CA (US)

(21) Appl. No.: 12/452,174

(22) PCT Filed: Jun. 18, 2008
(86) PCT No.: PCT/US08/07535

§ 371 Date: Dec. 18, 2009

Related U.S. Application Data
(60) Provisional application No. 60/929,240, filed on Jun. 19, 2007.

(51) Int. Cl. A61K 31/541; A61K 31/427; A61K 31/4439; C07D 417/12; A61K 31/496; A61K 31/5377; A61K 31/4545; A61K 31/444; C07D 498/04; A61K 31/5383; A61K 31/4985; C07D 401/14; A61P 3/04; A61P 3/10; C07D 417/14

(52) U.S. Cl. 514/228.2; 548/181; 514/370; 546/270.7; 514/338; 544/364; 514/253.1; 544/124; 514/234.5; 546/187; 514/316; 514/333; 546/256; 544/105; 514/230.5; 544/350; 514/249; 544/62; 546/275.7

(57) ABSTRACT

The present invention aims to provide a glucokinase activator useful as a pharmaceutical agent such as an agent for the prophylaxis or treatment of diabetes, obesity and the like, and the like.

A compound represented by the formula (I):

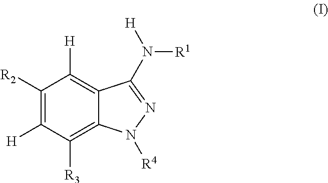

wherein R¹ is an optionally substituted 4- to 7-membered nitrogen-containing heterocyclic group, optionally substituted carbamoyl, or optionally substituted sulfamoyl; R² is optionally substituted alkyl, optionally substituted alkoxy, an optionally substituted 3- to 7-membered cyclic group, —SR′, —SOR′, or —SO₂R′ (R′ is a substituent); R³ is hydrogen, halogen, optionally substituted alkyl, optionally substituted alkenyl, optionally substituted alkoxy, —O-Cy (Cy is an optionally substituted 3- to 7-membered cyclic group which may be condensed with benzene), —SR″, —SOR″, or —SO₂R″ (R″ is a substituent), or an optionally substituted 3- to 7-membered cyclic group which may be condensed with benzene; R⁴ is hydrogen, or optionally substituted alkyl; provided that when R³ is hydrogen, halogen, or methoxy, then R² is not optionally substituted alkyl, or optionally substituted alkoxy; further provided that 5-[5-{[(2S)-2-amino-3-phenylpropyl]oxy}-2-(3-furyl)pyridin-3-yl]-N-pyridin-4-yl-1H-indazol-3-amine and 5-[5-{[(2S)-2-amino-3-phenylpropyl]oxy}-2-(3-furyl)pyridin-3-yl]-1-(4-methoxybenzyl)-N-pyridin-4-yl-1H-indazol-3-amine are excluded; or a salt thereof.

### TECHNICAL FIELD OF THE INVENTION

The present invention relates to an indazole compound having a glucokinase activating effect and useful as a therapeutic agent of diabetes and the like.

### BACKGROUND OF THE INVENTION

Glucokinase (sometimes to be abbreviated to as GK in the present specification) (EC2.7.1.1) is one of the four kinds of hexokinases found in mammals, and is also called hexokinase IV. GK is an enzyme that catalyzes the conversion of glucose to glucose-6-phosphate, which is the first Step of glycolysis. GK is mainly present in the pancreatic β cell and the liver, and acts in the pancreatic β cell as a sensor of extracellular glucose concentration that defines the glucose-stimulated insulin secretion. In the liver, the enzyme reaction of GK becomes a rate determining factor and regulates glycogen synthesis and glycolysis. The three hexokinases (I, II, III) other than GK reach the maximum enzyme activity at a glucose concentration of 1 mM or below. In contrast, GK shows low affinity for glucose and has a Km value of 8-15 mM which is close to a physiological blood glucose level. Accordingly, GK-mediated promotion of intracellular glucose metabolism occurs, which corresponds to blood glucose changes from normal blood glucose (5 mM) to postprandial hyperglycemia (10-15 mM).

The hypothesis proposed by Matschinsky et al. in 1984 that GK acts as a glucose sensor in the pancreatic β cell and hepatocytes has been demonstrated by the analysis of glucokinase gene manipulation mouse in recent years (see The Journal of Biological Chemistry (J. Biol. Chem.), 1995, vol. 270, page 30253-30256; The Journal of Biological Chemistry (J. Biol. Chem.), 1997, vol. 272, page 22564-22569; The Journal of Biological Chemistry (J. Biol. Chem.), 1997, vol. 272, page 22570-22575; NIHONRINSHO, 2002, vol. 60, page 523-534; and Cell, 1995, vol. 83, page 69-78). That is, GK heterozygous knockout mouse showed a hyperglycemic condition, and further, a disordered glucose-stimulated insulin secretion response. GK homozygous knockout mouse dies shortly after birth with manifestations of marked hyperglycemia and urinary sugar. On the other hand, GK overexpressed mouse (hetero type) showed decreased blood glucose level, increased blood glucose clearance rate, increased liver glycogen content and the like. From these findings, it has been clarified that GK plays an important role in the systemic glucose homeostasis. In other words, decreased GK activity causes insulin secretion failure and lower liver glucose metabolism, which develops impaired glucose tolerance and diabetes. Conversely, GK activation or increased GK activity due to overexpression causes promoted insulin secretion and promoted liver glucose metabolism, which in turn increases the systemic use of glucose to improve glucose tolerance.

In addition, it has been clarified from the analysis of a report on GK gene abnormality mainly in the family of MODY2 (Maturity Onset Diabetes of the Young) that GK also acts as a glucose sensor in human, and plays a key role in glucose homeostasis (see Nature, 1992, vol. 356, page 721-722). In GK gene abnormality, due to the decreased affinity of GK for glucose (increased Km value) and decreased Vmax, the blood glucose threshold value of insulin secretion increases and the insulin secretory capacity decreases. In the liver, due to the decreased GK activity, decreased glucose uptake, promoted gluconeogenesis, decreased glycogen synthesis and liver insulin resistance are observed. On the other hand, a family with a mutation increasing the GK activity has also been found. In such family, fasting hypoglycemia associated with increased plasma insulin concentration is observed (see New England Journal Medicine, 1998, vol. 338, page 226-230).

As mentioned above, GK acts as a glucose sensor in mammals including human, and plays an important role In blood glucose regulation. On the other hand, control of blood glucose utilizing the glucose sensor system of GK is considered to open a new way to treat diabetes in many type 2 diabetes patients. Particularly, since a GK activating substance is expected to show insulin secretagogue action in the pancreatic β cell and glucose uptake promotion and glucose release suppressive action in the liver, it will be useful as a prophylactic or therapeutic drug for type 2 diabetes.

In recent years, it has been clarified that pancreatic 13 cell type glucokinase expresses locally in the feeding center (Ventromedial Hypothalamus: VMH) of rat brain. A subset of nerve cell present in VMH is called glucose responsive neuron, and plays an important role in the body weight control. From electrophysiological experiments, the neuron is activated in response to physiological changes in the glucose concentration (5-20 mM). However, since the glucose concentration sensor system of VHM is assumed to have a mechanism mediated by glucokinase as in the case of insulin secretion in the pancreatic β cell, separately from pancreatic β cell and the liver, a pharmaceutical agent capable of activating glucokinase of VHM has a possibility of providing not only a blood glucose corrective effect but also improvement of obesity.

As mentioned above, a pharmaceutical agent capable of activating GK is useful as a prophylactic or therapeutic drug for diabetes and chronic diabetic complications such as retinopathy, nephropathy, neuropathy, ischemic cardiac diseases, arteriosclerosis and the like, and further, as a prophylactic or therapeutic drug for obesity.

On the other hand, as a 3-aminoindazole compound, the following compound has been reported.

WO 2003/028720 discloses that a compound represented by

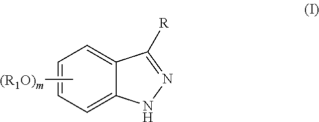

wherein R is —NHCONR′ and the like, has a kinase inhibitory action and is useful for cancer and the like.

WO 2002/022601 discloses a compound represented by

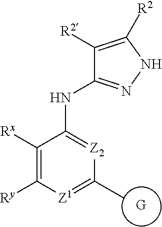

wherein R² and R^{2′} may form an unsaturated ring, has a GSK-3 activity-inhibitory and an Aurora activity-inhibitory action, and is useful for Alzheimer's disease and the like.

WO 2005/085227 discloses a compound represented by

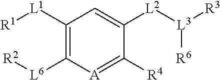

wherein R¹ is substituted heterocycle and the like, has a PKB/AKT kinase activity-inhibitory action, and is useful for cancer and the like.

The compound encompasses 5-[(5-{[(2S)-2-amino-3-phenylpropyl]oxy}-2-(3-furyl)pyridin-3-yl]-N-pyridin-4-yl-1H-indazol-3-amine, and 5-[5-{[(2S)-2-amino-3-phenylpropyl]oxy}-2-(3-furyl)pyridin-3-yl]-1-(4-methoxybenzyl)-N-pyridin-4-yl-1H-indazol-3-amine.

In addition, J Grimsby et al., Science, 301, 370-373, 2003, A. M. Efanov et al., Endocrinology, 146, 3696-3701, 2005, and M. Futamura et al., J. Biol. Chem. 281, 37668-37674 disclose, as GK activating drugs, compounds having structures different from the structure of the compound of the present invention.

### DISCLOSURE OF THE INVENTION

### Problems to be Solved by the Invention

The present invention aims to provide a glucokinase activator useful as a pharmaceutical agent such as an agent for the prophylaxis or treatment of diabetes, obesity and the like, and the like.

### Means of Solving the Problems

The present inventors have conducted intensive studies in an attempt to solve the aforementioned problems and found that a compound represented by the following formula (I) unexpectedly has a superior glucokinase activating effect, and further, superior properties as a pharmaceutical product, such as stability and the like, and can be a safe and useful pharmaceutical agent, which resulted in the completion of the present invention.

Accordingly, the present invention relates to the following.

is [1] A compound represented by the formula (I):

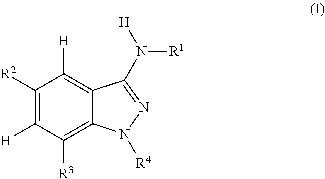

wherein

### R¹ is

an optionally substituted 4 to 7-membered nitrogen-containing heterocyclic group,
optionally substituted carbamoyl, or
optionally substituted sulfamoyl;

### R² is

optionally substituted alkyl,
optionally substituted alkoxy,
an optionally substituted 3 to 7-membered cyclic group, —SR′, —SOR′, or —SO₂R′ (R′ is a substituent);

### R³ is

hydrogen,
halogen,
optionally substituted alkyl,
optionally substituted alkenyl,
optionally substituted alkoxy,
—O-Cy (Cy is an optionally substituted 3 to 7-membered cyclic group which may be condensed with benzene),
—SR″, —SOR″, or —SO₂R″ (R″ is a substituent), or an optionally substituted 3 to 7-membered cyclic group which may be condensed with benzene;

### R⁴ is

hydrogen, or
optionally substituted alkyl;
   provided that
when R³ is hydrogen, halogen, or methoxy,
then R² is not optionally substituted alkyl, or optionally substituted alkoxy;
   further provided that 5-[(5-{[(2S)-2-amino-3-phenylpropyl]oxy}-2-(3-furyl)pyridin-3-yl]-N-pyridin-4-yl-1H-indazol-3-amine and 5-[5-{[(2S)-2-amino-3-phenylpropyl]oxy}-2-(3-furyl)pyridin-3-yl]-1-(4-methoxybenzyl)-N-pyridin-4-yl-1H-indazol-3-amine are excluded;
   or a salt thereof.
   [2] The compound of the above-mentioned [1],
   wherein

### R¹ is

an optionally substituted 4 to 7-membered nitrogen-containing heterocyclic group, or
optionally substituted sulfamoyl.
   [3] The compound of the above-mentioned [1],
   wherein

### R² is

an optionally substituted 3 to 7-membered cyclic group, —SR′, —SOR′, or —SO₂R′ (R′ is a substituent).
   [4] The compound of the above-mentioned [1],
   wherein

### R¹ is

(i) a 4 to 7-membered nitrogen-containing heterocyclic group optionally substituted by C₁₋₆ alkyl optionally substituted by one or more of the same or different substituents selected from
hydroxy,
cyano,
optionally substituted amino,
optionally substituted 5 to 6-membered cyclic amino,
carboxy,
C₁₋₆ alkoxycarbonyl, and
optionally substituted carbamoyl, or
   (ii) optionally substituted carbamoyl.
   [5] The compound of the above-mentioned [1],
   wherein

### R² is

(i) C₁₋₆ alkyl,
(ii) C₁₋₆ alkoxy optionally substituted by one or more of the same or different substituents selected from C₆₋₁₀ aryl and C₁₋₆ alkoxy,
(iii) —SR′, —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), or
(iv) an optionally substituted 3 to 7-membered cyclic group.
   [6] The compound of the above-mentioned [1],
   wherein

### R³ is

(i) hydrogen,
(ii) halogen,
(iii) C₁₋₆ alkyl,
(iv) C₂₋₆ alkenyl optionally substituted by 5 to 6-membered heterocyclic group,
(v) C₁₋₆ alkoxy optionally substituted by one or more of the same or different substituents selected from
   (a) optionally substituted amino,
   (b) phenyl optionally substituted by one or more of the same or different substituents selected from
      halogen, and
      C₁₋₆ alkylsulfonyl,
   (c) 5 to 6-membered heterocyclic ring which may be substituted by one or more of the same or different substituents selected from
      C₁₋₆ alkyl,
      C₁₋₆ alkylthio,
      C₁₋₆ alkylsulfonyl,
      carboxy,
      C₁₋₆ alkoxycarbonyl and
      oxo,
      and which may be condensed with benzene,
   (d) carbamoyl optionally substituted by C₁₋₆ alkyl, and
   (e) C₁₋₆ alkylsulfonyl,
(vi) phenoxy or 5 to 6-membered heteroaryloxy, each of which may be substituted by one or more of the same or different substituents selected from
   halogen,
   C₁₋₆ alkylsulfonyl, and
   optionally substituted carbamoyl, or
(vii) 5 to 6-membered heterocyclic ring which may be substituted by C₁₋₆ alkyl, and which may be condensed with benzene.
   [7] The compound of the above-mentioned [1],
   wherein

### R⁴ is

(i) hydrogen, or
(ii) C₁₋₆ alkyl optionally substituted by one or more of the same or different substituents selected from C₆₋₁₀ aryl and C₁₋₆ alkoxy.
   [8] The compound of the above-mentioned [1],
   wherein

### R¹ is

(i) a 4 to 7-membered nitrogen-containing heterocyclic group optionally substituted by C₁₋₆ alkyl optionally substituted by one or more of the same or different substituents selected from
   hydroxy,
   cyano,
   optionally substituted amino,
   optionally substituted 5 to 6-membered cyclic amino,
   carboxy,
   C₁₋₆ alkoxycarbonyl, and
   optionally substituted carbamoyl, or
(ii) optionally substituted carbamoyl;

### R² is

(i) C₁₋₆ alkyl,
(ii) C₁₋₆ alkoxy optionally substituted by one or more of the same or different substituents selected from C₆₋₄₀ aryl and C₁₋₆ alkoxy,
(iii)-SR′, —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), or
(iv) an optionally substituted 3 to 7-membered cyclic group;

### R³ is

(i) hydrogen,
(ii) halogen,
(iii) C₁₋₆ alkyl,
(iv) C₁₋₆ alkenyl optionally substituted by 5 to 6-membered heterocyclic group,
(v) C₁₋₆ alkoxy optionally substituted by one or more of the same or different substituents selected from
   (a) optionally substituted amino,
   (b) phenyl optionally substituted by one or more of the
   same or different substituents selected from
      halogen, and
      C₁₋₆ alkylsulfonyl,
   (c) 5 to 6-membered heterocyclic ring which may be substituted by one or more of the same or different substituents selected from
      C₁₋₆ alkyl,
      C₁₋₆ alkylthio,
      C₁₋₆ alkylsulfonyl,
      carboxy,
      C₁₋₆ alkoxycarbonyl and
      oxo,
   and which may be condensed with benzene,
   (d) carbamoyl optionally substituted by C₁₋₆ alkyl, and
   (e) C₁₋₆ alkylsulfonyl,
(vi) phenoxy or 5 to 6-membered heteroaryloxy optionally substituted by one or more of the same or different substituents selected from
   halogen,
   C₁₋₆ alkylsulfonyl, and
      optionally substituted carbamoyl, or
(vii) 5 to 6-membered heterocyclic ring which may be substituted by C₁₋₆ alkyl, and which may be condensed with benzene.

### R⁴ is

(i) hydrogen, or
(ii) C₁₋₆ alkyl optionally substituted by one or more of the same or different substituents selected from C₆₋₁₀ aryl and C₁₋₆ alkoxy.
   [9] The compound of the above-mentioned [1],
   wherein

### R³ is

optionally substituted alkyl,
optionally substituted alkenyl,
   C₂₋₆ alkoxy, or substituted C₁₋₆ alkoxy,
   —O-Cy (Cy is an optionally substituted 3 to 7-membered cyclic group which may be condensed with benzene),
   —SR″, —SOR″, or —SO₂R″ (R″ is a substituent), or an optionally substituted 3 to 7-membered cyclic group which may be condensed with benzene.
      [10] The compound of the above-mentioned [9],
      wherein

### R¹ is

(i) a 4 to 7-membered nitrogen-containing heterocyclic group optionally substituted by C₁₋₆ alkyl optionally substituted by one or more of the same or different substituents selected from
   hydroxy,
   cyano,
   optionally substituted amino,
   optionally substituted alkoxy,
   —SR″″, —SOR″″, or —SO₂R″″ (R″″ is a substituent), optionally substituted 5 to 6-membered cyclic amino,
   carboxy,
   C₁₋₆ alkoxycarbonyl, and
   optionally substituted carbamoyl, or
(ii) optionally substituted carbamoyl.
   [11] The compound of the above-mentioned [9],
   wherein

### R² is

(i) C₁₋₆ alkyl,
(ii) C₁₋₆ alkoxy optionally substituted by one or more of the same or different substituents selected from C₆₋₁₀ aryl and C₁₋₆ alkoxy,
(iii)-SOR′, or —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), or
(iv) an optionally substituted 3 to 7-membered cyclic group.
   [12] The compound of the above-mentioned [9],
   wherein

### R³ is

(i) C₁₋₆ alkyl,
(ii) C₂₋₆ alkenyl optionally substituted by 5 to 6-membered heterocyclic group,
(iii) C₂₋₆ alkoxy, or C₁₋₆ alkoxy substituted by one or more of the same or different substituents selected from
   (a) optionally substituted amino,
   (b) phenyl optionally substituted by one or more of the same or different substituents selected from
      halogen, and
      is C₁₋₆ alkylsulfonyl,
   (c) 5 to 6-membered heterocyclic group which may be substituted by one or more of the same or different substituents selected from
      C₁₋₆ alkyl,
      C₁₋₆ alkylthio,
      C₁₋₆ alkylsulfonyl,
      carboxy,
      C₁₋₆ alkoxycarbonyl and
      oxo,
      and which may be condensed with benzene,
   (d) carbamoyl optionally substituted by C₁₋₆ alkyl, and
   (e) C₁₋₆ alkylsulfonyl,
(iv) phenoxy or 5 to 6-membered heteroaryloxy, each of which may be substituted by one or more of the same or different substituents selected from
   halogen,
   C₁₋₆ alkylsulfonyl, and
   optionally substituted carbamoyl, or
(v) 5 to 6-membered heterocyclic group which may be substituted by C₁₋₆ alkyl, and which may be condensed with benzene.
   [13] The compound of the above-mentioned [9],
   wherein

### R⁴ is

(i) hydrogen, or
(ii) C₁₋₆ alkyl optionally substituted by one or more of the same or different substituents selected from C₆₋₁₀ aryl and C₁₋₆ alkoxy.
   [14] The compound of the above-mentioned [9],
   wherein

### R¹ is

(i) a 4 to 7-membered nitrogen-containing heterocyclic group optionally substituted by C₁₋₆ alkyl optionally substituted by one or more of the same or different substituents selected from
   hydroxy,
   cyano,
   optionally substituted amino,
   optionally substituted alkoxy,
   —SR″″, —SOR″″, or —SO₂R″″ (R″″ is a substituent), optionally substituted 5 to 6-membered cyclic amino,
   carboxy,
   C₁₋₆ alkoxycarbonyl, and
   optionally substituted carbamoyl, or
(ii) optionally substituted carbamoyl;

### R² is

(i) C₁₋₆ alkyl,
(ii) C₁₋₆ alkoxy optionally substituted by one or more of the same or different substituents selected from C₆₋₁₀ aryl and C₁₋₆ alkoxy,
(iii)-SR′, —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), or
(iv) an optionally substituted 3 to 7-membered cyclic group;

### R³ is

(i) C₁₋₆ alkyl,
(ii) C₂₋₆ alkenyl optionally substituted by 5 to 6-membered heterocyclic group,
(iii) C₂₋₆ alkoxy, or C₁₋₆ alkoxy substituted by one or more of the same or different substituents selected from
   (a) optionally substituted amino,
   (b) phenyl optionally substituted by one or more of the same or different substituents selected from
      halogen, and
      C₁₋₆ alkylsulfonyl,
   (c) 5 to 6-membered heterocyclic group which may be substituted by one or more of the same or different substituents selected from
      C₁₋₆ alkyl,
      C₁₋₆ alkylthio,
      C₁₋₆ alkylsulfonyl,
      carboxy,
      C₁₋₆ alkoxycarbonyl and
      oxo,
      and which may be condensed with benzene,
   (d) carbamoyl optionally substituted by C₁₋₆ alkyl, and
   (e) C₁₋₆ alkylsulfonyl,
(iv) phenoxy or 5 to 6-membered heteroaryloxy, each of which may be substituted by one or more of the same or different substituents selected from
   halogen,
   C₁₋₆ alkylsulfonyl, and
   optionally substituted carbamoyl, or
(v) 5 to 6-membered heterocyclic group which may be substituted by C₁₋₆ alkyl, and which may be condensed with benzene.

### R⁴ is

(i) hydrogen, or
(ii) C₁₋₆ alkyl optionally substituted by one or more of the same or different substituents selected from C₆₋₁₀ aryl and C₁₋₆ alkoxy.
   [15] 1-(3-(5-(3-chloropyridin-2-yl)-7-(4-s (methylsulfonyl)phenoxy)-1H-indazol-3-ylamino)-1H-pyrazol-1-yl)-3-methoxypropan-2-ol and a salt thereof.
   [16] 5-(isopropylsulfonyl)-N-(1-methyl-1H-pyrazol-3-yl)-7-(4-(methylsulfonyl)phenoxy)-1H-indazol-3-amine and a salt thereof.
   [17] 5-isopropoxy-N-(1-methyl-1H-pyrazol-3-yl)-7-(4-(methylsulfonyl)phenoxy)-1H-indazol-3-amine and a salt thereof.
   [18] 5-(3-chloropyridin-2-yl)-1-methyl-N-(1-methyl-1H-pyrazol-3-yl)-7-(4-(methylsulfonyl)phenoxy)-1H-indazol-3-amine and a salt thereof.
   [19] 3-(3-(5-(3-chloropyridin-2-yl)-7-(4-(methylsulfonyl)phenoxy)-1H-indazol-3-ylamino)-1H-pyrazol-1-yl)propane-1,2-diol and a salt thereof.
   [20] A prodrug of the compound of the above-mentioned [1].
   [21] A pharmaceutical composition which comprises the compound of the above-mentioned [1] or a prodrug thereof.
   [22] The pharmaceutical composition of the above-mentioned [21] which is an agent for activating glucokinase.
   [23] The pharmaceutical composition of the above-mentioned [21] which is an agent for preventing or treating diabetes or obesity.
   [24] A method of activating glucokinase which comprises administering to a subject a compound of the above-mentioned [1].
   [25] A method of preventing or treating diabetes or obesity which comprises administering to a subject a compound of the above-mentioned [1].
   [26] Use of a compound of the above-mentioned [1] for the manufacture of a medicament for activating glucokinase.
   [27] Use of a compound of the above-mentioned [1] for the manufacture of a medicament for preventing or treating diabetes or obesity.

### Effect of the Invention

The glucokinase activator of the present invention has a superior activity, and is useful as a pharmaceutical agent for the prophylaxis or treatment of diabetes, obesity and the like, and the like.

In the present specification, “Substituent group A” refers to a group consisting of

(1) C₃₋₁₀ cycloalkyl (e.g., cyclopropyl, cyclopentyl, cyclohexyl);
(2) C₆₋₁₄ aryl (e.g., C₆₋₁₀ aryl such as phenyl, naphthyl, etc.) optionally substituted by 1 to 3 of the same or different substituents selected from
(a) C₁₋₆ alkyl optionally substituted by 1 to 3 halogen atoms,
(b) hydroxy,
(c) C₁₋₆ alkoxy,
(d) a halogen atom, and
(e) C₁₋₆ alkylsulfonyl;
   (3) a 3 to 7-membered heterocyclic group (e.g., 5- or 6-membered heterocyclic group, 5- or 6-membered cyclic amino) which may be condensed with benzene, and which may be substituted with 1 to 3 of the same or different substituents selected from
(a) optionally substituted amino,
(b) halogen,
(c) C₁₋₆ alkyl,
(d) C₁₋₆ alkylthio,
(e) C₁₋₆ alkylsulfonyl,
(f) carboxy,
(g) C₁₋₆ alkoxy-carbonyl, and
(h) oxo;
   (4) optionally substituted amino;
   (5) halogen;
   (6) amidino;
   (7) C₁₋₆ alkyl-carbonyl (e.g., C₁₋₆ alkanoyl) optionally substituted by 1 to 3 halogen atoms;
   (8) C₁₋₆ alkoxy-carbonyl optionally substituted by 1 to 3 halogen atoms;
   (9) aromatic heterocyclyl-carbonyl (e.g., thienylcarbonyl, indolylcarbonyl) optionally substituted by 1 to 3 amino (said amino is optionally mono or di-substituted by substituents selected from C₁₋₆ alkyl and aromatic heterocyclyl-sulfonyl (e.g., thienylsulfonyl));
   (10) non-aromatic heterocyclyl-carbonyl (e.g., morpholinylcarbonyl);
   (11) C₁₋₆ alkylsulfonyl (e.g., methylsulfonyl) optionally substituted by 1 to 3 halogen atoms;
   (12) optionally substituted carbamoyl;
   (13) thiocarbamoyl optionally mono or di-substituted by C₁₋₆ alkyl optionally substituted by 1 to 3 halogen atoms;
   (14) optionally substituted sulfamoyl;
   (15) carboxy;
   (16) hydroxy;
   (17) C₁₋₆ alkoxy optionally substituted by 1 to 3 of the same or different substituents selected from
(a) a halogen atom,
(b) carboxy,
(c) C₁₋₆ alkoxy, and
(d) C₁₋₆ alkoxy-carbonyl;
   (18) C₂₋₆ alkenyloxy (e.g., ethenyloxy) optionally substituted by 1 to 3 halogen atoms;
   (19) C₃₋₁₀ cycloalkyloxy (e.g., cyclohexyloxy);
   (20) C₇₋₁₃ aralkyloxy (e.g., benzyloxy) optionally substituted by 1 to 3 halogen atoms;
   (21) C₆₋₁₄ aryloxy (e.g., phenyloxy, naphthyloxy);
   (22) C₁₋₆ alkyl-carbonyloxy (e.g., acetyloxy, tert-butylcarbonyloxy);
   (23) mercapto;
   (24) C₁₋₆ alkylthio optionally substituted by 1 to 3 of the same or different substituents selected from a halogen atom and C₆₋₁₄ aryl;
   (25) C₆₋₁₄ arylthio (e.g., phenylthio, naphthylthio);
   (26) aromatic heterocyclethio (e.g., tetrazolylthio) optionally-substituted by 1 to 3 C₁₋₆ alkyl;
   (27) sulfo;
   (28) cyano;
   (29) azide;
   (30) nitro;
   (31) nitroso;
   (32) formyl;
   (33) C₁₋₆ alkylsulfinyl (e.g., methylsulfinyl);
   (34) C₃₋₁₀ cycloalkyl-C₁₋₆ alkyloxy (e.g., cyclopropylmethyloxy);
   (35) C₁₋₃ alkylenedioxy; and
   (36) aromatic heterocyclyl-carbonylthio (e.g., indolylcarbonylthio) group optionally substituted by 1 to 3 amino (said amino is optionally mono or di-substituted by substituents selected from C₁₋₆ alkyl and aromatic heterocyclyl-sulfonyl (e.g., thienylsulfonyl)).

In the present specification, “Substituent group B” refers to a group consisting of

(1) C₁₋₆ alkyl optionally substituted by 1 to 3 of the same or different substituents selected from
(i) a halogen atom,
(ii) carboxy,
(iii) hydroxy,
(iv) C₁₋₆ alkoxy optionally substituted by 1 to 3 of the same or different substituents selected from carboxy and C₁₋₆ alkoxy-carbonyl,
(v) C₁₋₆ alkoxy-carbonyl,
(vi) C₁₋₆ alkyl-carbonyloxy (e.g., acetyloxy, tert-butylcarbonyloxy),
(vii) carbamoyl optionally mono or di-substituted by substituents selected from C₁₋₆ alkylsulfonyl and amino,
(viii) an aromatic heterocyclic group (e.g., thienyl, tetrazolyl),
(ix) a nonaromatic heterocyclic group (e.g., piperidino, piperazinyl, morpholinyl, dihydrooxadiazolyl, hexahydropyrazinooxazinyl (e.g., hexahydropyrazino[2,1-c][1,4]oxazinyl)) optionally substituted by 1 to 3 of the same or different substituents selected from C₁₋₆ alkyl-carbonyl and oxo,
(x) amino optionally mono or di-substituted by C₁₋₆ alkyl (said C₁₋₆alkyl is optionally substituted by 1 to 3 of the same or different substituents selected from a nonaromatic heterocyclic group (e.g., morpholinyl), C₁₋₆ alkoxy and C₁₋₆ alkylsulfonyl),
(xi) C₁₋₆ alkylsulfonyl optionally substituted by 1 to 3 carboxy,
(xii) C₁₋₆ alkylthio optionally substituted by 1 to 3 of the same or different substituents selected from carboxy, alkoxy-carbonyl, hydroxy and carbamoyl,
(xiii) phosphono optionally mono or di-substituted phosphono by C₁₋₆ alkyl,
(xiv) non-aromatic heterocyclyl-carbonyl (e.g., morpholinylcarbonyl),
(xv) cyano, and
(xvi) C₆₋₁₄ aryloxy optionally substituted by 1 to 3 of the same or different substituents selected from carboxy and C₁₋₆ alkoxy-carbonyl;
   (2) C₂₋₆ alkenyl (e.g., ethenyl, 1-propenyl) optionally substituted by 1 to 3 of the same or different substituents selected from a halogen atom, carboxy, C₁₋₆ alkoxy-carbonyl and carbamoyl;
   (3) C₇₋₁₃ aralkyl (e.g., benzyl) optionally substituted by 1 to 3 of the same or different substituents selected from C₁₋₆ alkyl optionally substituted by 1 to 3 halogen atoms, hydroxy, C₁₋₆ alkoxy and a halogen atom; and (4) oxo.

In the present specification, examples of the “optionally substituted amino”, “optionally substituted carbamoyl” and “optionally substituted sulfamoyl” may include amino, carbamoyl and sulfamoyl, each of which is optionally mono- or di-substituted by

(i) C₁₋₆ alkyl (e.g., methyl, ethyl, carboxymethyl) optionally substituted by 1 to 3 of the same or different substituents selected from halogen and carboxy,
(ii) C₁₋₆ alkoxy (e.g., methoxy),
(iii) C₁₋₆ alkoxy-C₁₋₆ alkyl (e.g., 2-methoxyethyl),
(iv) C₇₋₁₃ aralkyl (e.g., benzyl),
(v) C₆₋₁₄ aryl (e.g., phenyl),
(vi) aromatic heterocyclyl-C₁₋₆ alkyl (e.g., pyridylmethyl),
(vii) C₁₋₆ alkyl-carbonyl,
(viii) C₁₋₆ alkoxy-carbonyl,
(ix) C₆₋₁₄ aryl-carbonyl (e.g., benzoyl),
(x) C₇₋₁₃ aralkyl-carbonyl (e.g., benzylcarbonyl, phenethylcarbonyl),
(xi) carbamoyl optionally mono or di-substituted by substituents selected from C₁₋₆ alkyl, C₆₋₁₄ aryl and C₇₋₁₃ aralkyl (e.g., carbamoyl, methylcarbamoyl, benzylcarbamoyl, dimethylcarbamoyl),
(xii) C₁₋₆ alkylsulfonyl,
(xiii) C₆₋₁₄ arylsulfonyl (e.g., benzenesulfonyl, toluenesulfonyl, 1-naphthalenesulfonyl, 2-naphthalenesulfonyl), and
(xiv) C₇₋₁₃ aralkylsulfonyl (e.g., benzylsulfonyl).

In the present specification, the “C₁₋₆ alkyl” may include, for example, methyl, ethyl, propyl, isopropyl, butyl, isobutyl, tert-butyl, pentyl and hexyl.

In the present specification, the “C₂₋₆ alkenyl” may include, for example, vinyl, allyl, isopropenyl, buten-1-yl, buten-2-yl, buten-3-yl, 2-methylpropen-2-yl, 1-methylpropen-2-yl and 2-methylpropen-1-yl.

In the present specification, the “3 to 7-membered cyclic group” may be an aromatic group or a nonaromatic cyclic group.

Such “aromatic group” may include, for example, a phenyl and an aromatic heterocyclic group.

In the present specification, the “aromatic heterocyclic group” may include, for example, a 4 to 7-membered (preferably 5- or 6-membered) monocyclic aromatic heterocyclic group containing 1 to 4 heteroatoms selected from oxygen atom, sulfur atom and nitrogen atom besides carbon atoms as ring-constituting atoms.

In the present specification, the “nonaromatic cyclic group” may include, for example, a nonaromatic cyclic hydrocarbon group and a nonaromatic heterocyclic group.

In the present specification, the “nonaromatic cyclic hydrocarbon group” may include, for example, C₃₋₁₀ cycloalkyl, C₃₋₁₀ cycloalkenyl and C₄₋₁₀ cycloalkadienyl.

In the present specification, the “nonaromatic heterocyclic group” may include, for example, a 4 to 7-membered (preferably 5- or 6-membered) monocyclic nonaromatic heterocyclic group containing 1 to 4 heteroatoms selected from oxygen atom, sulfur atom and nitrogen atom besides carbon atoms as ring-constituting atoms.

In the present specification, the “4 to 7-membered monocyclic aromatic heterocyclic group” may include, for example, furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 3-isothiazolyl, 4-isothiazolyl, 5-isothiazolyl), oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,2,4-thiadiazol-5-yl, 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,3,5-triazin-2-yl, 1,3,5-triazin-4-yl, 1,2,3-triazin-4-yl, 1,2,4-triazin-3-yl).

In the present specification, the “4 to 7-membered monocyclic nonaromatic heterocyclic group” may include, for example, azetidinyl (e.g., 1-azetidinyl, 2-azetidinyl, 3-azetidinyl), pyrrolidinyl (e.g., 1-pyrrolidinyl, 2-pyrrolidinyl), piperidinyl (e.g., piperidino, 2-piperidinyl, 3-piperidinyl, 4-piperidinyl), morpholinyl (e.g., morpholino), thiomorpholinyl (e.g., thiomorpholino), piperazinyl (e.g., 1-piperazinyl, 2-piperazinyl, 3-piperazinyl), hexamethyleniminyl (e.g., hexamethyleneimin-1-yl), oxazolidinyl (e.g., oxazolidin-2-yl), thiazolidinyl (e.g., thiazolidin-2-yl), imidazolidinyl (e.g., imidazolidin-2-yl, imidazolidin-3-yl), oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), 2-thioxo-1,3-oxazolidin-5-yl, pyranyl (e.g., 4-pyranyl), tetrahydropyranyl (e.g., 2-tetrahydropyranyl, 3-tetrahydropyranyl, 4-tetrahydropyranyl), thiopyranyl (e.g., 4-thiopyranyl), tetrahydrothiopyranyl (e.g., 2-tetrahydrothiopyranyl, 3-tetrahydrothiopyranyl, 4-tetrahydrothiopyranyl), 1-oxide tetrahydrothiopyranyl (e.g., 1-s oxide tetrahydrothiopyran-4-yl), 1,1-dioxide tetrahydrothiopyranyl (e.g., 1,1-dioxide tetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl (e.g., tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl).

In the present specification, the “5- or 6-membered is heterocyclic group” may include, for example, 5- or 6-membered cyclic groups (e.g., thienyl, pyridyl, thiazolyl, imidazolyl, pyrazolyl, pyrrolidinyl) of the aforementioned “4 to 7-membered monocyclic aromatic heterocyclic group” and “4 to 7-membered monocyclic nonaromatic heterocyclic group”.

In the present specification, the “5- or 6-membered cyclic amino” may include, for example, 5- or 6-membered ones that attach via a ring nitrogen (e.g., 1-azetidinyl, 1-pyrrolidinyl, piperidino, morpholino, thiomorpholino, 1-piperazinyl) of the aforementioned “4 to 7-membered monocyclic nonaromatic heterocyclic group”.

In the present specification, the “5- or 6-membered aromatic heterocyclic group (5- or 6-membered heteroaryl)” may include, for example, 5- or 6-membered cyclic groups among the aforementioned “4 to 7-membered monocyclic aromatic heterocyclic group”.

In the present specification, the “C₃₋₆ cycloalkyl” may include, for example, cyclopropyl, cyclobutyl, cyclopentyl and cyclohexyl.

In the present specification, the “C₃₋₁₀ cycloalkenyl” may include, for example, cyclopropenyl, cyclobutenyl, cyclopentenyl, cyclohexenyl, cycloheptenyl, cyclooctenyl, cyclononenyl and cyclodecenyl.

In the present specification, the “C₄₋₁₀ cycloalkadienyl” may include, for example, cyclobutadienyl, cyclopentadienyl, cyclohexadienyl, cycloheptadienyl, cyclooctadienyl, cyclononadienyl and cyclodecadienyl.

In the present specification, the “C₁₋₆ alkoxy” may include, for example, methoxy, ethoxy, propoxy, isopropoxy and tert-butoxycarbonyl.

In the present specification, the “halogen (atom)” may include, for example, fluorine, chlorine, bromine and iodine.

In the present specification, the “C₁₋₆ alkoxy-carbonyl” may include, for example, methoxycarbonyl, ethoxycarbonyl, propoxycarbonyl and tert-butoxycarbonyl.

In the present specification, the “C₆₋₁₄ aryl” may include, for example, “C₆₋₁₀ aryl”, and the “C₆₋₁₀ aryl” may include phenyl, 1-naphthyl and 2-naphthyl.

In the present specification, the “5- or 6-membered heteroaryloxy” means 5- or 6-membered heteroaryl-O—. The “5- or 6-membered heteroaryl” may include, for example, the aforementioned ones.

In the present specification, the “C₁₋₆ alkylsulfonyl” may include, for example, methylsulfonyl, ethylsulfonyl and the like.

In the present specification, the “C₁₋₆ alkylthio” may include, for example, methylthio, ethylthio, propylthio, isopropylthio, butylthio, sec-butylthio, tert-butylthio and the like.

In the present specification, the “C₇₋₁₀ aralkyl” may include, for example, benzyl and phenethyl.

In the present specification, the “C₁₋₆ alkanoyl” may include, for example, acetyl, propionyl and pivaloyl.

In the present specification, the aromatic heterocycle in the “aromatic heterocyclyl-C₁₋₆ alkyl” may include, for example, 4 to 7-membered (preferably 5- or 6-membered) monocyclic aromatic heterocycle containing 1 to 4 heteroatoms selected from oxygen atom, sulfur atom and nitrogen atom besides carbon atoms as ring-constituting atoms (e.g., pyridine).

Hereinafter, the definitions of symbols in the formula (I) are explained in detail.

R¹ is an optionally substituted 4 to 7-membered nitrogen-containing heterocyclic group, optionally substituted carbamoyl, or optionally substituted sulfamoyl. R¹ is preferably an optionally substituted 4 to 7-membered nitrogen-containing heterocyclic group, or optionally substituted sulfamoyl.

R¹ is preferably
(i) a 4 to 7-membered nitrogen-containing heterocyclic group optionally substituted by C₁₋₆ alkyl optionally substituted by one or more of the same or different substituents selected from
   hydroxy,
   cyano,
   optionally substituted amino,
   optionally substituted 5 to 6-membered cyclic amino,
   carboxy,
   C₁₋₆ alkoxycarbonyl, and
   optionally substituted carbamoyl, or
(ii) optionally substituted carbamoyl.
   As R¹,
(i) a 4 to 7-membered nitrogen-containing heterocyclic group optionally substituted by C₁₋₆ alkyl optionally substituted by one or more of the same or different substituents selected from
   hydroxy,
   cyano,
   optionally substituted amino,
   optionally substituted alkoxy,
   —SR″″, —SOR″″, or —SO₂R″″ (R″″ is a substituent), optionally substituted 5 to 6-membered cyclic amino,
   carboxy,
   C₁₋₆ alkoxycarbonyl, and
   optionally substituted carbamoyl, and
(ii) optionally substituted carbamoyl
   are also preferable.

The “4 to 7-membered nitrogen-containing heterocyclic group” in the “optionally substituted 4 to 7-membered nitrogen-containing heterocyclic group” represented by R¹ may include, for example, a 4 to 7-membered (preferably 5- or 6-membered) aromatic or nonaromatic nitrogen-containing heterocyclic group containing at least one nitrogen atom and optionally containing 1 or 2 heteroatoms selected from oxygen atom, sulfur atom and nitrogen atom besides carbon atoms as ring-constituting atoms.

Preferable examples of such nitrogen-containing heterocyclic group may include thiazolyl (e.g., 2-thiazolyl, 4-thiazolyl, 5-thiazolyl), pyridyl (e.g., 2-pyridyl, 3-pyridyl, 4-pyridyl), pyrazolyl (e.g., 1-pyrazolyl, 3-pyrazolyl, 4-pyrazolyl), pyridazinyl (e.g., 3-pyridazinyl, 4-pyridazinyl), and pyrazinyl.

The “4 to 7-membered nitrogen-containing heterocyclic group” optionally has one or more (preferably 1 to 3) of the same or different substituents at the substitutable positions.

Preferable examples of such substituent may include optionally substituted C₁₋₆ alkyl.

The “C₁₋₆ alkyl” in the “optionally substituted C₁₋₆ alkyl” optionally has one or more (preferably 1 to 3) of the same or different substituents. Examples of such substituents are hydroxy, cyano, optionally substituted amino, optionally substituted alkoxy, optionally substituted 5- or 6-membered cyclic amino, optionally substituted 5- or 6-membered aromatic heterocyclic group, carboxy, C₁₋₆ alkoxy-carbonyl, and optionally substituted carbamoyl, —SR′″, —SOR′″, and —SO₂R′″ (R′″ is a substituent), —SR″″, —SOR″″, and —SO₂R″″ (R″″ is a substituent) and the like.

The “5- or 6-membered cyclic amino” as the substituent for the “C₁₋₆ alkyl” of the “optionally substituted C₁₋₆ alkyl” optionally has one or more (preferably 1 to 3) of the same or different substituents. Examples of such substituents may include the substituents selected from Substituent group A and Substituent group B. Among these, oxo, hydroxy, C₁₋₆ alkanoyl, 5- or 6-membered cyclic amino (e.g., piperazinyl, piperidino, morpholino, thiomorpholino), and C₁₋₆ carbamoyl optionally mono- or di-substituted by C₁₋₆ alkyl (e.g., dimethylcarbamoyl) and the like are preferable.

When the “5- or 6-membered cyclic amino” as the substituent for the “C₁₋₆ alkyl” of the “optionally substituted alkyl” has two or more substituents, two of the substituents may be together to forma 5- or 6-membered ring optionally having oxo (e.g., morpholine, morpholin-3-one, thiomorpholine, 1,3-dioxolane). The “5- or 6-membered ring” is may form a fused ring together with the ring of the 5- or 6-membered cyclic amino; or may form a spiro ring. Examples of such “optionally substituted 5- or 6-membered cyclic amino” may include (thiomorpholine 1,1-dioxide)-4-yl, 1,4-dioxa-8-azaspiro[4.5]deca-8-yl, 4-oxohexahydropyrazino[2,1-c][1,4]oxazin-8-yl, 3-oxohexahydro[1,3]oxazolo[3,4-a]pyrazine-7-yl and the like.

The “optionally substituted alkoxy” as the substituent for the “C₁₋₆ alkyl” of the “optionally substituted C₁₋₆ alkyl” is preferably C₁₋₆ alkoxy which optionally has one or more (preferably 1 to 3) of the same or different substituents at the substitutable positions. Examples of such substituents may include substituents selected from Substituent group A and Substituent group B.

The “5- or 6-membered aromatic heterocyclic group” as the substituent for the “C₁₋₆ alkyl” of the “optionally substituted C₁₋₆ alkyl” optionally has one or more (preferably 1 to 3) of the same or different substituents at the substitutable positions. Examples of such substituents may include substituents selected from Substituent group A and Substituent group B.

Examples of the “optionally substituted carbamoyl” and “optionally substituted sulfamoyl” represented by R¹ may each include those exemplified above. Among these, carbamoyl and sulfamoyl each optionally mono or di-substituted by the substituents selected from C₁₋₆ alkyl optionally substituted carboxy (e.g., methyl, ethyl, propyl, isopropyl, carboxymethyl), and C₆₋₁₄ aryl (e.g., phenyl) and the like are preferable.

Examples of the “substituent” represented by R′″ in —SR′″, —SOR′″, and —SO₂R′″ may include substituents selected from Substituent group A and Substituent group B.

Examples of the “substituent” represented by R″″ in —SR″″, —SOR″″, and —SO₂R″″ may include substituents selected from Substituent group A and Substituent group B.

R² is optionally substituted alkyl, optionally substituted alkoxy, an optionally substituted 3 to 7-membered cyclic group, —SR′, —SOR′, or —SO₂R′ (R′ is a substituent). Among these, R² is preferably an optionally substituted 3 to 7-membered cyclic group, —SR′, —SOR′, and —SO₂R′ (R′ is a substituent) and the like.

As R²,
(i) C₁₋₆ alkyl,
(ii) C₁₋₆ alkoxy optionally substituted by one or more of the same or different substituents selected from C₆₋₁₀ aryl and C₁₋₆ alkoxy,
(iii)-SR′, —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), and
(iv) an optionally substituted 3 to 7-membered cyclic group, and the like are also preferable.

The “alkyl” of the “optionally substituted alkyl” represented by R² may include, for example, C₁₋₆ alkyl.

The “alkyl” of the “optionally substituted alkyl” optionally has one or more (preferably 1 to 3) of the same or different substituents. Examples of such substituents may include the substituents selected from Substituent group A.

The “optionally substituted alkyl” represented by R² is preferably C₁₋₆ alkyl and the like.

The “alkoxy” of the “optionally substituted alkoxy” represented by R² is preferably C₁₋₆ alkoxy.

The “C₁₋₆ alkoxy” of the “optionally substituted alkoxy” optionally has one or more (preferably 1 to 3) of the same or different substituents. Examples of such substituents may include the substituents selected from Substituent group A.

The “optionally substituted alkoxy” represented by R² is preferably C₁₋₆ alkoxy optionally substituted by one or more of the same or different substituents selected from C₆₋₁₀ aryl and C₁₋₆ alkoxy and the like.

Examples of the “substituent” represented by R′ in —SR′, —SOR′, and —SO₂R′ may include the substituents selected from Substituent group A and Substituent group B. As such substituents, optionally substituted amino (e.g., amino-optionally monosubstituted with aromatic heterocyclyl-C₁₋₆ alkyl), an optionally substituted 3 to 7-membered cyclic group (e.g., nonaromatic heterocyclic group such as pyrrolidinyl, etc., aromatic heterocyclic group such as imidazolyl optionally substituted by 1 to 3 C₁₋₆ alkyl, etc.), C₁₋₆ alkyl, C₃₋₁₀ cycloalkyl and the like are preferable.

The “3 to 7-membered cyclic group” of the “optionally substituted 3 to 7-membered cyclic group” represented by R² may include those as exemplified above. The “3 to 7-membered cyclic group” optionally has one or more (preferably 1 to 3) of the same or different substituents at the substitutable positions. Examples of such substituents may include the substituents selected from Substituent group A and Substituent group B.

As the “optionally substituted 3 to 7-membered cyclic group” represented by R², phenyl and an aromatic heterocyclic group (e.g., pyridyl, pyrrolyl, imidazolyl, thienyl, thiazolyl, pyrazolyl) each optionally substituted by 1 to 3 of the same or different substituents selected from cyano, amino, halogen, C₁₋₆ alkyl, carboxy, an optionally substituted 3 to 7-membered cyclic group (e.g., a 4 to 7-membered monocyclic aromatic heterocyclic group optionally substituted by 1 to 3 C₁₋₆ alkyl, etc.), and C₁₋₆ alkoxy-carbonyl, etc., are preferable.

R³ is
hydrogen,
halogen,
optionally substituted alkyl,
optionally substituted alkenyl,
optionally substituted alkoxy,
—O-Cy (Cy is an optionally substituted 3 to 7-membered cyclic group which may be condensed with benzene),
—SR″, —SOR″, or —SO₂R″ (R″ is a substituent), or an optionally substituted 3 to 7-membered cyclic group which may be condensed with benzene.

R³ is preferably
(i) hydrogen,
(ii) halogen,
(iii) C₁₋₆ alkyl,
(iv) C₂₋₆ alkenyl optionally substituted by a 5 to 6-membered heterocyclic group,
(v) C₁₋₆ alkoxy optionally substituted by one or more of the same or different substituents selected from
   (a) optionally substituted amino,
   (b) phenyl optionally substituted by one or more of the same or different substituents selected from
      halogen, and
      C₁₋₆ alkylsulfonyl,
   (c) 5 to 6-membered heterocyclic ring which may be substituted by one or more of the same or different substituents selected from
      C₁₋₆ alkyl,
      C₁₋₆ alkylthio,
      C₁₋₆ alkylsulfonyl,
      carboxy,
      C₁₋₆ alkoxycarbonyl and
      oxo,
      and which may be condensed with benzene,
   (d) carbamoyl optionally substituted by C₁₋₆ alkyl, and
   (e) C₁₋₆ alkylsulfonyl,
(vi) phenoxy or 5 to 6-membered heteroaryloxy, each of which may be substituted by one or more of the same or different substituents selected from
   halogen,
   C₁₋₆ alkylsulfonyl, and
   optionally substituted carbamoyl, or
(vii) 5 to 6-membered heterocyclic ring which may be substituted by C₁₋₆ alkyl, and which may be condensed with benzene and the like.

Alternatively, in another embodiment, R³ is
optionally substituted-alkyl,
optionally substituted alkenyl,
C₂₋₆ alkoxy, or substituted C₁₋₆ alkoxy,
—O-Cy (Cy is an optionally substituted 3 to 7-membered cyclic group which may be condensed with benzene),
—SR″, —SOR″, or —SO₂R″ (R″ is a substituent), or
an optionally substituted 3 to 7-membered cyclic group which may be condensed with benzene.

In this case, R³ is preferably
(i) C₁₋₆ alkyl,
(ii) C₂₋₆ alkenyl optionally substituted by a 5 to 6-membered heterocyclic group,
(iii) C₂₋₆ alkoxy, or C₁₋₆ alkoxy substituted by one or more of the same or different substituents selected from
   (a) optionally substituted amino,
   (b) phenyl optionally substituted by one or more of the same or different substituents selected from
      halogen, and
      C₁₋₆ alkylsulfonyl,
   (c) a 5 to 6-membered heterocyclic group which may be substituted by one or more of the same or different substituents selected from
      C₁₋₆ alkyl,
      C₁₋₆ alkylthio,
      C₁₋₆ alkylsulfonyl,
      carboxy,
      C₁₋₆ alkoxycarbonyl and
      oxo,
   and which may be condensed with benzene,
   (d) carbamoyl optionally substituted by C₁₋₆ alkyl, and
   (e) C₁₋₆ alkylsulfonyl,
(iv) phenoxy or 5 to 6-membered heteroaryloxy, each of which may be substituted by one or more of the same or different substituents selected from
   halogen,
   C₁₋₆ alkylsulfonyl, and
   optionally substituted carbamoyl, or
(v) a 5 to 6-membered heterocyclic group which may be substituted by C₁₋₆ alkyl, and which may be condensed with benzene and the like.

The “alkyl” of the “optionally substituted alkyl” represented by R³ may include, for example, C₁₋₆ alkyl.

The “alkyl” in the “optionally substituted alkyl” optionally has one or more (preferably 1 to 3) of the same or different substituents. Examples of such substituents may include the substituents selected from Substituent group A.

The “optionally substituted alkyl” represented by R³ is preferably C₁₋₆ alkyl and the like.

The “alkenyl” of the “optionally substituted alkenyl” represented by R³ may include, for example, C₂₋₆ alkenyl.

The “alkenyl” in the “optionally substituted alkenyl” optionally has one or more (preferably 1 to 3) of the same or different substituents. Examples of such substituents may include the substituents selected from Substituent group A.

The “optionally substituted alkenyl” represented by R³ is preferably C₂₋₆ alkenyl optionally substituted by a 5- or 6-membered heterocyclic group (e.g., pyridyl) and the like.

The “alkoxy” of the “optionally substituted alkoxy” represented by R³ may include, for example, “C₁₋₆ alkoxy”.

The “alkoxy” of the “optionally substituted alkoxy” optionally has one or more (preferably 1 to 3) of the same or different substituents. Examples of such substituents may include the substituents selected from Substituent group A.

The “optionally substituted alkoxy” represented by R³ is preferably C₁₋₆ alkoxy optionally substituted by one or more (preferably 1 to 3) substituents selected from
(a) optionally substituted amino,
(b) C₆₋₁₀ aryl (e.g., phenyl) optionally substituted by one or more of the same or different substituents selected from is halogen, and C₁₋₆ alkylsulfonyl,
(c) a 5 to 6-membered heterocyclic group which may be substituted by one or more of the same or different substituents selected from
C₁₋₆ alkyl,
C₁₋₆ alkylthio,
C₁₋₆ alkylsulfonyl,
carboxy,
C₁₋₆ alkoxy-carbonyl and
oxo,
   and which may be condensed with benzene (e.g., pyridyl, pyrazolyl, imidazolyl, thiazolyl, thienyl, phthalimidyl),
(d) carbamoyl optionally substituted by C₁₋₆ alkyl, and
(e) C₁₋₆ alkylsulfonyl, or
   C₂₋₆ alkoxy and the like.

The “3 to 7-membered cyclic group” of the “optionally substituted 3 to 7-membered cyclic group which may be condensed with benzene” represented by Cy in —O-Cy may include those as exemplified above. The “3 to 7-membered cyclic group” optionally has one or more (preferably 1 to 3) of the same or different substituents at the substitutable positions. Examples of such substituents may include the substituents selected from Substituent group A and Substituent group B.

The —O-Cy is preferably phenoxy or 5- or 6-membered heteroaryloxy each optionally has one or more of the same or different substituents selected from

halogen,

alkylsulfonyl, and

optionally substituted carbamoyl and the like.

The “3 to 7-membered cyclic group” of the “optionally substituted 3 to 7-membered cyclic group which may be condensed with benzene” represented by R³ may include those as exemplified above. The “3 to 7-membered cyclic group” optionally has one or more (preferably 1 to 3) of the same or different substituents at the substitutable positions. Examples of such substituents may include the substituents selected from Substituent group A and Substituent group B.

The “optionally substituted 3 to 7-membered cyclic group which may be condensed with benzene” represented by R³ is preferably a 5- or 6-membered heterocyclic group which may be substituted by C₁₋₆ alkyl, and which may be condensed with benzene.

Examples of the substituent represented by R″ in —SR″, —SOR″, and —SO₂R″ may include the substituents selected from Substituent group A and Substituent group B.

R⁴ is hydrogen or optionally substituted alkyl.

R⁴ is preferably

(i) hydrogen, and
(ii) C₁₋₆ alkyl optionally substituted by one or more of the same or different substituents selected from C₆₋₁₀ aryl and C₁₋₆ alkoxy and the like.

The “alkyl” of the optionally substituted alkyl” represented by R⁴ may include, for example, C₁₋₆ alkyl.

The “alkyl” in the “optionally substituted alkyl” optionally has one or more (preferably 1 to 3) of the same or different substituents. Examples of such substituents may include the substituents selected from Substituent group A.

The “optionally substituted alkyl” represented by R⁴ is preferably C₁₋₆ alkyl optionally substituted by one or more of the same or different substituents selected from C₆₋₁₀ aryl and C₁₋₆ alkoxy and the like.

Preferable compounds of the formula (I) are

[A] a compound wherein

### R¹ is

(i) a 4 to 7-membered nitrogen-containing heterocyclic group optionally substituted by C₁₋₆ alkyl optionally substituted by one or more of the same or different substituents selected from
   hydroxy,
   cyano,
   optionally substituted amino,
   optionally substituted 5 to 6-membered cyclic amino,
   carboxy,
   C₁₋₆ alkoxycarbonyl, and
   optionally substituted carbamoyl, or
(ii) optionally substituted carbamoyl;

### R² is

(i) C₁₋₆ alkyl,
(ii) C₁₋₆ alkoxy optionally substituted by one or more of the same or different substituents selected from C₆₋₁₀ aryl and C₁₋₆ alkoxy,
(iii) —SR′, —SOR′, or —SO₂R′ (R′ is C₁₋₆ alkyl, C₃₋₇ cycloalkyl, or a 4 to 7-membered nitrogen-containing heterocyclic group optionally substituted by C₁₋₆ alkyl), or
(iv) an optionally substituted 3 to 7-membered cyclic group;

### R³ is

(i) hydrogen,
(ii) halogen,
(iii) C₁₋₆ alkyl,
(iv) C₁₋₆ alkenyl optionally substituted by a 5 to 6-membered heterocyclic group,
(v) C₁₋₆ alkoxy optionally substituted by one or more of the same or different substituents selected from
   (a) optionally substituted amino,
   (b) phenyl optionally substituted by one or more of the same or different substituents selected from
      halogen, and
      C₁₋₆ alkylsulfonyl,
   (c) 5 to 6-membered heterocyclic ring which may be substituted by one or more of the same or different substituents selected from
      C₁₋₆ alkyl,
      C₁₋₆ alkylthio,
      C₁₋₆ alkylsulfonyl,
      carboxy,
      C₁₋₆ alkoxycarbonyl and
      oxo,
   and which may be condensed with benzene,
   (d) carbamoyl optionally substituted by C₁₋₆ alkyl, and
   (e) C₁₋₆ alkylsulfonyl,
(vi) phenoxy or 5 to 6-membered heteroaryloxy optionally substituted by one or more of the same or different substituents selected from
   halogen,
   C₁₋₆ alkylsulfonyl, and
   optionally substituted carbamoyl, or
(vii) 5 to 6-membered heterocyclic ring which may be substituted by C₁₋₆ alkyl, and which may be condensed with benzene;

### R⁴ is

(i) hydrogen, or

(ii) C₁₋₆ alkyl optionally substituted by one or more of the same or different substituents selected from C₆₋₁₀ aryl and CO₁₋₆ alkoxy, and

[B] a compound wherein

### R¹ is

(i) a 4 to 7-membered nitrogen-containing heterocyclic group optionally substituted by C₁₋₆ alkyl optionally substituted by one or more of the same or different substituents selected from
hydroxy,
cyano,
optionally substituted amino,
optionally substituted alkoxy,
—SR″″, —SOR″″, or —SO₂R″″ (R″″ is a substituent), optionally substituted 5 to 6-membered cyclic amino,
carboxy,
C₁₋₆ alkoxycarbonyl, and
optionally substituted carbamoyl, and
(ii) optionally substituted carbamoyl;

### R² is

(i) C₁₋₆ alkyl,
(ii) C₁₋₆ alkoxy optionally substituted by one or more of the same or different substituents selected from C₆₋₁₀ aryl and C₁₋₆ alkoxy,
(iii)-SR′, —SOR′, or —SO₂R′ (R′ is C₁₋₆ alkyl, C₃₋₇ cycloalkyl, or a 4 to 7-membered nitrogen-containing heterocyclic group optionally substituted by C₁₋₆ alkyl), or
(iv) an optionally substituted 3 to 7-membered cyclic group;

### R³ is

(i) C₁₋₆ alkyl,
(ii) C₂₋₆ alkenyl optionally substituted by 5 to 6-membered heterocyclic group,
(iii) C₂₋₆ alkoxy, or C₁₋₆ alkoxy substituted by one or more of the same or different substituents selected from
   (a) optionally substituted amino,
   (b) phenyl optionally substituted by one or more of the same or different substituents selected from
      halogen, and
      C₁₋₆ alkylsulfonyl,
   (c) a 5 to 6-membered heterocyclic group which may be substituted by one or more of the same or different substituents selected from
      C₁₋₆ alkyl,
      C₁₋₆ alkylthio,
      C₁₋₆ alkylsulfonyl,
      carboxy,
      C₁₋₆ alkoxycarbonyl and
      oxo,
   and which may be condensed with benzene,
   (d) carbamoyl optionally substituted by C₁₋₆ alkyl, and
   (e) C₁₋₆ alkylsulfonyl,
(iv) phenoxy or 5 to 6-membered heteroaryloxy, each of which may be substituted by one or more of the same or different substituents selected from
   halogen,
   C₁₋₆ alkylsulfonyl, and
   optionally substituted carbamoyl, and
(v) a 5 to 6-membered heterocyclic group which may be substituted by C₁₋₆ alkyl, and which may be condensed with benzene.

### R⁴ is

(i) hydrogen, or
(ii) C₁₋₆ alkyl optionally substituted by one or more of the same or different substituents selected from C₆₋₁₀ aryl and C₁₋₆ alkoxy.

As salts of compound (I) (hereinafter to be collectively abbreviated as the compound of the present invention), a pharmacologically acceptable salt is preferable. As such salts, for example, a salt with inorganic base, a salt with organic base, a salt with inorganic acid, a salt with organic acid, a salt with basic or acidic amino acid and the like can be mentioned.

Preferable examples of salts with inorganic base include alkali metal salts such as sodium salt, potassium salt and the like; alkaline earth metal salts such as calcium salt, magnesium salt and the like; and aluminum salts; ammonium salts and the like.

As preferable examples of the salts with organic bases, salts with trimethylamine, triethylamine, pyridine, picoline, ethanolamine, diethanolamine, triethanolamine, dicyclohexylamine, N,N-dibenzylethylenediamine and the like can be mentioned.

As preferable examples of the salts with inorganic acids, salts with hydrochloric acid, hydrobromic acid, nitric acid, sulfuric acid, phosphoric acid and the like can be mentioned.

As preferable examples of the salts with organic acids, salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like can be mentioned.

As preferable examples of the salts with basic amino acid, salts with arginine, lysine, ornithine and the like can be mentioned.

As preferable examples of the salts with acidic amino acids, salts with aspartic acid, glutamic acid and the like can be mentioned.

A prodrug of the compound of the present invention means a compound which is converted to the present invention with a reaction due to an enzyme, an gastric acid and the like under the physiological condition in the living body, that is, a compound which is converted to the compound of the present invention with oxidation, reduction, hydrolysis and the like according to an enzyme; a compound which is converted to the compound of the present invention by hydrolysis etc. due to gastric acid and the like. A prodrug of the compound of the present invention may be a compound obtained by subjecting an amino group in the compound of the present invention to an acylation, alkylation or phosphorylation (e.g., a compound obtained by subjecting an amino group in the compound of the present invention to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation or tert-butylation); a compound obtained by subjecting a hydroxy group in the compound of the present invention to an acylation, alkylation, phosphorylation or boration (e.g., a compound obtained by subjecting an hydroxy group in the compound of the present invention to an acetylation, palmitoylation, propanoylation, pivaloylation, succinylation, fumarylation, alanylation or dimethylaminomethylcarbonylation); a compound obtained by subjecting a carboxyl group in the compound of the present invention to an esterification or amidation (e.g., a compound obtained by subjecting a carboxyl group in the compound of the present invention to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification or methylamidation) and the like. Any of these compounds can be produced from the compound of the present invention by a method known per se.

A prodrug of the compound of the present invention may also be one which is converted into the present invention under a physiological condition, such as those described in IYAKUHIN NO KAIHATSU (Development of Pharmaceuticals), Vol. 7, Design of Molecules, p. 163-198, Published by HIROKAWA SHOTEN (1990).

The compound of the present invention may be labeled with an isotope (e.g., ³H, ¹⁴C, ³⁵S, ¹²⁵I) and the like.

Furthermore, the compound represented by the formula (I) and a salt thereof generates tautomers, and all tautomers are encompassed in the present invention. The compound represented by the formula (I) and a salt thereof may be either of a solvate, a hydrate, a non-solvate and an anhydride.

The compound of the present invention or a prodrug thereof (hereinafter sometimes to be abbreviated as the compound of the present invention) shows low toxicity and can be used as an agent for the prophylaxis or treatment of various diseases to be mentioned later for mammals (e.g., humans, mice, rats, rabbits, dogs, cats, bovines, horses, pigs, monkeys) as they are or by admixing with a pharmacologically acceptable carrier and the like to give a pharmaceutical composition.

Here, various organic or inorganic carriers conventionally used as materials for pharmaceutical preparations are used as a pharmacologically acceptable carrier, which are added as excipient, lubricant, binder and disintegrant for solid preparations; or solvent, solubilizing agent, suspending agent, isotonicity agent, buffer and soothing agent for liquid preparations, and the like. Where necessary, an additive for pharmaceutical preparations such as preservative, antioxidant, colorant, sweetening agent and the like can be used.

Preferable examples of the excipient include lactose, sucrose, D-mannitol, D-sorbitol, starch, α-starch, dextrin, crystalline cellulose, low-substituted hydroxypropylcellulose, sodium carboxymethylcellulose, gum acacia, pullulan, light anhydrous silicic acid, synthetic aluminum silicate and magnesium aluminate metasilicate.

Preferred examples of the lubricant include magnesium stearate, calcium stearate, talc and colloidal silica.

Preferable examples of the binder include α-starch, saccharose, gelatin, gum acacia, methylcellulose, carboxymethylcellulose, sodium carboxymethylcellulose, crystalline cellulose, sucrose, D-mannitol, trehalose, dextrin, pullulan, hydroxypropylcellulose, hydroxypropylmethylcellulose and polyvinylpyrrolidone.

Preferable examples of the disintegrant include lactose, sucrose, starch, carboxymethylcellulose, calcium carboxymethylcellulose, sodium croscarmellose, sodium carboxymethyl starch, light anhydrous silicic acid and low-substituted hydroxypropylcellulose.

Preferable examples of the solvent include water for injection, physiological brine, Ringer's solution, alcohol, propylene glycol, polyethylene glycol, sesame oil, corn oil, olive oil and cottonseed oil.

Preferred examples of the solubilizing agents include polyethylene glycol, propylene glycol, D-mannitol, trehalose, benzyl benzoate, ethanol, trisaminomethane, cholesterol, triethanolamine, sodium carbonate, sodium citrate, sodium salicylate and sodium acetate.

Preferred examples of the suspending agent include surfactants such as stearyltriethanolamine, sodium lauryl sulfate, lauryl aminopropionate, lecithin, benzalkonium chloride, benzethonium chloride, glyceryl monostearate and the like; hydrophilic polymers such as polyvinyl alcohol; polyvinylpyrrolidone, sodium carboxymethylcellulose, methylcellulose, hydroxymethylcellulose, hydroxyethylcellulose, hydroxypropylcellulose and the like; polysorbates, polyoxyethylene and hydrogenated castor oil.

Preferred examples of the isotonicity agent include sodium chloride, glycerol, D-mannitol, D-sorbitol and glucose.

Preferred examples of the buffer include buffers such as phosphate, acetate, carbonate and citrate.

Preferred examples of the soothing agent include benzyl alcohol.

Preferred examples of the preservative include p-oxybenzoates, chlorobutanol, benzyl alcohol, phenethyl alcohol, dehydroacetate and sorbic acid.

Preferred examples of the antioxidant include sulfite and ascorbate.

Preferable examples of the colorant include aqueous edible tar pigments (e.g., foodcolors such as Food Color Red Nos. 2 and 3, Food Color Yellow Nos. 4 and 5, Food Color Blue Nos. 1 and 2 and the like), water insoluble lake pigments (e.g., aluminum salt of the aforementioned aqueous edible tar pigment) and natural pigments (e.g., beta carotene, chlorophyll, red iron oxide).

Preferable examples of the sweetening agent include saccharin sodium, dipotassium glycyrrhizinate, aspartame and stevia.

The dosage form of the aforementioned pharmaceutical composition is, for example, an oral agent such as tablets (inclusive of sublingual tablets and orally disintegrable tablets), capsules (inclusive of soft capsules and microcapsules), granules, powders, troches, syrups, emulsions, suspensions and the like; or a parenteral agent such as injections (e.g., subcutaneous injections, intravenous injections, intramuscular injections, intraperitoneal injections, drip infusions), external agents (e.g., transdermal preparations, ointments), suppositories (e.g., rectal suppositories, vaginal suppositories), pellets, nasal preparations, pulmonary preparations (inhalations), ophthalmic preparations and the like. These may be administered safely via an oral or parenteral route.

These agents may be controlled-release preparations such as rapid-release preparations and sustained-release preparations (e.g., sustained-release microcapsules).

The pharmaceutical composition can be produced according to a method conventionally used in the field of pharmaceutical preparation, such as the method described in Japan Pharmacopoeia and the like. Specific production methods of the preparation are described in detail in the following.

While the content of the compound of the present invention in the pharmaceutical composition varies depending on the dosage form, dose of the compound of the present invention and the like, it is, for example, about 0.1 to 100 wt %.

The compound of the present invention has a superior GK activating action, and can be used as an agent for the prophylaxis or treatment of various diseases for mammals (e.g., human, bovine, horse, dog, cat, monkey, mouse, rat, specifically human). In addition, as the compound of the present invention has a selective GK activating action, it shows low toxicity (e.g., acute toxicity, chronic toxicity, cardiotoxicity, carcinogenic, genetic toxicity), which causes fewer side effects.

The compound of the present invention can be used as an agent for the prophylaxis or treatment of diabetes (e.g., type 1 diabetes, type 2 diabetes, gestational diabetes, obese diabetes); an agent for the prophylaxis or treatment of hyperlipidemia (e.g., hypertriglyceridemia, hypercholesterolemia, hypo-HDL-emia, postprandial hyperlipidemia); an agent for the prophylaxis or treatment of arteriosclerosis; an agent for the prophylaxis or treatment of impaired glucose tolerance (IGT); and an agent for preventing progression of impaired glucose-tolerance into diabetes.

For diagnostic criteria of diabetes, Japan Diabetes Society reported new diagnostic criteria in 1999.

According to this report, diabetes is a condition showing any of a fasting blood glucose level (glucose concentration of venous plasma) of not less than 126 mg/dl, a 75 g oral glucose tolerance test (75 g OGTT) 2 h level (glucose concentration of venous plasma) of not less than 200 mg/dl, and a non-fasting blood glucose level (glucose concentration of venous plasma) of not less than 200 mg/dl. A condition not falling under the above-mentioned diabetes and different from “a condition showing a fasting blood glucose level (glucose concentration of venous plasma) of less than 110 mg/dl or a 75 g oral glucose tolerance test (75 g OGTT) 2 h level (glucose concentration of venous plasma) of less than 140 mg/dl” (normal type) is called a “borderline type”.

In addition, ADA (American Diabetes Association) and WHO reported new diagnostic criteria of diabetes.

According to these reports, diabetes is a condition showing a fasting blood glucose level (glucose concentration of venous plasma) of not less than 126 mg/dl or a 75 g oral glucose tolerance test 2 h level (glucose concentration of venous plasma) of not less than 200 mg/dl.

According to the reports of ADA and WHO, impaired glucose tolerance is a condition showing a 75 g oral glucose tolerance test 2 h level (glucose concentration of venous plasma) of not less than 140 mg/dl and less than 200 mg/dl. According to the report of ADA, a condition showing a fasting blood glucose level (glucose concentration of venous plasma) of not less than 100 mg/dl and less than 126 mg/dl is called IFG (Impaired Fasting Glucose). According to WHO, among the IFG (Impaired Fasting Glucose), a condition showing a fasting blood glucose level (glucose concentration of venous plasma) of not less than 110 mg/dl and less than 126 mg/dl is called IFG (Impaired is Fasting Glycemia).

The compound of can also be used as an agent for the prophylaxis or treatment of diabetes, borderline type, impaired glucose tolerance, IFG (Impaired Fasting Glucose) and IFG (Impaired Fasting Glycemia), as determined according to the above-mentioned new diagnostic criteria. Moreover, the compound of the present invention can prevent progress of borderline type, impaired glucose tolerance, IFG (Impaired Fasting Glucose) or IFG (Impaired Fasting Glycemia) into diabetes.

The compound of the present invention can also be used as an agent for the prophylaxis or treatment of, for example, diabetic complications [e.g., neuropathy, nephropathy, retinopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, peripheral blood circulation disorder], obesity, osteoporosis, cachexia (e.g., cancerous cachexia, tuberculous cachexia, diabetic cachexia, blood disease cachexia, endocrine disease cachexia, infectious disease cachexia or cachexia due to acquired immunodeficiency syndrome), fatty liver, hypertension, polycystic ovary syndrome, kidney disease (e.g., diabetic nephropathy, glomerular nephritis, glomerulosclerosis, nephrotic syndrome, hypertensive nephrosclerosis, end stage kidney disease), muscular dystrophy, myocardial infarction, angina pectoris, cerebrovascular accident (e.g., cerebral infarction, cerebral apoplexy), abnormal sugar metabolism, abnormal lipid metabolism, insulin resistance syndrome, Syndrome X, metabolic syndrome (state concurrently associated with at least one of type 2 diabetes, impaired glucose tolerance and insulin resistance, and at least two from obesity, abnormal lipid metabolism, hypertension and trace albumin urine), Cushing's syndrome, hyperinsulinemia, hyperinsulinemia-induced sensory disorder, tumor (e.g., leukemia; breast cancer, prostate cancer, skin cancer), irritable bowel syndrome, acute or chronic diarrhea, inflammatory diseases (e.g., chronic rheumatoid arthritis, spondylitis deformans, osteoarthritis, lumbago, gout, postoperative or traumatic inflammation, swelling, neuralgia, pharyngolaryngitis, cystitis, hepatitis (inclusive of non-alcoholic steatohepatitis), pneumonia, pancreatitis, inflammatory bowel disease, ulcerative colitis, stomach mucous membrane injury (including stomach mucous membrane injury caused by aspirin)), visceral fat syndrome, and the like.

The compound of the present invention can also be used for improvement of insulin resistance, promotion or increase of insulin secretion, decrease of visceral fat, suppression of accumulation of visceral fat, improvement of sugar metabolism, improvement of lipid metabolism, suppression of oxidative LDL production, improvement of lipoprotein metabolism, improvement of coronary metabolism, prophylaxis or treatment of cardiovascular complication, prophylaxis or treatment of heart failure complication, lowering of blood remnant, prophylaxis or treatment of anovulation, prophylaxis or treatment of hirsutism, prophylaxis or treatment of hyperandrogenism, improvement of pancreatic (β cell) function, regeneration of pancreas (β cell), promotion of regeneration of pancreas (β cell) and the like.

The compound of the present invention can also be used for the secondary prevention and suppression of progression of various diseases mentioned above (e.g., cardiovascular event such as myocardial infarction etc.).

The compound of the present invention is particularly useful as an agent for the prophylaxis or treatment of type 2 diabetes, obese diabetes and the like.

While the dose of the compound of the present invention varies depending on the administration subject, administration route, target disease, condition and the like, the compound of the present invention is generally given in a single dose of about 0.01-100 mg/kg body weight, preferably 0.05-30 mg/kg body weight, more preferably −0.1-10 mg/kg body weight, in the case of, for example, oral administration to adult diabetic patients. This dose is desirably given 1 to 3 times a day.

The compound of the present invention can be used in combination with drugs such as a therapeutic agent for diabetes, a therapeutic agent for diabetic complications, a therapeutic agent for hyperlipidemia, an antihypertensive agent, an antiobestic agent, a diuretic, a chemotherapeutic agent, an immunotherapeutic agent, an antithrombotic agent, a therapeutic agent for osteoporosis, a antidementia agent, an erectile dysfunction improver, a therapeutic agent for pollakiuria or urinary incontinence, a therapeutic agent for dysuria and the like (hereinafter to be referred to as a combination drug). In this case, the timing of administration of the compound of the present invention and a combination drug is not limited. These may be simultaneously administered to an administration subject or administered in a staggered manner. Moreover, the compound of the present invention and a combination drug may be administered as two kinds of preparations each containing an active ingredient, or may be administered as a single preparation containing both active ingredients.

The dose of the combination drug can be determined as appropriate based on the dose clinically employed. The proportion of the compound of the present invention and the combination drug can be appropriately determined depending on the administration subject, administration route, target disease, condition, combination and the like. When, for example, the administration subject is human, the combination drug is used in an amount of 0.01-100 parts by weight per 1 part by weight of the compound of the present invention.

Examples of the therapeutic agents for diabetes include insulin preparations (e.g., animal insulin preparations extracted from pancreas of bovine and swine; human insulin preparations genetically synthesized using Escherichia coli or yeast; zinc insulin; protamine zinc insulin; fragment or derivative of insulin (e.g., ═INS-1 etc.), oral insulin preparation and the like), insulin sensitizers (e.g., pioglitazone or a salt thereof (preferably hydrochloride), rosiglitazone or a salt thereof (preferably maleate), Reglixane (JTT-501), Netoglitazone (MCC-555), DRF-2593, Edaglitazone (BM-13.1258), KRP-297, R-119702, Rivoglitazone (CS-011), FK-614, compounds described in WO99/58510 (e.g., (E)-4-[4-(5-methyl-2-phenyl-4-oxazolylmethoxy)benzyloxyimino]-4-phenylbutyric acid), compounds described in WO01/38325, Tesaglitazar (AZ-242), Ragaglitazar (N,N-622), Muraglitazar (BMS-298585), ONO-5816, LM-4156, MBX-102, Naveglitazar (LY-519818), MX-6054, LY-510929, Balaglitazone (N,N-2344), T-131 or a salt thereof, THR-0921), PPARγ agonists, PPARγ antagonists, PPARγ/α dual agonists, α-glucosidase inhibitors (e.g., voglibose, acarbose, miglitol, emiglitate etc.), biguanides (e.g., phenformin, metformin, buformin or a salt thereof (e.g., hydrochloride, fumarate, succinate)), insulin secretagogues [sulfonylurea (e.g., tolbutamide, glibenclamide, gliclazide, chlorpropamide, tolazamide, acetohexamide, glyclopyramide, glimepiride, glipizide, glybuzole), repaglinide, senaglinide, nateglinide, mitiglinide or calcium salt hydrate thereof], GPR40 agonists, GLP-1 receptor agonists [e.g., GLP-1, GLP-1MR agent, N,N-2211, AC-2993 (exendin-4), BIM-51077, Aib(8,35)hGLP-1(7,37)NH₂, CJC-1131], amylin agonists (e.g., pramlintide), phosphotyrosine phosphatase inhibitors (e.g., sodium vanadate), dipeptidyl-peptidase IV inhibitors (e.g., NVP-DPP-278, PT-100, P32/98, Vidagliptin (LAF-237), P93/01, TS-021, Sitagliptin (MK-431), Saxagliptin (BMS-477118), β3 agonists (e.g., AJ-9677), gluconeogenesis inhibitors (e.g., glycogen phosphorylase inhibitors, glucose-6-phosphatase inhibitors, glucagon antagonists), SGLT (sodium-glucose cotransporter) inhibitors (e.g., T-1095), 11β-HSD1 inhibitors (e.g., BVT-3498), adiponectin or agonists thereof, IKK inhibitors (e.g., AS-2868), leptin resistance improving drugs, somatostatin receptor agonists (compounds described in WO01/25228, WO3/42204, WO98/44921, —WO98/45285 and WO99/22735) and the like.

Examples of the therapeutic agents for diabetic complications include aldose reductase inhibitors (e.g., Tolrestat, Epalrestat, Zenarestat, Zopolrestat, Minalrestat, Fidarestat, CT-112, ranirestat (AS-3201)), neurotrophic factors and increasing drugs thereof (e.g., NGF, NT-3, BDNF, neurotrophin production-secretion promoters described in WO01/14372 (e.g., 4-(4-chlorophenyl)-2-(2-methyl-1-imidazolyl)-5-[3-(2-methylphenoxy)propyl]oxazole)), nerve regeneration accelerator (e.g., Y-128), PKC inhibitors (e.g., ruboxistaurin mesylate), AGE inhibitors (e.g., ALT-946, pimagedine, N-phenacylthiazolium bromide (ALT-766), ALT-711, EXO-226, Pyridorin, Pyridoxamine), active oxygen scavengers (e.g., thioctic acid), cerebral vasodilators (e.g., tiapuride, mexiletine), somatostatin receptor agonists (BIM23190), apoptosis signal regulating kinase-1 (ASK-1) inhibitors and the like.

Examples of the therapeutic agents for hyperlipidemia include HMG-CoA reductase inhibitors (e.g., pravastatin, simvastatin, lovastatin, atorvastatin, fluvastatin, pitavastatin, rosuvastatin and salts thereof (e.g., sodium salt, calcium salt)), squalene synthase inhibitors (e.g., compounds described in WO97/10224, such as N—[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid), fibrate compounds (e.g., bezafibrate, clofibrate, simfibrate, clinofibrate), ACAT inhibitors (e.g., Avasimibe, Eflucimibe), anion exchange resins (e.g., colestyramine), probucol, nicotinic acid drugs (e.g., nicomol, niceritrol), ethyl icosapentate, phytosterols (e.g., soysterol, γ-oryzanol) and the like.

Examples of the antihypertensive agents include angiotensin converting enzyme inhibitors (e.g., captopril, enalapril, delapril), angiotensin II antagonists (e.g., candesartan cilexetil, losartan, eprosartan, valsartan, telmisartan, irbesartan, tasosartan, 1-[[2′-(2,5-dihydro-5-oxo-4H-1,2,4-oxadiazol-3-yl)biphenyl-4-yl]methyl]-2-ethoxy-1H-benzimidazole-7-carboxylic acid), calcium antagonists (e.g., manidipine, nifedipine, amlodipine, efonidipine, nicardipine), potassium channel openers (e.g., levcromakalim, L-27152, AL 0671, NIP-121), clonidine and the like.

Examples of the antiobesity agents include antiobesity agents acting on the central nervous system (e.g., dexfenfluramine, fenfluramine, phentermine, sibutramine, amfepramone, dexamphetamine, mazindol, phenylpropanolamine, clobenzorex; MCH receptor antagonists (e.g., SB-568849; SNAP-7941; compounds described in WO01/82925 and WO01/87834); neuropeptide Y antagonists (e.g., CP-422935); cannabinoid receptor antagonists (e.g., SR-141716, SR-147778); ghrelin antagonists); pancreatic lipase inhibitors (e.g., orlistat, ATL-962), β3 agonists (e.g., AJ-9677), peptide anorexiants (e.g., leptin, CNTF (Ciliary Neurotropic Factor)), cholecystokinin agonists (e.g., lintitript, FPL-15849), feeding deterrents (e.g., P-57) and the like.

Examples of the diuretics include xanthine derivatives (e.g., sodium salicylate and theobromine, calcium salicylate and theobromine), thiazide preparations (e.g., ethiazide, cyclopenthiazide, trichloromethiazide, hydrochlorothiazide, hydroflumethiazide, benzylhydrochlorothiazide, penflutizide, polythiazide, methyclothiazide), antialdosterone preparations (e.g., spironolactone, triamterene), carbonate dehydratase inhibitors (e.g., acetazolamide), chlorobenzenesulfonamide preparations (e.g., chlortalidone, mefruside, indapamide), azosemide, isosorbide, etacrynic acid, piretanide, bumetanide, furosemide and the like.

Examples of the chemotherapeutic agents include alkylating agents (e.g., cyclophosphamide, ifosfamide), metabolic antagonists (e.g., methotrexate, 5-fluorouracil and derivatives thereof), antitumor antibiotics (e.g., mitomycin, adriamycin), plant-derived antitumor agents (e.g., vincristine, vindesine, carboplatin, etoposide and the like. Of these, Furtulon or NeoFurtulon, which are 5-fluorouracil derivatives, and the like are preferable.

Examples of the immunotherapeutic agents include microorganism or bacterial components (e.g., muramyl dipeptide derivatives, Picibanil), polysaccharides having immunity potentiating activity (e.g., lentinan, schizophyllan, krestin), cytokines obtained by genetic engineering techniques (e.g., interferon, interleukin (IL)), colony stimulating factors (e.g., granulocyte colony stimulating factor, erythropoietin) and the like, with preference given to interleukins such as IL-1, IL-2, IL-12 and the like.

Examples of the antithrombotic agents include heparin (e.g., heparin sodium, heparin calcium, dalteparin sodium), warfarins (e.g., warfarin potassium), anti-thrombin drugs (e.g., aragatroban), thrombolytic agents (e.g., urokinase, tisokinase, alteplase, nateplase, monteplase, pamiteplase), platelet aggregation inhibitors (e.g., ticlopidine hydrochloride, cilostazol, ethyl icosapentate, beraprost sodium, sarpogrelate hydrochloride) and the like.

Examples of the therapeutic agents for osteoporosis include alfacalcidol, calcitriol, elcatonin, calcitonin salmon, estriol, ipriflavone, risedronate disodium, pamidronate disodium, alendronate sodium hydrate, incadronate disodium and the like.

Examples of the antidementia agents include tacrine, donepezil, rivastigmine, galanthamine and the like.

Examples of the erectile dysfunction improvers include apomorphine, sildenafil citrate and the like.

Examples of the therapeutic agents for pollakiuria or urinary incontinence include flavoxate hydrochloride, oxybutynin hydrochloride, propiverine hydrochloride and the like.

Examples of the therapeutic agents for dysuria include acetylcholine esterase inhibitors (e.g., distigmine) and the

Furthermore, drugs having a cachexia-improving action established in animal models and clinical situations, such as cyclooxygenase inhibitors (e.g., indomethacin), progesterone derivatives (e.g., megestrol acetate), glucosteroids (e.g., dexamethasone), metoclopramide agents, tetrahydrocannabinol agents, fat metabolism improving agents (e.g., eicosapentanoic acid), growth hormones, IGF-1, or antibodies to a cachexia-inducing factor such as TNF-α, LIF, IL-6, oncostatin M and the like, can be used in combination with the compound of the present invention.

The combination drug is preferably insulin preparation, insulin sensitizer, α-glucosidase inhibitor, biguanide, insulin secretagogue (preferably sulfonylurea) and the like.

Two or more kinds of the above-mentioned combination drugs may be used in an appropriate ratio.

When the compound of the present invention is used in combination with a combination drug, the amount thereof can be reduced within a safe range in consideration of counteraction of these agents. Particularly, the dose of an insulin sensitizer, an insulin secretagogue (preferably a sulfonylurea) and a biguanide can be reduced as compared with the normal dose. Therefore, an adverse effect which may be caused by these agents can be prevented safely. In addition, the dose of the therapeutic agent for diabetic complications, therapeutic agent for hyperlipemia and antihypertensive agent can be reduced whereby an adverse effect which may be caused by these agents can be prevented effectively.

Compound (I) can be produced, for example, according to a method shown in the following Reaction Schemes 1 to 9, or a method analogous thereto.

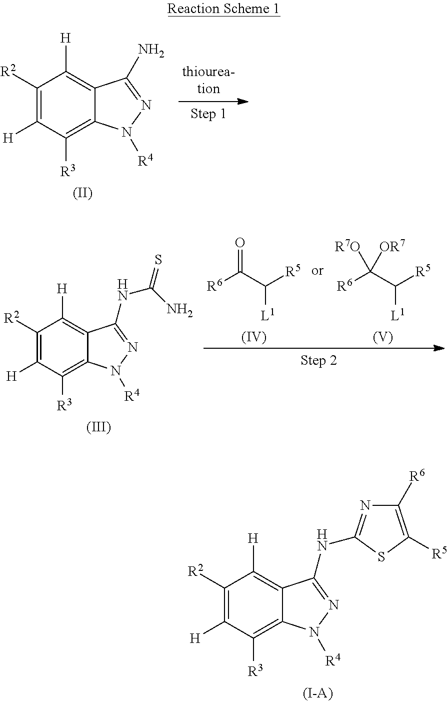

wherein R⁵ and R⁶ are each independently hydrogen or a substituent (e.g., optionally substituted C₁₋₆ alkyl), R⁷ is optionally substituted C₁₋₆ alkyl, L¹ is a leaving group (e.g., a halogen atom, alkylsulfonyloxy, arylsulfonyloxy etc.), and other symbols are as defined above.

### Step 1

Compound (III) can be produced by thioureating compound (II). This reaction is performed in the presence of, when desired, an acid or a base.

Examples of the thioureation agent include a thiocyanic acid salt (e.g., ammonium thiocyanate, sodium thiocyanate, potassium thiocyanate), thiocyanic acid ester (e.g., benzoyl isothiocyanate, ethoxycarbonyl isothiocyanate), and a thiocarbonyl compound (e.g., thiocarbonyl diimidazole, 1,1′-thiocarbonyl di-2(1H)-pyridone) and ammonia, or a combination with an ammonium salt (e.g., ammonium acetate, ammonium chloride), and the like.

Examples of the acid include mineral acids such as hydrochloric acid, sulfuric acid and the like; organic acids such as acetic acid, formic acid, propionic acid, trifluoroacetic acid, methanesulfonic acid and the like, and the like.

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like; metal hydrides such as sodium hydride, potassium hydride, calcium hydride and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like, and the like.

This reaction is advantageously performed using an inert solvent. Such solvent is not particularly limited as long as the reaction proceeds and, for example, solvents such as alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, dimethoxyethane, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; dimethylsulfoxide and the like; sulfolane; hexamethylphosphoramide; water and the like, and a mixed solvent thereof and the like are preferable.

The amount of the thioureation agent to be used is 1 to 10 mol, preferably 1 to 5 mol, relative to 1 mol of compound (II). When an acid or a base is used, the amount of the acid or the base to be used is 1 to 10 mol, preferably 1 to 5 mol, relative to 1 mol of compound (II). The reaction temperature is generally −30° C. to 100° C. The reaction time is generally 0.5 to 100 hr.

### Step 2

Compound (I-A) can be produced by reacting compound (III) with compound (IV) or compound (V) in the presence of an acid when desired.

Examples of the acid to be used in this reaction include mineral acids such as hydrochloric acid, sulfuric acid and the like; organic acids such as acetic acid, formic acid, trifluoroacetic acid, methanesulfonic acid and the like.

This reaction is advantageously performed without using a solvent, or in a solvent inert to the reaction. Such solvent is not particularly limited as long as the reaction proceeds and, for example, solvent such as alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethylsulfoxide and the like; sulfolane; hexamethyl phosphoramide; water and the like and a mixed solvent thereof and the like are preferable.

The amount of the compound (IV) or compound (V) to be used, and the amount of the acid to be used are 1 to 10 mol, preferably 1 to 5 mol, relative to compound (III). The reaction temperature is generally −30° C. to 100° C. The reaction time is generally 0.5 to 20 hr.

The thus-obtained compound (I-A) can be isolated and purified by a known separation and purification means, such as concentration, concentration under reduced pressure, solvent extraction, crystallization, recrystallization, phase transfer, chromatography and the like.

In Reaction Scheme 1, compound (II) to be used as a starting material can be produced by a method known per se, or Reaction Scheme 9 or Reaction Scheme 10.

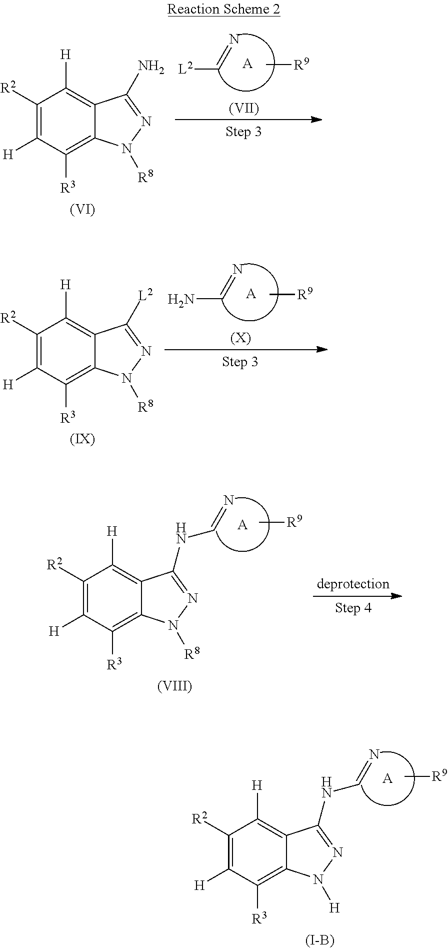

wherein R⁸ is optionally substituted C₁₋₆ alkyl, or a protecting group (e.g., tert-butoxycarbonyl, benzyloxycarbonyl, methoxymethyl, trimethylsilylethoxymethyl, formyl, p-toluenesulfonyl, methanesulfonyl etc.), R⁹ is hydrogen, or a substituent (e.g., optionally substituted C₁₋₆ alkyl), ring A is optionally substituted nitrogen-containing 5- or 6-membered heterocycle, L² is the aforementioned L¹ or boric acid, boric acid ester, and other symbols are as defined above.

### Step 3

Compound (VIII) can be produced by reacting compound (VI) with compound (VII), or compound (IX) with compound (X) in the presence of a metal catalyst and, when desired, in the presence of a ligand, a base, an oxidant and molecular sieves (trade name).

Examples of the metal catalyst include palladium catalysts (e.g., palladium(II) acetate, tris(dibenzylideneacetone)dipalladium(0), bis(dibenzylideneacetone)palladium(0), tetrakis(triphenylphosphine)palladium(0), [1,1′-bis(diphenylphosphino)ferrocene]dichloropalladium(II) dichloromethane adduct etc.) and nickel catalysts (e.g., tetrakis(triphenylphosphine)nickel(0), dichloro[1,3-bis(diphenylphosphino)propane]nickel(II), dichloro[1,4-bis(diphenylphosphino)butane]nickel(II) etc.) When L² is boric acid or boric acid ester, copper catalysts (e.g., copper(II) acetate, copper(I) iodide, copper(I) bromide, copper(I) chloride etc.) can be mentioned.

Examples of the ligand include phosphor ligands (e.g., 2,2′-bis(diphenylphosphino)-1,1′-binaphthyl, 4,5-bis(diphenylphosphino)-9,9-dimethylxanthene etc.)

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like; metal hydrides such as sodium hydride, potassium hydride, calcium hydride and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like, and the like.

When L² is boric acid, an oxidant and molecular sieves may be used when desired. Examples of the oxidant include gaseous oxygen, 2,2,6,6-tetramethylpiperidine 1-oxyl, pyridine 1-oxide and the like. Examples of the molecular sieves include 3A and 4A.

This reaction is advantageously performed without a solvent, or in a solvent inert to the reaction. Such solvent is not particularly limited as long as the reaction proceeds and, for example, alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, prepionitrile and the like; sulfoxides-such as dimethylsulfoxide and the like; sulfolane; hexamethylphosphoramide, and a mixed solvent thereof and the like, and the like are preferable.

The amount of the base or the oxidant to be used is generally 1 to 10 mol, preferably 1 to 5 mol, per 1 mol of compound (VI) or compound (IX).

The amount of the metal catalyst to be used is generally 0.01 to 0.5 mol, preferably 0.03 to 0.1 mol, per 1 mol of compound (VI) or compound (IX).

The amount of the ligand to be used is generally 0.01 to 1 mol, preferably 0.05 to 0.3 mol, per 1 mol of compound (VI) or compound (IX).

The amount of the molecular sieve to be used is 50 mg to 1000 mg relative to 1 g of compound (VI) or compound (IX).

The amount of compound (VII) or (X) to be used is generally 1 to 10 mol, preferably 1 to 3 mol, per 1 mol of compound (VI) or compound (IX).

The reaction temperature is generally −30° C. to 150° C., preferably 25° C. to 120° C. The reaction time is generally 0.5 to 20 hr.

### Step 4

When R⁸ is a protecting group (e.g., tert-butoxycarbonyl, benzyloxycarbonyl, methoxymethyl, trimethylsilylethoxymethyl, formyl, p-toluenesulfonyl etc.), compound (I-B) can be produced by deprotection of compound (VIII).

The reaction to eliminate a protecting group varies depending on the protecting group, and a method known per se or a method analogous thereto is used and, for example, the reaction can be performed according to the conditions described in “PROTECTIVE GROUPS IN ORGANIC SYNTHESIS” Second Edition (JOHN WILEY & SONS, INC.) and the like or in reference thereto.

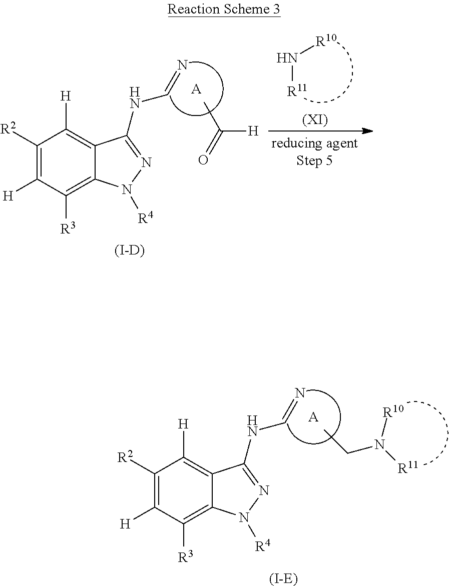

wherein R¹⁰ and R¹¹ are each independently hydrogen or a substituent, R¹⁰ and R¹¹ in combination may form an optionally substituted ring, and other symbols are as defined above.

Examples of the group represented by —NR¹⁰R¹¹ include “optionally substituted amino” and “optionally substituted 5- or 6-membered cyclic amino” exemplified above.

### Step 5

Compound (I-E) can be produced by what is called a reductive amination reaction comprising reacting compound (I-D) with compound (XI), and reducing the resulting imine or iminium ion to synthesize amines.

In this case, acid (e.g., mineral acids such as hydrochloric acid, phosphoric acid, sulfuric acid and the like, and organic acids such as toluenesulfonic acid, methanesulfonic acid, acetic acid and the like) may be added in 0.1 to 2 equivalent amount.

Examples of the reduction method include a method including reduction with a metal hydrogen complex compound such as sodium triacetoxyborohydrate, sodium borohydride, sodium cyanoborohydride, lithium aluminum hydride and the like or a reducing agent such as diborane and the like, electroreduction using lead or platinum as a cathode and the like. The amount of the reducing agent to be used is generally 1 to 10 mol, preferably 1 to 5 mol, per 1 mol of compound (I-D).

The reduction reaction can also be carried out by a hydrogenation reaction. In this case, for example, a catalyst such as palladium carbon, palladium black, platinum dioxide, Raney nickel, Raney cobalt, iron trichloride and the like is used. The amount of the catalyst to be used is generally about 5 to 1000 wt %, preferably about 10 to 300 wt %, relative to compound (I-D). The hydrogenation reaction can also be carried out using various hydrogen sources-instead of gaseous hydrogen; Examples of such hydrogen sources include formic acid, ammonium formate, triethylammonium formate, sodium phosphinate, hydrazine and the like. The amount of the hydrogen source to be used is generally about 1 to 100 mol, preferably about 1 to 5 mol, per 1 mol of compound (I-D).

Such solvent is not particularly limited as long as the reaction proceeds and, for example, solvents such as alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane;

hexamethylphosphoramide and the like, and a mixed solvent thereof and the like are preferable.

The amount of compound (XI) to be used is generally 1 to 10 mol, preferably 1 to 3 mol, per 1 mol of compound (I-D).

The reaction time is 0.5 to 72 hr, preferably 1 to 24 hr. The reaction temperature is −30° C. to 100° C., preferably 0° C. to 60° C.

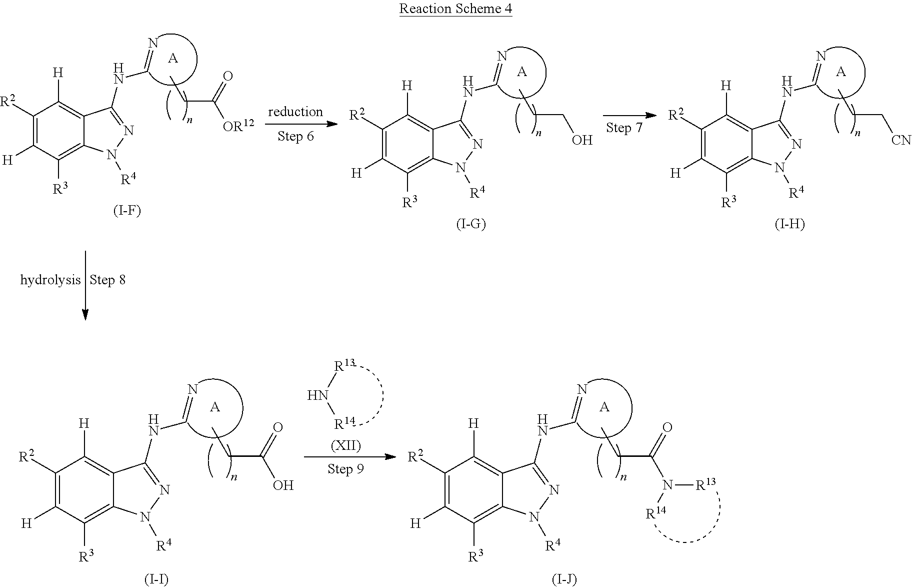

wherein R¹² is optionally substituted C₁₋₆ alkyl, R¹³ and R¹⁴ are each independently hydrogen or a substituent, R¹³ and R¹⁴ in combination may form an optionally substituted ring, and other symbols are as defined above.

Examples of the group represented by —CO—NR¹⁴R¹⁵ include “optionally substituted carbamoyl” exemplified above. Examples of the group represented by —NR¹⁴R¹⁵ include “optionally substituted amino” and “optionally substituted 5- or 6-membered cyclic amino” exemplified above.

### Step 6

Compound (I-G) can be produced by subjecting compound (I-F) to a reduction reaction.

The reduction reaction is performed using a reducing agent according to a conventional method. Examples of the reducing agent include metal hydrides such as aluminum hydride, diisobutylaluminum hydride, tributyltin hydride and the like; metal hydrogen complex compounds such as lithium aluminum hydride, sodium borohydride, lithium borohydride and the like; borane complexes such as borane tetrahydrofuran complex, borane dimethylsulfide complex and the like; alkylboranes such as thexylborane, dicyamylborane and the like; diborane; metals such as zinc, aluminum, tin, iron and the like; alkali metal/liquid ammonia (Birch reduction) such as sodium, lithium and the like, and the like.

The amount of the reducing agent to be used is appropriately determined depending on the kind of the reducing agent. For example, the amount of the metal hydride or metal hydrogen complex compound to be used is about 0.25 to about 10 mol, preferably about 0.5 to about 5 mol, relative to 1 mol of compound (I-F). The amount of the borane complex, alkylboranes or diborane to be used is about 1 to about 10 mol, preferably is about 1 to about 5 mol, relative to 1 mol of compound (I-F). The amount of the metal (including alkali metal to be used in Birch reduction) to be used is about 1 to about 20 mol, preferably about 1 to about 5 mol, relative to 1 mol of compound (I-F).

The reduction reaction is advantageously performed in a solvent inert to the reaction. Such solvent is not particularly limited as long as the reaction proceeds and, for example, solvents such as alcohols such as methanol, ethanol, 1-propanol, 2-propanol, tert-butyl alcohol and the like; ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoric triamide and the like; organic acids such as formic acid, acetic acid, propionic acid, trifluoroacetic acid, methanesulfonic acid and the like, and the like, a mixed solvent thereof and the like are preferable.

The reaction time varies depending on the kind and amount of the reducing agent to be used, and the activity and amount of the catalyst, and is generally about 1 hr to about 100 hr, preferably about 1 hr to about 50 hr. The reaction temperature is generally about −20° C. to about 120° C., preferably about 0° C. to about 80° C.

### Step 7

Compound (I-H) can be produced by subjecting compound (I-G) and a hydrogen cyanide or cyanohydrin compound (for example, acetonecyanhydrin) to a method known per se as Mitsunobu reaction, for example, the method described in Synthesis, 1981, 1-28, or a method analogous thereto. This reaction is generally carried out in the presence of an organic phosphorous compound and an electrophilic agent in a solvent that does not adversely influence the reaction.

Examples of the organic phosphorous compound include triphenylphosphine, tributylphosphine and the like. Examples of the electrophilic agent include-diethyl azodicarboxylate, diisopropyl azodicarboxylate, azodicarbonyl dipiperazine, 1,1′-(azodicarbonyl)dipiperidine and the like.

The amount of each of the organic phosphorous compound and electrophilic agent to be used is generally about 0.5 to 10 mol, preferably about 0.5 to 6 mol, per 1 mol of compound (I-G).

The Mitsunobu reaction is performed in a solvent inert to the reaction. Such solvent is not particularly limited as long as the reaction proceeds and, for example, ethers such as diethyl ether, tetrahydrofuran, 1,4-dioxane and the like; halogenated hydrocarbons such as chloroform, dichloromethane and the like; aromatic hydrocarbons such as benzene, toluene, xylene and the like; amides such as N,N-dimethylformamide and the like; sulfoxides such as dimethylsulfoxide and the like, and the like are preferable. These solvents may be used in a mixture at an appropriate ratio.

The reaction temperature is generally −50° C. to 150° C., preferably −10° C. to 100° C. The reaction time is generally 0.5 to 20 hr.

### Step 8

Compound (I-I) can be produced by subjecting compound (I-F) to hydrolysis. Hydrolysis is performed using an acid or a base according to a conventional method.

Examples of the acid include mineral acids such as hydrochloric acid, sulfuric acid and the like; Lewis acids such as boron trichloride, tribromide boron and the like; organic acids such as trifluoroacetic acid, p-toluenesulfonic acid and the like, and the like. Here, the Lewis acid can be used in combination with thiol or sulfide.

Examples of the base include alkali metal hydroxides such as lithium hydroxide, sodium hydroxide, potassium hydroxide, hydroxide barium and the like; alkali metal carbonates such as sodium carbonate, potassium carbonate and the like; alkali metal C₁₋₆ alkoxides such as sodium methoxide, sodium ethoxide, is potassium tert-butoxide and the like; organic bases such as triethylamine, imidazole, formamidine and the like, and the like.

The amount of the acid or base to be used is generally about 0.5 to 10 mol, preferably about 0.5 to 6 mol, per 1 mol of compound (I-F).

Hydrolysis is performed without a solvent or in a solvent inert to the reaction. Such solvent is not particularly limited as long as the reaction proceeds and, for example, alcohols such as methanol, ethanol, propanol and the like; aromatic hydrocarbons such as benzene, toluene and the like; saturated hydrocarbons such as cyclohexane, hexane and the like; organic acids such as formic acid, acetic acid and the like; ethers such as tetrahydrofuran, 1,4-dioxane, 1,2-dimethoxyethane and the like; amides such as N,N-dimethylformamide, N,N-dimethylacetamide and the like; halogenated hydrocarbons such as dichloromethane, chloroform, carbon tetrachloride, 1,2-dichloroethane and the like; nitriles such as acetonitrile, propionitrile and the like; ketones such as acetone, methylethylketone and the like; sulfoxides such as dimethylsulfoxide and the like; water and the like can be mentioned. These solvents may be used in a mixture of two or more kinds thereof at an appropriate ratio.

The reaction time is generally 10 min to 60 hr, preferably 10 min to 12 hr. The reaction temperature is generally −10° C. to 200° C., preferably 0° C. to 120° C.

### Step 9

Compound (I-J) can be produced by reacting compound (I-I) or a reactive derivative thereof at carboxyl or a salt thereof with compound (XII) or a salt thereof.

Examples of the reactive derivative at carboxyl of compound (I-I) include

1) acid chlorides;
2) acid azides;
3) mixed acid anhydrides with acids (e.g., substituted phosphoric acids such as dialkylphosphoric acid, phenylphosphoric acid, diphenylphosphoric acid, dibenzylphosphoric acid, halogenated phosphoric acid and the like; dialkylphosphorous acid; sulfurous acid; thiosulfuric acid; sulfuric acid; sulfonic acid such as methanesulfonic acid and the like; aliphatic carboxylic acids such as acetic acid, propionic acid, butyric acid, isobutyric acid, pivalic acid, pentanoic acid, isopentanoic acid, trichloroacetic acid and the like; aromatic carboxylic acids such as benzoic acid and the like) or chlorocarbonate esters (e.g., methyl chlorocarbonate, ethyl chlorocarbonate, isobutyl chlorocarbonate);
4) symmetric acid anhydrides;
5) active amides with imidazole, 4-substituted imidazole, dimethylpyrazole, triazole or tetrazole;
6) active esters such as cyanomethyl ester, methoxymethyl ester, dimethyliminomethyl ester, vinyl ester, propargyl ester, p-nitrophenyl ester, trichlorophenyl ester, pentachlorophenyl ester, mesylphenyl ester, phenylazophenyl ester, phenylthio ester, p-nitrophenyl ester, p-cresylthio ester, carboxymethylthio ester, pyranyl ester, pyridyl ester, piperidyl ester, 8-quinolylthio ester and the like;
7) esters with N-hydroxy compounds (e.g., N,N-dimethylhydroxyamine, 1-hydroxy-2-(1H)-pyridone, N-hydroxysuccinimide, N-hydroxyphthalimide, 1-hydroxy-1H-benzotriazole);
and the like. These reactive derivatives can be freely selected according to the kind of compound (I-I) to be used.

Examples of the preferable salt of a reactive derivative of compound (I-I) include basic salts such as alkali metal salt (e.g., sodium salt, potassium salt and the like); alkaline earth metal salt (e.g., calcium salt, magnesium salt and the like); ammonium salt; organic base salt (e.g., trimethylamine salt, triethylamine salt, pyridine salt, picoline salt, dicyclohexylamine salt, N,N-dibenzylethylenediamine salt and the like); and the like.

This reaction is preferably carried out in a solvent inert to the reaction. Such solvent is not particularly limited as long as the reaction proceeds and, for example, alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; water and the like can be mentioned. These solvents may be used in a mixture of two or more kinds thereof at an appropriate ratio.

In this reaction, when compound (I-I) is used in the form of a free acid or a salt thereof, the reaction is preferably performed in the presence of a conventionally used condensation agent such as Vilsmeier reagent and the like, which is prepared by reacting carbodiimide such as N,N′-dicyclohexylcarbodiimide, N-cyclohexyl-N′-morpholinoethylcarbodiimide, N-cyclohexyl-N′-(4-diethylaminocyclohexyl)carbodiimide, N,N′-s diethylcarbodiimide, N,N′-diisopropylcarbodiimide, N-ethyl-N′-(3-dimethylaminopropyl)carbodiimide and the like; N,N′-carbonylbis(2-methylimidazole); trialkyl phosphate; polyphosphoric acid ester such as ethyl polyphosphate, isopropyl polyphosphate and the like; phosphorus oxychloride; diphenylphosphoryl azide; thionyl chloride; oxalyl chloride; lower alkyl haloformates such as ethyl chloroformate, isopropyl chloroformate and the like; triphenylphosphine; 1-[bis(dimethylamino)methylene]-1H-1,2,3-triazolo(4,5-b) pyridinium 3-oxide hexafluorophosphate (HATU); N-hydroxybenzotriazole; 1-(p-chlorobenzenesulfonyloxy)-6-chloro-1-1H-benzotriazole; N,N′-dimethylformamide with thionyl chloride; phosgene, trichloromethyl chloroformate, phosphorus oxychloride and the like.

This reaction may be carried out in the presence of a base when desired. Examples of such base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like; organic lithiums such as methyllithium, n-butyllithium, sec-butyllithium, tert-butyllithium and the like; lithium amides such as lithium diisopropylamide and the like, and the like.

The amount of compound (XII) to be used is generally 1 to 10 mol, preferably 1 to 3 mol, per 1 mol of compound (I-I). The amount of the base to be used is generally 1 to 10 mol, preferably 1 to 3 mol, per 1 mol of compound (I-I).

The reaction temperature is generally −30° C. to 100° C. The reaction time is generally 0.5 to 20 hr.

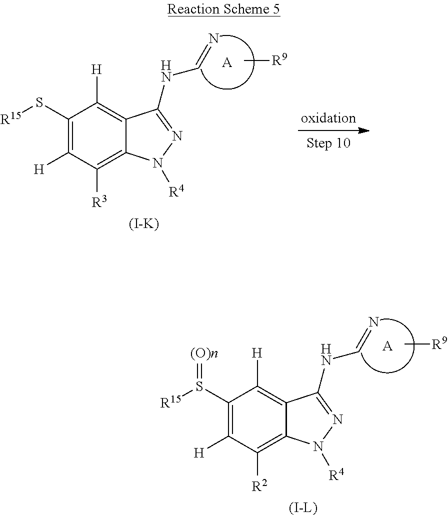

wherein R¹⁵ is a substituent, n is 1 or 2, and other symbols are as defined above.

### Step 10

Compound (I-L) can be produced by reacting compound (I-K) with an oxidant.

Examples of the oxidant include peracids such as peracetic acid, m-chloroperbenzoic acid and the like; hydrogen peroxide, sodium metaperiodate, hydroperoxide, ozone, selenium dioxide, potassium permanganate, chrome acid, iodine, bromine, N-bromosuccinic acid imide, iodosyl benzene, sodium hypochlorite, tert-butyl hypochlorite, potassium peroxomonosulfuric acid, ruthenium oxide and the like.

This reaction is preferably carried out in a solvent inert to the reaction. Such solvent is not particularly limited as long as the reaction proceeds and, for example, alcohols such as methanol, ethanol, propanol, isopropanol, butanol, tert-butanol and the like; halogenated hydrocarbons such as dichloromethane, chloroform, carbon tetrachloride, trichloroethylene and the like; hydrocarbons such as n-hexane, benzene, toluene and the like; water and the like can be mentioned. These solvents may be used in a mixture of two or more kinds thereof at an appropriate ratio.

The amount of the oxidant is 1 to 10 mol, preferably 1 to 3 mol, relative to 1 mol of compound (I-K). The reaction temperature is generally −30° C. to 100° C. The reaction time is generally 0.5 to 20 hr.

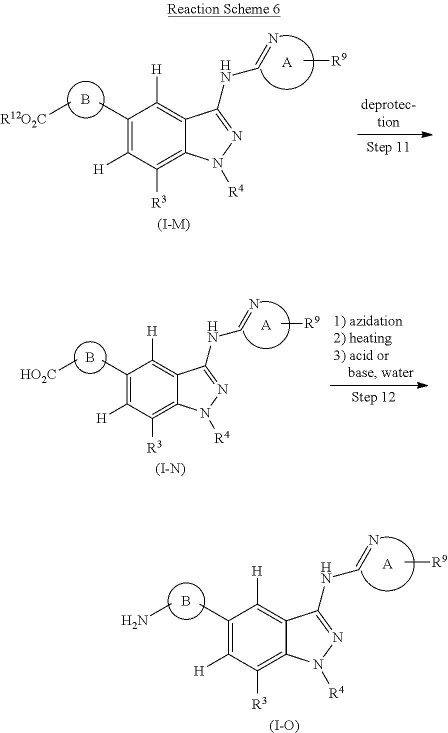

wherein R¹² is optionally substituted C₁₋₆ alkyl, ring B is an optionally substituted 3- to 7-membered ring, and other symbols are as defined above.

### Step 11

Compound (I-N) can be produced by subjecting an ester of compound (I-M) to deprotection according to a method analogous to the production method of compound (I-I) in Reaction Scheme 4.

### Step 12

Compound (I-O) can be produced by converting carboxyl of compound (I-N) to a reactive derivative and reacting the derivative with metal azide (e.g., sodium azide), or further heating acid azide obtained by using diphenylphosphoric acid azide to perform a rearrangement reaction, and subjecting the obtained isocyanate derivative to hydrolysis.

Examples of the reactive derivative at carboxyl of compound (I-O) include

1) acid chloride;
2) mixed acid anhydride with acid (e.g., substituted phosphoric acid such as dialkylphosphoric acid, phenylphosphoric acid, diphenylphosphoric acid, dibenzylphosphoric acid, halogenated phosphoric acid and the like; dialkylphosphorous acid; sulfurous acid; thiosulfuric acid; sulfuric acid; sulfonic acid such as methanesulfonic acid and the like; aliphatic carboxylic acid such as acetic acid, propionic acid, butyric acid, isobutyric acid, pivalic acid, pentanoic acid, isopentanoic acid, trichloroacetic acid and the like; aromatic carboxylic acid such as benzoic acid and the like) or chlorocarbonate ester such as chlorocarbonate (e.g., methyl chlorocarbonate, ethyl chlorocarbonate, isobutyl chlorocarbonate);
3) symmetric acid anhydride;
4) active amide with imidazole, 4-substituted imidazole, dimethylpyrazole, triazole or tetrazole;
5) activation ester such as cyanomethyl ester, methoxymethyl ester, dimethyliminomethyl ester, vinyl ester, propargyl ester, p-nitrophenyl ester, trichlorophenyl ester, pentachlorophenyl ester, mesylphenyl ester, phenylazophenyl ester, phenylthio ester, p-nitrophenyl ester, p-cresylthio ester, carboxymethylthio ester, pyranyl ester, pyridyl ester, piperidyl ester, 8-quinolylthio ester and the like;
7) ester with N-hydroxy compound (e.g., N,N-dimethylhydroxyamine, 1-hydroxy-2-(1H)-pyridone, N-hydroxysuccinimide, N-hydroxyphthalimide, 1-hydroxy-1H-benzotriazole);
and the like can be mentioned. These reactive derivatives can be freely selected according to the kind of compound (I-N) to be used.

This reaction is preferably carried out in a solvent inert to the reaction. Such solvent is not particularly limited as long as the reaction proceeds and, for example, ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like can be mentioned. These solvents may be used in a mixture of two or more kinds thereof at an appropriate ratio.

The amount of the metal azide (e.g., sodium azide) or diphenylphosphoric acid azide to be used is 1 to 10 mol, preferably 1 to 3 mol, relative to 1 mol of compound (I-N). The reaction temperature is −30° C. to 100° C., and the reaction time is generally 0.5 to 20 hr.

For hydrolysis, the reaction is performed by adding water. This reaction may be carried out in the presence of an acid or a base when desired.

Examples of the acid include mineral acids such as hydrochloric acid, phosphoric acid, sulfuric acid and the like, and organic acids such as toluenesulfonic acid, methanesulfonic acid, acetic acid and the like.

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like.

This reaction is preferably carried out in a solvent inert to the reaction. Such solvent is not particularly limited as long as the reaction proceeds and, for example, ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; water and the like can be mentioned. These solvents may be used in a mixture of two or more kinds thereof at an appropriate ratio.

The reaction temperature at which to carry out a m rearrangement reaction or hydrolysis is 30° C. to 200° C., preferably 50° C. to 150° C.

The reaction time is 0.5 to 50 hr, preferably 1 to 20 hr.

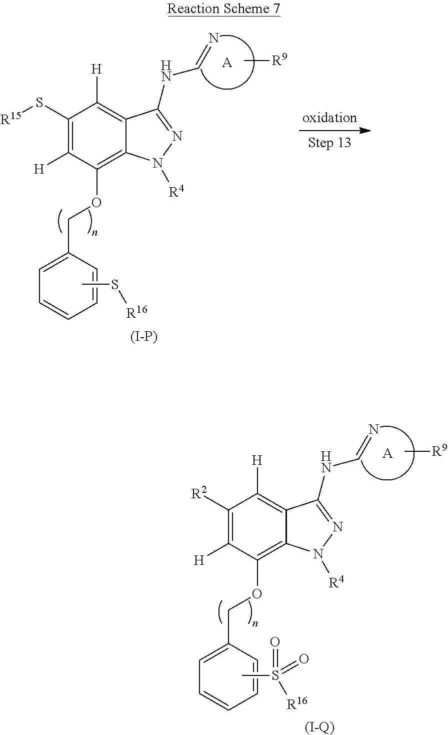

wherein R¹⁶ is optionally substituted C₁₋₆ alkyl, optionally substituted C₃₋₇ cycloalkyl, and other symbols are as defined above.

### Step 13

Compound (I-Q) can be produced by subjecting compound (I-P) to oxidation reaction according to a method analogous to the production method of compound (I-L) in Reaction Scheme 5.

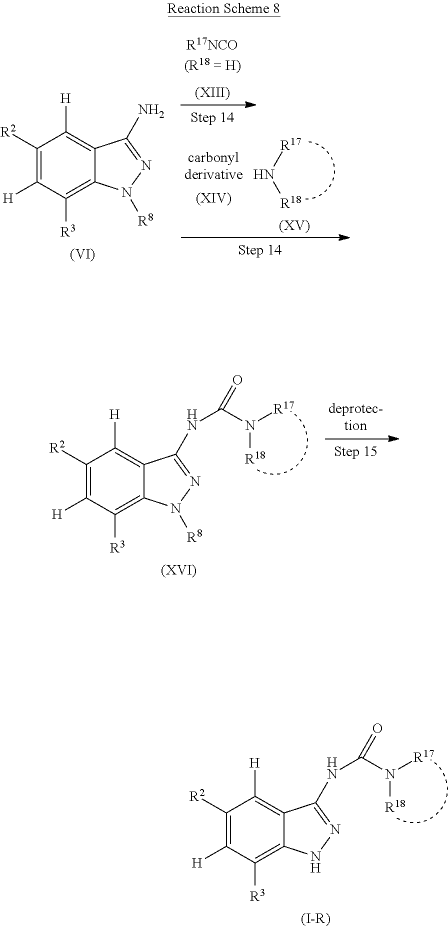

wherein R¹⁷ and R¹⁸ are each independently hydrogen or a substituent, R¹⁷ and R¹⁸ in combination may form an optionally substituted ring, and other symbols are as defined above.

Examples of the group represented by —CO—NR¹⁷R¹⁸ include “optionally substituted carbamoyl” exemplified above. Examples of the group represented by —NR″ R¹⁸ include “optionally substituted amino” and “optionally substituted 5- or 6-membered cyclic amino” exemplified above.

### Step 14

Compound (XVI) can be produced by reacting compound (VI) with compound (XIII).

This reaction is preferably carried out in a solvent inert to the reaction. Such solvent is not particularly limited as long as the reaction proceeds and, for example, ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like can be mentioned. These solvents may be used in a mixture of two or more kinds thereof at an appropriate ratio.

The amount of compound (XIII) to be used is generally 1 to 10 mol, preferably 1 to 3 mol, per 1 mol of compound (VI).

The reaction temperature is generally −20° C. to 150° C., preferably 0° C. to 100° C.

The reaction time is generally 0.5 to 100 hr, preferably 1 to 20 hr.

Alternatively, compound (XVI), can be produced by reacting a reactive carbonyl derivative produced by reacting compound (VI) with carbonyl derivative (XIV) in the presence of a base with compound (XV).

Examples of the carbonyl derivative (XIV) include phosgene, diphosgene, triphosgene, N,N′-carbonyldiimidazole, di(N-succinimidyl)carbonate and the like.

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and m the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like; metal hydrides such as sodium hydride, potassium is hydride, calcium hydride and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like, and the like.

This reaction is preferably carried out in a solvent inert to the reaction. Such solvent is not particularly limited as long as the reaction proceeds and, for example, alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; water and the like can be mentioned. These solvents may be used in a mixture of two or more kinds thereof at an appropriate ratio.

The amount of each of compound (XV) and carbonyl derivative (XIV) to be used is generally 1 to 10 mol, preferably 1 to 3 mol, per 1 mol of compound (VI).

The amount of the base to be used is generally 1 to 10 mol, preferably 1 to 3 mol, per 1 mol of compound (VI).

The reaction temperature is generally −20° C. to 150° C., preferably 0° C. to 100° C.

The reaction time is generally 0.5 to 100 hr, preferably 1 to 20 hr.

### Step 15

When R⁸ of compound (XVI) is a protecting group, compound (I-R) can be produced according to a general deprotection method such as acid treatment, alkali treatment, catalytic reduction and the like when desired.

In the following, production methods of a starting material compound and a reactive derivative thereof to be used in the present invention are explained in the following Reaction Scheme 9 or 10.

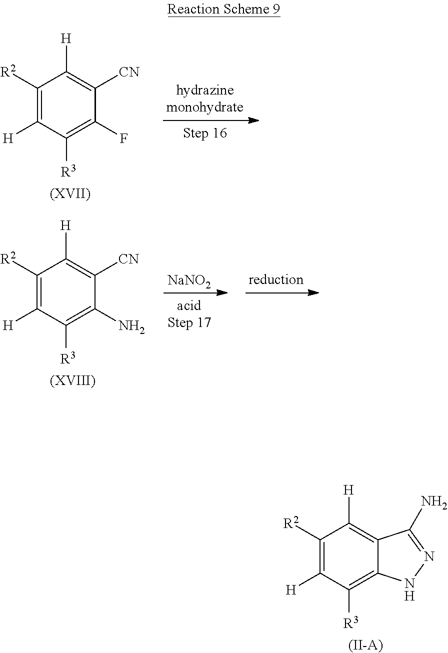

wherein each symbol is as defined above.

### Step 16

Compound (II-A) can be produced by reacting compound (XVII) with hydrazine monohydrate.

This reaction is advantageously performed in a solvent inert to the reaction. Such solvent is not particularly limited as long as the reaction proceeds and, for example, solvents such as alcohols such as methanol, ethanol, 1-propanol, 2-propanol, tert-butyl alcohol, 1-butanol and the like; ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like, and the like or a mixed solvent thereof and the like are preferable.

The amount of the hydrazine monohydrate to be used is generally 1 to 10 mol, preferably 1 to 3 mol, per 1 mol of compound (XVII).

The reaction temperature is generally −30° C. to 150° C., preferably 20° C. to 120° C. The reaction time is generally 1 to 100 hr.

### Step 17

Compound (II-A) can also be produced by diazotization of amino of compound (XVIII) with an acid and a nitrite salt (or organic nitrous acid compound) and, without isolation, subjecting the compound to a reduction reaction.

Examples of the nitrous acid compound include nitrite salts such as sodium nitrite, potassium nitrite and the like; organic nitrous acid compounds having 1 to 6 carbon atoms such as 1,1-dimethylethyl nitrite and the like, and the like.

The amount of the nitrite salt or organic nitrous acid compound to be used for diazotization is generally 1 to 5 mol, preferably 1 to 3 mol, per 1 mol of compound (XVIII).

Examples of the acid include mineral acids such as hydrochloric acid, hydrobromic acid, sulfuric acid and the like; organic acids such as acetic acid, formic acid, propionic acid, trifluoroacetic acid, methanesulfonic acid and the like, and the like.

The reaction temperature of diazotization is −5° C. to 10° C. The reaction time is 5 min to 2 hr.

The reduction reaction is performed by using, for example, a reducing agent. Examples of the reducing agent include metals such as iron, zinc, tin, tin dichloride and the like, and sulfides such as sodium dithionite, sodium sulfite and the like. The amount of the reducing agent to be used is appropriately determined according to the kind of the reducing agent. For example, the amount of the metal to be used is generally 1 to 20 mol, preferably 1 to 5 mol, per 1 mol of compound (XVIII). The amount of the sulfide to be used is generally 1 to 20 mol, preferably 1 to 5 mol, per 1 mol of compound (XVIII).

This reaction is preferably performed in a solvent inert to the reaction. Such solvent is not particularly limited as long as the reaction proceeds and, for example, alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; organic acids such as acetic acid, propionic acid and the like; water and the like can be mentioned. These solvents may be used in a mixture of two or more kinds thereof at an appropriate ratio.

The reaction time of the reduction reaction varies depending on the kind and amount of the reducing agent to be used, and is generally 0.5 to 20 hr. The reaction temperature is generally −20° C. to 100° C., preferably 0° C. to 100° C.

Compound (XVII) and compound (XVIII) to be used as starting materials in Reaction Scheme 9 can be produced according to, for example, the methods in Reaction Schemes 12 to 16.

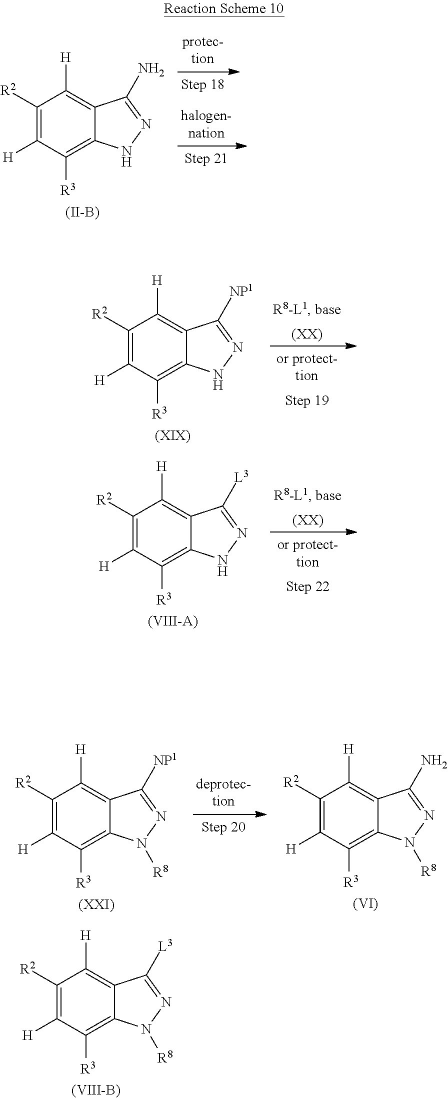

wherein P¹ is a protecting group (e.g., phthalimide), L³ is a halogen atom, and other symbols are as defined above.

For the steps of protection and deprotection in this Reaction Scheme, method known per se or a method analogous thereto is used. For example, the reaction can be performed according to the conditions described in “PROTECTIVE GROUPS IN ORGANIC SYNTHESIS” Second Edition (JOHN WILEY & SONS, INC.) and the like or in reference thereto.

### Step 18

Compound (XIX) can be produced by protecting an amino group of compound (II-B).

### Step 19

Compound (XXI) can be produced by alkylation of compound (XIX) using compound (XX) having a leaving group L¹ in the presence of a base. When R⁸ of compound (XIX) is a protecting group, compound (XXI) can be produced by subjecting the compound to a known protection reaction.

The amount of compound (XX) to be used is generally 1 to 5 mol, preferably 1 to 3 mol, per 1 mol of compound (XIX).

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like; metal hydrides such as sodium hydride, potassium hydride, calcium hydride and the like; and organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like.

The amount of the base to be used is generally 1 to 20 mol, preferably 1 to 5 mol, per 1 mol of compound (XIX).

This reaction is preferably carried out in a solvent inert to the reaction. Such solvent is not particularly limited as long as the reaction proceeds and, for example, ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; water and the like are preferable. These solvents may be used in a mixture of two or more kinds thereof at an appropriate ratio.

The reaction time is generally 0.5 to 20 hr. The reaction temperature is generally −20° C. to 150° C., preferably 0° C. to 100° C.

### Step 20

Compound (VI) can be produced by deprotecting compound (XXI) wherein R⁸ is a protecting group.

### Step 21

Compound (VIII-A) can be produced by halogenating compound (II-B).

Compound (II-B) can be halogenated by producing a diazonium salt of the amino group of compound (II-B) according to the production method of compound (II-A) in Reaction Scheme 9 and, without isolation, adding halogenated copper.

Diazotization can be carried out by a method analogous to the production method of compound (II-A) in Step 17 of Reaction Scheme 9.

Examples of the halogenated copper include copper bromide in the case of bromination, and copper iodide in the case of iodination. The amount thereof to be used is generally 1 to 20 mol, preferably 1 to 5 mol, per 1 mol of compound (II-B).

Examples of the solvent to be used in this reaction include those exemplified for the production method of compound (II-A) in Step 17 of Reaction Scheme 9.

The reaction time is generally 0.5 to 20 hr. The reaction temperature is generally −20° C. to 150° C., preferably 0 to 100° C.

### Step 22

Compound (VIII-B) can be produced by alkylating compound (VIII-A) according to a method analogous to the production method of compound (XXI) in Reaction Scheme 10, or by introducing a protecting group.

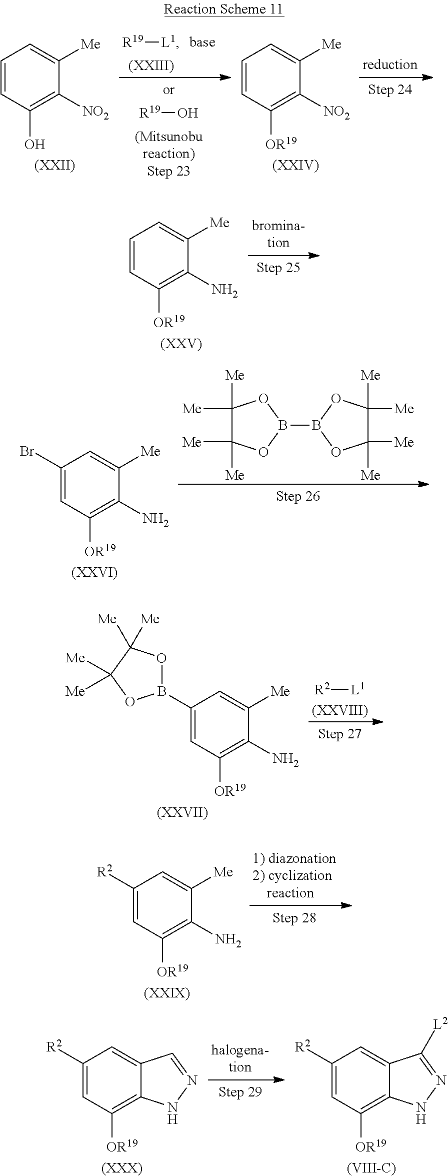

wherein R¹⁹ is optionally substituted C₁₋₆ alkyl or an optionally substituted 3- to 7-membered cyclic group optionally condensed with benzene, and other symbols are as defined above.

### Step 23

Compound (XXIV) can be produced by subjecting compound (XXII) and compound (XXIII) having a leaving group L1 to a substitution reaction in the presence of a base.

The amount of compound (XXIII) to be used is generally 1% to 10 mol, preferably 1 to 3 mol, per 1 mol of compound (XXII).

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal is carbonates such as sodium carbonate, potassium carbonate, cesium carbonate and the like; alkali metal hydrogen carbonates such as sodium hydrogen carbonate, potassium hydrogen carbonate and the like; alkali metal alkoxide having 1 to 6 carbon atoms such as sodium methoxide, sodium ethoxide, potassium tert-butoxide and the like; metal hydride such as sodium hydride, potassium hydride, calcium hydride and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like.

The amount of the base to be used is generally 1 to 20 mol, preferably 1 to 5 mol, per 1 mol of compound (XXII).

This reaction is preferably carried out in a solvent inert to the reaction. Such solvent is not particularly limited as long as the reaction proceeds and, for example, ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like can be mentioned. These solvents may be used in a mixture of two or more kinds thereof at an appropriate ratio.

The reaction time is generally 0.5 to 20 hr. The reaction temperature is generally −20° C. to 150° C., preferably 0° C. to 100° C.

Alternatively, compound (XXIV) can also be produced by Mitsunobu reaction with an alcohol form represented by the formula R¹⁹—OH wherein R¹⁹ is as defined above, for example, by the method described in Synthesis, 1-28 (1981), or a method analogous thereto. That is, this reaction can be generally carried out in a solvent that does not adversely influence the reaction in the presence of an organic phosphorous compound and an electrophilic agent.

Examples of the organic phosphorous compound include triphenylphosphine, tributylphosphine and the like. Examples of the electrophilic agent include diethyl azodicarboxylate, diisopropyl azodicarboxylate, azodicarbonyldipiperazine, 1,1′-(azodicarbonyl)dipiperidine and the like. The amount of each of the organic phosphorous compound and electrophilic agent to be used is preferably 1 to 5 mol, relative to 1 mol of compound (XXII).

The amount of the organic phosphorous compound and electrophilic agent to be used is generally about 0.5 to 10 mol, preferably 0.5 to 6 mol, per 1 mol of compound (XXII).

Mitsunobu reaction is carried out in a solvent inert to the reaction. Such solvent is not particularly limited as long as the reaction proceeds and, for example, ethers such as diethyl ether, tetrahydrofuran, 1,4-dioxane and the like; halogenated hydrocarbons such as chloroform, dichloromethane and the like; aromatic hydrocarbons such as benzene, toluene, xylene and the like; amides such as N,N-dimethylformamide and the like; sulfoxides such as dimethyl sulfoxide and the like, and the like can be mentioned. These solvents may be used in a is mixture at appropriate ratio.

The reaction-temperature is generally −50° C. to 150° C., preferably −10° C. to 100° C. The reaction time is generally 0.5 to 20 hr.

### Step 24

Compound (XXV) can be produced by subjecting compound (XXIV) to a reduction reaction.

The reduction reaction can be performed using, for example, a reducing agent. Examples of the reducing agent include metals such as iron, zinc, tin and the like; sulfides such as sodium dithionite and the like, and the like. The amount of the reducing agent to be used is appropriately determined according to the kind of the reducing agent. For example, the amount of the metal to be used is generally about 1 to about 20 equivalent amount, preferably about 1 to about 5 equivalent amount, per 1 mol of compound (XXIV). The amount of the sulfide to be used is generally 1 to 20 mol, preferably 1 to 5 mol, per 1 mol of compound (XXIV).

The reduction reaction is carried out according to a hydrogenation reaction. In this case, for example, a catalyst such as palladium carbon, palladium black, platinum dioxide, Raney nickel, Raney cobalt, iron trichloride and the like can be used. The amount of the catalyst to be used is generally about 5 to 1000 wt %, preferably about 10 to 300 wt %, relative to compound (XXIV). The hydrogenation reaction can also be performed using various hydrogen sources instead of gaseous hydrogen. Examples of such hydrogen sources include formic acid, ammonium formate, triethylammonium formate, sodium phosphinate, hydrazine and the like. The amount of the hydrogen source to be used is generally about 1 to 100 mol, preferably about 1 to 5 mol, per 1 mol of compound (XXIV).

The reduction reaction is preferably carried out in a solvent inert to the reaction. Such solvent is not particularly limited as long as the reaction proceeds and, for example, alcohols such as methanol, ethanol, 1-propanol, 2-propanol, tert-butyl alcohol and the like; ethers such as diethyl ether, diisopropyl ether, diphenyl ether, tetrahydrofuran, 1,4-dioxane, 1,2-dimethoxyethane and the like; aromatic hydrocarbons such asbenzene, toluene and the like; saturated hydrocarbons such as cyclohexane, hexane and the like; amides such as N,N-dimethylformamide, N,N-dimethylacetamide, hexamethylphosphoramide and the like; for example, mineral acid such as hydrochloric acid, sulfuric acid and the like; organic acids such as formic acid, acetic acid, propionic acid, trifluoroacetic acid, methanesulfonic acid and the like, and the like can be mentioned. These solvents may be used in a mixture of two or more kinds thereof at an appropriate ratio.

The reaction time varies depending on the kind and amount of the reducing agent to be used and is generally about 1 hr to about 100 hr, preferably about 1 hr to about 50 hr. The reaction temperature is generally about −20° C. to about 120° C., preferably about 0° C. to about 80° C.

### Step 25

Compound (XXVI) can be produced by bromination of compound (XXV).

Examples of the reaction agent to be used for bromination include bromine, N-bromosuccinimide, 1,4-dioxane-bromine complex and the like, and the amount thereof to be used is generally 1 to 5 mol, preferably 1 to 3 mol, per 1 mol of compound (XXV).

This reaction is preferably carried out in a solvent inert to the reaction. Such solvent is not particularly limited as long as the reaction proceeds and, for example, ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, -N,N-dimethylacetamide and the like; nitriles such as acetonitrile, propionitrile and the like; sulfoxides such as dimethyl sulfoxide and the like; sulfolane; hexamethylphosphoramide and the like can be mentioned. These solvents may be used in a mixture of two or more kinds thereof at an appropriate ratio.

The reaction time is generally 0.5 to 20 hr. The reaction temperature is generally −20° C. to 100° C., preferably 0° C. to 50° C.

### Step 26

Compound (XXVII) can be produced from compound (XXVI) according to a method analogous to the method described in Journal of Organic Chemistry, 60, 7508 (1995) and the like.

In this reaction, compound (XXVI) is reacted with bis(pinacolate)diboron in the presence of potassium acetate, using [1,1′-bis(diphenylphosphino)ferrocene]dichloropalladium (II) dichloromethane adduct as a catalyst.

This reaction is preferably carried out in a solvent inert to the reaction. Such solvent is not particularly limited as long as the reaction proceeds and, for example, ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like can be mentioned. These solvents may be used in a mixture of two or more kinds thereof at an appropriate ratio.

The amount of each of the bis(pinacolate)diboron, potassium acetate, and [1,1′-bis(diphenylphosphino)ferrocene]dichloropalladium(II) dichloromethane adduct to be used is generally 1 to 10 mol, 1 to 10 mol and 0.01 to 1 mol, preferably 1 to 3 mol, 1 to 3 mol and 0.03 to 0.2 mol, per 1 mol of compound (XXIV).

The reaction time is generally 0.5 to 50 hr, preferably 1 to 20 hr. The reaction temperature is generally 0° C. to 150° C., preferably 30° C. to 100° C.

### Step 27

Compound (XXIX) can be produced by subjecting compound (XXVII) and compound (XXVIII) to what is called Suzuki coupling in the presence of a metal catalyst and a base.

Examples of the metal catalyst include palladium catalysts (e.g., palladium(II) acetate, tris(dibenzylideneacetone)dipalladium(0), bis(dibenzylideneacetone)palladium(0), tetrakis(triphenylphosphine)palladium(0), (1,1′-bis(diphenylphosphino)ferrocene)dichloropalladium dichloromethane adduct(II) etc.) and nickel catalysts (e.g., tetrakis(triphenylphosphine)nickel(0), dichloro[1,3-bis(diphenylphosphino)propane]nickel(0), dichloro[1,4-bis(diphenylphosphino)butane]nickel(0) etc.).

This reaction may be carried out in the presence of a ligand when desired. Examples of such ligand include phosphor ligands (e.g., triphenylphosphine, 1,3-bis(diphenylphosphino)propane, 1,3-bis(diphenylphosphino)propane, 2,2′-bis(diphenylphosphino)-1,1′-binaphthyl, and 4,5-bis(diphenylphosphino)-9,9-dimethylxanthene etc.).

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like, and the like.

This reaction is preferably carried out in a solvent inert to the reaction. Such solvent is not particularly limited as long as the reaction proceeds and, for example, alcohols such as methanol, ethanol, propanol, isopropanol, butanol, tert-butanol and the like; ethers such as 1,4-dioxane, tetrahydrofuran, diethyl ether, dimethoxyethane, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; water and the like can be mentioned. These solvents may be used in a mixture of two or more kinds thereof at an appropriate ratio.

The amount of the base to be used is generally 1 to 10 mol, preferably 1 to 5 mol, per 1 mol of compound (XXVII).

The amount of the metal catalyst to be used is generally 0.01 to 0.5 mol, preferably 0.03 to 0.1 mol, per 1 mol of compound (XXVII).

The amount of the ligand to be used is generally 0.01 to 1 mol, preferably 0.05 to 0.3 mol, per 1 mol of compound (XXVII).

The amount of compound (XXVIII) to be used is generally 1 to 10 mol, preferably 1 to 3 mol, per 1 mol of compound (XXVII).

The reaction temperature is generally −30° C. to 150° C., preferably 25° C. to 120° C. The reaction time is generally 0.5 to 20 hr.

### Step 28

Compound (XXX) can be produced by producing compound (XXIX) according to the production method of compound (II-A) in Reaction Scheme 9 and, without isolation, subjecting the compound to a cyclization reaction.

A cyclization reaction of diazonium salt is carried out using a base. Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal alkoxide having 1 to 6 carbon atoms such as sodium methoxide, sodium ethoxide, potassium tert-butoxide and the like; acetate such as potassium acetate, sodium acetate and the like, and the like.

This reaction is preferably carried out in a solvent inert to the reaction. Such solvent is not particularly limited as long as the reaction proceeds and, for example, alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; organic acids such as acetic acid, propionic acid and the like; water and the like can be mentioned. These solvents may be used in a mixture of two or more kinds thereof at an appropriate ratio.

This reaction may be carried out in the presence of crown ether when desired. Examples of the crown ether include 18-crown-6,15-crown-5 and the like, and the kind of the crown ether is preferably selected according to the base to be used.

The amount of the base to be used is generally 1 to 10 mol, preferably 1 to 5 mol, per 1 mol of compound (XXIX).

Alternatively, compound (XXX) can also be produced by diazotization of compound (XXIX) in the presence of acetic anhydride and a base using a nitrous acid compound, and simultaneously performing a cyclization reaction. In this case, the resultant product may contain an acetyl form. However, acetyl is removed under a basic condition to afford compound (XXX).

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like; acetates such as potassium acetate, sodium acetate and the like, and the like.

This reaction is preferably carried out in a solvent inert to the reaction. Such solvent is not particularly limited as long as the reaction proceeds and ethers such as 1,4-dioxane, tetrahydrofuran, diethyl ether, dimethoxyethane, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like can be mentioned. These solvents may be used in a mixture of two or more kinds thereof at an appropriate ratio.

Examples of the nitrous acid compound include nitrite salts such as sodium nitrite, potassium nitrite and the like, C₁₋₆ nitrous acid organic compounds such as 1,1-dimethylethyl nitrite and the like, and the like.

The amount of the nitrite salt or organic nitrous acid compound is generally 1 to 5 mol, preferably 1 to 3 mol, per 1 mol of compound (XXIX).

The amount of the acetic anhydride or base to be used is generally 1 to 10 mol, preferably 1 to 3 mol, per 1 mol of compound (XXIX).

The reaction temperature is −5° C. to 100° C. The reaction time is 1 hr to 50 hr.

When the resultant product contains an acetyl form, acetyl is removed under a basic condition to afford compound (XXX).

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal alkoxide having 1 to 6 carbon atoms such as sodium methoxide, sodium ethoxide, potassium tert-butoxide and the like; acetate such as potassium acetate, sodium acetate and the like, and the like.

The amount of the base to be used is generally 1 to 20 mol, preferably 1 to 5 mol, per 1 mol of compound (XXIX). The reaction temperature is 25° C. to 100° C. The reaction time is 1 hr to 50 hr.

### Step 29

Compound (VIII-C) can be produced by halogenating compound (XXX) according to the production method of compound (XXVI) in Reaction Scheme 11.

This reaction is preferably carried out in a solvent inert to the reaction. Such solvent is not particularly limited as long as the reaction proceeds, and ethers such as 1,4-dioxane, tetrahydrofuran, diethyl ether, dimethoxyethane, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like can be mentioned. These solvents may be used in a mixture of two or more kinds thereof at an appropriate ratio.

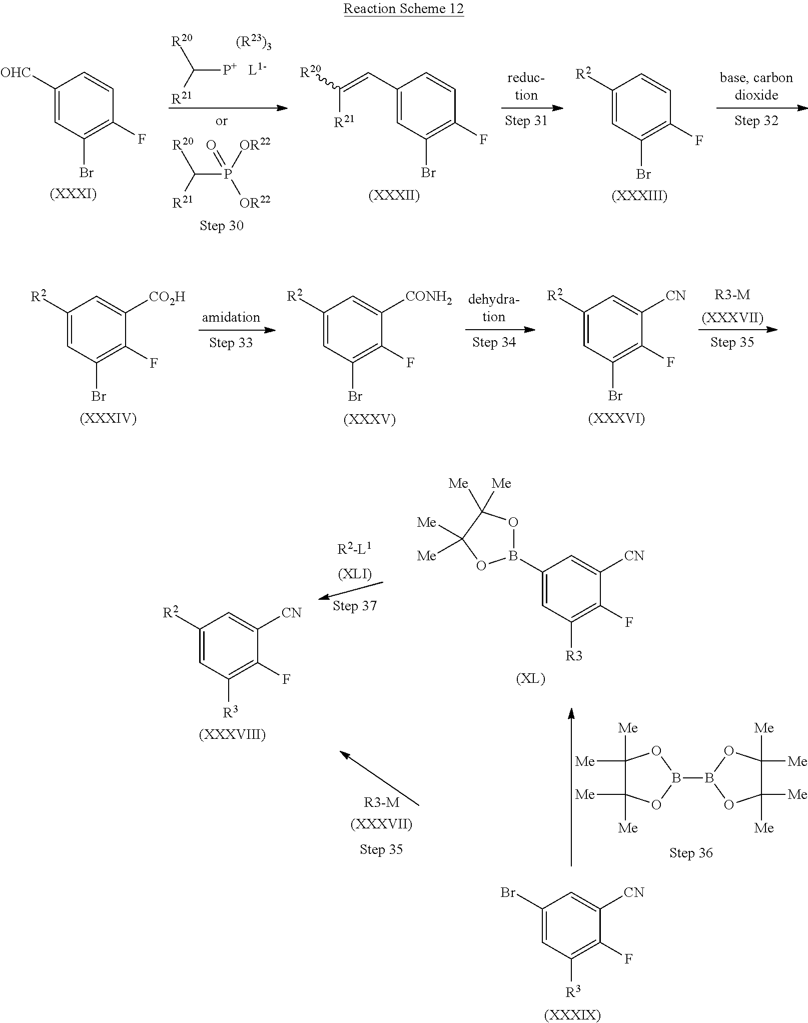

wherein R²⁰ and R²¹ are each independently hydrogen or optionally substituted C₁₋₆ alkyl; R²² is optionally substituted C₁₋₆ alkyl, R²³ is optionally substituted C₁₋₆ alkyl or phenyl, M is a metal (e.g., zinc, magnesium, boron, silicon, tin, copper etc., these may be substituted or complexed), and other symbols are as defined above.

### Step 30

Compound (XXXII) can be produced by what is called Wittig reaction wherein compound (XXXI) is reacted with phosphonium ylide induced from a phosphonium salt, or what is called Wittig-Horner-Emmons reaction, wherein compound (XXXI) is reacted with phosphonate carboanion induced from alkylphosphorous acid diester to give olefin.

This reaction is performed by developing phosphonium ylide or phosphonate carboanion in the system using a base in any case. Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like; metal hydrides such as sodium hydride, potassium hydride, calcium hydride; alkali metal alkoxides having 1 to 6 carbon atoms such as n-butyllithium, tert-butyllithium, sec-butyllithium; metal amides such as sodium amide, sodium hexamethyldisilazide, potassium hexamethyldisilazide, lithium diisopropylamide and the like.

This reaction is preferably carried out in a solvent inert to the reaction. Such solvent is not particularly limited as long as the reaction proceeds, and alcohols such as methanol, ethanol, 1-propanol, 2-propanol, tert-butyl alcohol and the like; ethers such as 1,4-dioxane, tetrahydrofuran, diethyl ether, dimethoxyethane, tert-butyl methyl ether, diisopropyl ether, ethylene-glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like can be mentioned. These solvents may be used in a mixture of two or more kinds thereof at an appropriate ratio.

The amount of the base to be used is generally 1 to 10 equivalent amount, preferably 1 to 5 mol equivalent amount, per 1 mol of compound (XXXI).

The amount of the phosphonium salt or phosphonate to be used is generally 1 to 5 mol, preferably 1 to 3 mol, per 1 molar equivalent amount of compound (XXXI).

The reaction temperature is generally −30° C. to 150° C., preferably 0° C. to 100° C. The reaction time is generally 0.5 to 20 hr.

### Step 31

Compound (XXXIII) can be produced by hydrogenation reaction of compound (XXXII).

For the hydrogenation reaction, for example, a catalyst such as palladium carbon, palladium black, platinum dioxide, Raney nickel, Raney cobalt and the like can be used. The amount of the catalyst to be used is about 5 to about 1000 wt %, preferably about 10 to about 300 wt %, per 1 mol of compound (XXXII). For the hydrogenation reaction, various hydrogen sources can be used instead of gaseous hydrogen. Examples of such hydrogen sources include formic acid, ammonium formate, triethylammonium formate, sodium phosphinate, hydrazine and the like. The amount of the hydrogen source to be used is 1 to 30 mol, preferably 1 to 10 mol, per 1 mol of compound (XXXII).

This reaction is advantageously performed in a solvent inert to the reaction. Such solvent is not particularly limited as long as the reaction proceeds and, for example, solvents such as alcohols such as methanol, ethanol, 1-propanol, 2-propanol, tert-butyl alcohol and the like; ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoric triamide and the like; organic acids such as formic acid, acetic acid, propanoic acid, trifluoroacetic acid, methanesulfonic acid and the like, and the like or a mixed solvent and the like are preferable. The reaction time varies depending on the reagent and solvent to be used, and is generally 10 min to 100 hr, preferably 30 min to 50 hr. The reaction temperature is generally −20 to 100° C., preferably 0 to 80° C. When gaseous hydrogen is used, the reaction internal pressure is generally 1 pressure to 100 pressure, preferably 1 pressure to 10 pressure.

### Step 32

Compound (XXXIV) can be produced by reacting compound (XXXIII) with a base and carbon dioxide.

Examples of the base include alkyl metals having 1 to 6 carbon atoms such as n-butyllithium, tert-butyllithium, sec-butyllithium; metal amides such as sodium hexamethyldisilazide, potassium hexamethyldisilazide, lithium diisopropylamide, and the like.

This reaction is preferably carried out in a solvent inert to the reaction. Such solvent is not particularly limited as long as the reaction proceeds, and examples thereof include ethers such as 1,4-dioxane, tetrahydrofuran, diethyl ether, dimethoxyethane, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like. These solvents may be used in a mixture of two or more kinds thereof at an appropriate ratio.

The amount of the base to be used is generally 1 to 10 mol, preferably 1 to 5 mol, per 1 mol of compound (XXXIII).

The reaction temperature is generally −30° C. to 150° C., preferably 25° C. to 120° C. The reaction time is generally 0.5 to 20 hr.

### Step 33

Compound (XXXV) can be produced by reacting compound (XXXIV) or a reactive derivative thereof at carboxyl or a salt thereof with ammonia or a salt thereof.

Examples of the reactive derivative at carboxyl of compound (XXXIV) include

1) acid chlorides;
2) acid azides;
3) mixed acid anhydrides with acids (e.g., substituted phosphoric acids such as dialkylphosphoric acid, phenylphosphoric acid, diphenylphosphoric acid, dibenzylphosphoric acid, halogenated phosphoric acid and the like; dialkylphosphorous acids; sulfurous acid; thiosulfuric acid; sulfuric acid; sulfonic acids such as methanesulfonic acid and the like; aliphatic carboxylic acids such as acetic acid, propionic acid, butyric acid, isobutyric acid, pivalic acid, pentanoic acid, isopentanoic acid, trichloroacetic acid and the like; aromatic carboxylic acids such as benzoic acid and the like) or chlorocarbonate esters (e.g., methyl chlorocarbonate, ethyl chlorocarbonate, isobutyl chlorocarbonate);
4) symmetric acid anhydrides;
5) active amides with imidazole, 4-substituted-imidazole, dimethylpyrazole, triazole or tetrazole;
6) active esters such as cyanomethyl ester, methoxymethyl ester, dimethyliminomethyl ester, vinyl ester, propargyl ester, p-nitrophenyl ester, trichlorophenyl ester, pentachlorophenyl ester, mesylphenyl ester, phenylazophenyl ester, phenylthio ester, p-nitrophenyl ester, p-cresylthio ester, carboxymethylthio ester, pyranyl ester, pyridyl ester, piperidyl ester, 8-quinolylthio ester and the like;
7) esters with N-hydroxy compounds (e.g., N,N-dimethylhydroxyamine, 1-hydroxy-2-(1H)-pyridone, N-hydroxysuccinimide, N-hydroxyphthalimide, 1-hydroxy-1H-benzotriazole);
and the like. These reactive derivatives can be freely selected according to the kind of compound (XXXIV) to be used.

Examples of the preferable salt of the reactive derivative of compound (XXXIV) include basic salts such as alkali metal salt (e.g., sodium salt, potassium salt and the like); alkaline earth metal salt (e.g., calcium salt, magnesium salt and the like); ammonium salt; organic base salt (e.g., trimethylamine salt, triethylamine salt, pyridine salt, picoline salt, dicyclohexylamine salt, N,N-dibenzylethylenediamine salt and the like); and the like.

This reaction is preferably carried out in a solvent inert to the reaction. Such solvent is not particularly limited as long as the reaction proceeds, and examples thereof include alcohols such as methanol, ethanol, propanol, isopropanol, butanol, tert-butanol and the like; ethers such as 1,4-dioxane, tetrahydrofuran, diethyl ether, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; water and the like. These solvents may be used in a mixture of two or more kinds thereof at an appropriate ratio.

In this reaction, when compound (XXXIV) is used in the form of a free acid or a salt thereof, the reaction is preferably performed in the presence of a conventionally used condensation agent such as so-called a Vilsmeier reagent, which is prepared by reacting carbodiimides such as N,N′-dicyclohexylcarbodiimide, N-cyclohexyl-N′-morpholinoethylcarbodiimide, N-cyclohexyl-N′-(4-diethylaminocyclohexyl)carbodiimide, N,N′-diethylcarbodiimide, N,N′-diisopropylcarbodiimide, N-ethyl-N′-(3-dimethylaminopropyl)carbodiimide and the like; N,N′-carbonylbis(2-methylimidazole); trialkyl phosphate; polyphosphorates such as ethyl polyphosphorate, isopropyl polyphosphorate and the like; phosphorus oxychloride; diphenylphosphoryl azide; thionyl chloride; oxalyl chloride; lower alkyl haloformate such as chloroethyl formate, chloroformic acid isopropyl and the like; triphenylphosphine; 1-[bis(dimethylamino)methylene]-1H-1,2,3-triazolo(4,5-b)pyridinium 3-oxide hexafluorophosphate (HATU); N-hydroxybenzotriazole; 1-(p-chlorobenzenesulfonyloxy)-6-chloro-1H-benzotriazole; N,N′-dimethylformamide, with thionyl chloride, phosgene, trichloromethyl chloroformate, phosphorus oxychloride and the like, and the like.

This reaction may be carried out in the presence of a base when desired. Examples of such base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like; organic lithiums such as methyllithium, n-butyllithium, sec-butyllithium, tert-butyllithium and the like; lithium amides such as lithium diisopropylamide and the like, and the like.

The amount of ammonia or a salt thereof to be used is generally 1 to 50 mol, preferably 1 to 10 mol, per 1 mol of compound (XXXIV). The amount of the base to be used is generally 1 to 10 mol, preferably 1 to 3 mol, per 1 mol of compound (XXXIV).

The reaction temperature is generally −30° C. to 100° C. The reaction time is generally 0.5 to 20 hr.

### Step 34

Compound (XXXVI) can be produced by subjecting compound (XXXV) to dehydrating reaction.

Examples of the dehydrating agent include chlorinating agents such as thionyl chloride, phosphoryl chloride and the like; sulfonylating agents such as methanesulfonyl chloride, methanesulfonic acid anhydride and the like; acylating agents such as acetyl chloride, acetic anhydride, trifluoroacetic anhydride and the like; cyanuric chloride and the like.

This reaction is performed without solvent or in a solvent inert to the reaction. Such solvent is not particularly limited as long as the reaction proceeds, and examples thereof include ethers such as diethyl ether, diisopropyl ether, diphenyl ether, tetrahydrofuran, 1,4-dioxane, 1,2-dimethoxyethane and the like; aromatic hydrocarbons such as benzene, toluene and the like; saturated hydrocarbons such as cyclohexane, hexane and the like; amides such as N,N-dimethylformamide, N,N-dimethylacetamide, hexamethylphosphoramide and the like; nitriles such as acetonitrile, propionitrile and the like; halogenated hydrocarbons such as dichloromethane, chloroform, 1,2-dichloroethane, carbon tetrachloride, trichloroethylene and the like; pyridine and the like. These solvents may be used in a mixture of two or more kinds thereof at an appropriate ratio.

This reaction may be carried out in the presence of a base when desired. Examples of such base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; alkali metal C₁₋₆ alkoxide such as sodium methoxide, sodium ethoxide, potassium tert-butoxide and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like; organic lithiums such as methyllithium, n-butyllithium, sec-butyllithium, tert-butyllithium and the like; lithium amides such as lithium diisopropylamide and the like, and the like.

The amount of the dehydrating agent to be used is generally 1 to 10 mol, preferably 1 to 3 mol, per 1 mol of compound (XXXV).

The amount of the base to be used is generally 1 to 10 mol, preferably 1 to 3 mol, per 1 mol of compound (XXXV).

The reaction temperature is generally −30° C. to 100° C. The reaction time is generally 0.5 to 20 hr.

### Step 35

Compound (XXXVIII) can be produced by subjecting compound (XXXVI) or compound (XXXIX) to coupling reaction with compound XXXVII) in the presence of a metal catalyst.

Examples of the metal catalyst include palladium catalysts (e.g., palladium(II) acetate, palladium acetylacetonate(0), tris(dibenzylideneacetone)dipalladium(0), bis(dibenzylideneacetone)palladium(0), tetrakis(triphenylphosphine)palladium(0), dichlorobis(triphenylphosphine)palladium(II), [1,1′-bis(diphenylphosphino)ferrocene]dichloropalladium(II) dichloromethane adduct and the like) and nickel catalysts (e.g., nickel acetylacetonate(0), dichlorobis(triphenylphosphine)nickel(0), tetrakis(triphenylphosphine)nickel(0), dichloro[1,3-bis(diphenylphosphino)propane]nickel(II), dichloro[1,4-bis(diphenylphosphino)butane]nickel(II) and the like).

This reaction may be carried out in the presence of a ligand when desired. Examples of such ligand include phosphor ligands (e.g., triphenylphosphine, 1,3-bis(diphenylphosphino)propane, 1,3-bis(diphenylphosphino)propane, 2,2′-bis(diphenylphosphino)-1,1′-binaphthyl, 4,5-bis(diphenylphosphino)-9,9-dimethylxanthene and the like).

This reaction may be carried out in the presence of a base when desired. Examples of such base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate, cesium carbonate and the like; alkali metal hydrogen carbonates such as sodium hydrogen carbonate, potassium hydrogen carbonate and the like; alkali metal alkoxides having 1 to 6 carbon atoms such as sodium methoxide, sodium ethoxide, potassium tert-butoxide and the like, and the like.

This reaction is preferably carried out in a solvent inert to the reaction. Such solvent is not particularly limited as long as the reaction proceeds, and example thereof include alcohols such as methanol, ethanol, propanol, isopropanol, butanol, tert-butanol and the like; ethers such as 1,4-dioxane, tetrahydrofuran, diethyl ether, dimethoxyethane, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; water and the like. These solvents may be used in a mixture of two or more kinds thereof at an appropriate ratio.

The amount of the base to be used is generally 1 to 10 mol, preferably 1 to 5 mol, per 1 mol of compound (XXXVI) or compound (XXXIX).

The amount of the metal catalyst to be used is generally 0.01 to 0.5 mol, preferably 0.03 to 0.1 mol, per 1 mol of compound (XXXVI) or compound (XXXIX).

The amount of the ligand to be used is generally 0.01 to 1 mol, preferably 0.05 to 0.3 mol, per 1 mol of compound (XXXVI) or compound (XXXIX).

The amount of the compound (XXXVII) to be used is generally 1 to 10 mol, preferably 1 to 3 mol, per 1 mol of compound (XXXVI) or compound (XXXIX).

The reaction temperature is generally −30° C. to 150° C., preferably 25° C. to 120° C. The reaction time is generally 0.5 to 20 hr.

### Step 36

Compound (XL) can be produced from compound (XXXIX) according to the production method of compound (XXVII) in is Reaction Scheme 11.

### Step 37

Compound (XXXVIII) can be also produced from compound (XL) and compound (XLI) according to the production method of compound (XXIX) in Reaction Scheme 11.

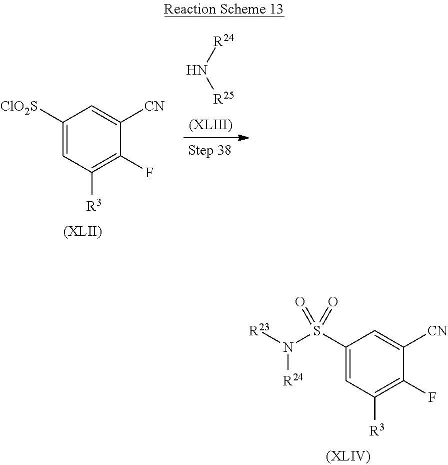

wherein R²⁴ and R²⁵ are each independently hydrogen, optionally substituted C₁₋₆ alkyl, optionally substituted C₃₋₇ cycloalkyl, optionally substituted aryl or optionally substituted heterocycle, or R²⁴ and R²⁵ in combination optionally form an optionally substituted ring, and other symbols are as defined above.

### Step 38

Compound (XLIV) can be produced by condensing compound (XLII) with compound (XLIII).

This reaction may be carried out in the presence of a base when desired. Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like; organic bases such as trimethylamine, triethylamine, diisopropylethylamine, pyridine, picoline, N-methylpyrrolidine, N-methylmorpholine, 1,5-diazabicyclo[4.3.0]-5-nonene, 1,4-diazabicyclo[2.2.2]octane, 1,8-diazabicyclo[5.4.0]-7-undecene and the like, and the like.

This reaction is preferably carried out in a solvent inert to the reaction. Such solvent is not particularly limited as long as the reaction proceeds, and examples thereof include alcohols such as methanol, ethanol, propanol, isopropanol, butanol, tert-butanol and the like; ethers such as 1,4-dioxane, tetrahydrofuran, diethyl ether, dimethoxyethane, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like. These solvents may be used in a mixture of two or more kinds thereof at an appropriate ratio.

The amount of compound (XLIII) to be used is generally 1 to 10 mol, preferably 1 to 5 mol, per 1 mol of compound (XLII).

The amount of the base to be used is generally 1 to 10 mol, preferably 1 to 5 mol, per 1 mol of compound (XLII).

The reaction temperature is generally −30° C. to 150° C., preferably 25° C. to 120° C. The reaction time is generally 0.5 to 20 hr.

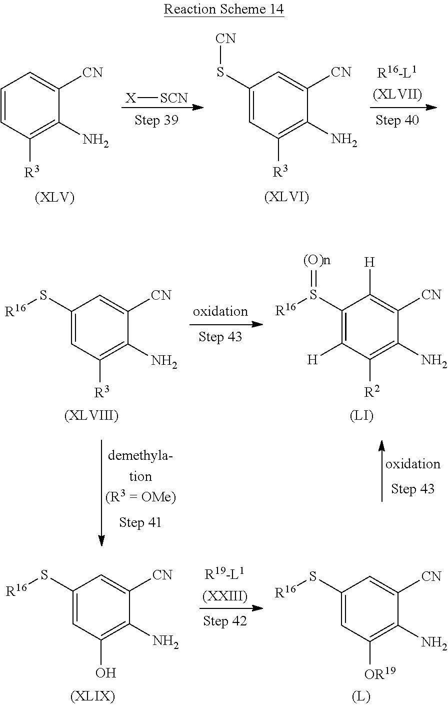

wherein X is sodium, potassium, ammonium (NH₄), trimethylsilyl and the like, and other symbols are as defined above.

### Step 39

Compound (XLVI) can be produced by reacting compound (XLV) with a thiocyanate in the presence of a halide source such as chlorine, bromine and N-bromosuccinimide.

Examples of the thiocyanic acid salt include sodium thiocyanate, potassium thiocyanate, ammonium thiocyanate, trimethylsilyl thiocyanate and the like.

Examples of the halogen source include chlorine, bromine, N-bromosuccinimide, N-chlorosuccinimide and the like.

This reaction is preferably carried out in a solvent inert to the reaction. Such solvent is not particularly limited as long as the reaction proceeds, and examples thereof include alcohols such as methanol, ethanol, propanol, isopropanol, butanol, tert-butanol and the like; ethers such as 1,4-dioxane, tetrahydrofuran, diethyl ether, dimethoxyethane, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide; water and the like. These solvents may be used in a mixture of two or more kinds thereof at an appropriate ratio.

The amount of the thiocyanic acid salt to be used is generally 1 to 10 mol, preferably 1 to 5 mol, per 1 mol of compound (XLV).

The amount of the halogen source to be used is generally 1 to 10 mol, preferably 1 to 5 mol, per 1 mol of compound (XLV).

The reaction temperature is generally −80° C. to 150° C., preferably −30° C. to 120° C. The reaction time is generally 0.5 to 20 hr.

### Step 40

Compound (XLVIII) can be produced by reacting compound (XLVI) with compound (XLVII) in the presence of a base or a metal hydrogen complex compound.

Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like, and the like.

This reaction can be performed using a metal hydrogen complex compound instead of the base. Examples of the metal hydrogen complex compound include sodium borohydride, potassium borohydride, lithium borohydride, lithium aluminum hydride, diisobutylaluminum hydride and the like.

This reaction is preferably carried out in a solvent inert to the reaction. Such solvent is not particularly limited as long as the reaction proceeds, and examples thereof include alcohols such as methanol, ethanol, propanol, isopropanol, butanol, tert-butanol and the like; ethers such as 1,4-dioxane, tetrahydrofuran, diethyl ether, dimethoxyethane, tert-butyl methyl ether, diisopropyl ether, ethylene glycol dimethyl ether and the like; esters such as ethyl formate, ethyl acetate, n-butyl acetate and the like; halogenated hydrocarbons such as dichloromethane, chloroform, carbon tetrachloride, trichloroethylene and the like; hydrocarbons such as n-hexane, benzene, toluene and the like; amides such as formamide, N,N-dimethylformamide, N,N-dimethylacetamide and the like; nitriles such as acetonitrile, propionitrile and the like; sulfoxides such as dimethyl sulfoxide and the like; sulfolane; hexamethylphosphoramide and the like. These solvents may be used in a mixture of two or more kinds thereof at an appropriate ratio.

The amount of compound (XLVII) to be used is generally 1 to 10 mol, preferably 1 to 5 mol, per 1 mol of compound (XLVI).

The amount of the base or metal hydrogen complex compound to be used is generally 1 to 10 mol, preferably 1 to 5 mol, per 1 mol of compound (XLVI).

The reaction temperature is generally −30° C. to 150° C., preferably 25° C. to 120° C. The reaction time is generally 0.5 to 20 hr.

### Step 41

Compound (XLIX) can be produced by demethylating compound (XLVIII).

Examples of the demethylation reaction agent include boron compounds such as triboron bromide, triboron bromide dimethylsulfide complex, triboron chloride and the like; Lewis acids such as aluminum chloride and the like, and the like. The Lewis acid can also be used together with a thiol or a sulfide.

This reaction is preferably carried out in a solvent inert to the reaction. Such solvent is not particularly limited as long as the reaction proceeds, and examples thereof include halogenated hydrocarbons such as dichloromethane, chloroform, carbon tetrachloride, trichloroethylene and the like; hydrocarbons such as n-hexane, benzene, toluene and the like, and the like. These solvents may be used in a mixture of two or more kinds thereof at an appropriate ratio.

The amount of the demethylation reaction agent to be used is generally 1 to 10 mol, preferably 1 to 5 mol, per 1 mol of compound (XLVIII).

The reaction temperature is generally −30° C. to 150° C., preferably 25° C. to 120° C. The reaction time is generally 0.5 to 20 hr.

### Step 42

Compound (L) can be produced from compound (XLIX) and compound (XXIII) according to the production method of compound (XXIV) in Reaction Scheme 11.

### Step 43

Compound (LI) can be produced from compound (XLVIII) or compound (L) according to the production method of compound (I-L) in Reaction Scheme 5.

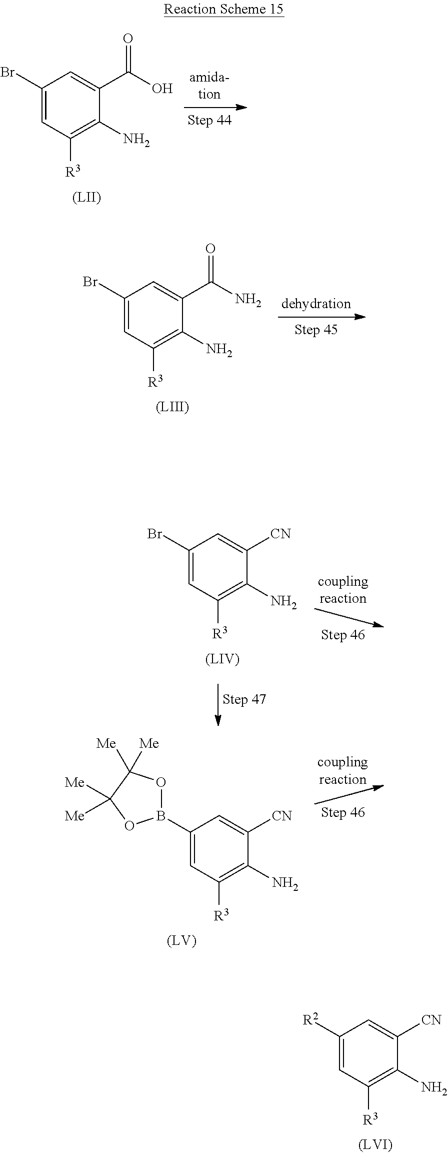

wherein each symbol is as defined above.

### Step 44

Compound (LIII) can be produced from compound (LII) according to the production method of compound (XXXV) in Reaction Scheme 12.

### Step 45

Compound (LIV) can be produced from compound (LIII) according to the production method of compound (XXXVI) in Reaction Scheme 12.

### Step 46

Compound (LVI) can be produced from compound (LIV) m according to the production method of compound (XXXVIII) in Reaction Scheme 12. Alternatively, compound (LVI) can be also produced from compound (LV) according to the production method of compound (XXIX) in Reaction Scheme 11.

### Step 47

Compound (LV) can be produced from compound (LIV) according to the production method of compound (XXVII) in Reaction Scheme 11.

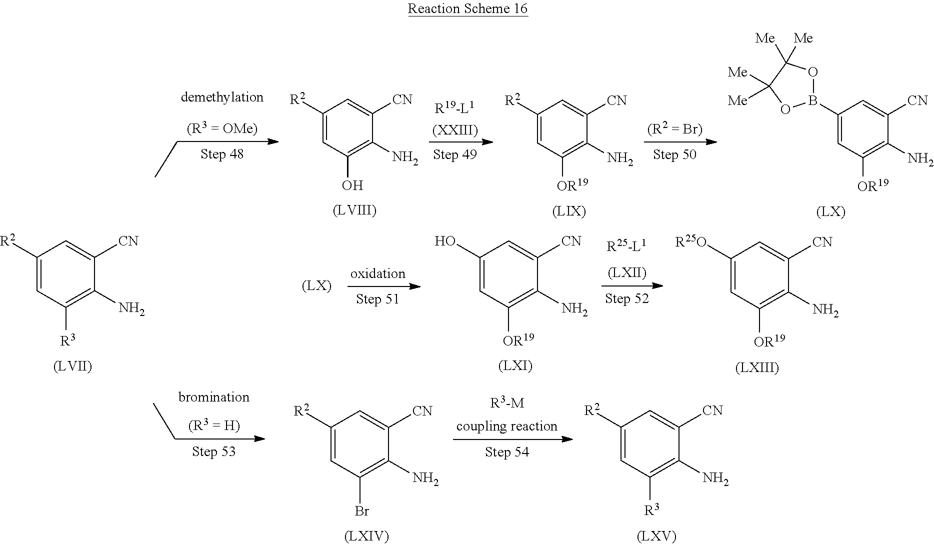

wherein R²⁵ is optionally substituted C₁₋₆ alkyl or optionally substituted C₃₋₇ cycloalkyl, and other symbols are as defined above.

### Step 48

Compound (LVIII) can be produced from compound (LVII) according to the production method of compound (XLIX) in Reaction Scheme 14.

Step 49

Compound (LIX) can be produced from compound (LVIII) and compound (XXIII) according to the production method of compound (XXIV) in Reaction Scheme 11.

### Step 50

Compound (LX) can be produced from compound (LIX) according to the production method of compound (XXVII) in Reaction Scheme 11.

### Step 51

Compound (LXI) can be produced by subjecting compound (LX) to oxidation reaction.

Examples of the oxidant include peracids such as peracetic acid, m-chloroperbenzoic acid and the like; hydrogen peroxide, sodium metaperiodate, hydroperoxide, ozone, selenium dioxide, potassium permanganate, chrome acid, iodine, bromine, N-bromosuccinimide, iodosyl benzene; sodium hypochlorite, tert-butyl hypochlorite, potassium peroxosulfate, ruthenium oxide and the like.

This reaction may be carried out in the presence of a base when desired. Examples of the base include alkali metal hydroxides such as lithium hydroxide, sodium hydroxide, potassium hydroxide and the like; alkaline earth metal hydroxides such as magnesium hydroxide, calcium hydroxide and the like; alkali metal carbonates such as sodium carbonate, potassium carbonate and the like; alkali metal hydrogen carbonates such as sodium hydrogen carbonate, potassium hydrogen carbonate and the like, and the like.

This reaction is preferably carried out in a solvent inert to the reaction. Such solvent is not particularly limited as long as the reaction proceeds, and examples thereof include alcohols such as methanol, ethanol, propanol, isopropanol, butanol, tert-butanol and the like; halogenated hydrocarbons such as dichloromethane, chloroform, carbon tetrachloride, trichloroethylene and the like; hydrocarbons such as n-hexane, benzene, toluene and the like; water and the like. These solvents may be used in a mixture of two or more kinds thereof at an appropriate ratio.

The amount of each of the base and oxidant to be used is 1 to 10 mol, preferably 1 to 3 mol, per 1 mol of compound (LX). The reaction temperature is generally −30° C. to 100° C. The reaction time is generally 0.5 to 20 hr.

### Step 52

Compound (LXIII) can be produced from compound (LXI) and compound (LXII) according to the production method of compound (XXIV) in Reaction Scheme 11.

### Step 53

Compound (LXIV) can be produced from compound (LVII) according to the production method of compound (XXVI) in Reaction Scheme 11.

### Step 54

Compound, (LXV) can be produced from compound (LXIV) according to the production method of compound (XXXVIII) in Reaction Scheme 12.

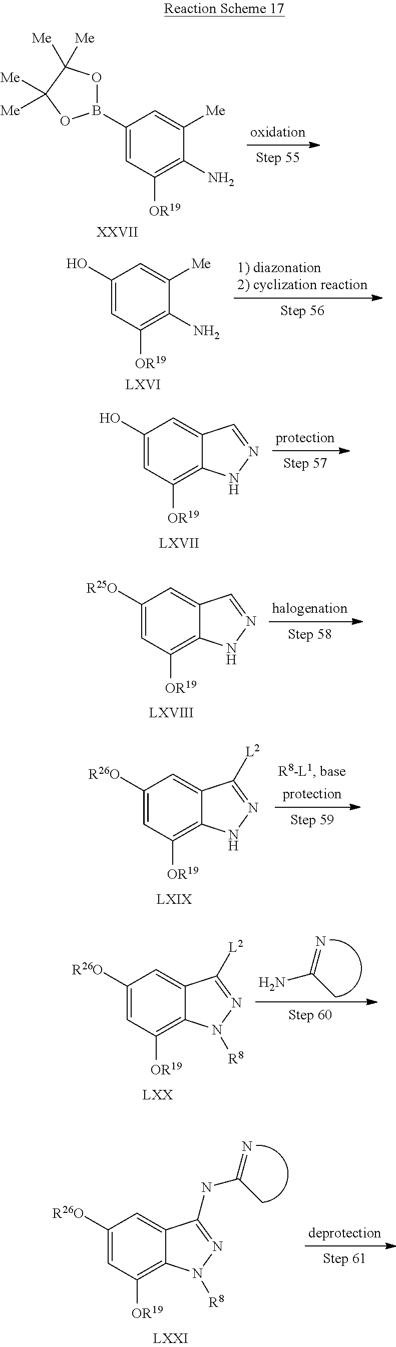

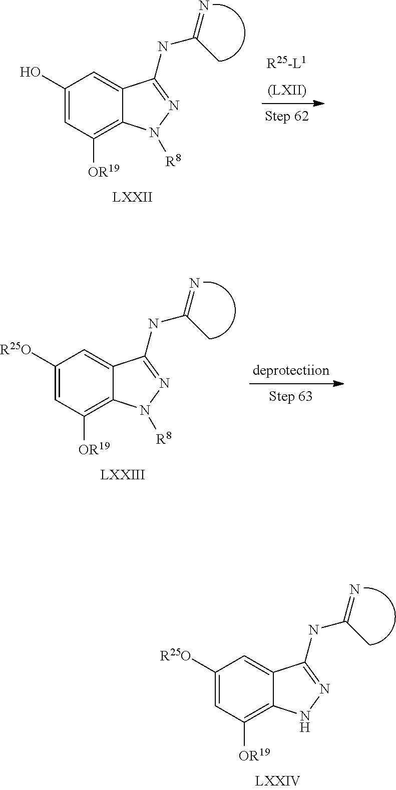

wherein R²⁶ is optionally substituted C₁₋₆ alkyl, or a protecting group (e.g., tert-butoxycarbonyl, benzyloxycarbonyl, methoxymethyl, trimethylsilylethoxymethyl, formyl, acetyl, pivaloyl, p-toluenesulfonyl, methanesulfonyl etc.) and other symbols are as defined above.

### Step 55

Compound (LXVI) can be produced from compound (XXVII) according to the production method of compound (LXI) in Reaction Scheme 16.

### Step 56

Compound (LXVII) can be produced from compound (LXVI) according to the production method of compound (XXX) in Reaction Scheme 11.

### Step 57

Compound (LXVIII) can be produced from compound (LXVII) according to a method analogous to the production method of is compound (XXI) in Reaction Scheme 10, or by introducing a protecting group.

### Step 58

Compound (LXIX) can be produced from compound (LXVIII) according to the production method of compound (XXVI) in Reaction Scheme 11.

### Step 59

Compound (LXX) can be produced from compound (LXIX) according to a method analogous to the production method of compound (XXI) in Reaction Scheme 10, or by introducing a protecting group.

### Step 60

Compound (LXXI) can be produced from compound (LXX) according to the production method of compound (VIII) in Reaction Scheme 2.

### Step 61

When R²⁶ is a protecting group (e.g., tert-butoxycarbonyl, benzyloxycarbonyl, methoxymethyl, trimethylsilylethoxymethyl, formyl, acetyl, pivaloyl, p-toluenesulfonyl etc.), compound (LXXII) can be produced by deprotection of compound (LXXI).

The reaction to eliminate a protecting group varies depending on the protecting group, and a method known per se or a method analogous thereto is used and, for example, the reaction can be performed according to the conditions described in “PROTECTIVE GROUPS IN ORGANIC SYNTHESIS” Second Edition (JOHN WILEY & SONS, INC.) and the like or in reference thereto.

### Step 62

Compound (LXXIII) can be produced from compound (LXXII) and compound (LXII) according to the production method of compound (XXIV) in Reaction Scheme 11.

### Step 63

Compound (LXXIV) can be produced from compound (LXXIII) according to the production method of compound (I-B) in Reaction Scheme 2.

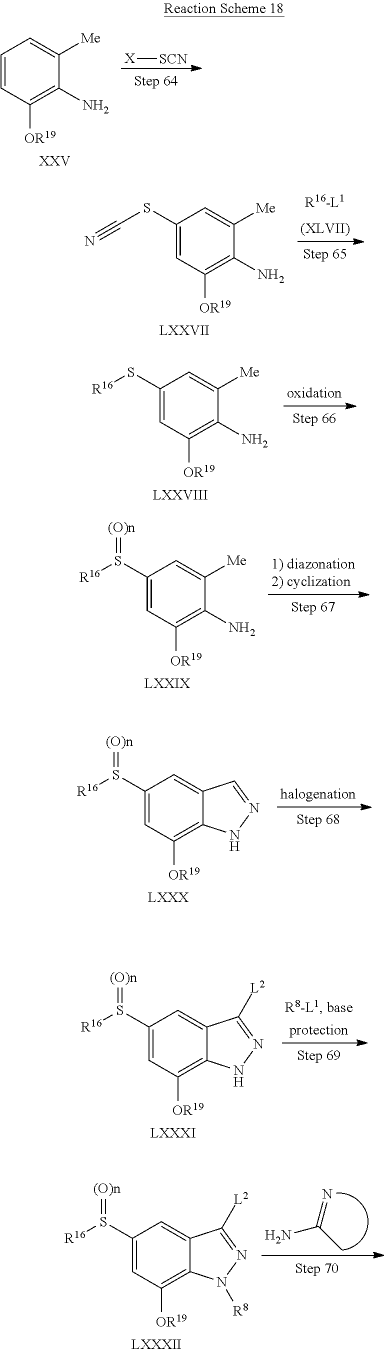

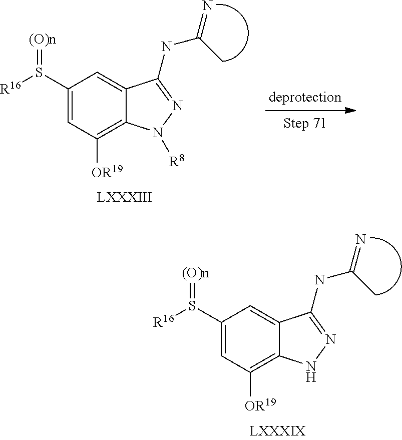

wherein each symbol is as defined above.

### Step 64

Compound (LXXVII) can be produced from compound (XXV) according to the production method of compound (XLVI) in Reaction Scheme 14.

### Step 65

Compound (LXXVIII) can be produced from compound (LXXVII) and compound (XLVII) according to the production method of compound (XLVIII) in Reaction Scheme 14.

### Step 66

Compound (LXXIX) can be produced from compound (LXXVIII) according to the production method of compound (I-L) in Reaction Scheme 5.

### Step 67

Compound (LXXX) can be produced from compound (LXXIX) according to the production method of compound (I-L) in Reaction Scheme 5.

### Step 68

Compound (LXXXI) can be produced from compound (LXXX) according to the production method of compound (XXVI) in Reaction Scheme 11.

### Step 69

Compound (LXXXII) can be produced from compound (LXXXI) according to a method analogous to the production method of compound (XXI) in Reaction Scheme 10; or by introducing a protecting group.

### Step 70

Compound (LXXXII) can be produced from compound (LXXXI) according to the production method of compound (VIII) in Reaction Scheme 2.

### Step 71

Compound (LXXXIII) can be produced from compound (LXXXII) according to the production method of compound (I-B) in Reaction Scheme 2.

The compound of the present invention obtained by each of the above-mentioned production methods can be isolated and purified by a known means such as concentration, concentration under reduced pressure, solvent extraction, crystallization, recrystallization, phase transfer, chromatography and the like. In addition, each starting material compound obtained by each of the above-mentioned production methods can be isolated and purified by a known means similar to the aforementioned means. Alternatively, such starting material compound can be used as a starting material for the next step directly without isolation or in the form of a reaction mixture.

When a starting material compound can form a salt during the production of the compound of the present invention, the compound may be used in the form of a salt. Examples of such salt include those exemplified as the salt of the compound of the present invention.

When the compound of the present invention contains an optical isomer, a stereoisomer, a regioisomer or a rotamer, these are also encompassed in the compound of the present invention, and can be obtained as a single product according to synthesis and separation methods known per se. For example, when the compound of the present invention has an optical isomer, an optical isomer resolved from this compound is also encompassed in the compound of the present invention.

The compound of the present invention may be a crystal.

The crystal of the compound of the present invention (hereinafter sometimes to be abbreviated as the crystal of the present invention) can be produced by crystallizing the compound of the present invention by a crystallization method known per se.

In the present specification, the melting point means that measured using, for example, a micromelting point apparatus (Yanako, MP-500D or Buchi, B-545) or a DSC (differential scanning calorimetry) device (SEIKO, EXSTAR6000) and the like.

In general, the melting point sometimes varies depending on the measurement device, measurement conditions and the like. The crystal of the present invention may show a different melting point from that described in the specification as long as it is within the normal error range.

The crystal of the present invention is superior in the physicochemical properties (e.g., melting point, solubility, stability) and biological properties (e.g., in vivo kinetics (absorbability, distribution, metabolism, excretion), efficacy expression), and extremely useful as a pharmaceutical agent.

### EXAMPLES

The present invention is explained in detail in the following by referring to the following Reference Examples, Examples, Experimental Examples and Formulation Examples, which are not to be construed as limitative. In addition, the present invention may be modified without departing from the scope of invention.

The term “room temperature” in the following Reference Examples and Examples indicates the range of generally from about 10° C. to about 35° C. As for “%”, the yield is in mol/mol %, the solvent used for chromatography is in % by volume and other “%” is in % by weight. OH proton, NH proton etc. on proton NMR spectrum that could not be confirmed due to broad peak are not included in the

The other symbols used herein mean the following:

s: singlet
d: doublet
t: triplet
q: quartet
m: multiplet
br: broad
J: coupling constant

### Hz: Hertz

CDCl₃: deuterated chloroform
DMSO-d₆: dimethyl sulfoxide-d₆
¹H-NMR: proton nuclear magnetic resonance
TFA: trifluoroacetic acid

In the following Reference Examples and Examples, mass spectrum (MS) and nuclear magnetic resonance spectrum (NMR) were measured under the following conditions.

MS measurement tools: Waters Corporation ZMD, Waters Corporation ZQ2000 or Micromass Ltd., platform II

Ionization method: Electron Spray Ionization (ESI) or Atmospheric Pressure Chemical Ionization (APCI). Unless specifically indicated, ESI was used.

NMR measurement tools: Varian Inc. Varian Gemini 200 (200 MHz), Varian Gemini 300 (300 MHz), Bruker BioSpin Corp. AVANCE 300.

In the following Reference Examples and Examples, purification by preparative HPLC was performed under the following conditions.

Preparative HPLC tools: Gilson, Inc., high through-put purification system
Column: YMC Combiprep ODS-A S-5 μm, 20×50 mm
Solvent: SOLUTION A; 0.1% trifluoroacetic acid-containing water,
   SOLUTION B; 0.1% trifluoroacetic acid containing-acetonitrile

Gradient cycle: 0.00 min (SOLUTION A/SOLUTION B=90/10), 1.20 min (SOLUTION A/SOLUTION B=90/10), 4.75 min (SOLUTION A/SOLUTION B=0/100), 7.30 min (SOLUTION A/SOLUTION B=0/100), 7.40 min (SOLUTION A/SOLUTION B=90/10), 7.50 min (SOLUTION A/SOLUTION B=90/10).

Flow rate: 25 ml/min, detection: UV 220 nm

### Reference Example 1

### A Construction of Glucokinase (GK) Expression Vector

Plasmid DNA to be used for the expression of a protein (GST-hLGK1) containing GST (Glutathione S-transferase) added to the amino terminal of human liver type GK in Escherichia coli was prepared as shown below.

First, PCR was performed using human liver cDNA (Clontech Laboratories, Inc. Marathon Ready cDNA) as a template and two kinds of synthetic DNAs (5′-CAGCTCTCCATCCAAGCAGCCGTTGCT-3′ and 5′-GGCGGCCTGGGTCCTGACAAG-3′). The obtained DNA fragment was cloned using a TOPO TA Cloning Kit (Invitrogen Corporation). PCR was performed using the obtained plasmid DNA as a template, and a synthetic DNA (5′-GGATCCATGCCCAGACCAAGATCCCAACTCCCACAACCCAACTCCCAGGTAGAGCA GATCCTGG CAGAG-3′) with a BamHI site added to immediately before the initiation codon and a synthetic DNA (5′-GAATTCCTGGCCCAGCATACAGGC-3′) with an EcoRI site added to immediately after the stop codon. The obtained DNA fragment was subcloned to pGEX6P-2 (Amersham Biosciences K.K.) cleaved with BamHI and EcoRI to give a plasmid (pGEX6P-2/hLGK1) for expression of human liver GK.

### Reference Example 2

### A Expression and Purification of GST-hLGK1

BL21 strain (Stratagene) transformed with pGEX6P-2/hLGK1 obtained in Reference Example 1A was cultured with shaking at 37° C. for 14 hr in a 200 ml Erlenmeyer flask containing 50 ml of 100 μg/ml ampicillin-containing LB medium. The culture medium (25 ml) was diluted with 225 ml of 100 μg/ml ampicillin-containing LB medium, and further cultured with shaking at 37° C. for 1 hr in a 1L Erlenmeyer flask. After culture, the Erlenmeyer flask was cooled on ice, 125 μL of 100 mM isopropyl-thio-β-D-galactopyranoside (IPTG) was added (final concentration 50 μM), and cultured at 17° C. for 20 hr. The culture medium was centrifuged, and the obtained fungus was disrupted by ultrasonication. The object protein (GST-hLGK1) was purified from the supernatant using Glutathione Sepharose 4B (Amersham Biosciences. K.K.).

### Reference Example 3

### A Expression and Purification of Recombinant Glucokinase

DNA encoding residues 12-465 of the full-length sequence of the human enzyme may be amplified by PCR and cloned into the HindIII and EcoRI sites of pFLAG-CTC (Sigma). SEQ.I.D. No. 1 corresponds to residues 12-465 of glucokinase.

The expression of recombinant glucokinase protein may be carried out by transformation and growth of DH10b-T1r E. coli cells incorporating the (pFLAG-CTC) plasmid in LB media. Protein expression can be induced in this system by the addition of IPTG to the culture medium.

Recombinant protein may be isolated from cellular extracts by passage over Sepharose Q Fast Flow resin (Pharmacia). This partially purified GK extract may then be further purified by a second passage over Poros HQ10 (Applied Biosystems). The purity of GK may be determined on denaturing SDS-PAGE gel. Purified GK may then be concentrated to a final concentration of 20.0 mg/ml. After flash freezing in liquid nitrogen, the proteins can be stored at −78° C. in a buffer containing 25 mM TRIS-HCl pH 7.6, 50 mM NaCl, and 0.5 mM TCEP.

### Reference Example 1

### 2-fluoro-5-(2-thienyl)benzonitrile

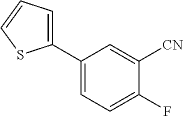

To a N,N-dimethylformamide solution (20 mL) of 5-bromo-2-fluorobenzonitrile (1.50 g) were added 2-(tributylstannyl)thiophene (4.20 g) and tetrakis(triphenylphosphine)palladium(0) (433 mg) under nitrogen stream, and the mixture was stirred at 80° C. overnight. The mixture was allowed to cool, and the reaction mixture was diluted with ethyl acetate, and washed with 1N hydrochloric acid, water and saturated brine. The mixture was dried over anhydrous magnesium sulfate, and concentrated under reduced pressure. The residue was subjected to NH-silica gel chromatography (eluate: ethyl acetate), and the obtained crude crystals were purified by recrystallization (hexane) to give the title compound (1.35 g, 89%) as colorless crystals. ¹H NMR (300 MHz, CDCl₃) δ ppm 7.11 (dd, J=5.09, 3.77 Hz, 1H) 7.19-7.32 (m, 2H) 7.36 (dd, J=5.09, 0.94 Hz, 1H) 7.73-7.92 (m, 2 H).

### Reference Example 2

### 5-(2-thienyl)-1H-indazole-3-amine

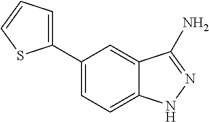

To an ethanol solution (20 mL) of 2-fluoro-5-(2-thienyl)benzonitrile (700 mg) was added hydrazine monohydrate (0.50 mL) and heated under reflux overnight. The solvent was evaporated under reduced pressure, and diluted with ethyl acetate. The organic layer was washed with water and saturated brine, dried over anhydrous magnesium sulfate, and concentrated under reduced pressure. The obtained crude crystals were purified by recrystallization (hexane-ethyl acetate) to give the title compound (638 mg, yield 86%) as colorless crystals.

MS: 216 (MH⁺).

### Reference Example 3

### N-[5-(2-thienyl)-1H-indazol-3-yl]thiourea

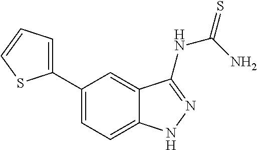

To a solution of 5-(2-thienyl)-1H-indazole-3-amine (400 mg) in tetrahydrofuran (10 mL) was added 1,1′-carbonothioyldipyridine-2(1H)-one (475 mg) at 0° C., stirred for 30 min, and concentrated aqueous ammonia (5 mL) was added. The reaction mixture was stirred for 1 hr at room temperature, water was added, and extracted with ethyl acetate. The ethyl acetate layer was washed with saturated brine, dried over anhydrous magnesium sulfate, and concentrated under reduced pressure. Purification by recrystallization (hexane-ethyl acetate) gave the title compound (383 mg, 75%) was obtained as colorless crystals. MS: 275 (MH⁺).

### Reference Example 4

### 2-fluoro-5-(3-thienyl)benzonitrile

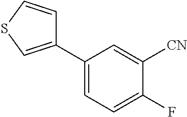

To a solution of 5-bromo-2-fluorobenzonitrile (1.0 g) in dimethoxyethane-water (20 mL-5 mL) were added 3-thiophene boronic acid (768 mg), tetrakis(triphenylphosphine)palladium(0) (289 mg) and sodium carbonate (1.06 g) under nitrogen stream, and the mixture was stirred overnight at 80° C. The mixture was allowed to cool, the reaction mixture was diluted with ethyl acetate, water and saturated brine, dried over anhydrous magnesium sulfate, and concentrated under reduced pressure. The residue was subjected to NH-silica gel column chromatography (eluate: ethyl acetate), and the obtained crystals were purified by recrystallization (hexane-ethyl acetate) to give the title compound (915 mg, yield 90%) as pale-yellow crystals. ¹H NMR (300 MHz, CDCl₃) δ ppm 7.20-7.29 (m, 1H) 7.32 (dd, J=4.62, 1.79 Hz, 1H) 7.40-7.48 (m, 2H) 7.74-7.86 (m, 2H).

### Reference Example 5

### 5-(3-thienyl)-1H-indazole-3-amine

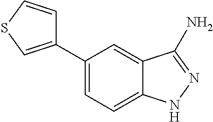

The title compound (619 mg, yield 64%) was obtained as colorless crystals from 2-fluoro-5-(3-thienyl)benzonitrile (915 mg) in the same-manner as in Reference Example 2. MS: 216 (MH⁺).

### Reference Example 6

### N-[5-(3-thienyl)-1H-indazol-3-yl]thiourea

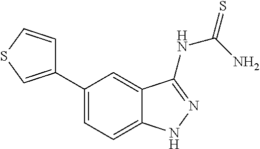

The title compound (767 mg, yield 97%) was obtained as colorless crystals from 5-(3-thienyl)-1H-indazole-3-amine (619 mg) in the same manner as in Reference Example 3. ¹H NMR (300 MHz, DMSO-d₆) δ ppm 7.47 (d, J=8.71 Hz, 1H) 7.51-7.59 (m, 1 H) 7.67 (dd, J=5.11, 2.84 Hz, 1H) 7.72-7.90 (m, 2H) 8.63 (s, 1H) 8.78 (br. s., 1H) 9.31 (br. s., 1H) 10.83 (br. s., 1H) 12.68 (br. s., 1H).

### Reference Example 7

### 5-(1,3-thiazol-2-yl)-1H-indazole-3-amine

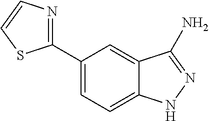

To a solution of 5-bromo-2-fluorobenzonitrile (1.0 g) in N,N-dimethylformamide (30 mL) were added 2-(tributylstannyl)thiazole (1.9 mL) and tetrakis(triphenylphosphine)palladium(0) (290 mg) under nitrogen stream, and the mixture was stirred at 80° C. overnight. The reaction mixture was allowed to cool, diluted with ethyl acetate, water and saturated brine, dried over anhydrous magnesium sulfate and concentrated under reduced pressure. The residue was subjected to NH-silica gel chromatography (eluate: ethyl acetate). The residue was brought into an ethanol solution (20 mL), hydrazine monohydrate (0.50 mL) was added, and the mixture was heated overnight under reflux. The solvent was evaporated under reduced pressure, and the residue was diluted with ethyl acetate. The organic layer was washed with water and saturated brine, dried over anhydrous magnesium sulfate, and concentrated under reduced pressure. Purification by silica gel column chromatography (hexane:ethyl acetate=1:1-0:1) gave the title compound (27.3 mg, yield 18%) as colorless crystals. ¹H NMR (300 MHz, DMSO-d₆) δ ppm 5.60 (s, 2H) 7.31 (d, J=8.71 Hz, 1H) 7.65 (d, J=3.41 Hz, 1H) 7.74-7.94 (m, 2H) 8.39 (s, 1H) 11.65 (br. s., 1H).

### Reference Example 8

### N-[5-(1,3-thiazol-2-yl)-1H-indazol-3-yl]thiourea

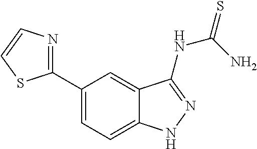

The title compound (29.1 mg, yield 84%) was obtained as colorless crystals from 5-(1,3-thiazol-2-yl)-1H-indazole-3-amine (27.3 mg) in the same manner as in Reference Example 3.

MS: 276 (MH⁺).

### Reference Example 9

### 2-fluoro-5-(1-methyl-1H-pyrazol-5-yl)benzonitrile

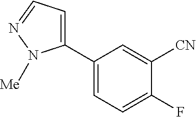

To a tetrahydrofuran solution (30 mL) of diisopropylamine (3.9 mL) was added dropwise n-butyllithium (1.6 M hexane solution, 17 mL) under ice-cooling, and the mixture was stirred for 1 hr. The reaction mixture was cooled to −78° C., and 1-methylpyrazole (2.1 mL) was added dropwise. The mixture was stirred at −78° C. for 1 hr, zinc chloride (0.5M tetrahydrofuran solution, 55 mL) was added dropwise, and the mixture was returned to room temperature and stirred for 1 hr. Tetrakis(triphenylphosphine)palladium(0) (580 mg) and 5-bromo-2-fluorobenzonitrile (2.0 g) were sequentially added, and the mixture was stirred at 60° C. overnight. The mixture was allowed to cool, water was added, insoluble materials were filtered, and the filtrate was diluted with ethyl acetate. The organic layer was washed water and saturated brine, dried over anhydrous magnesium sulfate, and concentrated under reduced pressure. The residue was purified by silica gel column chromatography (hexane:ethyl acetate=10:1-2:1) to give the title compound (95.1 mg, 4.7%) as colorless crystals. ¹H NMR (300 MHz, CDCl₃) δ ppm 3.89 (s, 3H) 6.33 (d, J=1.89 Hz, 1H) 7.34 (t, J=8.52 Hz, 1H) 7.54 (d, J=1.89 Hz, 1H) 7.58-7.75 (m, 2H).

### Reference Example 10

### 5-(1-methyl-1H-pyrazol-5-yl)-1H-indazole-3-amine

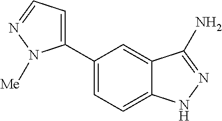

The title compound (51.9 mg, yield 51%) was obtained as colorless crystals from 2-fluoro-5-(1-methyl-1H-pyrazol-5-yl)benzonitrile (95.1 mg) in the same manner as in Reference Example 2. MS: 214 (MH⁺).

### Reference Example 11

### N-[5-(1-methyl-1H-pyrazol-5-yl)-1H-indazol-3-yl] thiourea

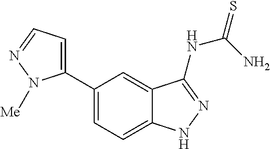

The title compound (29.3 mg, yield 42%) was obtained as colorless crystals from 5-(1-methyl-1H-pyrazol-5-yl)-1H-indazole-3-amine (51.9 mg) in the same manner as in Reference Example 3. MS: 273 (MH⁺).

### Reference Example 12

### 5-(3-chloropyridin-2-1)-2-fluorobenzonitrile

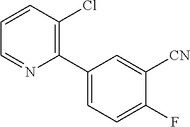

To a solution of 2,3-dichloropyridine (3.51 g) in dimethoxyethane (100 mL) were added (3-cyano-4-fluorophenyl)boronic acid (2.6 g), tetrakis(triphenylphosphine)palladium(0) (913 mg) and 2M aqueous sodium carbonate solution (20 mL) under nitrogen stream, and the mixture was stirred at 80° C. overnight. The reaction mixture was allowed to cool, diluted with ethyl acetate, water and saturated brine, dried over anhydrous magnesium sulfate and concentrated under reduced pressure. The residue was subjected to NH-silica gel column chromatography (eluate: ethyl acetate). The residue was purified by recrystallization (ethyl acetate-diisopropyl ether) to give the title compound (2.56 g, yield 70%) as pale-yellow crystals.

MS: 233 (MH⁺).

### Reference Example 13

### 5-(3-chloropyridin-2-yl)-1H-indazole-3-amine

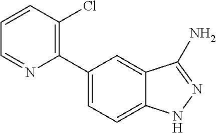

The title compound (2.20 g, yield 82%) was obtained as colorless crystals from 5-(3-chloropyridin-2-yl)-2-fluorobenzonitrile (2.56 g) in the same manner as in Reference Example 2. MS: 245 (MH⁺).

### Reference Example 14

### N-(5-(3-chloropyridin-2-yl)-1H-indazol-3-yl)thiourea

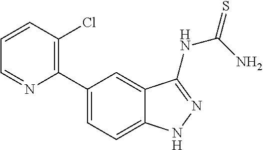

The title compound (562 mg, yield 91%) was obtained as colorless crystals from 5-(3-chloropyridin-2-yl)-1H-indazole-3-amine (0.50 g) in the same manner as in Reference Example 3.

MS: 304 (MH⁺).

### Reference Example 15

### 2-fluoro-5-(1-methyl-1H-pyrazol-4-yl)benzonitrile

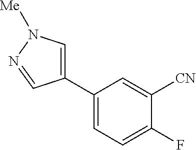

The title compound (1.79 g, yield 100%) was obtained as colorless crystals from 5-bromo-2-fluorobenzonitrile (1.76 g) and 1-methyl-4-(4,4,5,5-tetramethyl-1,3,2-dioxaborolan-2-yl)-1H-pyrazole (2.2 g) in the same manner as in Reference Example 4. MS: 202 (MH⁺).

### Reference Example 16

### 5-(1-methyl-1H-pyrazol-4-yl)-1H-indazole-m 3-amine

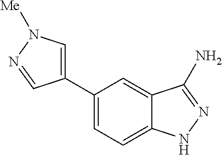

The title compound (347 mg, yield 19%) was obtained as pale-yellow crystals from 2-fluoro-5-(1-methyl-1H-pyrazol-4-yl)benzonitrile (1.79 g) in the same manner as in Reference Example 2. MS: 214 (MH⁺).

### Reference Example 17

### N-[5-(1-methyl-1H-pyrazol-4-yl)-1H-indazol-3-yl]thiourea

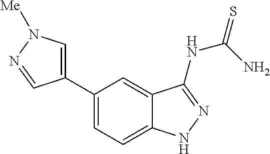

The title compound (327 mg, yield 74%) was obtained as pale-yellow crystals from 5-(1-methyl-1H-pyrazol-4-yl)-1H-indazole-3-amine (347 mg) in the same manner as in Reference Example 3. MS: 273 (MH⁺).

### Reference Example 18

### 2′-chloro-4-fluorobiphenyl-3-carbonitrile

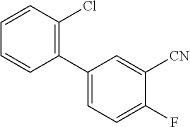

The title compound (790 mg, yield 68%) was obtained as colorless crystals from 5-bromo-2-fluorobenzonitrile (1.0 g) and (2-chlorophenyl)boronic acid (1.18 g) in the same manner as in Reference Example 4. MS: 232 (MH⁺).

### Reference Example 19

### 5-(2-chlorophenyl)-1H-indazole-3-amine

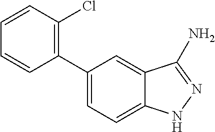

The title compound (228 mg, yield 27%) was obtained as pale-yellow crystals from 2′-chloro-4-fluorobiphenyl-3-carbonitrile (790 mg) in the same manner as in Reference Example 2. MS: 244 (MH⁺).

### Reference Example 20

### N-[5-(2-chlorophenyl)-1H-indazol-3-yl]thiourea

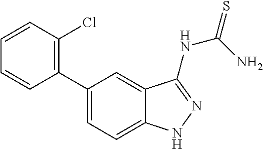

The title compound (215 mg, yield 76%) was obtained as pale-yellow crystals from 5-(2-chlorophenyl)-1H-indazole-3-amine (228 mg) in the same manner as in Reference Example 3.

¹H NMR (300 MHz, DMSO-d₆) δ ppm 7.36-7.55 (m, 5H) 7.56-7.61 (m, 1H) 8.35 (s, 1H) 8.77 (br. s., 1H) 9.30 (br. s., 1H) 10.92 (s, 1H) 12.78 (s, 1H).

### Reference Example 21

### 2-fluoro-5-(3-methylpyridin-2-yl)benzonitrile

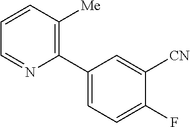

The title compound (343 mg, yield 53%) was obtained as pale-yellow crystals from (3-cyano-4-fluorophenyl)boronic acid (0.50 g) and 2-bromo-3-methylpyridine (0.51 mL) in the same manner as in Reference Example 12. MS: 213 (MH⁺).

### Reference Example 22

### 5-(3-methylpyridin-2-yl)-1H-indazole-3-amine

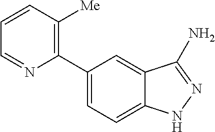

The title compound (256 mg, yield 70%) was obtained as pale-yellow crystals from 2-fluoro-5-(3-methylpyridin-2-yl)benzonitrile (343 mg) in the same manner as in Reference Example 2. MS: 225 (MH⁺).

### Reference Example 23

### N-[5-(3-methylpyridin-2-yl)-1H-indazol-3-yl]thiourea

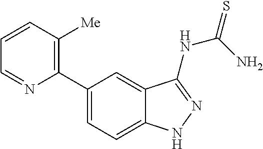

The title compound (260 mg, yield 80%) was obtained as pale-yellow crystals from 5-(3-methylpyridin-2-yl)-1H-indazole-3-amine (256 mg) in the same manner as in Reference Example 3.

MS: 284(MH⁺).

### Reference Example 24

### 5-(3-fluoropyridin-2-yl)-1H-indazole-3-amine

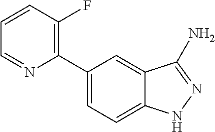

To a solution of 2-chloro-3-fluoropyridine (453 mg) in dimethoxyethane (20 mL) were added (3-cyano-4-fluorophenyl)boronic acid (0.50 g), tetrakis(triphenylphosphine)palladium(0) (263 mg) and 2M aqueous sodium carbonate solution (4.6 mL) under nitrogen stream, and the mixture was stirred overnight at 80° C. The reaction mixture was allowed to cool, diluted with ethyl acetate, water and saturated brine, dried over anhydrous magnesium sulfate, and concentrated under reduced pressure. The residue was subjected to NH-silica gel column chromatography (eluate: ethyl acetate). The obtained crude crystals were recrystallized (ethyl acetate-diisopropyl ether), and the obtained crystals were brought into n-butanol solution (10 mL), hydrazine monohydrate (0.22 mL) was added, and heated overnight under reflux. The solvent was evaporated under reduced pressure, and the residue was diluted with ethyl acetate. The organic layer was washed with water and saturated brine, dried over anhydrous magnesium sulfate, and concentrated under reduced pressure. The obtained crude crystals were purified by recrystallization (ethyl acetate-diisopropyl ether) to give the title compound (152 mg, yield 22%) as colorless crystals. MS: 229 (MH⁺).

### Reference Example 25

### N-[5-(3-fluoropyridin-2-yl)-1H-indazol-3-yl]thiourea

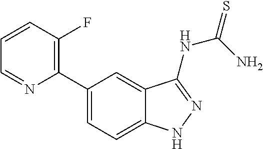

The title compound (162 mg, yield 85%) was obtained as pale-yellow crystals from 5-(3-fluoropyridin-2-yl)-1H-indazole-3-amine (152 mg) in the same manner as in Reference Example 3.

MS: 288 (MH⁺).

### Reference Example 26

### 5-(3,5-dimethyl-1H-pyrazol-1-yl)-2-fluorobenzonitrile

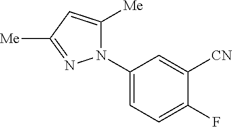

To a solution of (3-cyano-4-fluorophenyl)boronic acid (0.50 g) in N,N-dimethylformamide (10 mL) were added 3,5-dimethyl-1H-pyrazole (438 mg), copper (II) acetate (1.1 g) and pyridine (0.91 mL), and the mixture was stirred overnight at room temperature. The insoluble materials were filtered, and the filtrate was diluted with ethyl acetate, washed with water and saturated brine, dried over anhydrous magnesium sulfate, and concentrated under reduced pressure. The residue was purified by silica gel chromatography (hexane-ethyl acetate=10:1) to give the title compound (105 mg, yield 16%) as pale-yellow crystals. MS: 216 (MH⁺).

### Reference Example 27

### 5-(3,5-dimethyl-1H-pyrazol-1-yl)-1H-indazole-3-amine

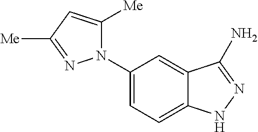

The title compound (66 mg, yield 61%) was obtained as non-crystalline powder from 5-(3,5-dimethyl-1H-pyrazol-1-yl)-2-fluorobenzonitrile (103 mg) in the same manner as in Reference Example 2. MS: 228 (MH⁺).

### Reference Example 28

### N-[5-(3,5-dimethyl-1H-pyrazol-1-yl)-1H-indazol-3-yl]thiourea

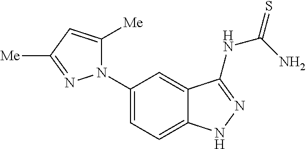

The title compound (65.7 mg, yield 85%) was obtained from 5-(3,5-dimethyl-1H-pyrazol-1-yl)-1H-indazole-3-amine (61.2 mg) as pale-yellow crystals in the same manner as in Reference Example 3. MS: 287 (MH⁺).

### Reference Example 29

### 5-(1-methyl-1H-imidazol-2-yl)-1H-indazole-3-amine

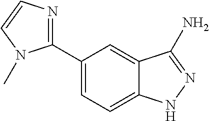

The title compound (26.5 mg, yield 3%) was obtained as pale-yellow crystals from (3-cyano-4-fluorophenyl)boronic acid (667 mg) and 2-bromo-1-methyl-1H-imidazole (781 mg) in the same manner as in Reference Example 24. MS: 214 (MH⁺)

### Reference Example 30

### N-[5-(1-methyl-1H-imidazol-2-yl)-1H-indazol-3-yl]thiourea

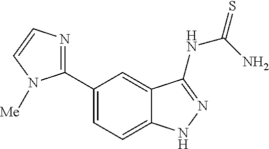

The title compound (37.7 mg, yield 100%) was obtained as colorless crystals from 5-(1-methyl-1H-imidazol-2-yl)-1H-indazole-3-amine (26.5 mg) in the same manner as in Reference Example 3. MS: 273 (MH⁺).

### Reference Example 31

### 5-(4-chloropyridin-3-yl)-2-fluorobenzonitrile

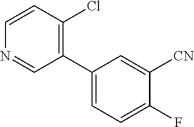

The title compound (300 mg, yield 40%) was obtained as colorless crystals from (3-cyano-4-fluorophenyl)boronic acid (640 mg) and 3-bromo-4-chloropyridine (0.62 g) in the same manner as in Reference Example 12. MS: 233 (MH⁺).

### Reference Example 32

### 5-(4-chloropyridin-3-yl)-1H-indazole-3-amine

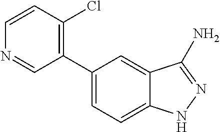

The title compound (164 mg, yield 52%) was obtained as colorless crystals from 5-(4-chloropyridin-3-yl)-2-fluorobenzonitrile (300 mg) in the same manner as in Reference Example 2; Melting point>285° C.

### Reference Example 33

### N-[5-(4-chloropyridin-3-yl)-1H-indazol-3-yl]thiourea

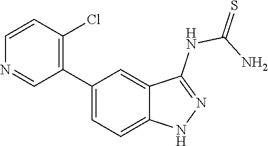

The title compound (107 mg, yield 56%) was obtained as colorless crystals from 5-(4-chloropyridin-3-yl)-1H-indazole-3-amine (155 mg) in the same manner as in Reference Example 3. Melting point>285° C.

### Reference Example 34

### 2-(3-cyano-4-fluorophenyl)nicotinonitrile

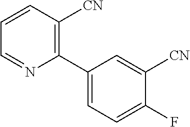

The title compound (833 mg, yield 60%) was obtained as colorless crystals from (3-cyano-4-fluorophenyl)boronic acid (1.02 g) and 2-chloronicotinonitrile (0.863 g) in the same manner as in Reference Example 12. Melting point 127-128° C.

### 2-(3-Amino-1H-indazol-5-yl)nicotinonitrile

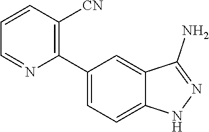

The title compound (144 mg, yield 16%) was obtained as pale-yellow crystals from 2-(3-cyano-4-fluorophenyl)nicotinonitrile (833 mg) in the same manner as in Reference Example 2. Melting point 203-204° C.

### Reference Example 35

### N-[5-(3-cyanopyridin-2-yl)-1H-indazol-3-yl]thiourea

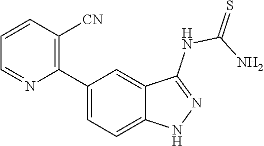

The title compound (89 mg, yield 50%) was obtained as colorless crystals from 5-(4-chloropyridin-3-yl)-1H-indazole-3-amine (144 mg) in the same manner as in Reference Example 3. Melting point 279-280° C.

### Reference Example 36

### tert-butyl 2-(3-cyano-4-fluorophenyl)nicotinate

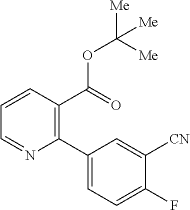

The title compound (3.12 g, yield 100%) was obtained as pale-yellow crystals from (3-cyano-4-fluorophenyl)boronic acid (1.74 g) and tert-butyl 2-chloronicotinate (2.47 g) in the same manner as in Reference Example 12. MS: 299 (MH⁺).

### Reference Example 37

### tert-butyl 2-(3-amino-1H-indazol-5-yl)nicotinate

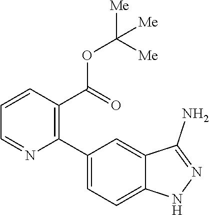

The title compound (410 mg, yield 49%) was obtained as pale-yellow crystals from tert-butyl 2-(3-cyano-4-fluorophenyl)nicotinate (798 mg) in the same manner as in Reference Example 2. Melting point 190-191° C.

### Reference Example 38

### tert-butyl-2-{3-[(aminocarbonothioyl)amino]-1H-indazol-5-yl}nicotinate

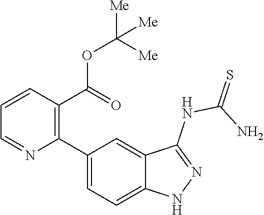

The title compound (453 mg, yield 94%) was obtained as colorless crystals from tert-butyl yl)nicotinate (406 mg) in the same manner as in Reference Example 3. Melting point 227-230° C.

### Reference Example 39

### 2-fluoro-5-pyridin-2-ylbenzonitrile

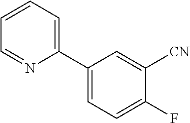

The title compound (95 mg, yield 10%) was obtained as colorless oil from 3-bromo-5-fluorobenzonitrile (945 mg) and 2-(tributylstannyl)pyridine (1.91 g) in the same manner as in Reference Example 1. ¹H NMR (300 MHz, CDCl₃) δ ppm 7.28-7.37 (m, 2H) 7.67-7.73 (m, 1H) 7.76-7.86 (m, 1H) 8.21-8.34 (m, 2H) 8.68-8.74 (m, 1H).

### Reference Example 40

### 5-pyridin-2-yl-1H-indazole-3-amine

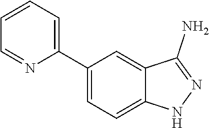

The title compound (41.8 mg, yield 42%) was obtained as colorless crystals from 2-fluoro-5-pyridin-2-ylbenzonitrile (95 mg) in the same manner as in Reference Example 2. ¹H NMR (300 MHz, DMSO-d₆) δ ppm 5.48 (s, 2H) 7.18-7.41 (m, 2H) 7.73-7.93 (m, 2H) 8.00 (dd, J=8.76, 1.60 Hz, 1H) 8.50 (s, 1H) 8.62 (d, J=4.52 Hz, 1H) 11.50 (s, 1H).

### Reference Example 41

### 5-[3-(2,5-dimethyl-1H-pyrrol-1-yl)pyridin-2-yl]-2-fluorobenzonitrile

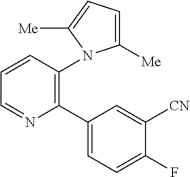

To a solution of 2-chloro-3-aminopyridine (585 mg) in dimethoxyethane (20 mL) were added (3-cyano-4-fluorophenyl)boronic acid (0.50 g), tetrakis(triphenylphosphine)palladium(0) (263 mg), 2M aqueous sodium carbonate solution (4.6 mL) under nitrogen stream, and the mixture was stirred overnight at 80° C. The reaction mixture was allowed to cool, diluted with ethyl acetate, washed with water and saturated brine, dried over anhydrous magnesium sulfate and concentrated under reduced pressure. The residue was subjected to NH-silica gel column chromatography (eluate: ethyl acetate). The obtained powder was collected and washed with isopropyl ether. To a toluene solution (15 mL) of this powder were added acetic acid (0.19 mL) and 2,5-hexanedione (0.32 mL), and the mixture was heated overnight under reflux in a reaction container attached to a Dean-Stark and diluted with ethyl acetate. The organic layer was washed with water and saturated brine, dried over anhydrous magnesium sulfate and concentrated under reduced pressure. The residue was purified by silica gel column chromatography (hexane:ethyl acetate=5:1) to give the title compound (307 mg, yield 35%) as pale-yellow crystals. ¹H NMR (300 MHz, CDCl₃) δ ppm 1.83 (6H, s) 5.96 (2H, s) 7.10 (1H, t, J=8.7 Hz) 7.28-7.38 (1H, m) 7.39-7.52 (2H, m) 7.69 (1H, dd, J=8.0, 1.5 Hz) 8.78 (1H, dd, J=4.5, 1.5 Hz).

### Reference Example 42

### 5-[3-(2,5-dimethyl-1H-pyrrol-1-yl)pyridin-2-yl]-1H-indazole-3-amine

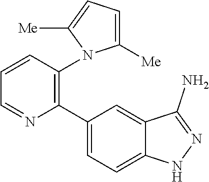

The title compound (209 mg, yield 66%) was obtained as colorless crystals from 5-[3-(2,5-dimethyl-1H-pyrrol-1-yl)pyridin-2-yl]-2-fluorobenzonitrile (307 mg) in the same manner as in Reference Example 2. Melting point 186-189° C.

### Reference Example 43

### N-{5-[3-(2,5-dimethyl-1H-pyrrol-1-yl)pyridin-2-yl]-1H-indazol-3-yl}thiourea

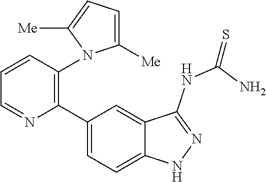

The title compound (234 mg, yield 94%) was obtained as colorless crystals from 5-[3-(2,5-dimethyl-1H-pyrrol-1-yl)pyridin-2-yl]-1H-indazole-3-amine (209 mg) in the same manner as in Reference Example 3. Melting point 236-238° C.

### Reference Example 44

### 3-cyano-4-fluoro-N-(pyridin-2-ylmethyl)benzenesulfonamide

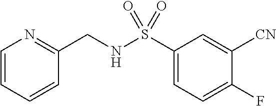

To a tetrahydrofuran solution (20 mL) of 3-cyano-4-fluorobenzenesulfonyl chloride (1.0 g) were added triethylamine (0.77 mL) and 2-picolylamine (0.52 mL), and the mixture was stirred at room temperature for 1 hr. The mixture was diluted with ethyl acetate, and the organic layer was washed with water and saturated brine, dried over anhydrous magnesium sulfate, and reduced pressure. The obtained crude crystals were purified by recrystallization (hexane-ethyl acetate) to give the title compound (1.21 g, yield 92%) as colorless crystals. MS: 292 (MH⁺).

### Reference Example 45

### 3-amino-N-(pyridin-2-ylmethyl)-1H-indazole-5-sulfonamide

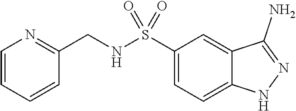

The title compound (1.07 g, yield 85%) was obtained as colorless crystals from 3-cyano-4-fluoro-N-(pyridin-2-ylmethyl)benzenesulfonamide (1.21 g) in the same manner as in Reference Example 2. ¹H NMR (300 MHz, DMSO-d₆) δ ppm 3.93-4.19 (2H, m) 5.70 (2H, s) 7.22 (1H, dd, J=6.4, 4.9 Hz) 7.36 (2H, t, J=8.9 Hz) 7.61 (1H, dd, J=8.9, 1.7 Hz) 7.66-7.80 (1 H, m) 8.00 (1H, br. s.) 8.32 (1H, s) 8.42 (1H, d, J=4.2 Hz) 11.87 (1H, s)

### Reference Example 46

### 3-[(aminocarbonothioyl)amino]-N-(pyridin-2-ylmethyl)-1H-indazole-5-sulfonamide

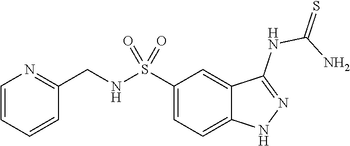

The title compound (493 mg, yield 38%) was obtained as colorless crystals from 3-amino-N-(pyridin-2-ylmethyl)-1H-indazole-5-sulfonamide (1.07 g) in the same manner as in Reference Example 3. MS: 363 (MH⁺).

### Reference Example 47

### 2-fluoro-5-(pyrrolidin-1-ylsulfonyl)benzonitrile

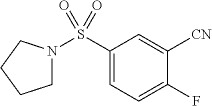

The title compound (1.06 g, yield 92%) was obtained as colorless crystals from 3-cyano-4-fluorobenzenesulfonyl chloride (1.0 g) and pyrrolidine (0.42 mL) in the same manner as in Example 44. MS: 255(MH⁺).

### Reference Example 48

### 5-(pyrrolidin-1-ylsulfonyl)-1H-indazole-3-amine

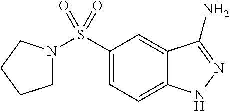

The title compound (947 mg, yield 85%) was obtained as colorless crystals from 2-fluoro-5-(pyrrolidin-1-ylsulfonyl)benzonitrile (1.06 g) in the same manner as in Reference Example 2. ¹H NMR (300 MHz, DMSO-d₆) S ppm 1.38-1.77 (4H, m) 2.95-3.23 (4H, m) 5.75 (2H, s) 7.38 (1H, d, J=8.3 Hz) 7.59 (1H, dd, J=8.7, 1.9 Hz) 8.33 (1H, s) 11.91 (1H, br. s.)

### Reference Example 49

### N-[5-(pyrrolidin-1-ylsulfonyl)-1H-indazol-3-yl]thiourea

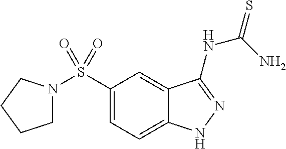

The title compound (252 mg, yield 22%) was obtained as colorless crystals from 5-(pyrrolidin-1-ylsulfonyl)-1H-indazole-3-amine (947 mg) in the same manner as in Reference Example 3. ¹H NMR (300 MHz, DMSO-d₆) δ ppm 1.51-1.73 (4H, m) 3.01-3.22 (4H, m) 7.52-7.68 (1H, m) 7.75 (1H, dd, J=8.9, 1.7 Hz) 8.66-9.07 (2H, m) 9.20 (1H, br. s.) 11.13 (1H, br. s.) 13.14 (1H, br. s.)

### Reference Example 50

### 3-bromo-2-fluorobenzamide

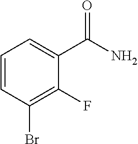

To a solution of 2-fluoro-3-bromobenzoic acid (1.36 g) in N,N-dimethylformamide (5 mL) were added 1-ethyl-3-(3-dimethylaminopropyl)carbodiimide hydrochloride (1.80 g) and ammonium 1-hydroxybenzotriazole (1.43 g), and the mixture was stirred overnight at room temperature. The mixture was diluted with ethyl acetate, saturated aqueous sodium hydrogen carbonate, washed with water and saturated brine, dried over anhydrous magnesium sulfate and concentrated under reduced pressure to give the title compound (867 mg, yield 64%) as colorless oil. ¹H NMR (300 MHz, CDCl₃) δ ppm 5.85 (1H, br. s.) 6.61 (1H, br. s.) 7.10-7.23 (1H, m) 7.64-7.81 (1H, m) 7.99-8.17 (1H, m)

### Reference Example 51

### 3-bromo-2-fluorobenzonitrile

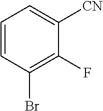

To a N,N-dimethylformamide solution (10 mL) of 3-bromo-2-fluorobenzamide (867 mg) was added cyanuric chloride (806 mg) under ice-cooling, and the mixture was stirred overnight at room temperature. The mixture was diluted with ethyl acetate, and the organic layer was washed with water and saturated brine, dried over anhydrous magnesium sulfate, and concentrated under reduced pressure. Purification by silica gel column chromatography (hexane:ethyl acetate=1:0-1:1) gave the title compound (760 mg, yield 96%) as colorless crystals. ¹H NMR (300 MHz, CDCl₃) δ ppm 7.17 (1H, t, J=8.5 Hz) 7.54-7.66 (1H, m) 7.76-7.88 (1H, m)

### Reference Example 52

### 2-fluoro-3-pyridin-4-ylbenzonitrile

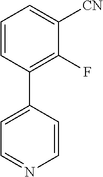

The title compound (568 mg, yield 77%) was obtained as colorless crystals from 3-bromo-2-fluorobenzonitrile (744 mg) and pyridin-4-yl boronic acid (550 mg) in the same manner as in Reference Example 4. MS: 199 (MH⁺).

### Reference Example 53

### 7-pyridin-4-yl-1H-indazole-3-amine

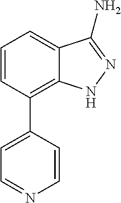

The title compound (240 mg, yield 40%) was obtained as colorless crystals from 2-fluoro-3-pyridin-4-ylbenzonitrile (568 mg) in the same manner as in Reference Example 2.

MS: 211 (MH⁺).

### Reference Example 54

### N-(7-pyridin-4-yl-1H-indazol-3-s yl)thiourea

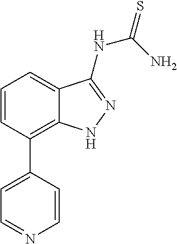

The title compound (280 mg, yield 91%) was obtained as colorless crystals from 7-pyridin-4-yl-1H-indazole-3-amine (240 mg) in the same manner as in Reference Example 3. ¹H NMR (300 MHz, DMSO-d₆) δ ppm 7.25 (1H, t, J=7.6 Hz) 7.59 (1H, d, J=7.2 Hz) 7.71 (2H, d, J=5.7 Hz) 8.31 (1H, d, J=8.3 Hz) 8.60-8.96 (3H, m) 9.28 (1H, br. s.) 10.93 (1H, br. s.) 12.78 (1H, br. s.)

### Reference Example 55

### 3-bromo-2-fluoro-5-propylbenzoic acid

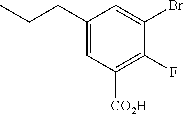

To a mixed solution of dimethyl sulfoxide (100 mL) and tetrahydrofuran (200 ml) was added sodium hydride (60%, 4.32 g) at room temperature. The mixture was stirred at 50° C. for 1.5 hr and cooled with ice, and ethyl triphenylphosphonium bromide (36.6 g) was added. The mixture was stirred at room temperature for 30 min, and dimethylsulfoxide solution (50 mL) of 3-bromo-4-fluorobenzaldehyde (10 g) was added. The reaction mixture was stirred while heating under reflux for 1.5 hr, and 1N hydrochloric acid (150 mL) was added under ice-cooling. The aqueous layer was extracted with ethyl acetate, and the organic layer was washed with saturated brine, dried over anhydrous magnesium sulfate, and concentrated under reduced pressure. The residue was subjected to silica gel column chromatography (eluate: hexane). The residue was dissolved in ethanol (500 mL), platinum oxide (200 mg) was added, and the mixture was stirred overnight under hydrogen atmosphere. The insoluble materials were filtered, and the mother liquor was concentrated under reduced pressure to give a residue (6.71 g).

A tetrahydrofuran solution (10 mL) of this residue (1.0 g) was added dropwise to lithium diisopropylamide (1.8M heptane-tetrahydrofuran-ethylbenzene solution, 3.1 mL) cooled to −78° C., and the mixture was stirred for 1 hr at room temperature. Carbon dioxide gas was blown for 1 hr, and the mixture was stirred for 1 hr at room temperature. To the reaction mixture was added 1N hydrochloric acid, and the aqueous layer was extracted with ethyl acetate. The organic layer was extracted twice with 1N aqueous sodium hydroxide solution (50 mL). The aqueous layer was acidified with 1N hydrochloric acid, and extracted with ethyl acetate. The organic layers were combined, washed with saturated brine, dried over anhydrous magnesium sulfate, and concentrated under reduced pressure to give the title compound (400 mg) as colorless oil.

¹H NMR (CDCl₃) δ ppm 0.95 (3H, t, J=7.4 Hz), 1.50-1.70 (2H, m), 2.50-2.62 (2H, m), 7.60 (1H, dd, J=6.0, 2.2 Hz), 7.74 (1H, dd, J=6.0, 2.2 Hz).

### Reference Example 56

### 3-bromo-2-fluoro-5-propylbenzamide

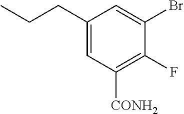

The title compound (260 mg, yield 67%) was obtained as colorless crystals from 3-bromo-2-fluoro-5-propylbenzoic acid (400 mg) in the same manner as in Reference Example 50. Melting point 158-159° C.

### Reference Example 57

### 3-bromo-2-fluoro-5-propylbenzonitrile

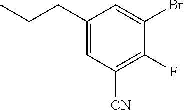

The title compound (160 mg, yield 84%) was obtained as colorless oil from 3-bromo-2-fluoro-5-propylbenzamido (200 mg) in the same manner as in Reference Example 51. ¹H NMR (CDCl₃) δ ppm. 0.95 (3H, t, J=7.4 Hz), 1.50-1.70 (2H, m), 2.56-2.61 (2H, m), 7.36 (1H, dd, J=5.2, 2.0 Hz), 7.61 (1H, dd, J=6.4, 2.0 Hz).

### Reference Example 58

### 2-fluoro-5-propyl-3-(2-thienyl)benzonitrile

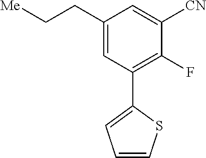

The title compound (421 mg, yield 82%) was obtained as colorless crystals from 3-bromo-2-fluoro-5-propylbenzonitrile (506 mg) and 2-thiopheneboronic acid (400 mg) in the same manner as in Reference Example 4. MS: 246 (MH⁺).

### Reference Example 59

### N-[5-propyl-7-(2-thienyl)-1H-indazol-3-yl]thiourea

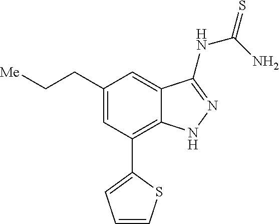

A reaction was carried out in the same manner as in Reference Example 2 from 2-fluoro-5-propyl-3-(2-thienyl)benzonitrile (568 mg), and the title compound (31.2 mg, 29%) was obtained as colorless crystals in the same manner as in Reference Example 3. MS: 317 (MH⁺).

### Reference Example 60

### 2-fluoro-5-propyl-3-pyridin-3-ylbenzonitrile

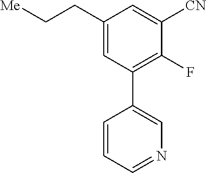

The title compound (490 mg, yield 98%) was obtained as colorless crystals from 3-bromo-2-fluoro-5-propylbenzonitrile (505 mg) and pyridin-3-ylboronic acid (385 mg) in the same manner as in Reference Example 4. MS: 241 (MH⁺).

### Reference Example 61

### 5-propyl-7-pyridin-3-yl-1H-indazole-3-amine

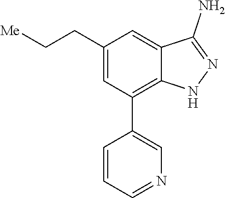

The title compound (182 mg, yield 35%) was obtained as colorless crystals from 2-fluoro-5-propyl-3-pyridin-3-ylbenzonitrile (490 mg) in the same manner as in Reference Example 2. MS: 253 (MH⁺).

### Reference Example 62

### N-(5-propyl-7-pyridin-3-yl-1H-indazol-3-yl)thiourea

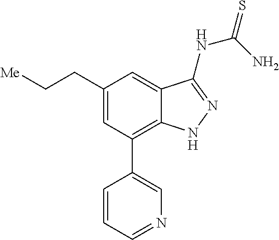

The title compound (156 mg, yield 70%) was obtained as colorless crystals from 5-propyl-7-pyridin-3-yl-1H-indazole-3-amine (182 mg) in the same manner as in Reference Example 3.

MS: 312 (MH⁺).

### Reference Example 63

### 2-fluoro-5-propyl-3-[(E)-2-pyridin-4-ylvinyl]benzonitrile

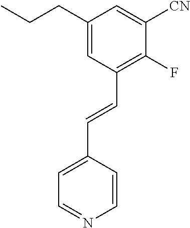

An N,N-dimethylformamide solution (10 mL) of 3-bromo-2-fluoro-5-propylbenzonitrile (472 mg), tetrakis(triphenylphosphine)palladium(0) (226 mg), diisopropylethylamine (1.7 mL) and 4-vinylpyridine (0.42 mL) was stirred overnight at 80° C., diluted with ethyl acetate, washed with water and saturated brine, dried over anhydrous magnesium sulfate and concentrated under reduced pressure. The residue was purified by NH-silica gel column chromatography (hexane:ethyl acetate=20:1-1:1) to give the title compound (385 mg, yield 74%) as colorless oil. MS: 267 (MH⁺).

### Reference Example 64

### 5-propyl-7-[(E)-2-pyridin-4-ylvinyl]-1H-indazole-3-amine

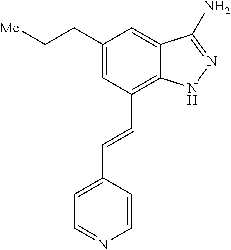

The title compound (69 mg, yield 53%) was obtained as pale-yellow crystals from 2-fluoro-5-propyl-3-[(E)-2-pyridin-4-ylvinyl]benzonitrile (124 mg) in the same manner as in Reference Example 2. MS: 279 (MH⁺).

### Reference Example 65

### N-{5-propyl-7-[(E)-2-pyridin-4-ylvinyl]-1H-indazol-3-yl}thiourea

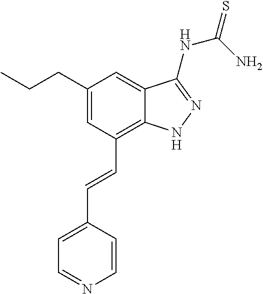

The title compound (69.7 mg, yield 83%) was obtained as pale-yellow crystals from 5-propyl-7-[(E)-2-pyridin-4-ylvinyl]-1H-indazole-3-amine (69 mg) in the same manner as in Reference Example 3. MS: 338 (MH⁺).

### Reference Example 66

### 2-fluoro-5-propyl-3-(1,3-thiazol-2-yl)benzonitrile

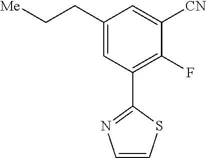

The title compound (263 mg, yield 51%) was obtained as colorless crystals from 3-bromo-2-fluoro-5-propylbenzonitrile (505 mg) and 2-(tributylstannyl)thiazole (0.94 g) in the same manner as in Reference Example 1. MS: 247 (MH⁺).

### Reference Example 67

### 5-propyl-7-(1,3-thiazol-2-yl)-1H-indazole-3-amine

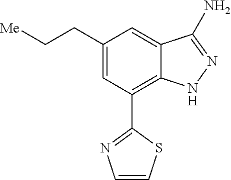

The title compound (203 mg, yield 73%) was obtained as pale-yellow crystals from 2-fluoro-5-propyl-3-(1,3-thiazol-2-yl)benzonitrile (263 mg) in the same manner as in Reference Example 2. MS: 259 (MH⁺).

### Reference Example 68

### N-[5-propyl-7-(1,3-thiazol-2-yl)-1H-indazol-3-yl]thiourea

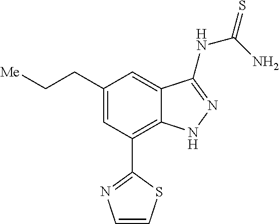

The title compound (224 mg, yield 90%) was obtained as pale-yellow crystals from 5-propyl-7-(1,3-thiazol-2-yl)-1H-indazole-3-amine (203 mg) in the same manner as in Reference Example 3. MS: 318(MW).

### Reference Example 69

### 2-fluoro-5-propyl-3-pyridin-4-ylbenzonitrile

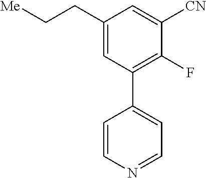

The title compound (156 mg, yield 31%) was obtained as colorless crystals from 3-bromo-2-fluoro-5-propylbenzonitrile (501 mg) and pyridin-4-ylboronic acid (509 mg) in the same manner as in Reference Example 4. MS: 241(MH⁺).

### Reference Example 70

### 5-propyl-7-pyridin-4-yl-1H-indazole-3-amine

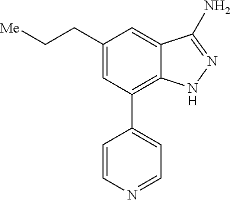

The title compound (61.6 mg, yield 38%) was obtained as pale-yellow crystals from 2-fluoro-5-propyl-3-pyridin-4-ylbenzonitrile (156 mg) in the same manner as in Reference Example 2. MS: 253(MH⁺).

### Reference Example 71

### N-(5-propyl-7-pyridin-4-yl-1H-indazol-3-yl)thiourea

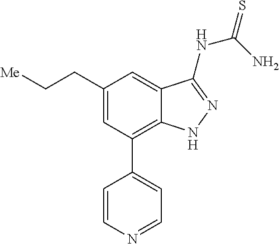

The title compound (61.1 mg, yield 80%) was obtained as colorless crystals from 5-propyl-7-pyridin-4-yl-1H-indazole-3-amine (61.6 mg) in the same manner as in Reference Example 3.

MS: 312 (MH⁺).

### Reference Example 72

### 2-fluoro-3-(1-methyl-1H-pyrazol-4-yl)-5-propylbenzonitrile

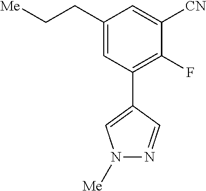

The title compound (266 mg, yield 52%) was obtained as colorless crystals from 3-bromo-2-fluoro-5-propylbenzonitrile (507 mg) and 1-methyl-4-(4,4,5,5-tetramethyl-1,3,2-dioxaborolan-2-yl)-1H-pyrazole (871 mg) in the same manner as in Reference Example 4. MS: 244 (MH⁺).

### Reference Example 73

### 7-(1-methyl-1H-pyrazol-4-yl)-5-propyl-1H-indazole-3-amine

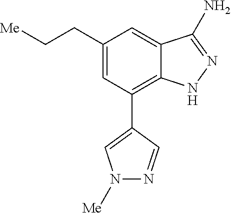

The title compound (61.2 mg, yield 22%) was obtained as pale-yellow crystals from 2-fluoro-3-(1-methyl-1H-pyrazol-4-yl)-5-propylbenzonitrile (266 mg) in the same manner as in Reference Example 2. ¹H NMR (300 MHz, DMSO-d₆) δ ppm 0.92 (3H, t, J=7.4 Hz) 1.43-1.77 (2H, m) 2.62 (2H, t, J=7.4 Hz) 3.89 (3H, s) 5.31 (2H, s) 7.15-7.49 (2H, m) 7.99 (1H, s) 8.29 (1H, s) 11.16 (1H, s)

### Reference Example 74

### N-[7-(1-methyl-1H-pyrazol-4-yl)-5-propyl-1H-indazol-3-yl]thiourea

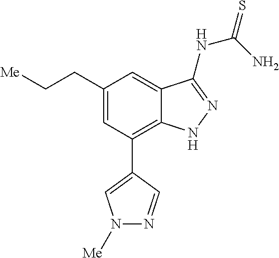

The title compound (55.3 mg, yield 73%) was obtained as pale-yellow crystals from 7-(1-methyl-1H-pyrazol-4-yl)-5-propyl-1H-indazole-3-amine (61.2 mg) in the same manner as in Reference Example 3. MS: 315 (MH⁺).

### Reference Example 75

### 7-(1-benzothien-2-yl)-5-propyl-1H-indazole-3-amine

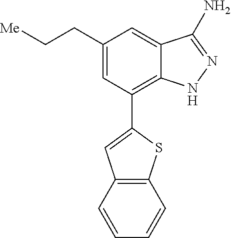

A reaction was carried out from 3-bromo-2-fluoro-5-propylbenzonitrile (495 mg) and 1-benzothien-2-ylboronic acid (728 mg) in the same manner as in Reference Example 4, and the title compound (53.4 mg, yield 8.5%) was obtained as colorless crystals in the same manner as in Reference Example 2.

MS: 308 (MH⁺).

### Reference Example 76

### N-[7-(1-benzothien-2-yl)-5-propyl-1H-indazol-3-yl]thiourea

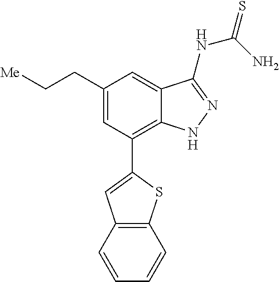

The title compound (40.6 mg, yield 64%) was obtained as pale-yellow crystals from 7-(1-benzothien-2-yl)-5-propyl-1H-indazole-3-amine (53.4 mg) in the same manner as in Reference Example 3. MS: 367 (MH⁺).

### Reference Example 77

### 2-[5-(3-chloropyridin-2-yl)-1H-indazol-3-yl]-1H-isoindole-1,3(2H)-dione

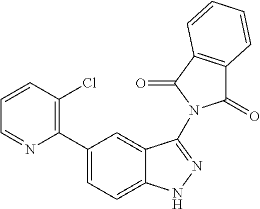

To a 1,4-dioxane solution of 5-(3-chloropyridin-2-yl)-1H-indazole-3-amine (4.60 g) was added phthalic anhydride, and the mixture was heated under reflux for 24 hr. The reaction is mixture was cooled to room temperature and concentrated under reduced pressure. The residue was suspended in diethyl ether and stirred at room temperature for 1 hr. The crystals were collected by filtration to give the title compound (6.40 g, yield 91%) as colorless crystals. MS: 375 (MH⁺).

### Reference Example 78

### 2-[5-(3-chloropyridin-2-yl)-1-methyl-1H-indazol-3-yl]-1H-isoindole-1,3(2H)-dione

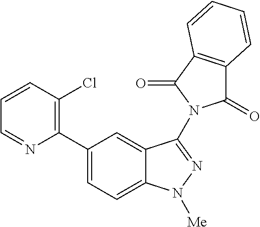

To an N,N-dimethylformamide solution (10 mL) of 2-[5-(3-chloropyridin-2-yl)-1H-indazol-3-yl]-1H-isoindole-1,3(2H)-dione (400 mg) were added cesium carbonate (418 mg) and methyl iodide (0.073 mL), and the mixture was stirred overnight at 50° C. The mixture was diluted with ethyl acetate, washed with water and saturated brine, dried over anhydrous magnesium sulfate and concentrated under reduced pressure. The obtained crude crystals were purified by recrystallization (ethyl acetate-diisopropyl ether) to give the title compound (207 mg, yield 50%) as colorless crystals. MS: 389 (MH⁺).

### Reference Example 79

### 5-(3-chloropyridin-2-yl)-1-methyl-1H-indazole-3-amine

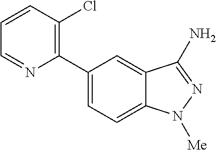

To an ethanol solution (3 mL) of 2-[5-(3-chloropyridin-2-yl)-1-methyl-1H-indazol-3-yl]-1H-isoindole-1,3(2H)-dione (197 mg) was added hydrazine monohydrate (0.074 mL) and the mixture was stirred for 2 hr at 80° C. The mixture was diluted with ethyl acetate, washed with saturated aqueous sodium hydrogen carbonate, water and saturated brine, dried over anhydrous magnesium sulfate, and concentrated under reduced pressure. The obtained crude crystal was purified by recrystallization (ethyl acetate-diisopropyl ether) to give the title compound as 78.1 mg (yield 60%) of primary crystals and as 40.5 mg (yield 31%) of secondary crystals, as colorless crystals. MS: 259 (MH⁺).

### Reference Example 80

### N-[5-(3-chloropyridin-2-yl)-1-methyl-1H-indazol-3-yl]thiourea

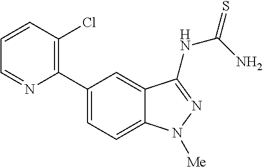

The title compound (100 mg, yield 75%) was obtained as pale-yellow crystals from 5-(3-chloropyridin-2-yl)-1-methyl-1H-indazole-3-amine (108 mg) in the same manner as in Reference Example 3. ¹H NMR (300 MHz, DMSO-d₆) δ ppm 4.00 (s, 3H) 7.43 (dd, J=7.95, 4.54 Hz, 1H) 7.61-7.69 (m, 1H) 7.73-7.81 (m, 1H) 8.03-8.11 (m, 1H) 8.53-8.73 (m, 2H) 8.82 (br. s., 1H) 9.18 (br. s., 1H) 11.03 (br. s., 1H).

### Reference Example 81

### 2-[5-(3-chloropyridin-2-yl)-1-ethyl-1H-indazol-3-yl]-1H-isoindole-1,3(2H)-dione

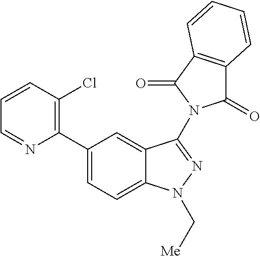

The title compound (204 mg, yield 47%) was obtained as pale-yellow crystals from 2-[5-(3-chloropyridin-2-yl)-1H-indazol-3-yl]-1H-isoindole-1,3(2H)-dione (400 mg) and ethyl iodide (0.094 mL) in the same manner as in Reference Example 78.

MS: 403 (MH⁺).

### Reference Example 82

### 5-(3-chloropyridin-2-yl)-1-ethyl-1H-indazole-3-amine

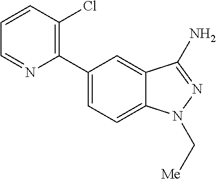

The title compound (125 mg, yield 95%) was obtained as pale-yellow crystals from 2-[(5-(3-chloropyridin-2-yl)-1-ethyl-1H-indazol-3-yl]-1H-isoindole-1,3(2H)-dione (194 mg) in the same manner as in Reference Example 79. MS: 273 (MH⁺).

### Reference Example 83

### N-[5-(3-chloropyridin-2-yl)-1-ethyl-1H-indazol-3-yl]thiourea

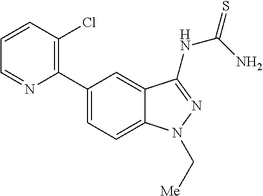

The title compound (109 mg, yield 78%) was obtained as pale-yellow crystals from 5-(3-chloropyridin-2-yl)-1-ethyl-1H-indazole-3-amine (0.115 mg) in the same manner as in Reference Example 3. MS: 332 (MH⁺).

### Reference Example 84

### 2-[1-benzyl-5-(3-chloropyridin-2-yl)-1H-indazol-3-yl]-1H-isoindole-1,3(2H)-dione

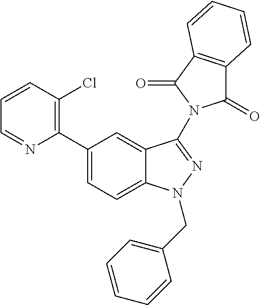

is The title compound (326 mg, yield 66%) was obtained as pale-yellow crystals from 2-[5-(3-chloropyridin-2-yl)-1H-indazol-3-yl]-1H-isoindole-1,3(2H)-dione (400 mg) and benzyl bromide (0.14 mL) in the same manner as in Reference Example 78.

MS: 465 (MH⁺).

### Reference Example 85

### 1-benzyl-5-(3-chloropyridin-2-yl)-1H-indazole-3-amine

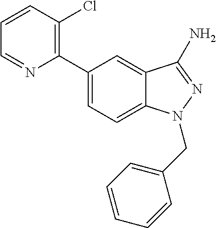

The title compound (230 mg, yield 98%) was obtained as pale-yellow crystals from 2-[1-benzyl-5-(3-chloropyridin-2-yl)-1H-indazol-3-yl]-1H-isoindole-1,3(2H)-dione (327 mg) in the same manner as in Reference Example 79. MS: 335 (MH⁺).

### Reference Example 86

### N-[1-benzyl-5-(3-chloropyridin-2-yl)-1H-indazol-3-yl]thiourea

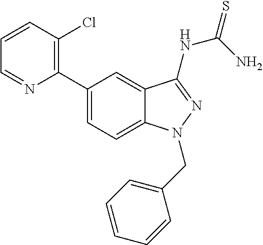

The title compound (231 mg, yield 85%) was obtained as pale-yellow crystals from 1-benzyl-5-(3-chloropyridin-2-yl)-1H-indazole-3-amine (230 mg) in the same manner as in Reference Example 3. MS: 394 (MH⁺).

### Reference Example 87

### 2-{[5-(3-chloropyridin-2-yl)-1H-indazol-3-yl]amino}-1,3-thiazole-5-carbaldehyde

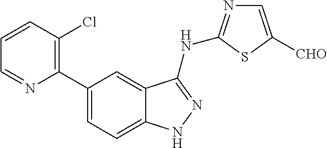

To an N,N-dimethylacetamide solution (120 mL) of N-[5-(3-chloropyridin-2-yl)-1H-indazol-3-yl]thiourea (3.20 g) was added bromomalonaldehyde (2.75 g) and the mixture was stirred at 80° C. for 2 hr. The mixture was allowed to cool, diluted with ethyl acetate-tetrahydrofuran, and washed with water and saturated brine. The organic layer was dried over anhydrous magnesium sulfate and concentrated under reduced pressure. The obtained crude crystal was purified by recrystallization (tetrahydrofuran-ethyl acetate) to give the title compound (3.20 g, yield 55%) as colorless solid. Melting point 259-261° C.

### Reference Example 88

### tert-butyl [(2-{[5-(3-chloropyridin-2-yl)-1H-indazol-3-yl]amino}-1,3-thiazol-5-yl)methyl]methylcarbamate

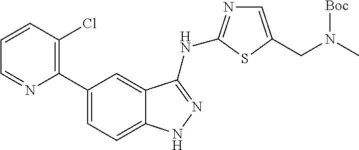

To a tetrahydrofuran solution (2 mL) of 2-{[5-(3-chloropyridin-2-yl)-1H-indazol-3-yl]amino}-1,3-thiazole-5-carbaldehyde (111 mg) were added methylamine (2M tetrahydrofuran solution, 0.8 mL) and sodium triacetoxyhydroborate (330 mg), and the mixture was stirred overnight at room temperature. Saturated aqueous sodium hydrogen carbonate and ethyl acetate were added, and the organic layer was washed with water and saturated brine, dried over anhydrous magnesium sulfate, and concentrated under reduced pressure. The residue was dissolved in tetrahydrofuran (2 mL), di-tert-butyl dicarbonate (68 mg) was added. The mixture was stirred for 30 min at room temperature, concentrated under reduced pressure and purified by silica gel column chromatography (eluate: ethyl acetate) to give the title compound (54.7 mg, 37%) as pale-yellow crystals. MS: 471 (MH⁺).

### Reference Example 89

### tert-butyl 5-(3-chloropyridin-2-yl)-3-[(5-formyl-1,3-thiazol-2-yl)amino]-1H-indazole-1-carboxylate

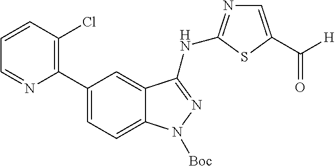

To a tetrahydrofuran solution (10 mL) of 2-{[5-(3-chloropyridin-2-yl)-1H-indazol-3-yl]amino}-1,3-thiazole-5-carbaldehyde (511 mg) were added 4-dimethylaminopyridine (18 mg) and di-tert-butyl dicarbonate (0.38 g), and the mixture was stirred overnight at room temperature. The mixture was diluted with ethyl acetate, washed with water and saturated brine, dried over anhydrous magnesium sulfate, and concentrated under reduced pressure. The obtained crude crystal was purified by recrystallization (ethyl acetate-diisopropyl ether) to give the title compound (354 mg, yield 54%) as colorless solid.

MS: 399 (MH⁺−C₄H₉)

### Reference Example 90

### tert-butyl 5-(3-chloropyridin-2-yl)-3-{[5-(hydroxymethyl)-1,3-thiazol-2-yl]amino}-1H-indazole-1-carboxylate

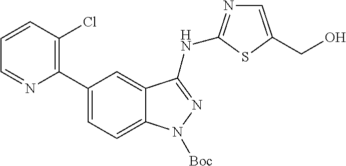

The title compound (65.9 mg, yield 33%) was obtained as pale-yellow crystals from tert-butyl 5-(3-chloropyridin-2-yl)-3-[(5-formyl-1,3-thiazol-2-yl)amino]-1H-indazole-1-carboxylate (201 mg) in the same manner as in Example 31. MS: 358(MH⁺-Boc).

### Reference Example 91

### tert-butyl 5-(3-chloropyridin-2-yl)-3-{[5-(cyanomethyl)-1,3-thiazol-2-yl]amino}-1H-indazole-1-carboxylate

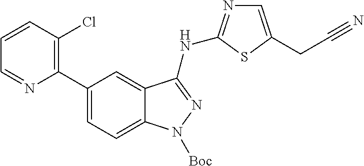

To a tetrahydrofuran solution (2 mL) of tert-butyl 5-(3-chloropyridin-2-yl)-3-{[5-(hydroxymethyl)-1,3-thiazol-2-yl]amino}-1H-indazole-1-carboxylate (51.8 mg) were added acetone cyanhydrin (0.016 mL), 1,1′-(azodicarbonyl)dipiperidine (57 mg) and tributylphosphine (0.057 mL), and the mixture was stirred overnight at room temperature. The mixture was concentrated under reduced pressure and purified by silica gel column chromatography (hexane:ethyl acetate=5:1-0:1) to give the title compound (13.4 mg, 25%) as pale-yellow crystals.

MS: 467 (MH⁺).

### Reference Example 92

### 2-(5-(3-chloropyridin-2-yl)-1-{[2-(trimethylsilyl)ethoxy]methyl}-1H-indazol-3-yl)-1H-isoindole-1,3(2H)-dione

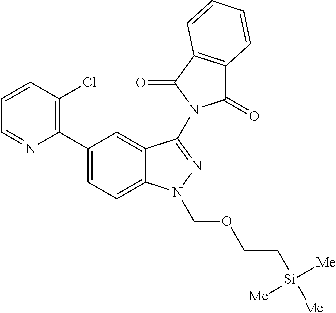

An N,N-dimethylformamide (170 mL) solution of 2-[5-(3-chloropyridin-2-yl)-1H-indazol-3-yl]-1H-isoindole-1,3(2H)-dione (6.40 g) was cooled to 0° C., sodium hydride (0.820 g) was added, and the mixture was stirred at 0° C. for 5 min. To the reaction solution was added dropwise an N,N-dimethylformamide (70 mL) solution of [2-(chloromethoxy)ethyl](trimethyl)silane (3.41 g) at 0° C. for 1 hr, and the mixture was stirred at 0° C. for 2 hr. To the reaction mixture was added saturated aqueous ammonium chloride solution, and the mixture was extracted with ethyl acetate (300 mL×2). The extract was washed successively with water and saturated brine, dried over anhydrous magnesium sulfate, and concentrated under reduced pressure. The residue was purified by silica gel column chromatography (ethyl acetate:hexane=9:1-1:1) to give the title compound (7.30 g, yield 85%) as a colorless non-crystalline solid. MS: 505(MW).

### Reference Example 93

### 5-(3-chloropyridin-2-yl)-1-{[2-(trimethylsilyl)ethoxy]methyl}-1H-indazole-3-amine

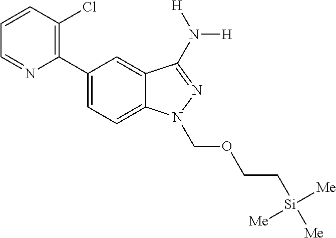

To a solution of 2-(5-(3-chloropyridin-2-yl)-1-{[2-(trimethylsilyl)ethoxy]methyl}-1H-indazol-3-yl)-1H-isoindole-1,3(2H)-dione (7.30 g) in ethanol (200 mL) was added hydrazine monohydrate (3.52 mL), and the mixture was heated under reflux for 2 hr. The reaction solution was filtered to remove insoluble materials, and the filtrate was concentrated under reduced pressure. The residue was purified by NH-silica gel column chromatography (ethyl acetate:hexane=1:1-1:0) to give the title compound (3.05 g, yield 48%) as a pale-yellow oily substance. MS: 375 (MH⁺).

### Reference Example 94

### 5-(3-chloropyridin-2-yl)-N-pyrazin-2-yl-1-{[2-(trimethylsilyl)ethoxy]methyl}-1H-indazole-3-amine

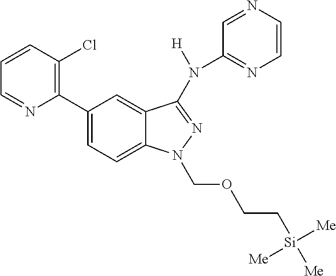

A suspension of 5-(3-chloropyridin-2-yl)-1-{[2-(trimethylsilyl)ethoxy]methyl}-1H-indazole-3-amine (0.440 g), 2-chloropyrazine (0.125 mL), tris(dibenzylideneacetone)dipalladium(0) (0.032 g), (9,9-dimethyl-9H-xanthen-4,5-diyl)bis(diphenylphosphine) (0.045 g) and cesium carbonate (0.531 g) in 1,4-dioxane (12 mL) was brought into argon atmosphere and heated under reflux at 100° C. for 20 hr. The reaction mixture was cooled to room temperature, saturated aqueous ammonium chloride solution was added, and the mixture was extracted with ethyl acetate (50 mL×2). The extract was washed successively with water and saturated brine, dried over anhydrous magnesium sulfate and concentrated under reduced pressure. The residue was purified by silica gel column chromatography (ethyl acetate:hexane=1:3-1:0) to give the title compound (0.255 g, yield 48%) as a colorless non-crystalline solid. MS: 453 (MH⁺).

### Reference Example 95

### 5-(3-chloropyridin-2-yl)-N-(6-methylpyridazin-3-yl)-1-{[2-(trimethylsilyl)ethoxy]methyl}-1H-indazole-3-amine

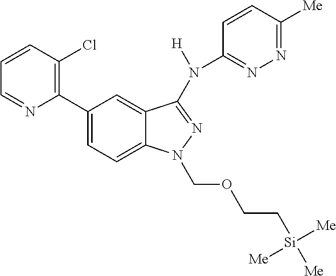

The title compound (0.172 g, yield 28%) was obtained as a pale-yellow oily substance from 5-(3-chloropyridin-2-yl)-1-{[2-(trimethylsilyl)ethoxy]methyl}-1H-indazole-3-amine (0.500 g) and 3-chloro-6-methylpyridazine (0.223 g) in the same manner as in Reference Example 94. MS: 467 (MH⁺).

### Reference Example 96

### 3-[(5-(3-chloropyridin-2-yl)-1-{[2-(trimethylsilyl)ethoxy]methyl}-1H-indazol-3-yl)amino]-N,N-dimethyl-1H-pyrazole-1-sulfonamide

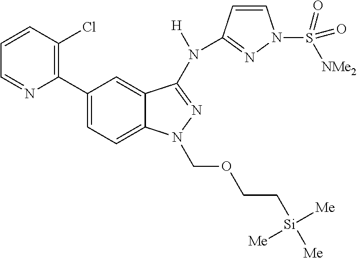

The title compound (0.287 g, yield 49%) was obtained as a pale-yellow oily substance from 5-(3-chloropyridin-2-yl)-1-{[2-(trimethylsilyl)ethoxy]methyl}-1H-indazole-3-amine (0.400 g) and 3-bromo-N,N-dimethyl-1H-pyrazole-1-sulfonamide (0.542 g) in the same manner as in Reference Example 94. MS: 548 (MH⁺).

### Reference Example 97

### 3-bromo-5-(3-chloropyridin-2-yl)-1H-indazole

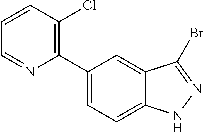

To an acetonitrile solution (30 mL) of 5-(3-chloropyridin-2-yl)-1H-indazole-3-amine (1.57 g) were added copper(II) bromide (1.58 g) and tert-butyl nitrite (0.728 g), and the mixture was stirred at 80° C. for 1 hr. The reaction solution was concentrated, and the residue was suspended in diethyl ether. The suspension was passed through Celite, and the filtrate was concentrated under reduced pressure. The residue was purified by silica gel column chromatography (ethyl acetate:hexane=1:9-7:3) to give the title compound (0.380 g, yield 19%) as colorless oil. MS: 310 (MH⁺).

### Reference Example 98

### tert-butyl 3-bromo-5-(3-chloropyridin-2-yl)-1H-indazole-1-carboxylate

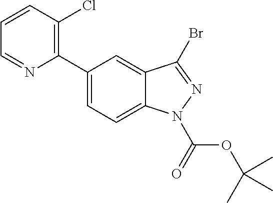

To a tetrahydrofuran solution (10 mL) of 3-bromo-5-(3-chloropyridin-2-yl)-1H-indazole (0.380 g) were added triethylamine (0.19 mL), 4-dimethylaminopyridine (0.008 g) and di-tert-dicarbonate (0.31 mL), and the mixture was stirred at room temperature for 1 hr. The reaction mixture was concentrated, and the residue was purified by silica gel column chromatography (ethyl acetate:hexane=2:98-15:85) to give the title compound (0.352 g, yield 70%) as colorless oil. ¹H NMR (300 MHz, CDCl₃) δ ppm 1.74 (9H, s), 7.25-7.32 (1H, m), 7.82-7.87 (1H, m), 7.97-8.06 (2H, m), 8.23 (1H, d, J=8.7 Hz), 8.62-8.66 (1H, m)

### Reference Example 99

### tert-butyl 5-(3-chloropyridin-2-yl)-3-[(1-methyl-1H-pyrazol-3-yl)amino]-1H-indazole-1-carboxylate

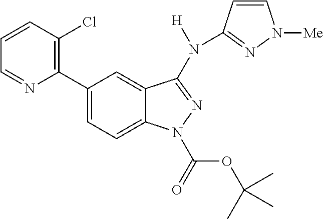

The title compound (0.238 g, yield 65%) was obtained as a pale-yellow oily substance from tert-butyl 3-bromo-5-(3-chloropyridin-2-yl)-1H-indazole-1-carboxylate (0.350 g) and 1-methyl-1H-pyrazole-3-amine (0.108 g) in the same manner as in Reference Example 94. ¹H NMR (300 MHz, CDCl₃) δ ppm 1.73 (9H, s), 3.82 (3H, s), 6.80 (1H, d, J=2.3 Hz), 7.07 (1H, s), 7.22-7.31 (2H, m), 7.83 (1H, dd, J=8.1, 1.5 Hz), 7.93-8.00 (2H, m), 8.13-8.20 (1H, m), 8.62 (1H, dd, J=4.5, 1.5 Hz)

### Reference Example 100

### 2-amino-5-isobutyl-3-methoxybenzonitrile

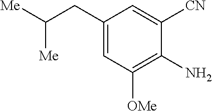

To a tetrahydrofuran solution (60 mL) of 2-amino-5-bromo-3-methoxybenzonitrile (3.41 g) and 1,1′-bis(diphenylphosphino)ferrocenepalladium(II) dichloromethane complex (3.41 g) was added dropwise 2-methylpropylzinc bromide (0.5M tetrahydrofuran solution, 75 mL) at room temperature and the mixture was stirred for 2 hr. To the reaction mixture was added water, and the insoluble materials were filtered and washed with ethyl acetate. The organic layer in the mother liquor was washed with water and saturated brine, dried over anhydrous magnesium sulfate, and concentrated under reduced pressure. The residue was purified by silica gel column chromatography (hexane:ethyl acetate=10:1-5:1) to give the title compound (2.97 g, yield 97%) pale-yellow oil.

MS: 205(M⁺).

### Reference Example 101

### 2-amino-3-hydroxy-5-isobutylbenzonitrile

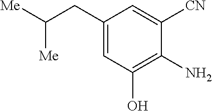

To a dichloromethane solution (5 mL) of 2-amino-5-isobutyl-3-methoxybenzonitrile (233 mg) was added dropwise boron tribromide (1.0M dichloromethane solution, 3.4 mL) under ice-cooling, and the mixture was stirred overnight room temperature. Under ice-cooling, saturated aqueous sodium hydrogen carbonate was added to make the mixture basic, and the mixture was extracted with ethyl acetate. The organic layer was washed with water and saturated brine, dried over anhydrous magnesium sulfate, and concentrated under reduced pressure. The residue was purified by silica gel column chromatography (hexane:ethyl acetate=10:1-3:1) to give the title compound (155 mg, yield 71%) as pale-yellow crystals. MS: 191(MH⁺).

### Reference Example 102

### 2-amino-3-(benzyloxy)-5-isobutylbenzonitrile

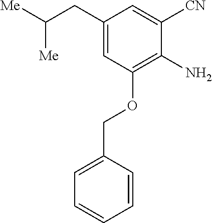

To an N,N-dimethylformamide solution (3 mL) of 2-amino-3-hydroxy-5-isobutylbenzonitrile (59.5 mg) were added potassium carbonate (48 mg) and benzyl bromide (0.037 ml), and the mixture was stirred overnight at 50° C. The mixture was diluted with ethyl acetate, washed with water and saturated brine, dried over anhydrous magnesium sulfate and concentrated under reduced pressure. The residue was purified by silica gel column chromatography (hexane:ethyl acetate-=10:1-3:1) to give the title compound (81.5 mg, yield 100%) as pale-yellow crystals. MS: 281 (MH⁺).

### Reference Example 103

### 7-(benzyloxy)-5-isobutyl-1H-indazole-3-amine

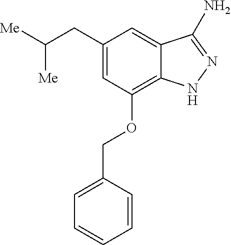

To a solution of 2-amino-3-(benzyloxy)-5-isobutylbenzonitrile (163 g) in concentrated hydrochloric acid (5 mL) was added an aqueous solution (1 mL) of sodium nitrite (0.041 g) at −2 to 0° C. for 15 min, and the mixture was stirred at 0° C. for 30 min. The obtained reaction solution was added to a solution of tin(II) chloride (310 mg) in concentrated hydrochloric acid (5 mL) at 0° C. for 10 min, and the mixture was stirred overnight at room temperature. To the reaction mixture was added 8N aqueous sodium hydroxide solution to neutralize, diluted with ethyl acetate, washed with water and saturated brine, dried over anhydrous magnesium sulfate, and concentrated under reduced pressure. The residue was purified by silica gel column chromatography (hexane:ethyl acetate=10:1-0:1) to give the title compound (132 mg, yield 83%) pale-yellow oil.

MS: 296 (MH⁺).

### Reference Example 104

### N-[7-(benzyloxy)-5-isobutyl-1H-indazol-3-yl]thiourea

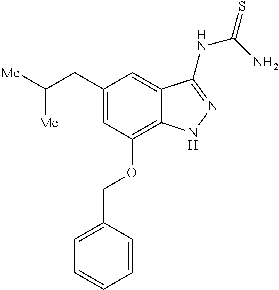

The title compound (144 mg, yield-97%) was obtained as pale-yellow crystals from 7-(benzyloxy)-5-isobutyl-1H-indazole-3-amine (124 mg) in the same manner as in Reference Example 3.

MS: 355 (MH⁺).

### Reference Example 105

### 2-amino-5-isobutyl-3-(pyridin-2-ylmethoxy)benzonitrile

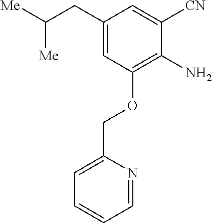

The title compound (218 mg, yield 100%) was obtained as pale-yellow crystals from 2-amino-3-hydroxy-5-isobutylbenzonitrile (147 mg) and 2-(bromomethyl)pyridinehydrobromide (215 mg) in the same manner as in Reference Example 102. MS: 282 (MH⁺).

### Reference Example 106

### 5-isobutyl-7-(pyridin-2-ylmethoxy)-1H-indazole-3-amine

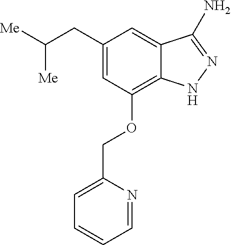

The title compound (183 mg, yield 80%) was obtained as a pale-yellow oily substance from 2-amino-5-isobutyl-3-(pyridin-2-ylmethoxy)benzonitrile (218 mg) in the same manner as in Reference Example 103. MS: 297 (MH⁺).

### Reference Example 107

### N-[5-isobutyl-7-(pyridin-2-ylmethoxy)-1H-indazol-3-yl]thiourea

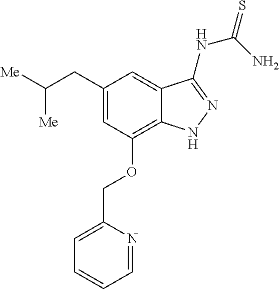

The title compound (154 mg, yield 80%) was obtained as pale-yellow crystals 5-isobutyl-7-(pyridin-2-ylmethoxy)-1H-indazole-3-amine (159 mg) in the same manner as in Reference Example 3. Melting point 210-212° C.

### Reference Example 108

### 2-amino-5-isobutyl-3-(pyridin-3-ylmethoxy)benzonitrile

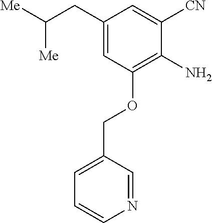

The title compound (170 mg, yield 77%) was obtained as pale-yellow crystals from 2-amino-3-hydroxy-5-isobutylbenzonitrile (150 mg) and 3-(chloromethyl)pyridine hydrochloride (142 mg) in the same manner as in Reference Example 102. MS: 282 (MH⁺).

### Reference Example 109

### 5-isobutyl-7-(pyridin-3-ylmethoxy)-1H-indazole-3-amine

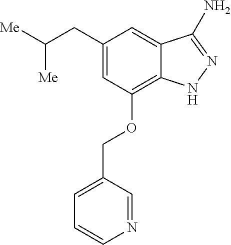

The title compound (121 mg, yield 76%) was obtained as a pale-yellow oily substance from 2-amino-5-isobutyl-3-(pyridin-3-ylmethoxy)benzonitrile (150 mg) in the same manner as in Reference Example 103. MS: 297 (MH⁺).

### Reference Example 110

### N-[5-isobutyl-7-(pyridin-3-ylmethoxy)-1H-indazol-3-yl]thiourea

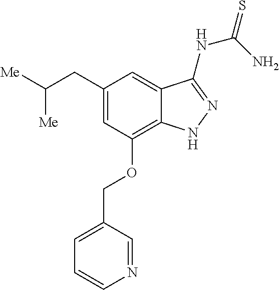

The title compound (120 mg, yield 86%) was obtained as pale-yellow crystals from 5-isobutyl-7-(pyridin-3-ylmethoxy)-1H-indazole-3-amine (116 mg) in the same manner as in Reference Example 3. Melting point 212-215° C.

### Reference Example 111

### 2-amino-5-isobutyl-3-(pyridin-4-ylmethoxy)benzonitrile

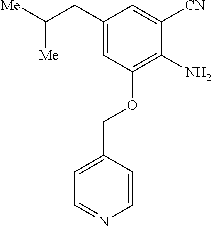

The title compound (189 mg, yield 84%) was obtained as pale-yellow crystals from 2-amino-3-hydroxy-5-isobutylbenzonitrile (152 mg) and 4-(chloromethyl)pyridine hydrochloride (142 mg) in the same manner as in Reference Example 102. MS: 282 (MH⁺).

### Reference Example 112

### 5-isobutyl-7-(pyridin-4-ylmethoxy)-1H-indazole-3-amine

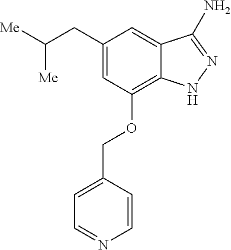

The title compound (152 mg, yield 85%) was obtained as a pale-yellow oily substance from 2-amino-5-isobutyl-3-(pyridin-4-ylmethoxy)benzonitrile (169 mg) in the same manner as in Reference Example 103. MS: 297(MW).

### Reference Example 113

### N-[5-isobutyl-7-(pyridin-4-ylmethoxy)-1H-indazol-3-yl]thiourea

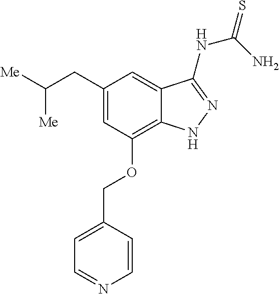

The title compound (175 mg, yield 100%) was obtained as pale-yellow crystals from 5-isobutyl-7-(pyridin-4-ylmethoxy)-1H-indazole-3-amine (139 mg) in the same manner as in Reference Example 3. Melting point 229-230° C.

### Reference Example 115

### 2-amino-5-isobutyl-3-[(1-methyl-1H-imidazol-2-yl)methoxy]benzonitrile

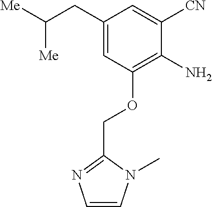

The title compound (64.2 mg, yield 29%) was obtained as a pale-yellow oily substance from 2-amino-3-hydroxy-5-isobutylbenzonitrile (147 mg) and 2-(chloromethyl)-1-methyl-1H-imidazolehydrochloride (142 mg) in the same manner as in Reference Example 102. MS: 285(MH⁺).

### Reference Example 116

### 5-isobutyl-7-[(1-methyl-1H-imidazol-2-yl)methoxy]-1H-indazole-3-amine

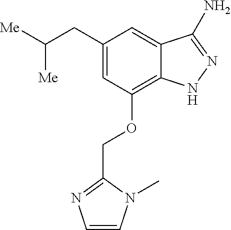

The title compound (56.5 mg, yield 84%) was obtained as non-crystalline powder from 2-amino-5-isobutyl-3-[(1-methyl-1H-imidazol-2-yl)methoxy]benzonitrile (64.2 mg) in the same manner as in Reference Example 103. MS: 300(MW).

### Reference Example 117

### N-{5-isobutyl-7-[(1-methyl-1H-imidazol-2-yl)methoxy]-1H-indazol-3-yl}thiourea

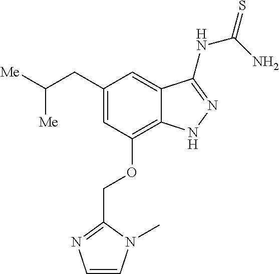

The title compound (52.6 mg, yield 82%) was obtained as pale-yellow crystals from 5-isobutyl-7-[(1-methyl-1H-imidazol-2-yl)methoxy]-1H-indazole-3-amine (53.7 mg) in the same manner as in Reference Example 3. MS: 359(MH⁺).

### Reference Example 118

### 2-amino-5-isobutyl-3-{[4-(methylsulfonyl)benzyl]oxy}benzonitrile

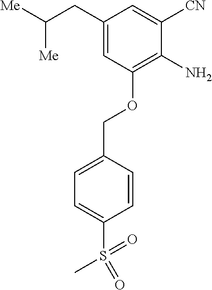

The title compound (260 mg, yield 92%) was obtained as a pale-yellow oily substance from 2-amino-3-hydroxy-5-isobutylbenzonitrile (150 mg) and 1-(chloromethyl)-4-(methylsulfonyl)benzene (178 mg) in the same manner as in Reference Example 102. ¹H NMR (300 MHz, CDCl₃) δ ppm 0.71-0.94 (6H, m) 1.64-1.89 (1H, m) 2.33 (2H, d, J=7.2 Hz) 3.07 (3H, s) 4.48 (2H, s) 5.18 (2H, s) 6.71 (1H, s) 6.81 (1H, s) 7.55-7.68 (2H, m) 7.83-8.09 (2H, m)

### Reference Example 119

### 5-isobutyl-7-{[4-(methylsulfonyl)benzyl]oxy}-1H-indazole-3-amine

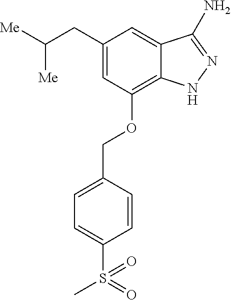

The title compound (215 mg, yield 79%) was obtained as a pale-yellow oily substance from 2-amino-5-isobutyl-3-{[4-(methylsulfonyl)benzyl]oxy}benzonitrile (260 mg) in the same manner as in Reference Example 103. Melting point 187-188° C.

### Reference Example 120

### N-(5-isobutyl-7-{[4-(methylsulfonyl)benzyl]oxy}-1H-indazol-3-yl)thiourea

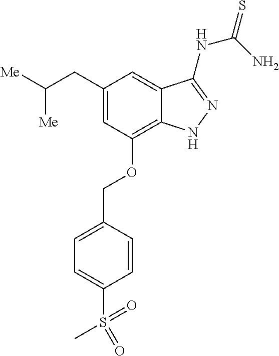

The title compound (183 mg, yield 73%) was obtained as pale-yellow crystals from 5-isobutyl-7-{[4-(methylsulfonyl)benzyl]oxy}-1H-indazole-3-amine (215 mg) in the same manner as in Reference Example 3. Melting point 220-221° C.

### Reference Example 121

### 2-amino-3-[(2-fluorobenzyl)oxy]-5-isobutylbenzonitrile

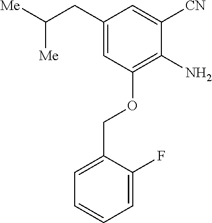

The title compound (230 mg, yield 84%) was obtained as pale-yellow crystals from 2-amino-3-hydroxy-5-isobutylbenzonitrile (175 mg) 1-(chloromethyl)-2-fluorobenzene (0.12 mL) in the same manner as in Reference Example 102.

MS: 299 (MH⁺).

### Reference Example 122

### 7-[(2-fluorobenzyl)oxy]-5-isobutyl-1H-indazole-3-amine

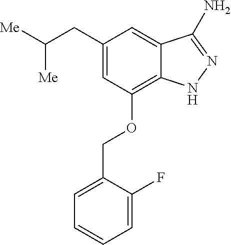

The title compound (99.4 mg, yield 42%) was obtained as a pale-yellow oily substance from 2-amino-3-[(2-fluorobenzyl)oxy]-5-isobutylbenzonitrile (224 mg) in the same manner as in Reference Example 103. Melting point 142-143° C.

### Reference Example 123

### N-{7-[(2-fluorobenzyl)oxy]-5-isobutyl-1H-indazol-3-yl}thiourea

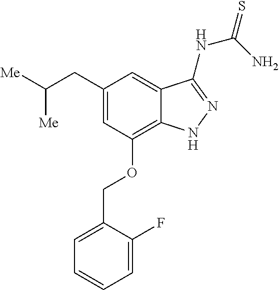

The title compound (135 mg, yield 100%) was obtained as a pale-yellow oily substance from 7-[(2-fluorobenzyl)oxy]-5-isobutyl-1H-indazole-3-amine (99.4 mg) in the same manner as in Reference Example 3. MS: 373 (MH⁺).

### Reference Example 124

### 2-amino-5-isobutyl-3-(1,3-thiazol-2-ylmethoxy)benzonitrile

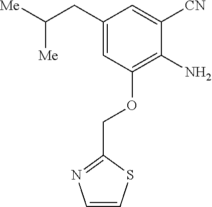

The title compound (152 mg, yield 85%) was obtained as a brown oily substance from 2-amino-3-hydroxy-5-isobutylbenzonitrile (169 mg) and 2-(chloromethyl)-1,3-thiazolehydrochloride (141 mg) in the same manner as in Reference Example 102. MS: 288 (MH⁺).

### Reference Example 125

### 5-isobutyl-7-(1,3-thiazol-2-ylmethoxy)-1H-indazole-3-amine

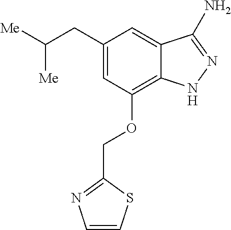

The title compound (90.1 mg, yield 52%) was obtained, as a pale-yellow oily substance from 2-amino-5-isobutyl-3-(1,3-thiazol-2-ylmethoxy)benzonitrile (165 mg) in the same manner as in Reference Example 103. MS: 303 (MH⁺).

### Reference Example 126

### N-[5-isobutyl-7-(1,3-thiazol-2-ylmethoxy)-1H-indazol-3-yl]thiourea

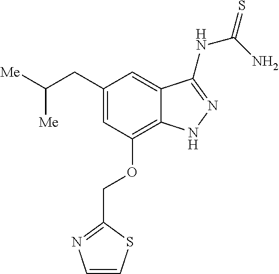

The title compound (115 mg, yield 100%) was obtained as pale-yellow crystals from 5-isobutyl-7-(1,3-thiazol-2-ylmethoxy)-1H-indazole-3-amine (90.1 mg) in the same manner as in Reference Example 3. MS: 303 (MH⁺).

### Reference Example 127

### 2-amino-5-isobutyl-3-[2-(3-thienyl)ethoxy]benzonitrile

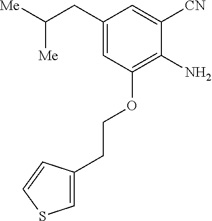

To an ethyl acetate solution (5 mL) of thiophene-2-ethanol (0.21 mL) were added triethylamine (0.31 mL) and methanesulfonyl chloride (0.16 mL) under ice-cooling, and stirred for 1 hr. The insoluble materials were filtered through Celite, and the mother liquor was concentrated under reduced pressure. To an N,N-dimethylformamide solution (10 mL) of the residue were added potassium carbonate (0.33 g) and 2-amino-3-hydroxy-5-isobutylbenzonitrile (300 mg), and the mixture was stirred at 80° C. for 4 hr. The mixture was diluted with ethyl acetate, washed with water and saturated brine, dried over anhydrous magnesium sulfate, and concentrated under reduced pressure. The residue was purified by silica gel column chromatography (hexane:ethyl acetate=20:1-10:1) to give the title compound (314 mg, yield 66%) as a brown oily substance. MS: 301 (MH⁺).

### Reference Example 128

### 5-isobutyl-7-[2-(3-thienyl)ethoxy]-1H-indazole-3-amine

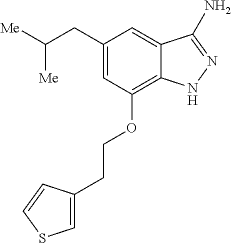

The title compound (195 mg, yield 59%) was obtained as pale-yellow crystals from 2-amino-5-isobutyl-3-[2-(3-thienyl)ethoxy]benzonitrile (314 mg) in the same manner as in Reference Example 103. MS: 316 (MH⁺).

### Reference Example 129

### N-{5-isobutyl-7-[2-(3-thienyl)ethoxy]-1H-indazol-3-yl}thiourea

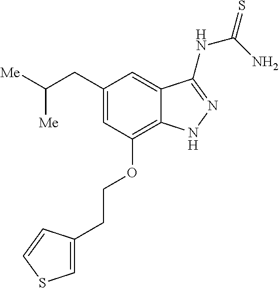

The title compound (244 mg, yield 100%) was obtained as a pale-yellow oily substance from 5-isobutyl-7-[2-(3-thienyl)ethoxy]-1H-indazole-3-amine (195 mg) in the same manner as in Reference Example 3. MS: 375 (MH⁺).

### Reference Example 130

### 2-(2-amino-3-cyano-5-isobutylphenoxy)-N,N-dimethylacetamide

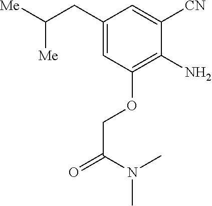

The title compound (202 mg, yield 93%) was obtained as a brown oily substance from 2-amino-3-hydroxy-5-isobutylbenzonitrile (201 mg) and 2-chloro-N,N-dimethylacetamide (0.12 mL) in the same manner as in Reference Example 102. MS: 276(MH⁺).

### Reference Example 131

### 2-[(3-amino-5-isobutyl-1H-indazole-7-yl)oxy]-N,N-dimethylacetamide

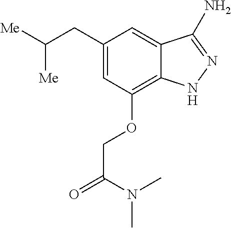

The title compound (174 mg, yield 61%) was obtained as pale-yellow crystals from 2-(2-amino-3-cyano-5-isobutylphenoxy)-N,N-dimethylacetamide (272 mg) in the same manner as in Reference Example 103. Melting point 175-176° C. (MH⁺).

### Reference Example 132

### 2-({3-[(aminocarbonothioyl)amino]-5-isobutyl-1H-indazole-7-yl}oxy)-N,N-dimethylacetamide

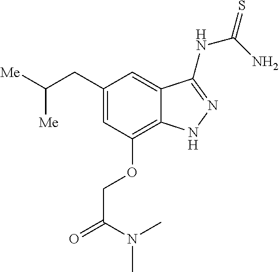

The title compound (183 mg, yield 87%) was obtained as pale-yellow crystals from 2-[(3-amino-5-isobutyl-1H-indazole-7-yl)oxy]-N,N-dimethylacetamide (174 mg) in the same manner as in Reference Example 3. Melting point 197-198° C.

### Reference Example 133

### 2-amino-5-isobutyl-3-(2-pyridin-2-ylethoxy)benzonitrile

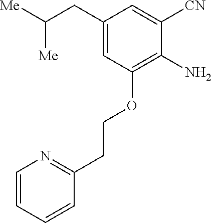

The title compound (56.5 mg, yield 18%) was obtained as pale-yellow crystals from 2-amino-3-hydroxy-5-isobutylbenzonitrile (200 mg) and 2-pyridin-2-ylethanol (0.14 mL) in the same manner as in Reference Example 127.

MS: 296 (MH⁺).

### Reference Example 134

### 5-isobutyl-7-(2-pyridin-2-ylethoxy)-1H-indazole-3-amine

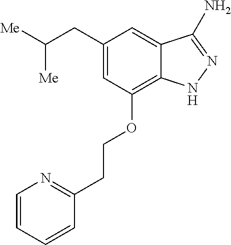

The title compound (41.4 mg, yield 70%) was obtained as a pale-yellow oily substance from 2-amino-5-isobutyl-3-(2-pyridin-2-ylethoxy)benzonitrile (56 mg) in the same manner as in Reference Example 103. MS: 312(We).

### Reference Example 135

### N-[5-isobutyl-7-(2-pyridin-2-ylethoxy)-1H-indazol-3-yl]thiourea

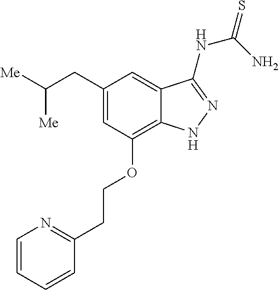

The title compound (46.0 mg, yield 93%) was obtained as pale-yellow crystals from 5-isobutyl-7-(2-pyridin-2-ylethoxy)-1H-indazole-3-amine (41.4 mg) in the same manner as in Reference Example 3. MS: 312 (MH⁺).

### Reference Example 136

### 2-{[5-isobutyl-7-(pyridin-2-ylmethoxy)-1H-indazol-3-yl]amino}-1,3-thiazole-5-carbaldehyde

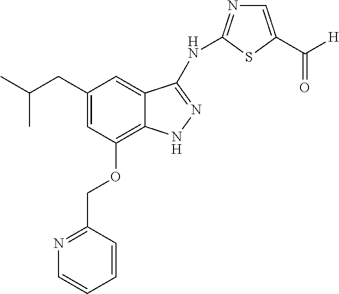

The title compound (276 mg, yield 71%) was obtained as brown crystals from N-[5-isobutyl-7-(pyridin-2-ylmethoxy)-1H-indazol-3-yl]thiourea (340 mg) in the same manner as in Reference Example 87. Melting point 176-178° C.

### Reference Example 137

### 2-({5-isobutyl-7-[(2-(3-thienyl)ethoxy]-1H-indazol-3-yl}amino)-1,3-thiazole-5-carbaldehyde

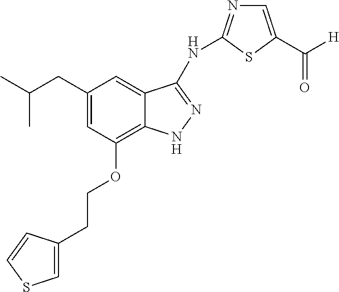

The title compound (50.0 mg, yield 27%) was obtained as brown crystals from N-{5-isobutyl-7-[2-(3-thienyl)ethoxy]-1H-indazol-3-yl}thiourea (166 mg) in the same manner as in Reference Example 87. MS: 427 (MH⁺).

### Reference Example 138

### 2-amino-5-(4,4,5,5-tetramethyl-1,3,2-dioxaborolan-2-yl)benzonitrile

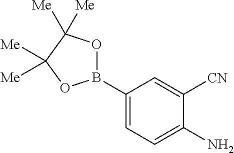

A dimethylsulfoxide suspension (50 mL) of 2-amino-5-bromobenzonitrile (3.53 g), bis(pinacolate)diboron (5 g), 1,1′-bis(diphenylphosphino)ferrocenepalladium(0) dichloromethane complex (730 mg) and potassium acetate (5.27 g) was stirred overnight at 80° C. The suspension was diluted with toluene and water, and the insoluble materials were filtered through Celite. The aqueous layer in the mother liquor was extracted with toluene, washed with water and saturated brine, dried over anhydrous magnesium sulfate, and concentrated under reduced pressure. The residue was purified by NH-silica gel column chromatography (hexane:ethyl acetate=1:1) to give the title compound (2.83 g, yield 65%) was obtained as colorless crystals. Melting point 172-173° C.

### Reference Example 139

### 2-amino-5-(3-chloropyridin-2-yl)benzonitrile

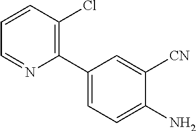

The title compound (1.71 g, yield 64%) was obtained as colorless crystals from 2-amino-5-(4,4,5,5-tetramethyl-1,3,2-dioxaborolan-2-yl)benzonitrile (2.83 g) in the same manner as in Reference Example 12. Melting point 139-140° C.

### Reference Example 140

### 2-amino-3-bromo-5-(3-chloropyridin-2-yl)benzonitrile

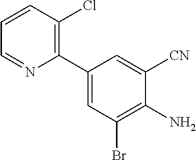

To an acetic acid solution (10 mL) of 2-amino-5-(3-chloropyridin-2-yl)benzonitrile (503 mg) was added N-bromosuccinimide (390 mg) by portions, and the mixture was stirred at room temperature for 1 hr. 8N sodium hydroxide was added to make the mixture basic, and the precipitated crystals were collected by filtration and washed with water. The obtained crude crystals were recrystallized (ethyl acetate-diisopropyl ether) to give the title compound (420 mg, yield 62%) as pale-yellow crystals. MS: 310 (MH⁺+1).

### Reference Example 141

### 2-amino-5-(3-chloropyridin-2-yl)-3-(1-methyl-1H-pyrazol-4-yl)benzonitrile

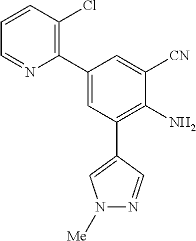

The title compound (325 mg, yield 77%) was obtained as pale-yellow crystals from 2-amino-3-bromo-5-(3-chloropyridin-2-yl)benzonitrile (420 mg) and 1-methyl-4-(4,4,5,5-tetramethyl-1,3,2-dioxaborolan-2-yl)-1H-pyrazole (340 mg) in the same manner as in Reference Example 4. Melting point>250° C.

### Reference Example 142

### 5-(3-chloropyridin-2-yl)-7-(1-methyl-1H-pyrazol-4-yl)-1H-indazole-3-amine

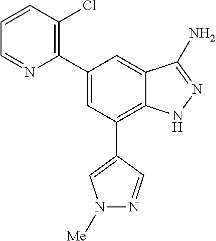

The title compound (214 mg, yield 63%) was obtained as pale-yellow crystals from 2-amino-5-(3-chloropyridin-2-yl)-3-(1-methyl-1H-pyrazol-4-yl)benzonitrile (325 mg) in the same manner as in Reference Example 103. Melting point 238-239° C.

### Reference Example 143

### N-[5-(3-chloropyridin-2-yl)-7-(1-methyl-1H-pyrazol-4-yl)-1H-indazol-3-yl]thiourea

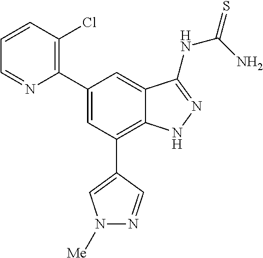

The title compound (184 mg, yield 74%) was obtained as pale-yellow crystals from 5-(3-chloropyridin-2-yl)-7-(1-methyl-1H-pyrazol-4-yl)-1H-indazole-3-amine (211 mg) in the same manner as in Reference Example 3. Melting point 256-257° C.

### Reference Example 144

### 7-bromo-5-(3-chloropyridin-2-yl)-1H-indazole-3-amine

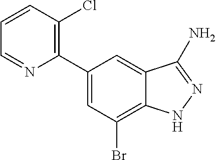

The title compound (210 mg, yield 40%) was obtained as pale-yellow crystals from 2-amino-3-bromo-5-(3-chloropyridin-2-yl)benzonitrile (506 mg) in the same manner as in Reference Example 103. MS: 325 (MH⁺+1).

### Reference Example 145

### N-[7-bromo-5-(3-chloropyridin-2-yl)-1H-indazol-3-yl]thiourea

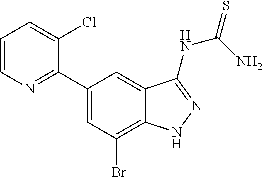

The title compound (234 g, yield 95%) was obtained as pale-yellow crystals from 7-bromo-5-(3-chloropyridin-2-yl)-1H-indazole-3-amine (209 mg) in the same manner as in Reference Example 3. Melting point 236-237° C.

### Reference Example 146

### 2-amino-5-bromo-3-methylbenzamide

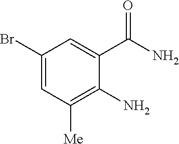

The title compound (8.1 g, yield 41%) was obtained as pale-yellow crystals from 2-amino-5-bromo-3-methylbenzoic acid (20 g) in the same manner as in Reference Example 56.

MS: 231 (MH⁺+1).

### Reference Example 147

### 2-amino-5-bromo-3-methylbenzonitrile

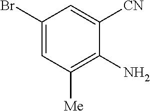

To a tetrahydrofuran solution (10 mL) of 2-amino-5-bromo-3-methylbenzamide (580 mg) and triethylamine (1.6 mL) was added dropwise trifluoroacetic acid anhydride (0.91 mL) under ice-cooling, and the mixture was stirred for 3 hr. Water was added, and the mixture was diluted with ethyl acetate, washed with water and saturated brine, dried over anhydrous magnesium sulfate, and concentrated under reduced pressure. The residue was dissolved in methanol (5 mL), water (5 mL) and potassium carbonate (700 mg) were added, and the mixture was stirred overnight at 70° C. Methanol was evaporated under reduced pressure, and diluted with ethyl acetate. The organic layer was washed with water and saturated brine, dried over anhydrous magnesium sulfate, and concentrated under reduced pressure. The residue was subjected to NH-silica gel column chromatography (eluate: ethyl acetate), and the obtained crude crystal was recrystallized (diisopropyl ether-hexane) to give the title compound (240 mg, yield 45%) as colorless crystals.

MS: 213 (MH⁺+1).

### Reference Example 148

### 2-amino-5-(3-chloropyridin-2-yl)-3-methylbenzonitrile

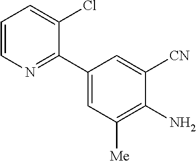

A reaction was carried out in the same manner as in Reference Example 138, a crude product of 2-amino-3-methyl-5-(4,4,5,5-tetramethyl-1,3,2-dioxaborolan-2-yl)benzonitrile was obtained from 2-amino-5-bromo-3-methylbenzonitrile (3.27 g). The title compound (1.38 g, yield 48%) was obtained as colorless crystals by subjecting the compound to a similar reaction as in Reference Example 12 without purification. ¹H NMR (300 MHz, DMSO-d₆) δ ppm 2.18 (3H, s) 6.11 (2H, s) 7.36 (1H, dd, J=8.1, 4.7 Hz) 7.61 (1H, d, J=1.3 Hz) 7.67 (1H, d, J=1.9 Hz) 7.99 (1H, dd, J=8.1, 1.5 Hz) 8.57 (1H, dd, J=4.5, 1.5 Hz)

### Reference Example 149

### 5-(3-chloropyridin-2-yl)-7-methyl-1H-indazole-3-amine

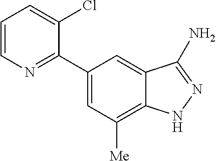

The title compound (299 mg, yield 40%) was obtained as pale-yellow crystals from 2-amino-5-(3-chloropyridin-2-yl)-3-methylbenzonitrile (707 mg) in the same manner as in Reference Example 103. MS: 259 (MH⁺).

### Reference Example 150

### 2-amino-3-methoxy-5-(4,4,5,5-tetramethyl-1,3,2-dioxaborolan-2-yl)benzonitrile

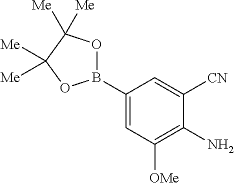

The title compound (24.51 g, yield 100%) was obtained as colorless crystals from 2-amino-5-bromo-3-methoxybenzonitrile (17.96 g) in the same manner as in Reference Example 138. Melting point 120-121° C.

### Reference Example 151

### 2-amino-5-(3-chloropyridin-2-yl)-3-methoxybenzonitrile

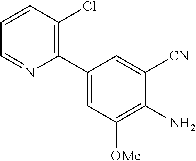

The title compound (18.0 g, yield 78%) was obtained as colorless crystals from 2-amino-3-methoxy-5-(4,4,5,5-tetramethyl-1,3,2-dioxaborolan-2-yl)benzonitrile (24.5 g) in the same manner as in Reference Example 12. Melting point 154-156° C.

### Reference Example 152

### 2-amino-5-(3-chloropyridin-2-yl)-3-hydroxybenzonitrile

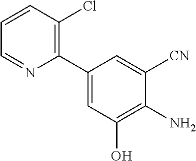

The title compound (11.63 g, yield 63%) was obtained as colorless crystals from 2-amino-5-(3-chloropyridin-2-yl)-3-methoxybenzonitrile (19.37 g) in the same manner as in Reference Example 101. Melting point 218-220° C.

### Reference Example 153

### 2-amino-5-(3-chloropyridin-2-yl)-3-(pyridin-2-ylmethoxy)benzonitrile

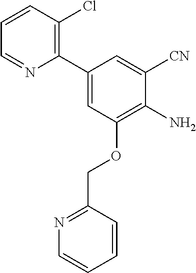

The title compound (102 mg, yield 72%) was obtained as colorless crystals from 2-amino-5-(3-chloropyridin-2-yl)-3-hydroxybenzonitrile (104 mg) and 2-(bromomethyl)pyridinehydrobromide (215 mg) in the same manner as in Reference Example 102. Melting point 178-180° C.

### Reference Example 154

### 5-(3-chloropyridin-2-yl)-7-(pyridin-2-ylmethoxy)-1H-indazole-3-amine

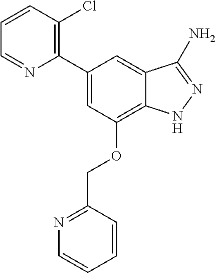

The title compound (75.8 mg, yield 71%) was obtained as colorless crystals from 2-amino-5-(3-chloropyridin-2-yl)-3-(pyridin-2-ylmethoxy)benzonitrile (102 mg) in the same manner as in Reference Example 103. Melting point 199-202° C.

### Reference Example 155

### N-[5-(3-chloropyridin-2-yl)-7-(pyridin-2-ylmethoxy)-1H-indazol-3-yl]thiourea

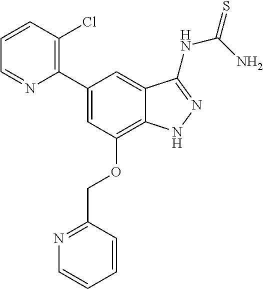

The title compound (85.7 mg, yield 99%) was obtained as pale-yellow crystals from 5-(3-chloropyridin-2-yl)-7-(pyridin-2-ylmethoxy)-1H-indazole-3-amine (74.4 mg) in the same manner as in Reference Example 3. MS: 411 (MH⁺).

### Reference Example 156

### 2-amino-5-(3-chloropyridin-2-yl)-3-(2-pyridin-2-ylethoxy)benzonitrile

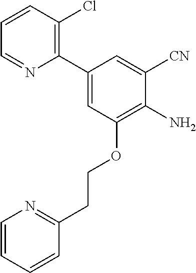

The title compound (350 mg, yield 25%) was obtained as colorless oil from 2-amino-5-(3-chloropyridin-2-yl)-3-(pyridin-2-ylmethoxy)benzonitrile (1.0 g) and 2-pyridin-2-ylethanol (0.55 mL) in the same manner as in Reference Example 127.

MS: 351 (MH⁺).

### Reference Example 157

### 5-(3-chloropyridin-2-yl)-7-(2-pyridin-2-ylethoxy)-1H-indazole-3-amine

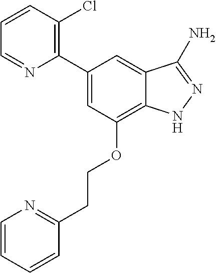

The title compound (243 mg, yield 66%) was obtained as pale-yellow non-crystalline powder from 2-amino-5-(3-chloropyridin-2-yl)-3-(2-pyridin-2-ylethoxy)benzonitrile (350 mg) in the same manner as in Reference Example 103.

MS: 366(MH⁺).

### Reference Example 158

### N-[5-(3-chloropyridin-2-yl)-7-(2-pyridin-2-ylethoxy)-1H-indazol-3-yl]thiourea

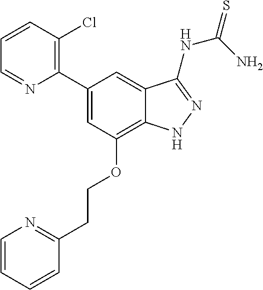

The title compound (2.09 g, yield 84%) was obtained as pale-yellow crystals from 5-(3-chloropyridin-2-yl)-7-(2-pyridin-2-ylethoxy)-1H-indazole-3-amine (2.15 g) in the same manner as in Reference Example 3. MS: 425 (MH⁺).

### Reference Example 159

### 2-amino-5-(3-chloropyridin-2-yl)-3-[3-(1,3-dioxo-1,3-dihydro-2H-isoindol-2-yl)propoxy]benzonitrile

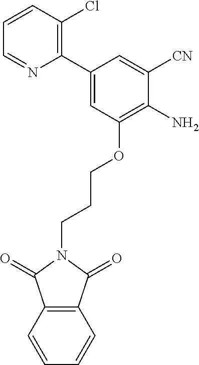

The title compound (230 mg, yield 45%) was obtained as pale-yellow non-crystalline powder from 2-amino-5-(3-chloropyridin-2-yl)-3-(pyridin-2-ylmethoxy)benzonitrile (287 mg) and 2-(3-bromopropyl)-1H-isoindole-1,3(2H)-dione (345 mg) in the same manner as in Reference Example 102. MS: 433 (MH⁺).

### Reference Example 160

### 2-(3-{[3-amino-5-(3-chloropyridin-2-yl)-1H-indazole-7-yl]oxy}propyl)-1H-isoindole-1,3(2H)-dione

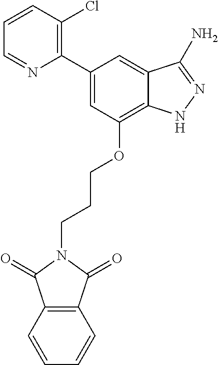

The title compound (114 mg, yield 48%) was obtained as pale-yellow non-crystalline powder from 2-amino-5-(3-chloropyridin-2-yl)-3-(pyridin-2-ylmethoxy)benzonitrile (230 mg) in the same manner as in Reference Example 103.

MS: 448 (MH⁺).

### Reference Example 161

### N-{5-(3-chloropyridin-2-yl)-7-[3-(1,3-dioxo-1,3-dihydro-2H-isoindol-2-yl)propoxy]-1H-indazol-3-yl}thiourea

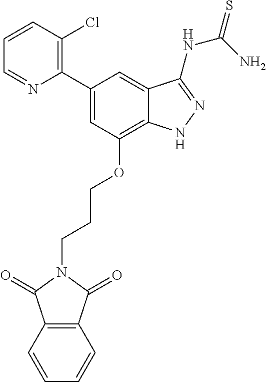

The title compound (320 mg, yield 100%) was obtained as pale-yellow non-crystalline powder from 2-(3-{[3-amino-5-(3-chloropyridin-2-yl)-1H-indazole-7-yl]oxy}propyl)-1H-isoindole-1,3(2H)-dione (265 mg) in the same manner as in Reference Example 3. MS: 507(MW).

### Reference Example 162

### 2-{[5-(3-chloropyridin-2-yl)-7-(2-pyridin-2-ylethoxy)-1H-indazol-3-yl]amino}-1,3-thiazole-5-carbaldehyde

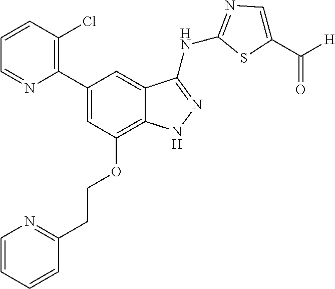

The title compound (1.37 g, yield 62%) was obtained as brown crystals from N-[5-(3-chloropyridin-2-yl)-7-(2-pyridin-2-ylethoxy)-1H-indazol-3-yl]thiourea (1.98 g) in the same manner as in Reference Example 87. MS: 477(MH⁺).

### Reference Example 163

### ethyl (2-{[5-(3-chloropyridin-2-yl)-7-(2-pyridin-2-ylethoxy)-1H-indazol-3-yl]amino}-1,3-thiazol-5-yl)acetate

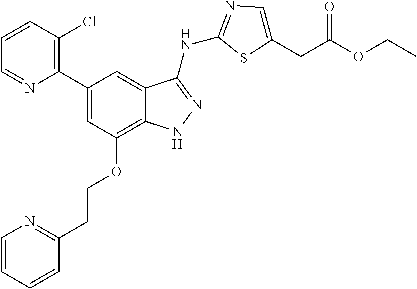

An ethanol-tetrahydrofuran solution (2 mL to 1 mL) of N-[5-(3-chloropyridin-2-yl)-7-(2-pyridin-2-ylethoxy)-1H-indazol-3-yl]thiourea (100 mg) and ethyl 3-bromo-4-oxobutanate (54 mg) was stirred overnight at 80° C. Saturated aqueous sodium hydrogen carbonate was added, and tetrahydrofuran was extracted with ethyl acetate. The organic layer was washed with water and saturated brine, dried over anhydrous magnesium sulfate, and concentrated under reduced pressure. The precipitated solid was washed with diisopropyl ether to give the title compound (108 mg, yield 86%) as brown crystals. MS: 535 (MH⁺).

### Reference Example 164

### methyl 6-{[2-amino-5-(3-chloropyridin-2-yl)-3-cyanophenoxy]methyl}nicotinate

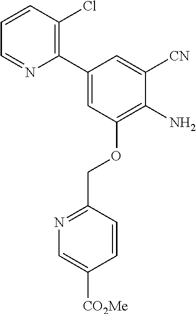

The title compound (970 mg, yield 63%) was obtained as colorless crystals from 2-amino-5-(3-chloropyridin-2-yl)-3-(pyridin-2-ylmethoxy)benzonitrile (900 mg) and methyl 6-(bromomethyl)nicotinate (0.90 g) in the same manner as in Reference Example 102. ¹H NMR (300 MHz, DMSO-d₆) δ ppm 3.90 (3H, s) 5.38 (2H, s) 6.25 (2H, s) 7.36 (1H, dd, J=8.0, 4.5 Hz) 7.45 (2H, d, J=2.7 Hz) 7.86 (1H, d, J=8.3 Hz) 7.98 (1H, dd, J=8.0, 1.5 Hz) 8.34 (1H, dd, J=8.1, 2.1 Hz) 8.57 (1H, dd, J=4.5, 1.5 Hz) 9.08 (1H, d, J=1.9 Hz)

### Reference Example 165

### methyl 6-({[3-amino-5-(3-chloropyridin-2-yl)-1H-indazole-7-yl]oxy}methyl)nicotinate

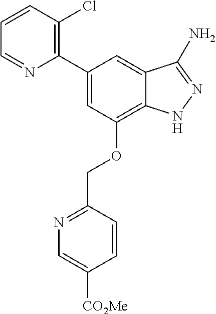

The title compound (900 mg, yield 89%) was obtained as colorless crystals from methyl 6-{[2-amino-5-(3-chloropyridin-2-yl)-3-cyanophenoxy]methyl}nicotinate (970 mg) in the same manner as in Reference Example 103. MS: 410 (MH⁺).

### Reference Example 166

### methyl 6-({[3-[(aminocarbonothioyl)amino]-5-(3-chloropyridin-2-yl)-1H-indazole-7-yl]oxy}methyl)nicotinate

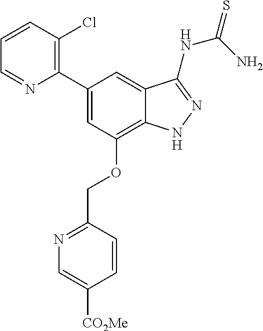

The title compound (1.13 g, yield 100%) was obtained as pale-yellow non-crystalline powder from methyl 6-({[3-amino-5-(3-chloropyridin-2-yl)-1H-indazole-7-yl]oxy}methyl)nicotinate (900 mg) in the same manner as in Reference Example 3.

MS: 469 (MH⁺).

### Reference Example 167

### 2-[5-(3-chloropyridin-2-yl)-7-(2-pyridin-2-ylethoxy)-1H-indazol-3-yl]-1H-isoindole-1,3(2H)-dione

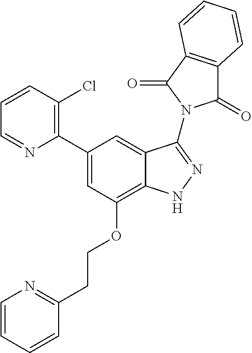

To a mixture of 5-(3-chloropyridin-2-yl)-7-(2-pyridin-2-ylethoxy)-1H-indazole-3-amine (38.6 mg), phthalic acid (19.3 mg), 1H-1,2,3-benzotriazol-1-ol (39 mg) and N,N-dimethylformamide (2 mL) was added N-[3-(dimethylamino)propyl]-N′-ethylcarbodiimide hydrochloride (49 mg) at room temperature, and the mixture was stirred at 50° C. for 2 hr. To the reaction mixture was added saturated aqueous sodium hydrogen carbonate, and the mixture was extracted with ethyl acetate. The ethyl acetate layer was washed with saturated brine, dried over anhydrous magnesium sulfate, and concentrated under reduced pressure. The residue was purified by silica gel column chromatography (eluate: ethyl acetate) to give the title compound (72 mg, yield 100%) as yellow amorphous crystals.

MS: 496 (MH⁺).

### Reference Example 168

### 2-[5-(3-chloropyridin-2-yl)-1-methyl-7-(2-pyridin-2-ylethoxy)-1H-indazol-3-yl]-1H-isoindole-1,3(2H)-dione

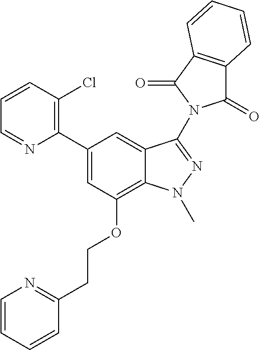

The title compound (19.7 mg, yield 20%) was obtained as pale-yellow non-crystalline powder from 2-[(5-(3-chloropyridin-2-yl)-7-(2-pyridin-2-ylethoxy)-1H-indazol-3-yl]-1H-isoindole-1,3(2H)-dione (91 mg) in the same manner as in Reference Example 78. MS: 510 (MH⁺).

### Reference Example 169

### 5-(3-chloropyridin-2-yl)-1-methyl-7-(2-pyridin-2-ylethoxy)-1H-indazole-3-amine

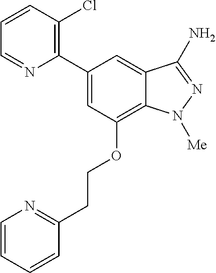

The title compound (17.5 mg, yield 100%) was obtained as pale-yellow non-crystalline powder from 2-[(5-(3-chloropyridin-2-yl)-1-methyl-7-(2-pyridin-2-ylethoxy)-1H-indazol-3-yl]-1H-isoindole-1,3(2H)-dione (18.7 mg) in the same manner as in Reference Example 79. MS: 379 (MH⁺).

### Reference Example 170

### N-[5-(3-chloropyridin-2-yl)-1-methyl-7-(2-pyridin-2-ylethoxy)-1H-indazol-3-yl]thiourea

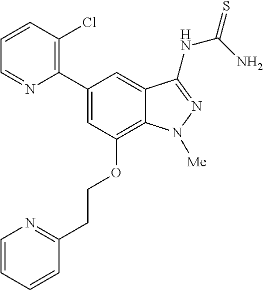

The title compound (14.7 mg, yield 90%) was obtained as pale-yellow crystals from 5-(3-chloropyridin-2-yl)-1-methyl-7-(2-pyridin-2-ylethoxy)-1H-indazole-3-amine (17.5 mg) in the same manner as in Reference Example 3. Melting point 170-172° C.

### Reference Example 171

### 2-[5-(3-chloropyridin-2-yl)-1-(methoxymethyl)-7-(2-pyridin-2-ylethoxy)-1H-indazol-3-yl]-1H-isoindole-1,3(2H)-dione

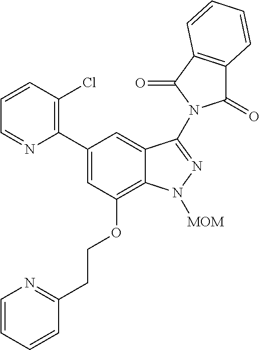

To a mixture of 2-[5-(3-chloropyridin-2-yl)-7-(2-pyridin-2-ylethoxy)-1H-indazol-3-yl]-1H-isoindole-1,3(2H)-dione (1.14 g) and N,N-dimethylformamide (30 mL) was added sodium hydride (60%, oily, 0.11 g) under ice-cooling, and the mixture was stirred for 30 min. To the reaction mixture was added chloromethyl methyl ether (0.20 mL) at 0° C. and stirred for 3 hr, water was added, and the mixture was extracted with ethyl acetate. The ethyl acetate layer was washed with saturated brine, dried over anhydrous magnesium sulfate, and concentrated under reduced pressure. The residue was purified by silica gel column chromatography (hexane:ethyl acetate=1:1-0:1, ethyl acetate:methanol=30:1) to give the title compound (956 mg, yield 76%) as pale-yellow non-crystalline powder. MS: 539 (MH⁺).

### Reference Example 172

### 5-(3-chloropyridin-2-yl)-1-(methoxymethyl)-7-(2-pyridin-2-ylethoxy)-1H-indazole-3-amine

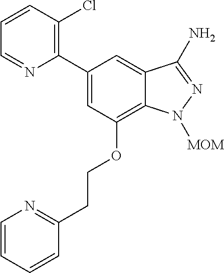

The title compound (514 mg, yield 72%) was obtained as pale-yellow crystals from 2-[(5-(3-chloropyridin-2-yl)-1-(methoxymethyl)-7-(2-pyridin-2-ylethoxy)-1H-indazol-3-yl]-1H-isoindole-1,3(2H)-dione (940 mg) in the same manner as in Reference Example 79. Melting point 120-122° C.

### Reference Example 173

### 3-bromo-5-(3-chloropyridin-2-yl)-7-(2-pyridin-2-ylethoxy)-1H-indazole

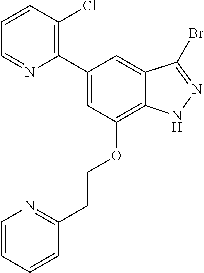

To an acetic acid—hydrobromic acid (48%) solution (2 mL-2 mL) of 5-(3-chloropyridin-2-yl)-7-(2-pyridin-2-ylethoxy)-1H-indazole-3-amine (180 mg) was added dropwise an aqueous solution (0.5 mL) of sodium nitrite (38 mg) under ice-cooling, and the mixture was stirred for 30 min. Copper (I) bromide (140 mg) was added under ice-cooling, and the mixture was m stirred for 30 min. The mixture was basified by saturated aqueous sodium hydrogen carbonate, and tetrahydrofuran was extracted with ethyl acetate. The organic layer was washed with water and saturated brine, dried over anhydrous magnesium sulfate, and concentrated under reduced pressure. The obtained crude crystals were recrystallized (tetrahydrofuran-diisopropyl ether) to give the title compound (97.5 mg, yield 46%) as colorless crystals. Melting point>250° C.

### Reference Example 174

### tert-butyl 3-bromo-5-(3-chloropyridin-2-yl)-7-(2-pyridin-2-ylethoxy)-1H-indazole-1-carboxylate

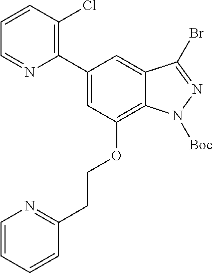

The title compound (71.7 mg, yield 60%) was obtained as pale-yellow non-crystalline powder from 3-bromo-5-(3-chloropyridin-2-yl)-7-(2-pyridin-2-ylethoxy)-1H-indazole (97 mg) in the same manner as in Reference Example 98.

MS: 531 (MH⁺+1).

### Reference Example 175

### tert-butyl 5-(3-chloropyridin-2-yl)-3-[(1-methyl-1H-pyrazol-3-yl)amino]-7-(2-pyridin-2-ylethoxy)-1H-indazole-1-carboxylate

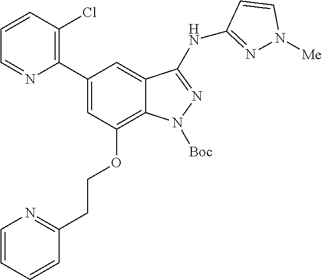

A mixture of tert-butyl 3-bromo-5-(3-chloropyridin-2-yl)-7-(2-pyridin-2-ylethoxy)-1H-indazole-1-carboxylate (69.8 mg), 1-methyl-1H-pyrazole-3-amine (15.3 mg), cesium carbonate (86% mg), tris(dibenzylidene)dipalladium(0)(6 mg), (9,9-dimethyl-9H-xanthen-4,5-diyl)bis(diphenylphosphine)(11.5 mg) and 1,4-dioxane (2 mL) was stirred at 100° C. for 3 hr under nitrogen atmosphere. The mixture was diluted with ethyl acetate, washed with water and saturated brine, dried over anhydrous magnesium sulfate, and concentrated under reduced pressure. The residue was purified by silica gel column chromatography (hexane:ethyl acetate=2:1-1:2) to give the title compound (55.9 mg, yield 78%) as colorless non-crystalline powder. MS: 546 (MH⁺).

### Reference Example 176

### tert-butyl 3-amino-5-(3-chloropyridin-2-yl)-1H-indazole-1-carboxylate

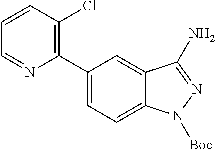

A tetrahydrofuran solution (5 mL) of 5-(3-chloropyridin-2-yl)-1H-indazole-3-amine (175 mg), triethylamine (0.12 mL), 4-dimethylaminopyridine (8.7 mg) and di-tert-butyl dicarbonate (68 mg) was stirred overnight at room temperature. The solution was diluted with ethyl acetate, and the organic layer was washed with water and saturated brine, dried over anhydrous magnesium sulfate, and concentrated under reduced pressure. The residue was purified by silica gel column chromatography (hexane:ethyl acetate=2:1-1:5) to give the title compound (160 mg, yield 65%) as brown oil. MS: 345 (MH⁺).

### Reference Example 177

### 2-methyl-6-[4-(methylsulfonyl)phenoxy]aniline

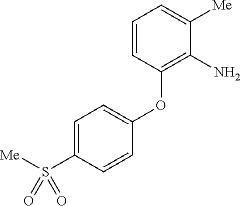

An N,N-dimethylformamide solution (50 mL) of 3-methyl-2-nitrophenol (4.28 g), 1-fluoro-4-(methylsulfonyl)benzene (4.63 g) and potassium carbonate (5.52 g) were stirred overnight at 130° C. The solution was diluted with ethyl acetate, washed with water and saturated brine, dried over anhydrous magnesium sulfate, and concentrated under reduced pressure. The residue was purified by NH-silica gel column chromatography (hexane:ethyl acetate=10:1-1:1). The product was brought into an ethyl acetate solution (50 mL), 10% palladium-carbon (0.5 g) was added, and the mixture was stirred for 2 hr at room temperature under hydrogen atmosphere. The insoluble materials were filtered off, and the filtrate was concentrated under reduced pressure. The residue was purified by silica gel column chromatography (hexane:ethyl acetate=10:1-0:1) to give the title compound (2.83 g, yield 38%) as colorless oil.

MS: 278 (MH⁺).

### Reference Example 178

### 4-bromo-2-methyl-6-[4-(methylsulfonyl)phenoxy]aniline

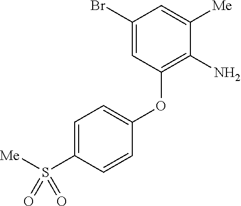

To an N,N-dimethylformamide solution (50 mL) of 2-methyl-6-[4-(methylsulfonyl)phenoxy]aniline (2.85 g) was added an N,N-dimethylformamide solution (10 mL) of N-bromosuccinimide (1.92 g) under ice-cooling, and the mixture was stirred for 4 hr. The mixture was diluted with ethyl acetate, washed with water and saturated brine, dried over anhydrous magnesium sulfate, and concentrated under reduced pressure. The obtained crude crystals were recrystallized (toluene) to give the title compound (1.81 g, yield 49%) as a brown solid. MS: 358(MH⁺).

### Reference Example 179

### 4-(3-chloropyridin-2-yl)-2-methyl-6-[4-(methylsulfonyl)phenoxy]aniline

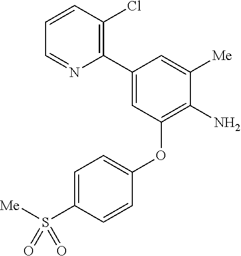

4-Bromo-2-methyl-6-[4-(methylsulfonyl)phenoxy]aniline (1.81 g) was converted to a boric acid ester in the same manner as in Reference Example 138, and the product was subjected to Suzuki coupling reaction in the same manner as in Reference Example 12 without purification to give the title compound (0.95 g, yield 49%) as colorless non-crystalline powder.

MS: 389(MH⁺).

### Reference Example 180

### 5-(3-chloropyridin-2-yl)-7-[4-(methylsulfonyl)phenoxy]-1H-indazole

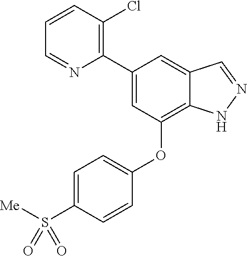

To a toluene solution (8 mL) of 4-(3-chloropyridin-2-yl)-2-methyl-6-[4-(methylsulfonyl)phenoxy]aniline (578 mg) and potassium acetate (162 mg) was added acetic anhydride (0.57 mL) under ice-cooling, and the mixture was stirred at room temperature for 30 min. To the reaction solution was added dropwise isoamyl nitrite (0.40 mL), and the mixture was stirred at 80° C. overnight. To the reaction mixture were added potassium carbonate (1.65 g) and methanol (30 mL), and the m mixture was stirred at 60° C. overnight. The mixture was diluted with ethyl acetate, washed with water and saturated brine, dried over anhydrous magnesium sulfate, and concentrated under reduced pressure. The residue was purified by silica gel column chromatography (hexane:ethyl acetate=3:1-0:1) to is give the title compound (424 mg, yield 72%) as pale-yellow non-crystalline powder. MS: 400 (MH⁺).

### Reference Example 181

### 3-bromo-5-(3-chloropyridin-2-yl)-7-[4-(methylsulfonyl)phenoxy]-1H-indazole

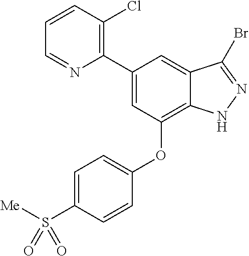

The title compound (407 mg, yield 85%) was obtained as pale-yellow non-crystalline powder from 5-(3-chloropyridin-2-yl)-7-[4-(methylsulfonyl)phenoxy]-1H-indazole (401 mg) in the same manner as in Reference Example 140. MS: 480 (MH⁺+1).

### Reference Example 182

### 3-bromo-5-(3-chloropyridin-2-yl)-1-(methoxymethyl)-7-[4-(methylsulfonyl)phenoxy]-1H-indazole

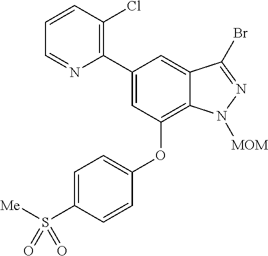

The title compound (368 mg, yield 83%) was obtained as colorless non-crystalline powder from 3-bromo-5-(3-chloropyridin-2-yl)-7-[4-(methylsulfonyl)phenoxy]-1H-indazole (405 mg) in the same manner as in Reference Example 171.

MS: 524 (MH⁺+1).

### Reference Example 183

### 5-(3-chloropyridin-2-yl)-1-(methoxymethyl)-N-(1-methyl-1H-pyrazol-3-yl)-7-[4-(methylsulfonyl)phenoxy]-1H-indazole-3-amine

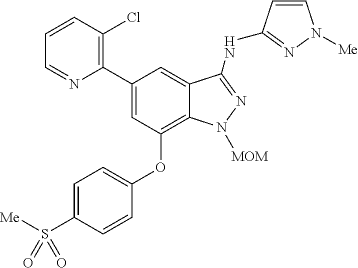

The title compound (294 mg, yield 76%) was obtained as colorless non-crystalline powder from 3-bromo-5-(3-chloropyridin-2-yl)-1-(methoxymethyl)-7-[4-(methylsulfonyl)phenoxy]-1H-indazole (368 mg) in the same manner as in Reference Example 175. MS: 539(MH⁺).

### Reference Example 184

### 2-amino-5-bromo-3-(2-pyridin-2-ylethoxy)benzonitrile

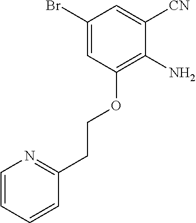

The title compound (7.0 g, yield 37%) was obtained as pale-yellow crystals from 2-amino-5-bromo-3-hydroxybenzonitrile (12.48 g) in the same manner as in Reference Example 127.

MS: 320 (MH⁺+1)

### Reference Example 185

### 2-amino-3-(2-pyridin-2-ylethoxy)-5-(4,4,5,5-tetramethyl-1,3,2-dioxaborolan-2-yl)benzonitrile

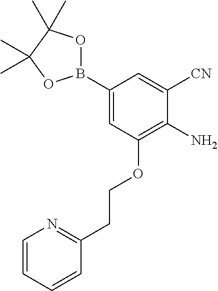

The title compound (7.28 g, yield 95%) was obtained as pale-yellow non-crystalline powder from 2-amino-5-bromo-3-(2-pyridin-2-ylethoxy)benzonitrile (6.68 g) in the same manner as in Reference Example 138. MS: 366(MH⁺).

### Reference Example 186

### 2-amino-5-hydroxy-3-(2-pyridin-2-ylethoxy)benzonitrile

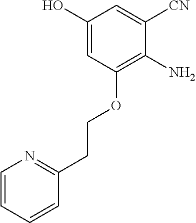

To a methanol solution (200 ml) of 2-amino-3-(2-pyridin-2-ylethoxy)-5-(4,4,5,5-tetramethyl-1,3,2-dioxaborolan-2-yl)benzonitrile (6.28 g) were added dropwise 1N sodium hydroxide (18 mL) and hydrogen peroxide (35%, 1.6 mL) under ice-cooling, and the mixture was stirred for 30 min. The mixture was acidified by adding 1N hydrochloric acid (30 mL), and weakly basified by adding saturated aqueous sodium hydrogen carbonate. The aqueous layer was extracted with ethyl acetate, washed with water and saturated brine, dried over anhydrous magnesium sulfate, and concentrated under reduced pressure. The obtained crude crystals were recrystallized (ethyl acetate-tetrahydrofuran) to give the title compound (3.41 g, yield 78%) as yellow crystals. Melting point 149-150° C.

### Reference Example 187

### 2-amino-5-(benzyloxy)-3-(2-pyridin-2-ylethoxy)benzonitrile

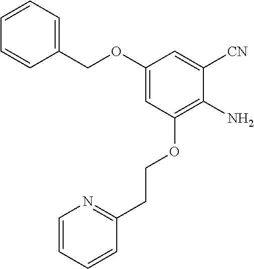

To a tetrahydrofuran solution (10 mL) of 2-amino-5-hydroxy-3-(2-pyridin-2-ylethoxy)benzonitrile (203 mg), tributylphosphine (0.39 mL) and benzyl alcohol (0.12 mL) was added 1,1′-(azodicarbonyl)dipiperidine (399 mg), and the mixture was stirred overnight at 60° C. The mixture was concentrated under reduced pressure, diisopropyl ether was added, and insoluble materials were filtered through Celite. The mother liquor was concentrated under reduced pressure, and the obtained residue was purified by silica gel column chromatography (hexane:ethyl acetate=5:1-1:1) to give the title compound (305 mg, yield 100%) as a pale-yellow oily substance. MS: 346 (MH⁺).

### Reference Example 188

### 5-(benzyloxy)-7-(2-pyridin-2-ylethoxy)-1H-indazole-3-amine

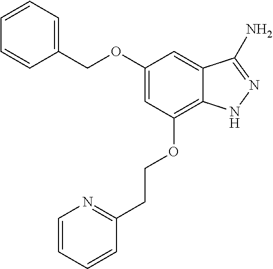

The title compound (342 mg, yield 52%) was obtained as pale-yellow non-crystalline powder from 2-amino-5-hydroxy-3-(2-pyridin-2-ylethoxy)benzonitrile (358 mg) in the same manner as in Reference Example 103. MS: 361 (MH⁺).

### Reference Example 189

### N-[5-(benzyloxy)-7-(2-pyridin-2-ylethoxy)-1H-indazol-3-yl]thiourea

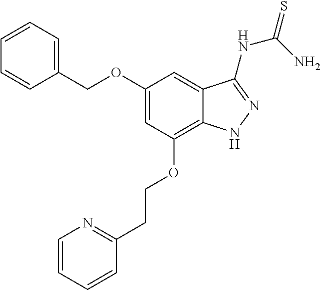

The title compound (447 mg, yield 100%) was obtained as colorless crystals from 5-(benzyloxy)-7-(2-pyridin-2-ylethoxy)-1H-indazole-3-amine (342 mg) in the same manner as in Reference Example 3. Melting point 109-110° C.

### Reference Example 190

### 2-amino-5-isopropoxy-3-(2-pyridin-2-ylethoxy)benzonitrile

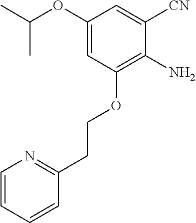

An N,N-dimethylformamide solution (200 mL) of 2-amino-5-hydroxy-3-(2-pyridin-2-ylethoxy)benzonitrile (300 mg), potassium carbonate (212 mg) and isopropyl iodide (0.13 mL) was stirred overnight at 80° C. The mixture was diluted with ethyl acetate, washed with water and saturated brine, dried over anhydrous magnesium sulfate, and concentrated under reduced pressure. The residue was subjected to NH-silica gel column chromatography (eluate: ethyl acetate) and silica gel column chromatography (hexane:ethyl acetate=10:1-3:1) to give the title compound (129 mg, yield 37%) as a yellow oily substance.

MS: 298 (MH⁺).

### Reference Example 191

### 5-isopropoxy-7-(2-pyridin-2-ylethoxy)-1H-indazole-3-amine

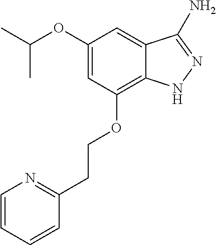

The title compound (108 mg, yield 84%) was obtained as a pale-yellow oily substance from 2-amino-5-isopropoxy-3-(2-pyridin-2-ylethoxy)benzonitrile (121 mg) in the same manner as in Reference Example 103. MS: 313 (MH⁺).

### Reference Example 192

### N-[5-isopropoxy-7-(2-pyridin-2-ylethoxy)-1H-indazol-3-yl]thiourea

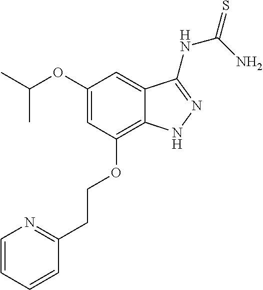

The title compound (102 mg, yield 97%) was obtained as a pale-yellow oily substance from 5-isopropoxy-7-(2-pyridin-2-ylethoxy)-1H-indazole-3-amine (88.1 mg) in the same manner as in Reference Example 3. MS: 372 (MH⁺).

### Reference Example 193

### 2-amino-5-[(1S)-2-methoxy-1-methylethoxy]-3-(2-pyridin-2-ylethoxy)benzonitrile

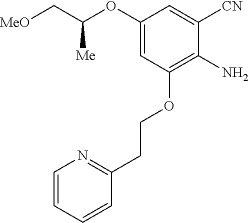

To a tetrahydrofuran solution (10 mL) of 2-amino-5-hydroxy-3-(2-pyridin-2-ylethoxy)benzonitrile (303 mg), tributylphosphine (0.60 mL) and (2R)-1-methoxypropane-2-ol (0.18 mL) was added 1,1′-(azodicarbonyl)dipiperidine (600 mg), and the mixture was stirred at 60° C. for 4 hr. The reaction solution was concentrated under reduced pressure, diisopropyl ether was added, and insoluble materials were filtered through Celite. The mother liquor was concentrated under reduced pressure, and purified by NH-silica gel column chromatography (hexane:ethyl acetate=5:1-1:1) and silica gel column chromatography (hexane:ethyl acetate=2:1-1:4) to give the title compound (405 mg, yield 100%) as a brown oily substance.

MS: 328 (MH⁺).

### Reference Example 194

### 5-[(1S)-2-methoxy-1-methylethoxy]-7-(2-pyridin-2-ylethoxy)-1H-indazole-3-amine

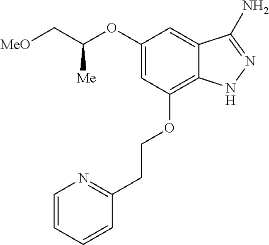

The title compound (160 mg, yield 42%) was obtained as pale-yellow non-crystalline powder from 2-amino-5-[(1S)-2-methoxy-1-methylethoxy]-3-(2-pyridin-2-ylethoxy)benzonitrile (365 mg) in the same manner as in Reference Example 103.

MS: 343 (MH⁺).

### Reference Example 195

### N-[5-[(1S)-2-methoxy-1-methylethoxy]-7-(2-pyridin-2-ylethoxy)-1H-indazol-3-yl]thiourea

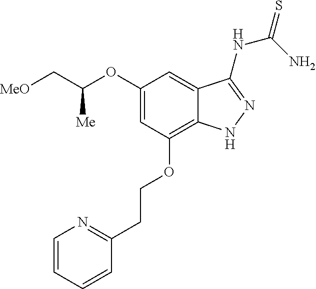

The title compound (188 mg, yield 99%) was obtained as a pale-yellow oily substance from 5-[(1S)-2-methoxy-1-methylethoxy]-7-(2-pyridin-2-ylethoxy)-1H-indazole-3-amine (154 mg) in the same manner as in Reference Example 3. MS: 402 (MH⁺).

### Reference Example 196

### 5-[(1-methyl-1H-imidazol-2-yl)thio]-1H-indazole-3-amine

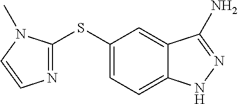

To a dichloromethane solution (20 mL) of (3-cyano-4-fluorophenyl)boronic acid (660 mg) were added 1-methyl-1H-imidazole-2-thiol (913 mg), copper(II) acetate (1.45 g) and pyridine (1 mL), and the mixture was stirred at room temperature for 3 days. The insoluble materials were removed by filtration. The filtrate was diluted with ethyl acetate and water. The organic layer was washed with saturated aqueous sodium hydrogen carbonate and saturated brine, dried over anhydrous magnesium sulfate, and concentrated under reduced pressure. The residue was purified by NH-silica gel column chromatography (eluate: ethyl acetate) to give a yellow oily substance. The obtained a yellow oily substance was dissolved in 1-butanol (10 mL), hydrazine monohydrate (0.5 mL) was added, and the mixture was stirred at 130° C. for 4 hr. The reaction mixture was concentrated, and washed with diluted with ethyl acetate and water. The organic layer was washed with saturated aqueous sodium hydrogen carbonate and saturated brine, dried over anhydrous magnesium sulfate, and concentrated under reduced pressure to give the title compound (120 mg, yield 13%) as pale-yellow crystals. Melting point 174° C.

### Reference Example 197

### 4-amino-3-cyanophenylthiocyanate

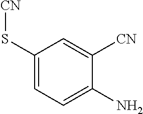

To a solution of 2-aminobenzonitrile (10.0 g) and potassium thiocyanate (12.3 g) in methanol (160 mL) was added dropwise bromine (4.6 mL) at 0° C. for 40 min. The reaction suspension was stirred at room temperature for 1 hr and poured into water (300 mL). The precipitated crystal was collected by filtration, washed with water and dried. The obtained crude crystals were recrystallized (ethyl acetate-hexane) to give the title compound (9.89 g, yield 67%) as pale-yellow crystals. Melting point 120-121° C.

### Reference Example 198

### 2-amino-5-(isopropylthio)benzonitrile

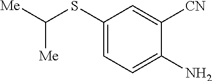

A tetrahydrofuran solution (20 mL) of 4-amino-3-cyanophenylthiocyanate (3.86 g), 2-iodopropane (3.0 mL), 4N aqueous sodium hydroxide solution (5.8 mL) and 15-crown-5 (0.44 mL) was stirred for 2 hr at room temperature, sodium borohydride (0.46 g) was then added, and the mixture was stirred overnight at room temperature. The reaction mixture was concentrated, and the residue was diluted ethyl acetate, washed with water and saturated brine, dried over anhydrous magnesium sulfate, and concentrated under reduced pressure. The residue was purified by NH-silica gel column chromatography (ethyl acetate:hexane=2:1) to give the title compound (3.97 g, yield 94%) as pale-yellow crystals. Melting point 67-68° C.

### Reference Example 199

### 5-(isopropylthio)-1H-indazole-3-amine

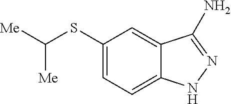

The title compound (1.77 g, yield 69%) was obtained as pale-yellow crystals from 2-amino-5-(isopropylthio)benzonitrile (2.37 g) in the same manner as in Reference Example 103. Melting point 160-161° C.

### Reference Example 200

### N-[5-(isopropylthio)-1H-indazol-3-yl]thiourea

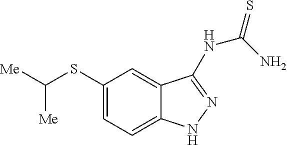

The title compound (1.29 g, yield 100%) was obtained as pale-yellow crystals from 5-(isopropylthio)-1H-indazole-3-amine (1.00 g) in the same manner as in Reference Example 3. Melting point 168-170° C.

### Reference Example 201

### 2-amino-5-(isobutylthio)benzonitrile

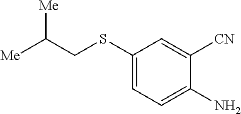

The title compound (2.86 g, yield 81%) was obtained as yellow crystals from 4-amino-3-cyanophenylthiocyanate (3.0 g) and isobutyl iodide (2.4 mL) in the same manner as in Reference Example 198. Melting point 54-55° C.

### Reference Example 202

### 5-(isobutylthio)-1H-indazole-3-amine

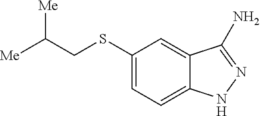

The title compound (1.75 g, yield 50%) was obtained as pale-yellow crystals from 2-amino-5-(isobutylthio)benzonitrile (3.23 g) in the same manner as in Reference Example 103. Melting point 138-139° C.

### Reference Example 203

### N-[5-(isobutylthio)-1H-indazol-3-yl]thiourea

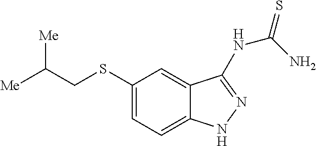

The title compound (440 mg, yield 100%) was obtained as pale-yellow non-crystalline powder from 5-(isobutylthio)-1H-indazole-3-amine (300 mg) in the same manner as in Reference Example 3. MS: 281 (MH⁺).

### Reference Example 204

### 2-amino-5-(cyclopentylthio)benzonitrile

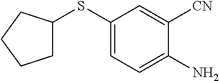

The title compound (2.99 g, yield 80%) was obtained as yellow crystals from 4-amino-3-cyanophenylthiocyanate (3.00 g) and cyclopentyl iodide (2.4 mL) in the same manner as in Reference Example 198. Melting point 59-60° C.

### Reference Example 205

### 5-(cyclopentylthio)-1H-indazole-3-amine

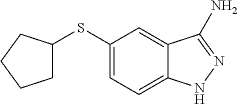

The title compound (278 mg, yield 9%) was obtained as pale-yellow crystals from 2-amino-5-(cyclopentylthio)benzonitrile (2.99 g) in the same manner as in Reference Example 103. Melting point 167-168° C.

### Reference Example 206

### 4-amino-3-cyano-5-methoxyphenylthiocyanate

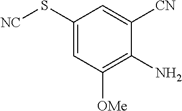

To a solution of sodium thiocyanate (4.06 g) in methanol (70 mL) was added bromine (1.35 mL) at −70° C. The reaction mixture was stirred for 10 min, and 2-amino-3-methoxybenzonitrile (3.71 g) was added. The temperature was risen to room temperature, and the mixture was stirred for 2 hr. The reaction mixture was poured into ice water, and the precipitated crystals were collected by filtration, washed with water and dried to give the title compound (4.28 g, yield 84%) as colorless crystals. Melting point 121-122° C.

### Reference Example 207

### 2-amino-5-(isopropylthio)-3-methoxybenzonitrile

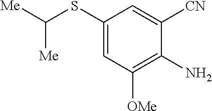

To a mixture of 4-amino-3-cyano-5-methoxyphenyl thiocyanate (4.86 g), isopropyl iodide (3.2 mL), tetrahydrofuran (10 mL) and 2-propanol (10 mL) was added 2N aqueous sodium hydroxide solution (12 mL), and the mixture was stirred for 30 min. Sodium borohydride (0.50 g) was added, and the reaction mixture was stirred overnight at room temperature. The reaction mixture was concentrated, water was added, and extracted with ethyl acetate. The ethyl acetate layer was washed with saturated brine, dried over anhydrous magnesium sulfate, and concentrated under reduced pressure. The residue was purified by silica gel column chromatography (hexane:ethyl acetate=2:1) to give the title compound (4.76 g, yield 90%) as colorless crystals. Melting point 81-82° C.

### Reference Example 208

### 2-amino-3-hydroxy-5-(isopropylthio)benzonitrile

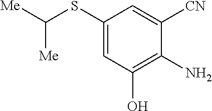

A mixture of 2-amino-5-(isopropylthio)-3-methoxybenzonitrile (3.76 g) and boron tribromide (1M dichloromethane solution; 51.0 mL) was stirred overnight at room temperature. The reaction mixture was neutralized by adding saturated aqueous sodium hydrogen carbonate, and the dichloromethane layer was separated and concentrated to give a residue. The aqueous layer was extracted with ethyl acetate, combined with the above-mentioned residue, washed with saturated brine, dried over anhydrous magnesium sulfate, and concentrated under reduced pressure. The residue was purified by silica gel column chromatography (hexane:ethyl acetate=2:1) to give the title compound (2.44 g, yield 69%) as yellow crystals. Melting point 145-146° C.

### Reference Example 209

### 2-amino-3-hydroxy-5-(isopropylsulfonyl)benzonitrile

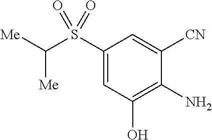

To a mixture of 2-amino-3-hydroxy-5-(isopropylthio)benzonitrile (2.44 g), tetrahydrofuran (15 mL), methanol (15 mL) and water (5 mL) was added Oxone (7.91 g), and the mixture was stirred at room temperature for 2 hr. The residual Oxone was decomposed with sodium sulfite and concentrated. To the residue was added water and extracted with ethyl acetate. The ethyl acetate layer was washed with saturated brine, dried over anhydrous magnesium sulfate, and concentrated under reduced pressure. The residual solid was washed with isopropyl ether and dried to give the title compound (2.81 g, yield 99%) as yellow crystals. Melting point 166-167° C.

### Reference Example 210

### 2-amino-5-(isopropylsulfonyl)-3-(2-pyridin-2-ylethoxy)benzonitrile

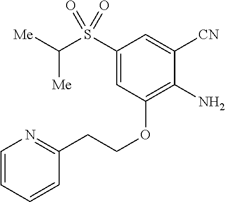

To a solution of 2-pyridin-2-ylethanol (0.55 g), triethylamine (0.70 mL) and tetrahydrofuran (8 mL) was added methanesulfonyl chloride (0.38 mL) at 0° C., and the mixture was stirred for 1 hr. The precipitated solid was removed by filtration, and the filtrate was concentrated to give a pale-yellow oily substance. A mixture of the obtained oily substance, 2-amino-3-hydroxy-5-(isopropylsulfonyl)benzonitrile (0.55 g), potassium carbonate (0.46 g) and N,N-dimethylformamide (30 mL) was stirred at 70° C. for 5 hr. To the reaction mixture was added water, and extracted with ethyl acetate. The ethyl acetate layer was washed with saturated brine, dried over anhydrous magnesium sulfate, and concentrated under reduced pressure. The residue was purified by NH-silica gel column chromatography (hexane:ethyl acetate=1:2) to give the title compound (0.59 g, yield 52%) as colorless crystals. Melting point 122-123° C.

### Reference Example 211

### 5-(isopropylsulfonyl)-7-(2-pyridin-2-ylethoxy)-1H-indazole-3-amine

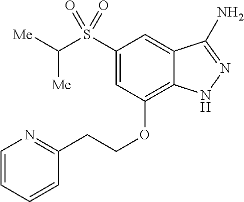

The title compound (0.33 g, yield 54%) was obtained as yellow non-crystalline powder from 2-amino-5-(isopropylsulfonyl)-3-(2-pyridin-2-ylethoxy)benzonitrile (0.59 g) in the same manner as in Reference Example 103. MS: 361(MH⁺).

### Reference Example 212

### 2-amino-5-(isopropylsulfonyl)-3-[3-(methylsulfonyl)propoxy]benzonitrile

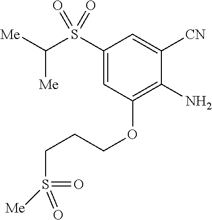

A mixture of 2-amino-3-hydroxy-5-(isopropylsulfonyl)benzonitrile (0.73 g), 3-(methylsulfonyl)propyl 4-methylbenzenesulfonate (1.06 g), potassium carbonate (0.50 g) and N,N-dimethylformamide (10 mL) was stirred overnight at 70° C. Water was added to the reaction mixture and extracted with ethyl acetate. The ethyl acetate layer was washed with saturated brine, dried over anhydrous magnesium sulfate, and concentrated under reduced pressure. The residue was purified by silica gel column chromatography (eluate: ethyl acetate) to give the title compound (0.95 g, yield 87%) as colorless crystals. Melting point 142-143° C.

### Reference Example 213

### 5-(isopropylsulfonyl)-7-[3-(methylsulfonyl)propoxy]-1H-indazole-3-amine

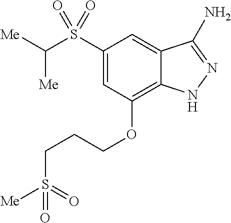

The title compound (0.80 g, yield 81%) was obtained as pale-yellow crystals from 2-amino-5-(isopropylsulfonyl)-3-[3-(methylsulfonyl)propoxy]benzonitrile (0.95 g) in the same manner as in Reference Example 103. Melting point 233-235° C.

### Reference Example 214

### 2-{[5-(isopropylsulfonyl)-7-(2-pyridin-2-ylethoxy)-1H-indazol-3-yl]amino}-1,3-thiazole-5-carbaldehyde

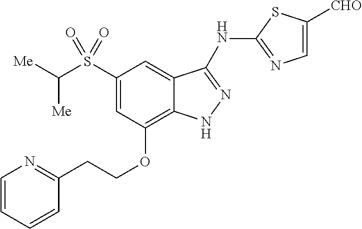

To a solution of 5-(isopropylsulfonyl)-7-(2-pyridin-2-ylethoxy)-1H-indazole-3-amine (0.23 g) in tetrahydrofuran (8 mL) was added 1,1′-carbonothioyldipyridine-2(1H)-one (0.16 g) at 0° C., stirred for 30 min, and concentrated aqueous ammonia (0.19 mL) was added. The reaction mixture was stirred at room is temperature for 1 hr, water was added, and the mixture was extracted with ethyl acetate. The ethyl acetate layer was washed with saturated brine, dried over anhydrous magnesium sulfate, and concentrated under reduced pressure to give yellow crude crystals. A mixture of the obtained crude crystals, bromomalonaldehyde (0.11 g), N,N-dimethylacetamide (8 mL) and ethanol (8 mL) was stirred overnight at 60° C. The reaction mixture was neutralized with saturated aqueous sodium hydrogen carbonate, and extracted with ethyl acetate. The ethyl acetate layer was washed with saturated brine, dried over anhydrous magnesium sulfate, and concentrated under reduced pressure. The residue was purified by silica gel column chromatography (hexane:ethyl acetate=1:2) to give the title compound (0.15 g, yield 64%) as pale-yellow crystals. Melting point>185° C. (decomposition).

### Reference Example 215

### ethyl 5-(methylthio)pyridine-2-carboxylate

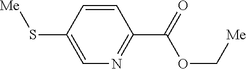

A mixture of 2-bromo-5-(methylthio)pyridine (5.41 g), palladium (II) acetate (0.60 g), 1,3-bis(diphenylphosphino)propane (1.37 g), triethylamine (18.5 mL), ethanol (30 mL) and N,N-dimethylformamide (30 mL) was stirred for 4 hrs at 70° C. under carbon monooxide atmosphere. The reaction mixture was concentrated, water was added, and the mixture was extracted with ethyl acetate. The ethyl acetate layer was washed with saturated brine, dried over anhydrous magnesium sulfate, and concentrated under reduced pressure. The residue was purified by silica gel column chromatography (ethyl acetate:hexane=1:1) to give the title compound (4.73 g, yield 91%) as yellow crystals. Melting point 44-46° C.

### Reference Example 216

### [5-(methylthio)pyridin-2-yl]methanol

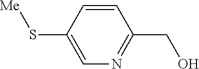

A mixture of ethyl 5-(methylthio)pyridine-2-carboxylate (4.73 g), sodium borohydride (1.00 g), ethanol (10 mL) and tetrahydrofuran (10 mL) was stirred overnight at 50° C. Water was added to the reaction mixture, and the mixture was extracted with ethyl acetate. The ethyl acetate layer was washed with saturated brine, dried over anhydrous magnesium sulfate, and concentrated under reduced pressure. The residue was purified by silica gel column chromatography (hexane:ethyl acetate=1:4) to give the title compound (2.53 g, yield 68%) as a yellow oily substance. MS: 156(MH⁺).

### Reference Example 217

### 2-amino-5-(isopropylsulfonyl)-3-{[5-(methylthio)pyridin-2-yl]methoxy}benzonitrile

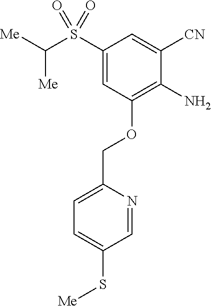

A mixture of 2-amino-3-hydroxy-5-(isopropylsulfonyl)benzonitrile (0.30 g), [5-(methylthio)pyridin-2-yl]methanol (0.20 g), tributylphosphine (0.62 mL), 1,1′-(azodicarbonyl)dipiperidine (0.63 g) and tetrahydrofuran (20 ml) was stirred overnight at room temperature. The reaction mixture was concentrated, and the residue was purified by silica gel column chromatography (hexane:ethyl acetate=1:2) to give the title compound (0.35 g, yield 78%) as colorless crystals. Melting point 156-157° C.

### Reference Example 218

### 5-(isopropylsulfonyl)-7-{[4-(methylthio)benzyl]oxy}-1H-indazole-3-amine

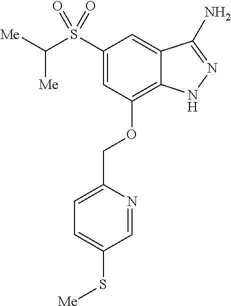

The title compound (0.46 g, yield 67%) was obtained as yellow crystals from 2-amino-5-(isopropylsulfonyl)-3-{[5-(methylthio)pyridin-2-yl]methoxy}benzonitrile (0.69 g) in the same manner as in Reference Example 103. Melting point 208-209° C.

### Reference Example 219

### 3-(benzyloxy)-2-nitrobenzaldehyde oxime

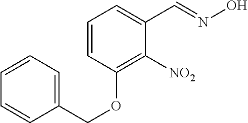

To a mixture of 1-(benzyloxy)-3-methyl-2-nitrobenzene (39.82 g), butyl nitrite (22.0 g) and N,N-dimethylformamide (300 mL) was gradually added potassium tert-butoxide (48.5 g) at −10 to 0° C. The reaction mixture was stirred at 0° C. for 1 hr, acidified by adding 10% aqueous citric acid solution, and extracted with ethyl acetate. The ethyl acetate layer was washed with saturated brine, dried over anhydrous magnesium sulfate, and concentrated under reduced pressure. The residue was purified by silica gel column chromatography (hexane:ethyl acetate=3:2) to give the title compound (36.62 g, yield 82%) as pale-yellow crystals. Melting point 141-142° C.

### Reference Example 220

### 3-(benzyloxy)-2-nitrobenzonitrile

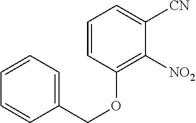

To a solution of 3-(benzyloxy)-2-nitrobenzaldehyde oxime (36.42 g) in N,N-dimethylformamide (300 mL) was added dropwise thionyl chloride (10.8 mL) at 0° C. for 20 min. After stirring at 0° C. for 15 min, the reaction mixture was poured into ice water. The precipitated crystals were collected by filtration, washed with water, and dried to give the title compound (31.62 g, yield 93%) as yellow crystals. Melting point 94-95° C.

### Reference Example 221

### 2-amino-3-(benzyloxy)benzonitrile

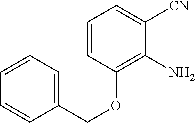

A mixture of 3-(benzyloxy)-2-nitrobenzonitrile (5.00 g), acetic acid (15 mL) and ethanol (15 mL) was heated to 80° C., and iron powder (5.50 g) was gradually added. The reaction mixture was stirred at 80° C. for 1 hr, and the insoluble materials were removed by filtration. The filtrate was concentrated and water was added. The precipitation solid was collected by filtration, washed with water and dried. The obtained solid was purified by silica gel column chromatography (hexane:ethyl acetate=1:1) to give the title compound (3.02 g, yield 69%) as yellow crystals. Melting point 102-103° C.

### Reference Example 222

### 4-amino-3-(benzyloxy)-5-cyanophenylthiocyanate

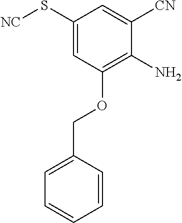

The title compound (12.4 g, quantitatively) was obtained as pale-yellow crystals from 2-amino-3-(benzyloxy)benzonitrile (9.89 g) in the same manner as in Reference Example 206. Melting point 129-130° C.

### Reference Example 223

### 2-amino-3-(benzyloxy)-5-(isopropylthio)benzonitrile

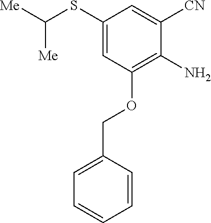

The title compound (11.35 g, yield 89%) was obtained as a pale-yellow oily substance from 4-amino-3-(benzyloxy)-5-cyanophenylthiocyanate (12.00 g) in the same manner as in Reference Example 207. MS: 297 (MH⁺).

### Reference Example 224

### 2-amino-3-(benzyloxy)-5-(isopropylsulfonyl)benzonitrile

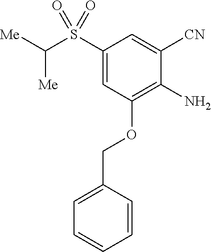

The title compound (10.72 g, yield 85%) was obtained as colorless crystals from 2-amino-3-(benzyloxy)-5-(isopropylthio)benzonitrile (11.35 g) in the same manner as in Reference Example 209., Melting point 151-152° C.

### Reference Example 225

### 7-(benzyloxy)-5-(isopropylsulfonyl)-1H-indazole-3-amine

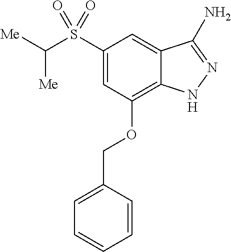

To a mixture of 2-amino-3-(benzyloxy)-5-(isopropylsulfonyl)benzonitrile (1.00 g), concentrated hydrochloric acid (8 mL) and acetic acid (8 mL) was added a solution of sodium nitrite (0.25 g) in water (3 mL) at −2 to 0° C. for 15 min, and the mixture was stirred at 0° C. for 30 min. The obtained reaction solution was added to a solution of tin(II) chloride (1.72 g) in concentrated hydrochloric acid (3 mL) at 0° C. for 10 min, and the mixture was stirred overnight at room temperature. The reaction mixture was neutralized by adding 8N aqueous sodium hydroxide solution, and the precipitated solid was filtered and washed with water. The obtained solid was eluted with tetrahydrofuran, and the eluate was concentrated. The obtained crude crystals were purified by NH silica gel column chromatography (eluate: tetrahydrofuran) to give the title compound (0.73 g, yield 69%) as colorless crystals. Melting point 179-180° C.

### Reference Example 226

### 5-(isopropylsulfonyl)-3-(1,3-thiazol-2-ylamino)-1H-indazol-7-ol

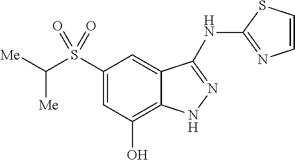

A mixture of 7-(benzyloxy)-5-(isopropylsulfonyl)-N-1,3-thiazol-2-yl-1H-indazole-3-amine (0.64 g), acetic acid (2 mL) and concentrated hydrochloric acid (15 mL) was heated under reflux for 6 hr. Water was added to the reaction mixture, and the precipitated crystals were collected by filtration, washed with water and ethyl acetate and dried to give the title compound (0.46 g, yield 93%) as grayish white crystals. Melting point>250° C. (decomposition).

### Reference Example 227

### 2-[5-(isopropylsulfonyl)-7-(2-pyridin-2-ylethoxy)-1H-indazol-3-yl]-1H-isoindole-1,3(2H)-dione

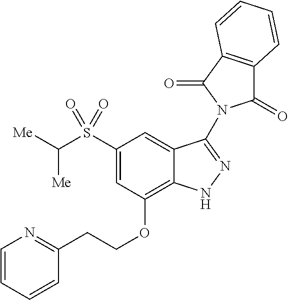

To a mixture of 5-(isopropylsulfonyl)-7-(2-pyridin-2-ylethoxy)-1H-indazole-3-amine (1.25 g), phthalic acid (0.60 g), 1H-1,2,3-benzotriazol-1-ol (1.28 g) and N,N-dimethylformamide (12 mL) was added N-[3-(dimethylamino)propyl]-N′-ethylcarbodiimide hydrochloride (1.60 g) at room temperature, and the mixture was stirred at 50° C. for 2 hr. To the reaction mixture was added saturated aqueous sodium hydrogen carbonate, and the mixture was extracted with ethyl acetate. The ethyl acetate layer was washed with saturated brine, dried over anhydrous magnesium sulfate, and concentrated under reduced pressure. The residue was purified by silica gel column chromatography (eluate: ethyl acetate) to give the title compound (1.02 g, yield 60%) as yellow amorphous crystals.

MS: 491 (MH⁺).

### Reference Example 228

### 2-[5-(isopropylsulfonyl)-1-(methoxymethyl)-7-(2-pyridin-2-ylethoxy)-1H-indazol-3-yl]-1H-isoindole-1,3(2H)-dione

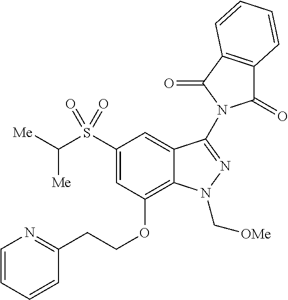

To a mixture of 2-[5-(isopropylsulfonyl)-7-(2-pyridin-2-ylethoxy)-1H-indazol-3-yl]-1H-isoindole-1,3(2H)-dione (1.02 g) in N,N-dimethylformamide (6 mL) was added sodium hydride (60%, oily, 0.10 g) at 0° C., and the mixture was stirred for 30 min at room temperature. To the reaction mixture was added chloromethyl methylether (0.18 mL) at 0° C. for 15 min. The reaction mixture was stirred at room temperature for 2 hr, water was added, and extracted with ethyl acetate. The ethyl acetate layer was washed with saturated brine, dried over anhydrous magnesium sulfate, and concentrated under reduced pressure to give the title compound (0.84 g, yield 77%) as pale-yellow crystals. Melting point 160-161° C.

### Reference Example 229

### 5-(isopropylsulfonyl)-1-(methoxymethyl)-7-(2-pyridin-2-ylethoxy)-1H-indazole-3-amine

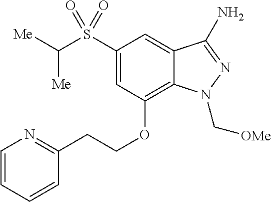

A mixture of 2-[5-(isopropylsulfonyl)-1-(methoxymethyl)-7-(2-pyridin-2-ylethoxy)-1H-indazol-3-yl]-1H-isoindole-1,3(2H)-dione (0.84 g), hydrazine monohydrate (0.24 g) and ethanol (12 mL) was stirred for 1 hr at 50° C. The precipitated solid was removed by filtration, and the filtrate was concentrated. The residue was purified by NH-silica gel column chromatography (eluate: ethyl acetate) to give the title compound (1.02 g, yield 60%) as yellow non-crystalline powder. Melting point 140-142° C.

### Reference Example 230

### tert-butyl 3-bromo-5-(3-chloropyridin-2-yl)-7-(4-(methylsulfonyl)phenoxy)-1H-indazole-1-carboxylate

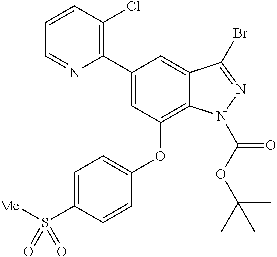

3-Bromo-5-(3-chloropyridin-2-yl)-7-(4-(methylsulfonyl)phenoxy)-1H-indazole (5.4 g, 11.3 mmol, 1 eq) and 4-(dimethylamino)pyridine (138 mg, 1.13 mmol) were dissolved in acetonitrile (150 ml). Di-tert-butyl dicarbonate (1 M in tetrahydrofuran, 12.4 ml, 12.4 mmol, 1.1 eq) was added and the mixture was stirred at room temperature overnight. The solution was concentrated in vacuo. The residue was purified with silica gel column chromatography using 5-50% ethyl acetate in hexane as an eluent to give 6.2 g of the product (95%) as a colorless solid. ¹H NMR (400 MHz, DMSO-d₆) δ ppm 1.39 (s, 9H) 3.17 (s, 3H) 7.14 (d, J=8.84 Hz, 2H) 7.51 (dd, J=8.08, 4.80 Hz, 1H) 7.83 (d, J=1.52 Hz, 1H) 7.89 (d, J=9.09 Hz, 2H) 7.98 (d, J=1.52 Hz, 1H) 8.11 (dd, J=8.08, 1.52 Hz, 1H) 8.67 (dd, is J=4.55, 1.52 Hz, 1H). [M⁺] calc'd for C₂₄H₂₁BrClN₃O₅S, 578; found, 578.

### Reference Example 231

### 5-(3-chloropyridin-2-yl)-1-(methoxymethyl)-7-(4-(methylsulfonyl)phenoxy)-N-(pyrazin-2-yl)-1H-indazol-3-amine

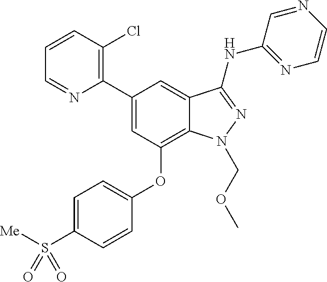

3-Bromo-5-(3-chloropyridin-2-yl)-1-(methoxymethyl)-7-(4-(methylsulfonyl)phenoxy)-1H-indazole (174.9 mg, 0.334 mmol, 1 eq), aminopyrazine (39 mg, 0.40 mmol, 1.2 eq), tris(dibenzylidene-acetone)dipalladium(0) (16 mg, 0.0167 mmol, 0.05 eq), 4,5-bis(diphenylphosphino)-9,9-dimethylxanthene (29 mg, 0.05 mmol, 0.15 eq), and cesium carbonate (218 mg, 0.668 mmol, 2 eq) were suspended in degassed anhydrous 1,4-dioxane (10 ml). The mixture was heated under N₂ at 100° C. overnight. After cooling to room temperature, the mixture was diluted with ethyl acetate, the organic layer was washed with water and saturated brine, dried over magnesium sulfate, filtered, and concentrated in vacuo. The residue was purified with NH-silica gel column chromatography using 33-66% ethyl acetate in hexane as an eluent to give 91.9 mg of the product (51%) as a light yellow oil. ¹H NMR (400 MHz, DMSO-d₆) δ ppm 3.21 (s, 3H) 3.22 (s, 3H) 5.62 (s, 2H) 7.35 (d, J=9.09 Hz, 2H) 7.44 (dd, J=8.08, 4.80 Hz, 1H) 7.48 (d, J=1.26 Hz, 1H) 7.98 (d, J=9.09 Hz, 2H) 8.06 (dd, J=8.21, 1.39 Hz, 1H) 8.16 (d, J=2.78 Hz, 1H) 8.28 (dd, J=2.53, 1.52 Hz, 1H) 8.51 (d, J=1.52 Hz, 1H) 8.62 (dd, J=4.55, 1.52 Hz, 1H) 9.30 (d, J=1.52 Hz, 1H) 10.45 (s, 1H). [M+H] calc'd for C₂₅H₂₁ClN₆O₄S, 537; found, 537.

### Reference Example 232

### 4-amino-3-methyl-5-(4-(methylsulfonyl)phenoxy)phenol

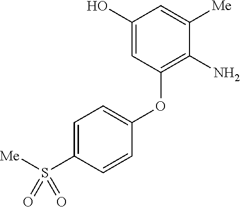

To a stirred solution of 2-methyl-6-(4-(methylsulfonyl)phenoxy)-4-(4,4,5,5-tetramethyl-1,3,2-s dioxaborolan-2-yl)aniline (1.0 g, 2.48 mmol, 1 eq) in methanol (20 ml) were added 1N NaOH (2.5 ml, 2.5 mmol, 1 eq) and 30% hydrogen peroxide (0.3 ml, 2.5 mmol, 1 eq) at 0° C., and the mixture was stirred at 0° C. for 1 h. To the mixture was added 1N HCl (5 mL, 5 mmol), and then the mixture was neutralized with saturated aqueous NaHCO₃. The mixture was extracted with ethyl acetate. The organic layer was washed with water and saturated brine, dried over magnesium sulfate, filtered, and concentrated in vacuo. The residue was recrystallized from ethyl acetate-ether to give 328 mg of the title compound (45%) as a light yellow solid. ¹H NMR (400 MHz, DMSO-d₆) δ ppm 2.09 (s, 3H) 3.17 (s, 3H) 4.05 (br. s., 2H) 6.15-6.25 (m, 1H) 6.42 (s, 1H) 6.90-7.22 (m, 2H) 7.65-8.07 (m, 2H) 8.51-8.83 (m, 1H). [M+H] calc'd for C₁₄H₁₅NO₄S, 294; found, 294.

### Reference Example 233

### 7-(4-(methylsulfonyl)phenoxy)-1H-indazol-5-ol

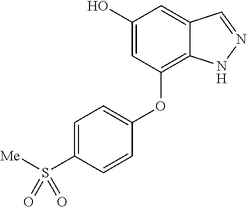

4-Amino-3-methyl-5-(4-(methylsulfonyl)phenoxy)phenol (97.7 mg, 0.353 mmol) was suspended in toluene (3 ml). Potassium acetate (82 mg, 0.883 mmol, 2.5 eq) and acetic anhydride (0.142 ml, 1.5 mmol, 4.5 eq) were added to the mixture at room temperature and the mixture was heated at 80° C. for overnight. Isoamyl nitrite (0.05 ml, 0.366 mmol, 2 eq) was added and the mixture was heated at 80° C. for overnight. The solution was diluted with ethyl acetate and washed with water and brine. After drying over magnesium sulfate and filtration, the filtrate was concentrated in vacuo. The residue was dissolved in methanol and potassium carbonate (69 mg, 0.50 mmol, 1.5 eq) was added. The mixture was stirred at 50° C. for 30 min. After filtration and evaporation, the residue was purified with silica gel column chromatography eluting with 20 to 66% ethyl acetate in haxane to give 68.1 mg of the title compound (67%) as a brown oil. ¹H NMR (400 MHz, DMSO-d₆) δ ppm 3.21 (s, 3H) 6.58 (d, J=2.02 Hz, 1H) 6.90 (d, J=1.52 Hz, 1H) 7.21 (d, J=8.84 Hz, 2H) 7.93 (d, J=8.84 Hz, 2H) 7.96 (d, J=1.52 Hz, 1H) 9.34 (s, 1H) 13.10 (s, 1H). [M+H] calc'd for C₁₄H₁₂N₂O₄S, 305; found, 305.

### Reference Example 234

### 7-(4-(methylsulfonyl)phenoxy)-1H-indazol-5-yl pivalate

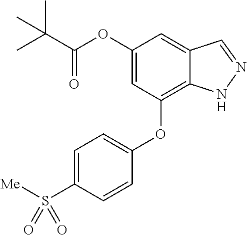

To a stirred solution of 7-(4-(methylsulfonyl)phenoxy)-1H-indazol-5-ol (1.005 g, 3.3 mmol, 1 eq) in dichloromethane (10 ml) were added triethylamine (0.51 ml, 3.63 mmol, 1.2 eq) and pivaloyl chloride (0.43 ml, 3.47 mmol, 1.1 eq) at room temperature. The mixture was stirred at room temperature for overnight, diluted with ethyl acetate. The mixture was washed with water and saturated brine, dried over magnesium sulfate, filtered, and concentrated in vacuo. The residue was purified with NH-silica gel column chromatography eluting with 50 to 66% ethyl acetate in haxane to give 943 mg of the title compound (74%) as a colorless solid. ¹H NMR (400 MHz, DMSO-d₆) δ ppm 1.30 (s, 9H) 3.20 (s, 3H) 6.95 (d, J=1.77 Hz, 1H) 7.22 (d, J=9.09 Hz, 2H) 7.43 (d, J=1.77 Hz, 1H) 7.94 (d, J=8.84 Hz, 2H) 8.18 (d, J=1.52 Hz, 1H) 13.55 (s, 1H). [M+H] calc'd for C₁₉H₂₀N₂O₅S, 389; found, 389.

### Reference Example 235

### 3-bromo-7-(4-(methylsulfonyl)phenoxy)-1H-indazol-5-yl pivalate

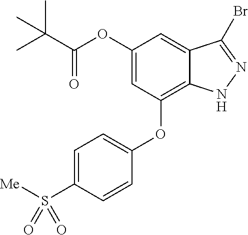

7-(4-(Methylsulfonyl)phenoxy)-1H-indazol-5-yl pivalate (940 mg, 2.42 mmol, 1 eq) was dissolved in anhydrous DMF (10 ml) at 0° C. N-bromosuccinimide (453 mg, 2.54 mmol, 1.05 eq) was added and the mixture was stirred at 0° C. for 3 h. The mixture was diluted with ethyl acetate and washed with saturated aqueous NaHCO₃ and saturated brine. The organic layer was dried over magnesium sulfate, filtered, and concentrated in vacuo. The residue was purified with silica gel column chromatography eluting with 10 to 50% ethyl acetate in hexane to give 1.106 g of the title compound (98%) as a colorless solid. ¹H NMR (400 MHz, DMSO-d₆) δ ppm 1.30 (s, 9H) 3.21 (s, 3H) 7.08 (d, J=1.77 Hz, 1H) 7.22-7.31 (m, 3H) 7.95 (d, J=9.09 Hz, 2H) 13.97 (s, 1H). [M+1+H] calc'd for C₁₉H₁₉BrN₂O₅S, 469; found, 469.

### Reference Example 236

### 3-bromo-1-(methoxymethyl)-7-(4-(methylsulfonyl)phenoxy)-1H-indazol-5-yl pivalate

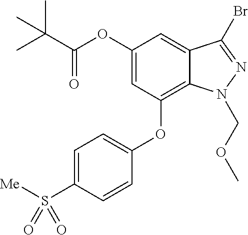

To a stirred solution of 3-bromo-7-(4-(methylsulfonyl)phenoxy)-1H-indazol-5-yl pivalate (567 mg, 1.21 mmol, 1 eq) in DMF (10 ml) was added sodium hydride (60% oil dispersion, 54 mg, 1.33 mmol, 1.1 eq) at 0° C. After the mixture was stirred for 30 min at 0° C., chloromethyl methyl ether (0.11 ml, 1.33 mmol, 1.1 eq) was added to the mixture. The mixture was stirred at room temperature for overnight. The reaction was quenched with water at 0° C. The mixture was diluted with ethyl acetate. The organic layer was washed with water and saturated brine, dried over magnesium sulfate, filtered, and concentrated in vacuo. The residue was purified with silica gel column chromatography eluting with 5 to 100% ethyl acetate in hexane to give 480 mg of the title compound (78%) as a colorless oil. ¹H NMR (400 MHz, DMSO-d₆) δ ppm 1.29 (s, 9H) 3.15 (s, 3H) 3.22 (s, 3H) 5.64 (s, 2H) 7.15 (d, J=1.77 Hz, 1H) 7.28-7.38 (m, 3H) 7.97 (d, J=8.84 Hz, 2H). [M+1+H] calc'd for C₂₁H₂₃BrN₂O₆S, 513; found, 513.

### Reference Example 237

### 1-(methoxymethyl)-3-(1-methyl-1H-pyrazol-3-ylamino)-7-(4-(methylsulfonyl)phenoxy)-1H-indazol-5-yl pivalate

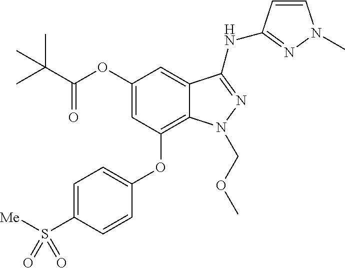

3-Bromo-1-(methoxymethyl)-7-(4-(methylsulfonyl)phenoxy)-1H-indazol-5-yl pivalate (450 mg, 0.88 mmol, 1 eq), 3-amino-1-methylpyrazole (103 mg, 1.06 mmol, 1.2 eq), tris(dibenzylidene-acetone)dipalladium(0) (41 mg, 0.044 mmol, 0.05 eq), 4,5-bis(diphenylphosphino)-9,9-dimethylxanthene (77 mg, 0.132 mmol, 0.15 eq), and cesium carbonate (574 mg, 1.76 mmol, 2 eq) were suspended in degassed anhydrous 1,4-dioxane (10 ml). The mixture was heated under N₂ at 100° C. overnight. After cooling m to room temperature, the mixture was diluted with ethyl acetate, washed with water and saturated brine, dried over magnesium sulfate, filtered, and concentrated in vacuo. The residue was purified with NH-silica gel column chromatography using 33-60% ethyl acetate in hexane as an eluent to give 263 mg of the is product (57%) as a light yellow oil. ¹H NMR (400 MHz, DMSO-d₆) δ ppm 1.30 (s, 9H) 3.13 (s, 3H) 3.21 (s, 3H) 3.75 (s, 3H) 5.43 (s, 2H) 6.57 (d, J=2.27 Hz, 1H) 7.01 (d, J=2.02 Hz, 1H) 7.25 (d, J=8.84 Hz, 2H) 7.54 (d, J=2.02 Hz, 1H) 7.83 (d, J=1.77 Hz, 1H) 7.95 (d, J=9.09 Hz, 2H) 9.52 (s, 1H). [M+H] calc'd for C₂₅H₂₉N₅O₆S, 528; found, 528.

### Reference Example 238

### 1-(methoxymethyl)-3-(1-methyl-1H-pyrazol-3-ylamino)-7-(4-(methylsulfonyl)phenoxy)-1H-indazol-5-ol

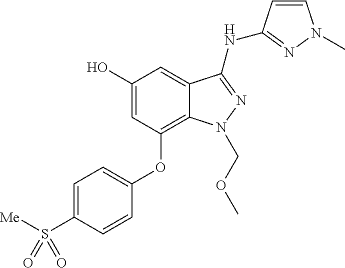

To a stirred solution of 1-(methoxymethyl)-3-(1-methyl-1H-pyrazol-3-ylamino)-7-(4-(methylsulfonyl)phenoxy)-1H-indazol-5-yl pivalate (163 mg, 0.31 mmol, 1 eq) in methanol (3 ml) was added potassium carbonate (64 mg, 0.46 mmol, 1.5 eq) at room temperature. The mixture was stirred at room temperature for 1 h. After diluted with ethyl acetate, the mixture was washed with water and saturated brine, dried over magnesium sulfate, filtered, and concentrated in vacuo. The residue was purified with NH-silica gel column chromatography using 0-10% MeOH in ethyl acetate as an eluent to give 127 mg of the product (92%) as a light yellow solid. ¹H NMR (400 MHz, DMSO-d₆) δ ppm 3.09 (s, 3H) 3.21 (s, 3H) 3.74 (s, 3H) 5.34 (s, 2H) 6.53 (d, J=2.02 Hz, 1H) 6.60 (d, J=1.77 Hz, 1H) 7.24 (d, J=8.84 Hz, 2H) 7.28 (d, J=2.02 Hz, 1H) 7.51 (d, J=2.27 Hz, 1H) 7.94 (d, J=8.84 Hz, 2H) 9.33 (s, 1H) 9.39 (s, 1H). [M+H] calc'd for C₂₀H₂₁N₅O₅S, 444; found, 444.

### Reference Example 239

### 3-bromo-5-(3-chloropyridin-2-yl)-1-methyl-7-(4-(methylsulfonyl)phenoxy)-1H-indazole

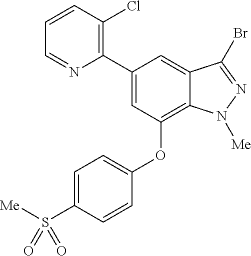

To a stirred solution of 3-bromo-5-(3-chloropyridin-2-yl)-7-(4-(methylsulfonyl)phenoxy)-1H-indazole (315 mg, 0.658 mmol) in DMF (5 ml) was added potassium carbonate (110 mg, 0.79 mmol, 1.2 eq) and iodomethane (0.36 ml, 0.72 mmol, 1.1 eq) at room temperature. The mixture was stirred at 50° C. for overnight. After diluted with ethyl acetate, the mixture was washed with water and saturated brine, dried over magnesium sulfate, filtered, and concentrated in vacuo. The residue was purified with silica gel column chromatography using 25-50% ethyl acetate in hexanes as an eluent to give 273 mg of the product (84%) as a colorless solid. ¹H NMR (400 MHz, DMSO-d₆) δ ppm 3.22 (s, 3H) 4.10 (s, 3H) 7.36 (d, J=8.84 Hz, 2H) 7.46 (dd, J=8.08, 4.55 Hz, 1H) 7.48 (d, J=1.26 Hz, 1H) 7.82 (d, J=1.26 Hz, 1H) 7.98 (d, J=8.84 Hz, 2H) 8.07 (dd, J=8.08, 1.52 Hz, 1H) 8.63 (dd, J=4.55, 1.52 Hz, 1H). [M+1+H] calc'd for C₂₀H₁₅BrClN₃O₃S, 494; found, 494

### Reference Example 240

### 2-methyl-6-(4-(methylsulfonyl)phenoxy)-4-thiocyanatoaniline

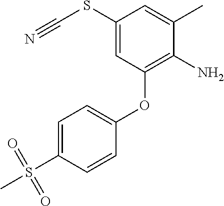

Sodium thiocyanate (2.93 g, 36 mmol, 2 eq) was dissolved in methanol (90 ml) at −78° C. Bromine (1.11 ml, 21.7 mmol, 1.2 eq) was added and the mixture was stirred for 10 min. 2-Methyl-6-(4-(methylsulfonyl)phenoxy)aniline (5 g, 18 mmol, 1 eq) was added and the mixture stirred at −78° C. for 30 min before allowing to room temperature. Upon the completion of the reaction the solution was poured into ice water. After 10 minutes a pale pink precipitate formed which was filtered and dried to give 4.67 g of the title compound (77%) as a pale pink solid. ¹H NMR (400 MHz, DMSO-d₆) δ ppm 2.18 (s, 3H) 3.18 (s, 3% H) 5.5 (br. s, 2H) 7.02-7.14 (m, 2H) 7.18 (d, J=2.27 Hz, 1H) 7.28 (d, J=3.03 Hz, 1H) 7.71-7.99 (m, 2H) 1 eq [M+H] calc'd for C₁₅H₁₄N₂O₃S₂, 335; found, 335.

### Reference Example 241

### 4-(isopropylthio)-2-methyl-6-(4-(methylsulfonyl)phenoxy)aniline

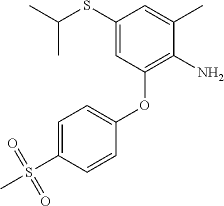

2-Methyl-6-(4-(methylsulfonyl)phenoxy)-4-thiocyanatoaniline (4.67 g, 13.98 mmol, 1 eq) was dissolved in tetrahydrofuran (50 ml) and 2-propanol (50 ml). 2-Iodopropane (1.89 ml, 18.87 mmol, 1.35 eq) and 2N NaOH (8.4 ml, 16.8 mmol) were added to the solution at room temperature. After stirring at room temperature for 30 min, sodium borohydride (264 mg, 6.99 mmol, 0.5 eq) was added and the mixture was stirred at room temperature for 12 h. Upon completion the solvent was removed in vacuo, and the residue was re-dissolved in ethyl acetate and washed with water. After drying over magnesium sulfate, filtration, and concentrating, the residue was purified with flash silica gel column chromatography eluting with 30% ethyl acetate in hexane to give 1.77 g of the title compound (37%) as a tan solid. [M+H] calc'd for C₁₇H₂₁NO₃S₂₁352; found, 352.

### Reference Example 242

### 4-(isopropylsulfonyl)-2-methyl-6-(4-(methylsulfonyl)phenoxy)aniline

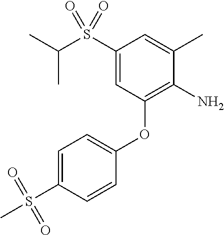

4-(Isopropylthio)-2-methyl-6-(4-(methylsulfonyl)phenoxy)aniline (4.84 g, 13.8 mmol, 1 eq) was dissolved in tetrahydrofuran (30 ml), methanol (30 ml) and water (10 ml). Oxone (9.32 g, 15.1 mmol, 1.1 eq) was added and the mixture was stirred at room temperature for 2 h. Solid sodium sulfite was added to quench the reaction. After filtration, the filtrate was concentrated in vacuo and the residue was purified with flash silica gel column chromatography eluting with 30 to 80% ethyl acetate in hexane to yield 4.13 g of the title compound as a brown solid (78%). [M+H] calc'd for C₁₇H₂₁NO₅S₂, 384; found, 384.

### Reference Example 243

### 5-(isopropylsulfonyl)-7-(4-(methylsulfonyl)phenoxy)-1H-indazole

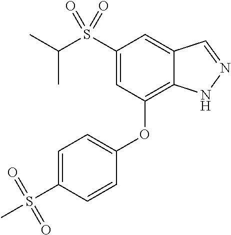

4-(Isopropylsulfonyl)-2-methyl-6-(4-(methylsulfonyl)phenoxy)aniline (4.13 g, 10.8 mmol) was dissolved in toluene (40 ml). Potassium acetate (1.16 g, 11.86 mmol, 1.1 eq) and acetic anhydride (4.1 ml, 43 mmol, 4 eq) were added and the mixture was heated at 80° C. for overnight. Isoamyl nitrite (2.88 ml, 21.6 mmol, 2 eq) was added and the mixture was heated at 80° C. for 12 h. The solution was diluted with ethyl acetate and washed with water and brine. After drying over magnesium sulfate and filtration, the filtrate was concentrated in vacuo. The residue was dissolved in methanol and solid potassium carbonate added to remove the N-acetyl group. After filtration and evaporation, the residue was purified with flash silica gel column chromatography eluting with 30 to 100% ethyl acetate in hexane to give 2.5 g of the title compound (59%) as an orange solid. ¹H NMR (400 MHz, DMSO-d₆) δ ppm 1.16 (dd, 6H) 3.24 (s, 3H) 3.32 (s, 1H) 7.27-7.41 (m, 3H) 7.98 (d, J=9.09 Hz, 2H) 8.27 (s, 1H) 8.45 (s, 1H) 14.05 (s, 1H). [M+H] calc'd for C₁₇H₁₈N₂O₅S₂, 395; found, 395.

### Reference Example 244

### 3-bromo-5-(isopropylsulfonyl)-7-(4-(methylsulfonyl)phenoxy)-1H-indazole

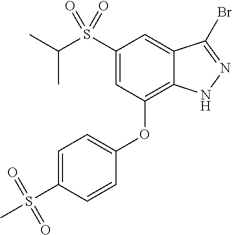

5-(Isopropylsulfonyl)-7-(4-(methylsulfonyl)phenoxy)-1H-indazole (2.5 g, 6.34 mmol, 1 eq) was dissolved in anhydrous DMF (15 ml) at 0° C. N-bromosuccinimide (1.18 g, 6.66 mmol, 1.05 eq) in DMF (5 ml) was added and the mixture stirred at 0° C. for 4 h. A further 50 mg of N-bromosuccinimide was added and the mixture was stirred at room temperature for 2 h. The mixture was diluted with ethyl acetate and washed with saturated sodium bicarbonate and brine. The organic layer was dried over magnesium sulfate, filtered, and concentrated in vacuo. The residue was purified with flash silica gel column chromatography eluting with 30 to 100% ethyl acetate in hexane to give 2.5 g of the title compound (83%) as a light yellow oil. [M+1+H] calc'd for C₁₇H₁₇BrN₂O₅S₂, 474; found, 474.

### Reference Example 245

### tert-butyl 3-bromo-5-(isopropylsulfonyl)-7-(4-(methylsulfonyl)phenoxy)-1H-indazole-1-carboxylate

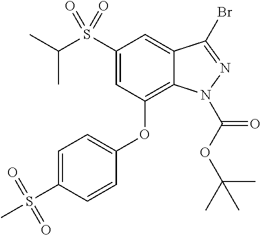

3-Bromo-5-(isopropylsulfonyl)-7-(4-(methylsulfonyl)phenoxy)-1H-indazole (2.5 g, 5.29 mmol, 1 eq) was dissolved in anhydrous DMF (10 ml) and triethylamine (880 μl, 6.34 mmol, 1.2 eq). Di-tert-butyl dicarbonate (1.27 g, 5.81 mmol, 1.1 eq) was added and the mixture was stirred at room temperature for overnight. The solution was diluted with ethyl acetate and washed with saturated sodium bicarbonate. After drying over magnesium sulfate, the filtrate was concentrated in vacuo to give 2.1 g of the title compound as a tan solid (68%). ¹H NMR (400 MHz, DMSO-d₆) δ ppm 1.18 (d, J=6.82 Hz, 6H) 1.39 (s, 9H) 3.20 (s, 3H) 3.33 (s, 1H) 3.57-3.72 (m, 1H) 7.20 (d, J=8.84 Hz, 2H) 7.81 (d, J=1.52 Hz, 1H) 7.90 (d, J=9.09 Hz, 2H) 8.05 (d, J=1.52 Hz, 1H). [M+1+H] calc'd for C₂₂H₂₅BrN₂O₇S₂, 574; found, 574.

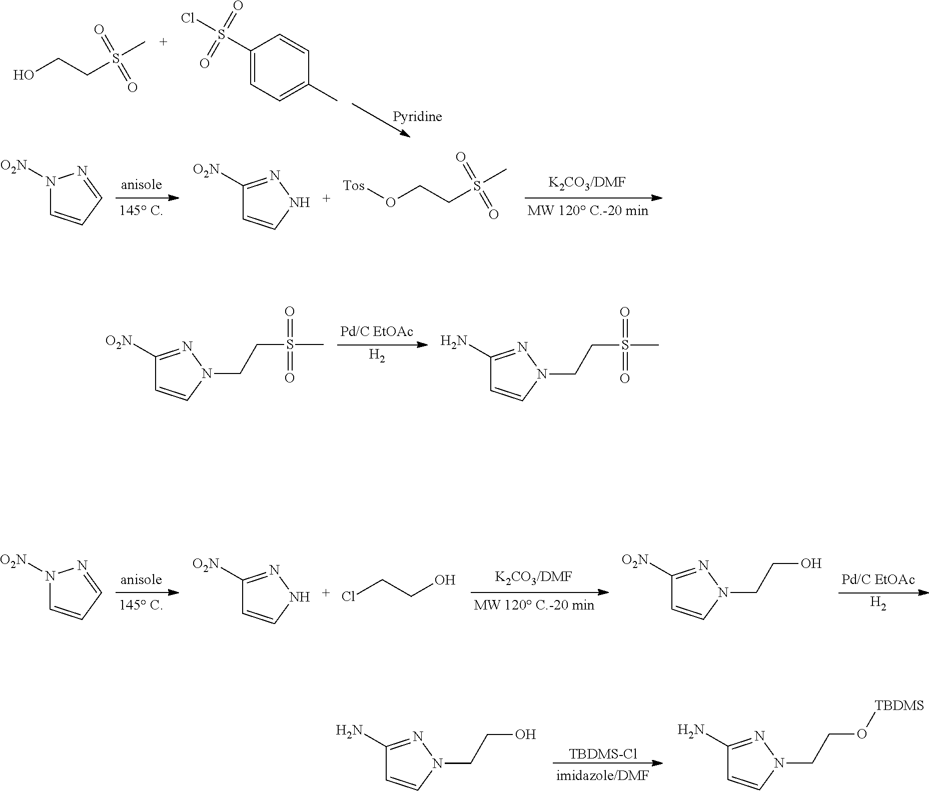

### Reference Example 246

### 3-nitro-1H-pyrazole

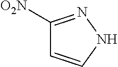

1-Nitropyrazole (10 g, 88 mmol) was heated in 600 ml of anisole at 145° C. overnight. The mixture was cooled in a freezer which caused a precipitate to form. The precipitate was collected to give 5.075 g of the title compound as a tan solid (51%). [M+H] calc'd for C₃H₃N₃O₂, 114; found, 114.

### Reference Example 247

### 2-(methylsulfonyl)ethyl 4-methylbenzenesulfonate

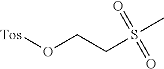

2-(Methylsulfonyl)ethanol (2 g, 16.1 mmol, 1 eq) and p-toluenesulfonyl chloride (3.38 g, 17.7 mmol, 1.1 eq) were stirred in pyridine (10 ml) at room temperature for overnight. The mixture was diluted with ethyl acetate and wash with water. After drying over magnesium sulfate and evaporation, the residue was purified with flash silica gel column chromatography using 20-80% ethyl acetate in hexane as an eluent to give 1.1 g of the product as a colorless oil (25%). [M+H] calc'd for C₁₀H₁₄O₅S₂, 279; found, 279.

### Reference Example 248

### 1-(2-(methylsulfonyl)ethyl)-3-nitro-1H-pyrazole

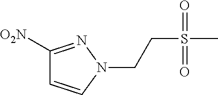

3-Nitro-1H-pyrazole (410 mg, 3.60 mmol, 1 eq), 2-(methylsulfonyl)ethyl 4-methylbenzenesulfonate (1.1 g, 3.96 mmol, 1.1 eq), and potassium carbonate (1.49 g, 10.7 mmol, 3 eq) were mixed in DMF (3 ml) and subjected to microwave irradiation at 120° C. for 20 min. The mixture was diluted with ethyl acetate and washed with water then 1N HCl. The organic layer was dried over magnesium sulfate, filtered, and concentrated in vacuo to give 640 mg of title compound (81%) as a light yellow oil. [M+H] calc'd for C₆H₉N₃O₄S, 220; found, 220.

### Reference Example 249

### 1-(2-(methylsulfonyl)ethyl)-1H-pyrazol-3-amine

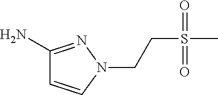

1-(2-(Methylsulfonyl)ethyl)-3-nitro-1H-pyrazole (460 mg, 2.10 mmol) was dissolved in ethyl acetate (5 ml). 10% wt Palladium on carbon (1 g) was added to the solution and the reaction mixture was stirred under H₂ gas at room temperature for overnight. The suspension was filtered through celite and the filtrate was concentrated to give 470 mg of the title compound (quantitative) as a light yellow oil. [M+H] calc'd for C₆H₁₁N₃O₂S, 190 found, 190.

### Reference Example 250

### 2-(3-nitro-1H-pyrazol-1-yl)ethanol

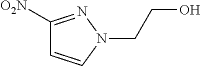

3-Nitro-1H-pyrazole (3 g, 26 mmol, 1 eq), 2-chloroethanol (2.23 g, 27 mmol, 1.05 eq), and potassium carbonate (7.32 g 54 mmol, 2 eq) were mixed in DMF (5 ml). The mixture was subjected to microwave irradiation at 120° C. for 20 minutes. The mixture was diluted with ethyl acetate and washed with water. The organic layer was dried over magnesium sulfate, filtered, and concentrated in vacuo, and the residue was purified with flash silica gel column chromatography using 20 to 60% ethyl acetate in hexane to give 2.48 g of the title compound (59%) as a light yellow oil. [M+H] calc'd for C₅H₇N₃O₃, 158 found, 158.

### Reference Example 251

### 2-(3-amino-1H-pyrazol-1-yl)ethanol

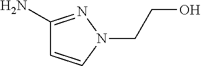

2-(3-Nitro-1H-pyrazol-1-yl)ethanol (2.48 g, 15.8 mmol) was dissolved in ethyl acetate (15 ml). 10% wt Pd/C (1 g) was added and the suspension was stirred under hydrogen gas at room temperature for overnight. The suspension was filtered through celite and concentrated in vacuo to give 1.8 g of the title compound (90%) as a light yellow oil. [M+H] calc'd for C₅H₉N₃O, 128 found, 128.

### Reference Example 252

### 1-(2-(tert-butyldimethylsilyloxy)ethyl)-1H-pyrazol-3-amine

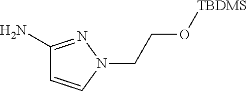

2-(3-Amino-1H-pyrazol-1-yl)ethanol (1.8 g, 14.2 mmol, 1 eq), tert-butyldimethylsilylchloride (10.6 g, 71 mmol, 5 eq), and imidazole (9.6 g, 141 mmol, 10 eq) were stirred in DMF (7 ml) at room temperature for overnight. The solid was removed by filtration and the filtrate was diluted with dichloromethane and washed with water. The organic layer was dried over magnesium sulfate, filtered, and concentrated in vacuo, and the residue was purified with flash silica gel column chromatography using 30 to 90% ethyl acetate in hexane as an eluent to give 2.1 g of title compound (61%) as a brown oil. [M+H] calc'd for C₁₁H₂₃N₃O₉S₂, 242 found, 242.

### Reference Example 253

### methyl 2-(3-amino-1H-pyrazol-1-yl)acetate

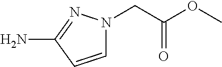

The title compound was prepared according to the procedure outlined in Reference Examples 20 and 21, using 3-nitro-1H-pyrazole and methyl chloroacetate. [M+H] calc'd for C₆H₉N₃O₂, 156; found, 156.

### Reference Example 254

### tert-butyl 2-(3-amino-1H-pyrazol-1-yl)ethylcarbamate

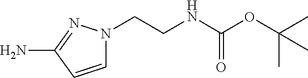

The title compound was prepared according to the procedure outlined in Reference Examples 20 and 21, using 3-nitro-1H-pyrazole and tert-butyl 2-hydroxyethylcarbamate. [M+H] calc'd for C₁₀H₁₈N₄O₂, 227; found, 227.

### Reference Example 255

### 1-((2,2-dimethyl-1,3-dioxolan-4-yl)methyl)-1H-pyrazol-3-amine

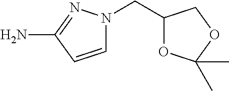

The title compound was prepared according to the procedure outlined in Reference Examples 20 and 21, using 3-nitro-1H-pyrazole and (2,2-dimethyl-1,3-dioxolan-4-yl)methanol. [M+H] calc'd for C₉H₁₅N₃O₂, 198; found, 198.

### Reference Example 256

### 1-methoxy-3-(3-nitro-1H-pyrazol-1-yl)propan-2-ol

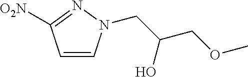

3-Nitro-1H-pyrazole (3 g, 26.5 mmol, 1 eq), 3-chloro-1-methoxy-2-propanol (3.92 g, 31.9 mmol, 1.2 eq), potassium iodide (5 mg, cat.), and cesium carbonate (17.2 g, 53.1 mmol, 2 eq) were mixed in 1,4-dioxane (18 ml). The mixture was subjected to microwave irradiation at 120° C. for 30 min. The mixture was diluted with ethyl acetate and washed with water. The organic layer was dried over magnesium sulfate, filtered, and concentrated in vacuo. The residue was purified with silica gel column chromatography using 10-60% ethyl acetate in hexane as an eluent to give 2.74 g of the title compound (51%) as a yellow oil. [M+H] calc'd for C₇H₁₁N₃O₄ 202, found 202.

### Reference Example 257

### 1-(3-amino-1H-pyrazol-1-yl)-3-methoxypropan-2-ol

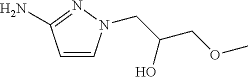

1-Methoxy-3-(3-nitro-1H-pyrazol-1-yl)propan-2-ol (2.74 g, 14.6 mmol, 1 eq) was dissolved in ethyl acetate (15 ml). 10% wt palladium on carbon (1 g) was added and the mixture purged with hydrogen. The mixture was stirred at room temperature for overnight under hydrogen and then filtered through celite, and the filtrate was concentrated in vacuo to give 1.41 g of the title compound (60%) as a colorless oil. [M+H] calc'd for C₇H₁₃N₃O₂ 172, found 172.

### Reference Example 258

### 1-(2-(tert-butyldimethylsilyloxy)-3-methoxypropyl)-1H-pyrazol-3-amine

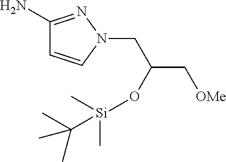

The title compound was prepared according to the procedure outlined in Reference Example 23, using 1-(3-amino-1H-pyrazol-1-yl)-3-methoxypropan-2-ol. [M+H] calc'd for C₁₃H₂₇N₃O₂Si, 286; found, 286.

### Reference Example 259

### 1-(3-methylbut-2-enyl)-3-nitro-1H-pyrazole

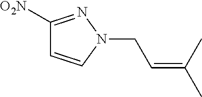

3-Nitro-1H-pyrazole (3 g, 26.5 mmol, 1 eq), 1-chloro-3-methylbut-2-ene (4.16 g 39.8 mmol, 1.5 eq), potassium iodide (5 mg, 0.03 mmol, 0.001 eq.), and cesium carbonate (17.2 g, 53 mmol, 2 eq) were mixed in 1,4-dioxane (305 ml). The mixture was subjected to microwave irradiation at 120° C. for 30 min. The mixture was diluted with ethyl acetate and washed with water. The organic layer was dried over magnesium sulfate, filtered, and concentrated in vacuo. The residue was purified with silica gel column chromatography using 0 to 40% ethyl acetate in hexane as an eluent to give 4.18 g of the title compound (87%) as a brown oil. [M+H] calc'd for C₈H₁₁N₃O₂, 182 found, 182.

### Reference Example 260

### 3-Methyl-1-(3-nitro-1H-pyrazol-1-yl)butane-2,3-diol

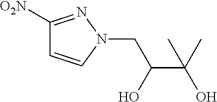

1-(3-Methylbut-2-enyl)-3-nitro-1H-pyrazole (3.646 g, 20.1 mmol, 1 eq), osmium tetroxide-2.5 wt % in water (3 ml, 0.4 mmol, 0.02 eq), and N-methylmorpholine-N-oxide (2.36 g, 20.1 mmol, 1 eq) were stirred in a 3:1 mixture of acetone:water (12 ml) at room temperature for 1 h. The reaction mixture was diluted with ethyl acetate and washed with water and 1N HCl. The organic layer was dried over magnesium sulfate, filtered, and concentrated in vacuo to give 3.3 g of the title compound (76%) as a brown solid. [M+H] calc'd for C₈H₁₃N₃O₄, 216 found, 216.

### Reference Example 261

### 3-Nitro-1-((2,2,5,5-tetramethyl-1,3-dioxolan-4-yl)methyl)-1H-pyrazole

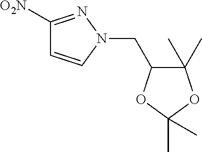

3-Methyl-1-(3-nitro-1H-pyrazol-1-yl)butane-2,3-diol (700 mg, 3.26 mmol, 1 eq), 2,2-dimethoxypropane (4 ml, 32.6 mmol, 10 eq), and p-toluene sulfonic acid (248 mg, 1.3 mmol, 0.4 eq) were stirred in acetone (5 ml) at room temperature for 3 h. The reaction mixture was diluted with ethyl acetate and washed with 1 M sodium thiosulfate solution and water. The organic layer was dried over magnesium sulfate, filtered, and concentrated in vacuo. The residue was purified with silica gel column chromatography using 10 to 70% ethyl acetate in hexane as an eluent to give 730 mg of the title compound (87%) as a white solid. ¹H NMR (400 MHz, DMSO-d₆) δ ppm 1.16 (s, 3H) 1.23 (s, 3H) 1.27 (s, 3H) 1.36 (s, 3H) 4.18 (dd, J=9.22, 3.16 Hz, 1H) 4.34-4.43 (m, 1H) 4.45-4.56 (m, 1H) 7.08 (d, J=2.53 Hz, 1H) 8.08 (d, J=2.78 Hz, 1H). [M+H] calc'd for C₁₁H₁₇N₃O₄, 256 found, 256.

### Reference Example 262

### 1-((2,2,5,5-tetramethyl-1,3-dioxolan-4-yl)methyl)-1H-pyrazol-3-amine

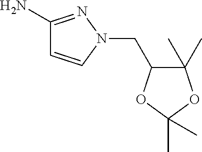

3-Nitro-1-((2,2,5,5-tetramethyl-1,3-dioxolan-4-yl)methyl)-1H-pyrazole (740 mg, 2.9 mmol) was dissolved in a 1:1 mixture of ethyl acetate:methanol (10 ml) and the solution was cooled to 0° C. Raney 2800 Nickel slurry in water (1 ml) was added. Hydrazine monohydrate (1 ml) was added dropwise and the mixture was stirred at room temperature for 30 minutes after the effervescence ceased. The mixture was filtered through celite, diluted with ethyl acetate and washed with water and brine. The organic layer was dried over magnesium sulfate, filtered, and concentrated in vacuo to give 543 mg of the title compound (83%) as a colorless solid. ¹H NMR (400 MHz, DMSO-d₆) δ ppm 1.12 (d, J=4.80 Hz, 6H) 1.22 (s, 3H) 1.34 (s, 3H) 3.88-4.02 (m, 2H) 4.02-4.14 (m, 1H) 4.57 (s, 2H) 5.37 (d, J=2.27 Hz, 1H) 7.34 (d, J=2.27 Hz, 1H). [M+H] calc'd for C₁₁H₁₉N₃O₂, 226 found, 226.

### Example 1

### N-1,3-thiazol-2-yl-5-(2-thienyl)-1H-indazole-3-amine

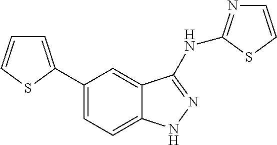

To a ethanol-water mixed solution (8 mL-2 mL) of N-[5-(2-thienyl)-1H-indazol-3-yl]thiourea (383 mg) was added 1,2-dichloroethylethyl ether (0.66 mL), and the mixture was stirred overnight at 90° C. The mixture was diluted with ethyl acetate, saturated aqueous sodium hydrogen carbonate, washed with water and saturated brine, dried over anhydrous magnesium sulfate, and concentrated under reduced pressure. The obtained crude crystals were recrystallized (tetrahydrofuran-diisopropyl to ether) to give the title compound (74.7 mg, yield 18%) as colorless crystals. ¹H NMR (300 MHz, DMSO-d₆) δ ppm 7.01 (d, J=3.41 Hz, 1H) 7.05-7.21 (m, 1H) 7.24-7.55 (m, 4H) 7.71 (d, J=8.71 Hz, 1H) 8.47 (s, 1H) 11.40 (brs, 1H) 12.42 (s, 1H).

### Example 2

### N-1,3-thiazol-2-yl-5-(3-thienyl)-1H-indazole-3-amine

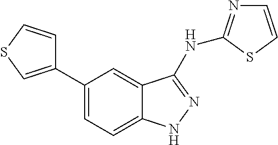

To a ethanol-1N hydrochloric acid (10 mL-3 mL) solution of N-[5-(3-thienyl)-1H-indazol-3-yl]thiourea (316 mg) was added 2-bromo-1,1-diethoxyethane (0.28 mL), and the mixture was stirred at 80° C. for 4 hr. The reaction mixture was basified using saturated aqueous sodium hydrogen carbonate, diluted with ethyl acetate-tetrahydrofuran, washed with saturated brine, dried over anhydrous magnesium sulfate, and concentrated. The obtained crude crystals were recrystallized (tetrahydrofuran) to give the title compound (93.2 mg, yield 27%) as colorless crystals. ¹H NMR (300 MHz, DMSO-d₆) δ ppm 7.01 (d, J=3.79 Hz, 1H) 7.37 (d, J=3.79 Hz, 1H) 7.43 (d, J=8.71 Hz, 1H) 7.52 (d, J=4.92 Hz, 1H) 7.67 (dd, J=4.92, 3.03 Hz, 1H) 7.70-7.83 (m, 2H) 8.49 (s, 1H) 11.31 (brs, 1H) 12.36 (s, 1H).

### Example 3

### N,5-di-1,3-thiazol-2-yl-1H-indazole-3-amine

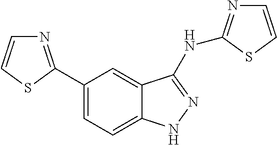

The title compound (3.6 mg, yield 11%) was obtained as colorless crystals from N-[5-(1,3-thiazol-2-yl)-1H-indazol-3-yl]thiourea (29.1 mg) in the same manner as in Example 2.

MS: 300 (MH⁺).

### Example 4

### 5-(1-methyl-1H-pyrazol-5-yl)-N-1,3-thiazol-2-yl-1H-indazole-3-amine

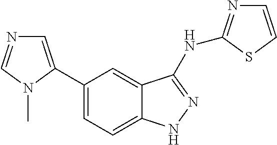

The title compound (12.5 mg, yield 39%) was obtained as colorless crystals from N-[5-(1-methyl-1H-pyrazol-5-yl)-1H-indazol-3-yl]thiourea (29.3 mg) in the same manner as in Example 2. MS: 297 (MH⁺).

### Example 5

### 5-(3-chloropyridin-2-yl)-N-1,3-thiazol-2-yl-1H-indazole-3-amine

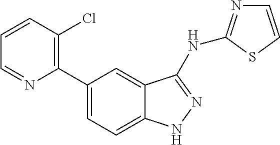

The title compound (79.5 mg, yield 24%) was obtained as colorless crystals from N-[5-(3-chloropyridin-2-yl)-1H-indazol-3-yl]thiourea (311 mg) in the same manner as in Example 2.

MS: 328 (MH⁺).

### Example 6

### 5-(1-methyl-1H-pyrazol-4-yl)-N-1,3-thiazol-2-yl-1H-indazole-3-amine

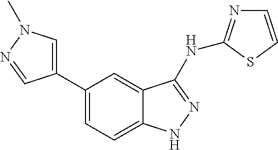

The title compound (109 mg, yield 24%) was obtained as colorless crystals from N-[5-(1-methyl-1H-pyrazol-4-yl)-1H-indazol-3-yl]thiourea (327 mg) in the same manner as in Example 2. MS: 297 (MH⁺).

### Example 7

### 5-(2-chlorophenyl)-N-1,3-thiazol-2-yl-1H-indazole-3-amine

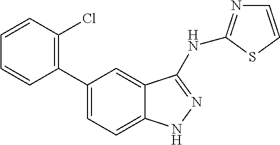

is The title compound (165 mg, yield 71%) was obtained as colorless crystals from N-[5-(2-chlorophenyl)-1H-indazol-3-yl]thiourea (214 mg) in the same manner as in Example 2.

MS: 327 (MH⁺).

### Example 8

### 5-(3-methylpyridin-2-yl)-N-1,3-thiazol-2-yl-1H-indazole-3-amine

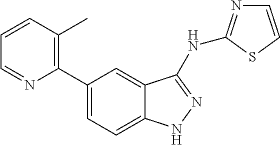

The title compound (82 mg, yield 76%) was obtained as colorless crystals from N-[5-(3-methylpyridin-2-yl)-1H-indazol-3-yl]thiourea (100 mg) in the same manner as in Example 2.

MS: 308 (MH⁺).

### Example 9

### 5-(3-fluoropyridin-2-yl)-N-1,3-thiazol-2-yl-1H-indazole-3-amine

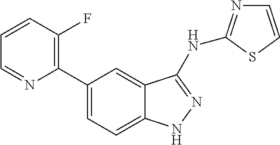

The title compound (110 mg, yield 76%) was obtained as colorless crystals from N-[5-(3-fluoropyridin-2-yl)-1H-indazol-3-yl]thiourea (162 mg) in the same manner as in Example 2. Melting point 264-265° C.

### Example 10

### 5-(3,5-dimethyl-1H-pyrazol-1-yl)-N-1,3-thiazol-2-yl-1H-indazole-3-amine

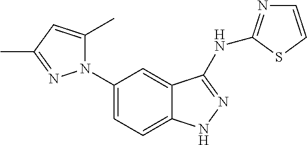

The title compound (43.0 mg, yield 60%) was obtained as colorless crystals from N-[5-(3,5-dimethyl-1H-pyrazol-1-yl)-1H-indazol-3-yl]thiourea (65.7 mg) in the same manner as in Example 2. Melting point>250° C.

### Example 11

### 5-(1-methyl-1H-imidazol-2-yl)-N-1,3-thiazol-2-yl-1H-indazole-3-amine

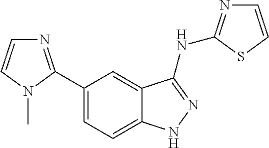

The title compound (23.0 mg, yield 34%) was obtained as colorless crystals from N-[5-(1-methyl-1H-imidazol-2-yl)-1H-indazol-3-yl]thiourea (35.7 mg) in the same manner as in Example 2. Melting point 191-193° C.

### Example 12

### 5-(4-chloropyridin-3-yl)-N-1,3-thiazol-2-yl-1H-indazole-3-amine

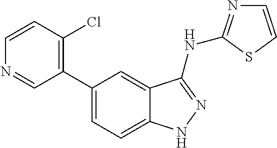

The title compound (69.5 mg, yield 60%) was obtained as colorless crystals from N-[5-(4-chloropyridin-3-yl)-1H-indazol-3-yl]thiourea (107 mg) in the same manner as in Example 2. Melting point>285° C.

### Example 13

### 2-[3-(1,3-thiazol-2-ylamino)-1H-indazol-5-yl]nicotinonitrile

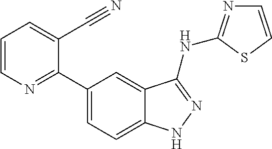

The title compound (62 mg, yield 66%) was obtained as colorless crystals from N-[5-(3-cyanopyridin-2-yl)-1H-indazol-3-yl]thiourea (87.1 mg) in the same manner as in Example 2. Melting point>285° C.

### Example 14

### tert-butyl 2-[3-(1,3-thiazol-2-ylamino)-1H-indazol-5-yl]nicotinate

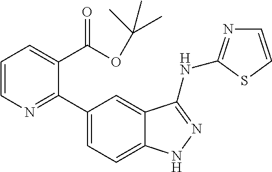

The title compound (69 mg, yield 64%) was obtained as colorless crystals from tert-butyl 2-{3[(aminocarbonothioyl)amino]-1H-indazol-5-yl}nicotinate (101 mg) in the same manner as in Example 2. Melting point 238-239° C.

### Example 15

### 5-pyridin-2-yl-N-1,3-thiazol-2-yl-1H-indazole-3-amine

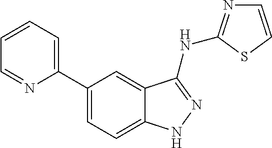

To a solution of 5-pyridin-2-yl-1H-indazole-3-amine (41.8 mg) in tetrahydrofuran (3 mL) was added 1,1′-carbonothioyldipyridine-2(1H)-one (52 mg) at 0° C., stirred for 30 min, and concentrated aqueous ammonia (1 mL) was added. The reaction mixture was stirred at room temperature for 1 hr, water was added, and extracted with ethyl acetate. The ethyl acetate layer was washed with saturated brine, dried over anhydrous magnesium sulfate, and concentrated to give yellow crude crystals. A mixture of the obtained crude crystals, 2-bromo-1,1-diethoxyethane (0.09 mL), 1N hydrochloric acid (1.5 mL) and ethanol (4.5 mL) was heated overnight under reflux. The reaction mixture was neutralized with saturated aqueous sodium hydrogen carbonate, and extracted with ethyl acetate. The ethyl acetate layer was washed with saturated brine, dried over anhydrous magnesium sulfate, and concentrated. The residue was subjected to NH silica gel column chromatography (ethyl acetate) to give the title compound (29 mg, yield 50%) as colorless crystals. Melting point 234-236° C.

### Example 16

### 5-[3-(2,5-dimethyl-1H-pyrrol-1-yl)pyridin-2-yl]-N-1,3-thiazol-2-yl-1H-indazole-3-amine

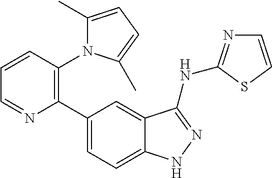

The title compound (89 mg, yield 36%) was obtained as colorless crystals from N-{5-[3-(2,5-dimethyl-1H-pyrrol-1-yl)pyridin-2-yl]-1H-indazol-3-yl}thiourea (232 mg) in the same manner as in Example 2. Melting point 149-150° C.

### Example 17

### 2-[3-(1,3-thiazol-2-ylamino)-1H-indazol-5-yl]nicotinic acid

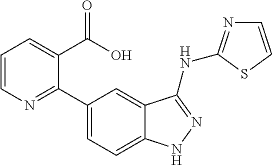

To tert-butyl 2-[3-(1,3-thiazol-2-ylamino)-1H-indazol-5-yl]nicotinate (60 mg) was added 4N hydrogen chloride-ethyl acetate (2 mL), and the mixture was stirred overnight at room temperature. The solvent was evaporated, and the obtained solid was washed with ethyl acetate to give the title compound (58.5 mg, yield 100%) as pale-yellow crystals. Melting point 267-268° C.

### Example 18

### 5-(3-aminopyridin-2-yl)-N-1,3-thiazol-2-yl-1H-indazole-3-amine

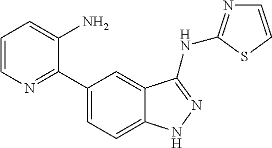

To an N,N-dimethylformamide solution (2 mL) of 2-[3-(1,3-thiazol-2-ylamino)-1H-indazol-5-yl]nicotinic acid (83 mg) were added triethylamine (0.11 mL) and diphenylphosphoric acid azide (0.059 mL), and the mixture was stirred at room temperature for 1 hr. Water was added, and the mixture was stirred overnight at 100° C. The mixture was allowed to cool, diluted with ethyl acetate, washed with water and saturated brine, dried over anhydrous magnesium sulfate, and concentrated under reduced pressure. The residue was purified by NH-silica gel column chromatography (ethyl acetate) to give the title compound (13.3 mg, yield 18%) as a colorless solid. Melting point 282-283° C.

### Example 19

### N-(pyridin-2-ylmethyl)-3-(1,3-thiazol-2-ylamino)-1H-indazole-5-sulfonamide

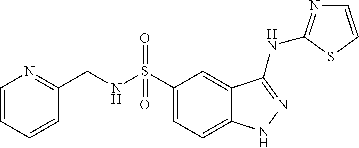

The title compound (148 mg, yield 54%) was obtained as pale-yellow crystals from 3-[(aminocarbonothioyl)amino]-N-(pyridin-2-ylmethyl)-1H-indazole-5-sulfonamide (258 mg) in the same manner as in Example 2. MS: 387 (MH⁺).

### Example 20

### 5-(pyrrolidin-1-ylsulfonyl)-N-1,3-thiazol-2-yl-1H-indazole-3-amine

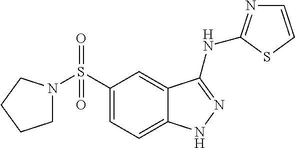

To an ethanol-1N hydrochloric acid (6 mL-2 mL) solution of N-[5-(pyrrolidin-1-ylsulfonyl)-1H-indazol-3-yl]thiourea (252 mg) was added 2-bromo-1,1-diethoxyethane (0.19 mL), and the mixture was stirred at 80° C. for 4 hr. The reaction mixture was basified with saturated aqueous sodium hydrogen carbonate, diluted with ethyl acetate-tetrahydrofuran, washed with saturated brine, dried over anhydrous magnesium sulfate, and concentrated. The obtained crude crystals were recrystallized (ethanol) to give the title compound (210 mg, yield 78%) as colorless crystals. MS: 349 (MH⁺), ¹H NMR (300 MHz, DMSO-d₆) δ ppm 1.46-1.82 (4H, m) 2.99-3.26 (4H, m) 7.05 (1H, d, J=3.8 Hz) 7.38 (1H, d, J=3.4 Hz) 7.58 (1H, d, J=8.7 Hz) 7.73 (1H, dd, J=8.9, 1.7 Hz) 8.78 (1H, s) 11.58 (1H, br. s.) 12.84 (1H, br. s.).

### Example 21

### 7-pyridin-4-yl-N-1,3-thiazol-2-yl-1H-indazole-3-amine

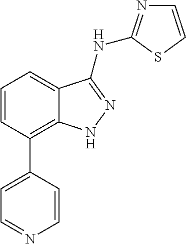

The title compound (359 mg, yield 100%) was obtained as pale-yellow crystals from N-(7-pyridin-4-yl-1H-indazol-3-yl)thiourea (280 mg) in the same manner as in Example 2.

MS: 294 (MH⁺).

### Example 22

### 5-propyl-N-1,3-thiazol-2-yl-7-(2-thienyl)-1H-indazole-3-amine

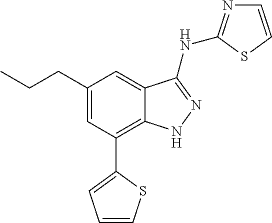

The title compound (18.3 mg, yield 57%) was obtained as pale-yellow crystals from N-[5-propyl-7-(2-thienyl)-1H-indazol-3-yl]thiourea (30 mg) in the same manner as in Example 2.

MS: 341 (MH⁺).

### Example 23

### 5-propyl-7-pyridin-3-yl-N-1,3-thiazol-2-yl-1H-indazole-3-amine

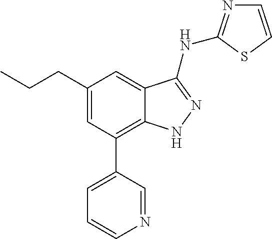

The title compound (114.4 mg, yield 68%) was obtained as colorless crystals from N-(5-propyl-7-pyridin-3-yl-1H-indazol-3-yl)thiourea (156 mg) in the same manner as in Example 2.

MS: 336 (MH⁺).

### Example 24

### 5-propyl-7-[(E)-2-pyridin-4-ylvinyl]-N-1,3-thiazol-2-yl-1H-indazole-3-amine

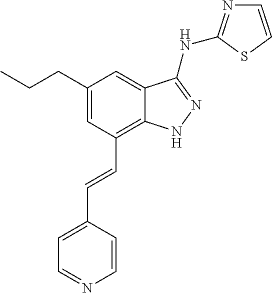

The title compound (54.3 mg, yield 73%) was obtained as pale-yellow crystals from N-{5-propyl-7-[(E)-2-pyridin-4-ylvinyl]-1H-indazol-3-yl}thiourea (69.7 mg) in the same manner as in Example 2. MS: 362 (MH⁺).

### Example 25

### 5-propyl-N,7-di-1,3-thiazol-2-yl-1H-indazole-3-amine

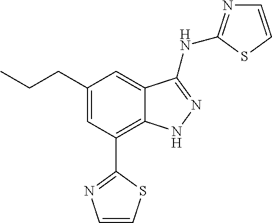

The title compound (180 mg, yield 75%) was obtained as pale-yellow crystals from N-[5-propyl-7-(1,3-thiazol-2-yl)-1H-indazol-3-yl]thiourea (224 mg) in the same manner as in Example 2. MS: 342 (MH⁺).

### Example 26

### 5-propyl-7-pyridin-4-yl-N-1,3-thiazol-2-yl-1H-indazole-3-amine

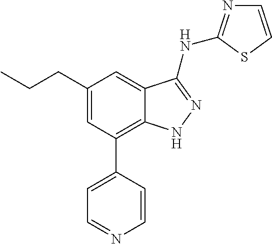

The title compound (46.2 mg, yield 70%) was obtained as pale-yellow crystals from N-(5-propyl-7-pyridin-4-yl-1H-indazol-3-yl)thiourea (61.1 mg) in the same manner as in Example 2. MS: 336 (MH⁺).

### Example 27

### 7-(1-methyl-1H-pyrazol-4-yl)-5-propyl-N-1,3-thiazol-2-yl-1H-indazole-3-amine

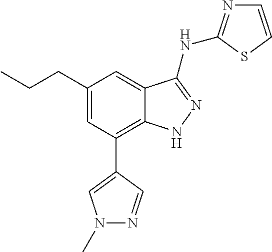

The title compound (34.5 mg, yield 58%) was obtained as pale-yellow crystals from N-[7-(1-methyl-1H-pyrazol-4-yl)-5-propyl-1H-indazol-3-yl]thiourea (55.3 mg) in the same manner as in Example 2. MS: 339 (MH⁺).

### Example 28

### 7-(1-benzothien-2-yl)-5-propyl-N-1,3-thiazol-2-yl-1H-indazole-3-amine

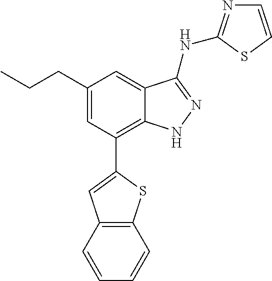

To a solution of N-[7-(1-benzothien-2-yl)-5-propyl-1H-indazol-3-yl]thiourea (40.6 mg) in ethanol-1N hydrochloric acid (3 mL-1 mL) was added 2-bromo-1,1-diethoxyethane (0.04 ml), and the mixture was stirred overnight at 80° C. The reaction mixture was basified with saturated aqueous sodium hydrogen carbonate, diluted with ethyl acetate-tetrahydrofuran, washed with saturated brine, dried over anhydrous magnesium sulfate, and concentrated. The obtained crude crystals were recrystallized (tetrahydrofuran-diisopropyl ether) to give the title compound (20.9 mg, yield 49%) as pale-yellow crystals.

MS: 391 (MH⁺), ¹H NMR (300 MHz, DMSO-d₆) δ ppm 0.97 (3H, t, J=7.3 Hz) 1.59-1.81 (2H, m) 2.73 (2H, t, J=7.4 Hz) 7.03 (1H, d, J=3.6 Hz) 7.36-7.53 (4H, m) 7.83-7.92 (1H, m) 7.96-8.12 (3H, m) 11.38 (1H, s) 12.61 (1H, s).

### Example 29

### 5-(3-chloropyridin-2-yl)-1-methyl-N-1,3-thiazol-2-yl-1H-indazole-3-amine

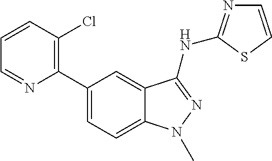

The title compound (47.5 mg, yield 50%) was obtained as colorless crystals from N-[5-(3-chloropyridin-2-yl)-1-methyl-1H-indazol-3-yl]thiourea (88.6 mg) in the same manner as in Example 2. MS: 342 (MH⁺).

### Example 30

### 5-(3-chloropyridin-2-yl)-1-ethyl-N-1,3-thiazol-2-yl-1H-indazole-3-amine

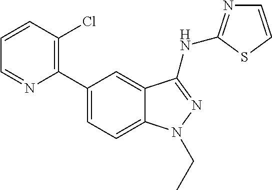

To a solution of N-[5-(3-chloropyridin-2-yl)-1-ethyl-1H-indazol-3-yl]thiourea (100 mg) in ethanol-1N hydrochloric acid (3 mL-1 mL) was added 2-bromo-1,1-diethoxyethane (0.091 mL), and the mixture was stirred overnight at 80° C. The reaction mixture was basified with saturated aqueous sodium hydrogen carbonate, diluted with ethyl acetate-tetrahydrofuran, washed with saturated brine, dried over anhydrous magnesium sulfate, and concentrated. The obtained residue was subjected to NH-silica gel column chromatography (eluate: ethyl acetate), and the obtained crude crystals were recrystallized (ethyl acetate-diisopropyl ether) to give the title compound (66 mg, yield 62%) as colorless crystals. MS: 356 (MH⁺), ¹H NMR (300 MHz, DMSO-d₆) δ ppm 1.45 (3H, t, J=7.2 Hz) 4.37 (2H, q, J=7.0 Hz) 7.01 (1H, d, J=3.6 Hz) 7.37 (1H, d, J=3.6 Hz) 7.43 (1H, dd, J=8.1, 4.5 Hz) 7.61-7.67 (1H, m) 7.70-7.79 (1H, m) 8.06 (1H, dd, J=8.0, 1.4 Hz) 8.55 (1H, s) 8.65 (1H, dd, J=4.7, 1.5 Hz) 11.52 (1H, s).

### Example 31

### 1-benzyl-5-(3-chloropyridin-2-yl)-N-1,3-thiazol-2-yl-1H-indazole-3-amine

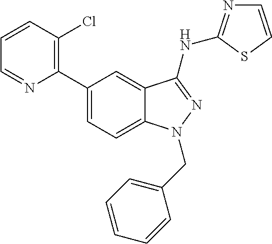

The title compound (64.2, yield 60%) was obtained as colorless crystals from N-[1-benzyl-5-(3-chloropyridin-2-yl)-1H-indazol-3-yl]thiourea (100 mg) in the same manner as in Example 2. MS: 418 (MH⁺).

### Example 32

### (2-{[5-(3-chloropyridin-2-yl)-1H-indazol-3-yl]amino}-1,3-thiazol-5-yl)methanol

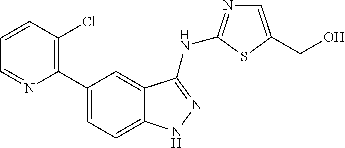

To an ethanol-tetrahydrofuran suspension (10 mL-20 mL) of 2-{[5-(3-chloropyridin-2-yl)-1H-indazol-3-yl]amino}-1,3-thiazole-5-carbaldehyde (1.67 g) was added sodium borohydride (266 mg) under ice-cooling, and the mixture was stirred at room temperature for 3 hr. Water was added, and the organic solvent was evaporated under reduced pressure. The precipitated solid was collected by filtration, washed with water, dissolved in tetrahydrofuran-ethyl acetate, washed with saturated aqueous sodium hydrogen carbonate and water, and dried over anhydrous magnesium sulfate. The solvent was evaporated under reduced pressure, and the precipitated solid was washed with ethyl acetate to give the title compound (927 mg, yield 55%) as a colorless solid. Furthermore, mother liquor was concentrated, and the precipitated solid was washed with diisopropyl ether to give the title compound (304 mg, yield 18%) as colorless crystals. MS: 358 (MH⁺).

### Example 33

### N-{5-[(4-acetylpiperazin-1-yl)methyl]-1,3-thiazol-2-yl}-5-(3-chloropyridin-2-yl)-1H-indazole-3-amine

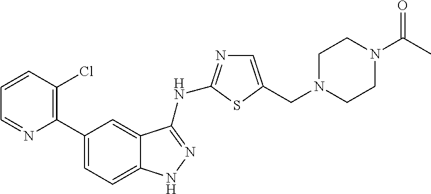

To a tetrahydrofuran solution (2 mL) of 2-{[5-(3-chloropyridin-2-yl)-1H-indazol-3-yl]amino}-1,3-thiazole-5-carbaldehyde (79.8 mg) were added 1-acetylpiperazine (35 mg) and sodium triacetoxyhydroborate (143 mg), and the mixture was room stirred overnight at room temperature. Saturated aqueous sodium hydrogen carbonate and ethyl acetate were added, and the organic layer was washed with water and saturated brine and dried over anhydrous magnesium sulfate. The solvent was evaporated under reduced pressure, and the obtained crude crystals were recrystallized (tetrahydrofuran-ethyl acetate) to give the title compound (68.7 mg, yield 66%) as a colorless solid. MS: 468 (MH⁺).

### Example 34

### 5-(3-chloropyridin-2-yl)-N-[5-(morpholin-4-ylmethyl)-1,3-thiazol-2-yl]-1H-indazole-3-amine

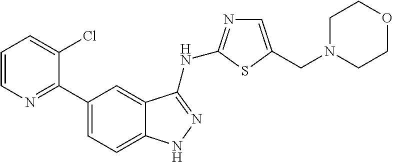

To a tetrahydrofuran solution (2 mL) of 2-{[5-(3-chloropyridin-2-yl)-1H-indazol-3-yl]amino}-1,3-thiazole-5-carbaldehyde (150 mg) were added morpholine (51 mg) and sodium triacetoxyhydroborate (270 mg), and the mixture was stirred overnight at room temperature. Saturated aqueous sodium hydrogen carbonate and ethyl acetate were added, and the organic layer was washed with water and saturated brine and dried over anhydrous magnesium sulfate. The solvent was evaporated under reduced pressure, and the obtained crude crystals were recrystallized (tetrahydrofuran-ethyl acetate) to give the title compound (95.7 mg, yield 58%) as colorless crystals. Melting point 217° C., MS: 427 (MH⁺), ¹H NMR (300 MHz, DMSO-d₆) δ ppm 2.39-2.42 (m, 4H) 3.57-3.63 (m, 6H) 7.17 (s, 1H) 7.42 (dd, 1H, J=8.1, 4.8 Hz) 7.49 (d, 1H, J=8.7 Hz) 7.70 (dd, 1H, J=8.7, 1.5 Hz) 8.06 (dd, 1H, J=8.1, 1.5 Hz) 8.54 (s, 1H) 8.64 (dd, 1H, J=4.5, 1.5 Hz) 11.32 (s, 1H) 12.46 (s, 1H).

### Example 35

### N-{5-[(benzylamino)methyl]-1,3-thiazol-2-yl}-5-(3-chloropyridin-2-yl)-1H-indazole-3-amine

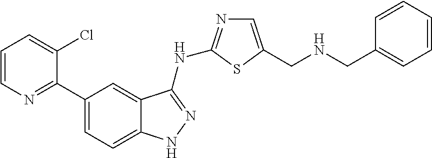

The title compound (87 mg, yield 43%) was obtained as colorless crystals from 2-{[5-(3-chloropyridin-2-yl)-1H-indazol-3-yl]amino}-1,3-thiazole-5-carbaldehyde (150 mg) and benzylamine (63 mg) in the same manner as in Example 33. Melting point 159° C.

### Example 36

### N-[5-(1,4′-bipiperidine-1′-ylmethyl)-1,3-thiazol-2-yl]-5-(3-chloropyridin-2-yl)-1H-indazole-3-amine

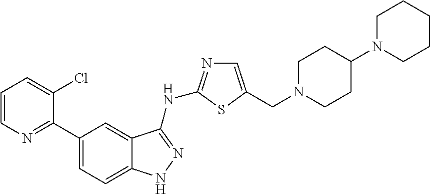

The title compound (111 mg, 28%) was obtained as colorless crystals from 2-{[5-(3-chloropyridin-2-yl)-1H-indazol-3-yl]amino}-1,3-thiazole-5-carbaldehyde (150 mg) and 1,4′-bipiperidine (98 mg) in the same manner as in Example 33. Melting point 167° C.

### Example 37

### 5-(3-chloropyridin-2-yl)-N-{5-[(dimethylamino)methyl]-1,3-thiazol-2-yl}-1H-indazole-3-amine

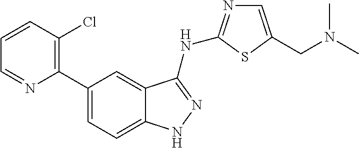

The title compound (67.2 mg, yield 58%) was obtained as colorless crystals from 2-{[5-(3-chloropyridin-2-yl)-1H-indazol-3-yl]amino}-1,3-thiazole-5-carbaldehyde (107 mg) and dimethylamine (2M tetrahydrofuran solution, 0.3 mL) in the same manner as in Example 33. Melting point 283-285° C.

### Example 38

### N-(5-{[benzyl(methyl)amino]methyl}-1,3-thiazol-2-yl)-5-(3-chloropyridin-2-yl)-1H-indazole-3-amine

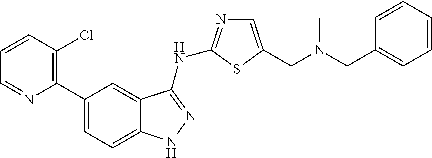

is The title compound (122 mg, yield 62%) was obtained as colorless crystals from 2-{[5-(3-chloropyridin-2-yl)-1H-indazol-3-yl]amino}-1,3-thiazole-5-carbaldehyde (152 mg) and benzyl(methyl)amine (0.067 mL) in the same manner as in Example 33. Melting point 223-224° C.

### Example 39

### 5-(3-chloropyridin-2-yl)-N-(5-{[methyl(pyridin-2-ylmethyl)amino]methyl}-1,3-thiazol-2-yl)-1H-indazole-3-amine

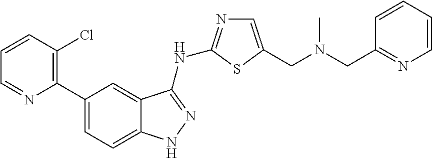

The title compound (102 mg, yield 50%) was obtained as colorless crystals from 2-{[5-(3-chloropyridin-2-yl)-1H-indazol-3-yl]amino}-1,3-thiazole-5-carbaldehyde (157 mg), methyl(pyridin-2-ylmethyl)amine hydrochloride (0.084 mL) and triethylamine (0.087 mL) in the same manner as in Example 33. Melting point>250° C.

### Example 40

### 5-(3-chloropyridin-2-yl)-N-{5-[(dimethylamino)methyl]-1,3-thiazol-2-yl}-1H-indazole-3-amine trihydrochloride

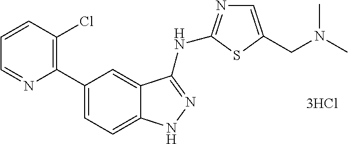

To an ethyl acetate solution (1 mL) of 5-(3-chloropyridin-2-yl)-N-{5-[(dimethylamino)methyl]-1,3-thiazol-2-yl}-1H-indazole-3-amine (30.4 mg) was added 4N hydrogen is chloride -ethyl acetate (2 mL), and the mixture was stirred and concentrated under reduced pressure. The precipitated solid was collected by filtration, and washed with ethyl acetate to give the title compound (33.2 mg, yield 85%) as pale-yellow crystals. Melting point 202-204° C.

### Example 41

### N-(5-{[benzyl(methyl)amino]methyl}-1,3-thiazol-2-yl)-5-(3-chloropyridin-2-yl)-1H-indazole-3-amine trihydrochloride

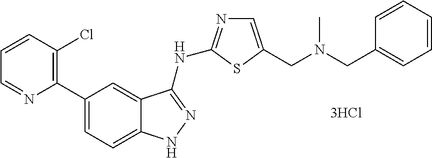

The title compound (72.6 mg, yield 98%) was obtained as pale-yellow crystals from N-(5-{[benzyl(methyl)amino]methyl}-1,3-thiazol-2-yl)-5-(3-chloropyridin-2-yl)-1H-indazole-3-amine (59.7 mg) in the same manner as in Example 40. MS: 461(MH⁺-3HCl).

### Example 42

### 5-(3-chloropyridin-2-yl)-N-(5-{[methyl(pyridin-2-ylmethyl)amino]methyl}-1,3-thiazol-2-yl)-1H-indazole-3-amine tetrahydrochloride

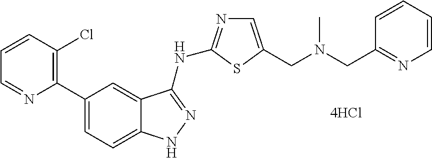

The title compound (57.5 mg, yield 95%) was obtained as pale-yellow crystals from 5-(3-chloropyridin-2-yl)-N-(5-{[methyl(pyridin-2-ylmethyl)amino]methyl}-1,3-thiazol-2-yl)-1H-indazole-3-amine (46.5 mg) in the same manner as in Example 40.

MS: 462 (MH⁺−4HCl)

### Example 43

### 8-[(2-{[5-(3-chloropyridin-2-yl)-1H-indazol-3-yl]amino}-1,3-thiazol-5-yl)methyl]hexahydropyrazino[2,1-c][1,4]oxadin-4(3H)-one

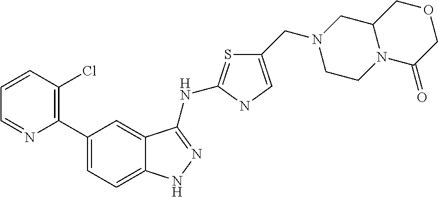

The title compound (27 mg, yield 19%) was obtained as colorless crystals from 2-{[5-(3-chloropyridin-2-yl)-1H-indazol-3-yl]amino}-1,3-thiazole-5-carbaldehyde (100 mg), hexahydropyrazino[2,1-c][1,4]oxadine-4(3H) monohydrochloride (67 mg) and triethylamine (0.070 mL) in the same manner as in Example 33. Melting point 166° C.

### Example 44

### 7-[(2-{[5-(3-chloropyridin-2-yl)-1H-indazol-3-yl]amino}-1,3-thiazol-5-yl)methyl]hexahydro[1,3]oxazolo[3,4-a]pyrazin-3-one

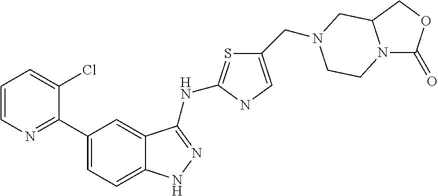

The title compound (37 mg, yield 28%) was obtained as colorless crystals from 2-{[5-(3-chloropyridin-2-yl)-1H-indazol-3-yl]amino}-1,3-thiazole-S-carbaldehyde (100 mg), hexahydro[1,3]oxazolo[3,4-a]pyrazin-3-one1hydrochloride (62 mg) and triethylamine (0.070 mL) in the same manner as in Example 33. Melting point 164° C.

### Example 45

### 2-{4-[(2-{[5-(3-chloropyridin-2-yl)-1H-indazol-3-yl]amino}-1,3-thiazol-5-yl)methyl]piperazin-1-yl}-N,N-dimethylacetamide

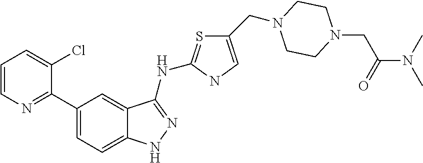

The title compound (103 mg, yield 48%) was obtained as colorless crystals from 2-{[5-(3-chloropyridin-2-yl)-1H-indazol-3-yl]amino}-1,3-thiazole-5-carbaldehyde (100 mg) and N,N-dimethyl-2-piperazin-1-ylacetamide (87 mg) in the same manner as in Example 33. Melting point 166° C.

### Example 46

### 5-(3-chloropyridin-2-yl)-N-[5-(thiomorpholin-4-ylmethyl)-1,3-thiazol-2-yl]-1H-indazole-3-amine

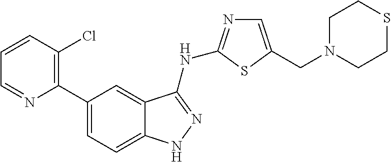

The title compound (119 mg, yield 64%) was obtained as colorless crystals from 2-{[5-(3-chloropyridin-2-yl)-1H-indazol-3-yl]amino}-1,3-thiazole-5-carbaldehyde (150 mg) and thiomorpholine (0.051 mL) in the same manner as in Example 33. Melting point 206-208° C.

### Example 47

### 5-(3-chloropyridin-2-yl)-N-{5-[(1,1-dioxidethiomorpholin-4-YL)methyl]-1,3-thiazol-2-yl}-1H-indazole-3-amine

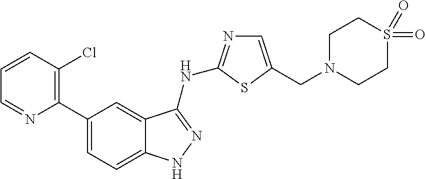

The title compound (113 mg, yield 57%) was obtained as colorless crystals from 2-{[5-(3-chloropyridin-2-yl)-1H-indazol-3-yl]amino}-1,3-thiazole-5-carbaldehyde (150 mg) and thiomorpholine 1,1-dioxide (68 mg) in the same manner as in Example 33. Melting point>250° C.

### Example 48

### 5-(3-chloropyridin-2-yl)-N-[5-(1,4-dioxa-8-azaspiro[4.5]deca-8-ylmethyl)-1,3-thiazol-2-yl]-1H-indazole-3-amine

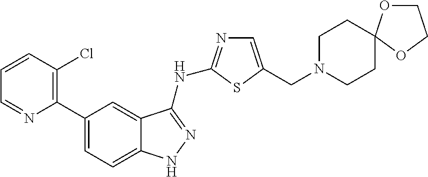

The title compound (126 mg, yield 62%) was obtained as pale-yellow crystals from 2-{[5-(3-chloropyridin-2-yl)-1H-indazol-3-yl]amino}-1,3-thiazole-5-carbaldehyde (150 mg) and 1,4-dioxa-8-azaspiro[4.5]decane (0.065 mL) in the same manner as in Example 33. Melting point 220-222° C.

### Example 49

### 5-(3-chloropyridin-2-yl)-N-(5-{[methoxy(methyl)amino]methyl}-1,3-thiazol-2-yl)-1H-indazole-3-amine

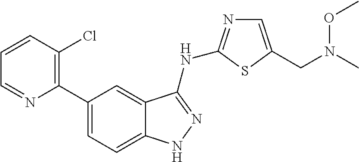

The title compound (53.5 mg, yield 31%) was obtained as colorless crystals from 2-{[5-(3-chloropyridin-2-yl)-1H-indazol-3-yl]amino}-1,3-thiazole-5-carbaldehyde (151 mg), N-methoxymethaneamine hydrochloride (62 mg) and triethylamine (0.11 mL) in the same manner as in Example 33. Melting point 230-232° C.

### Example 50

### N-(5-{[bis(2-methoxyethyl)amino]methyl}-1,3-thiazol-2-yl)-5-(3-chloropyridin-2-yl)-1H-indazole-3-amine

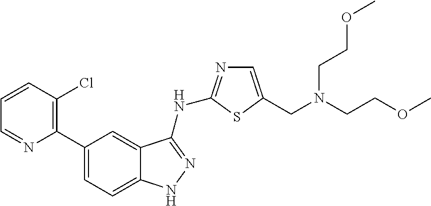

The title compound (103 mg, yield 51%) was obtained as colorless crystals from 2-{[5-(3-chloropyridin-2-yl)-1H-indazol-3-yl]amino}-1,3-thiazole-5-carbaldehyde (154 mg) and bis(2-methoxyethyl)amine (0.077 mL) in the same manner as in Example 33. Melting point 250-251° C.

### Example 51

### 1-[(2-{[5-(3-chloropyridin-2-yl)-1H-indazol-3-yl]amino}-1,3-thiazol-5-yl)methyl]piperidine-4-one

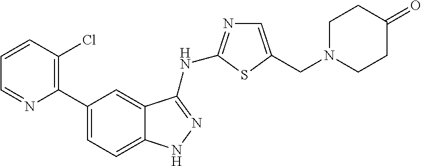

A 6N hydrochloric acid solution (2 mL) of 5-(3-chloropyridin-2-yl)-N-[5-(1,4-dioxa-8-azaspiro[4.5]deca-8-ylmethyl)-1,3-thiazol-2-yl]-1H-indazole-3-amine (74 mg) was stirred at 80 to 90° C. for 6 hr. The mixture was basified by adding saturated aqueous sodium hydrogen carbonate, and extracted, with: ethyl acetate-tetrahydrofuran. The organic layer was washed with saturated brine, and dried over anhydrous magnesium sulfate. The solvent was evaporated under reduced pressure, and the obtained crude crystals were recrystallized (ethyl acetate) to give the title compound (30.3 mg, yield 45%) as a colorless solid. Melting point 187-188° C.

### Example 52

### 1-[(2-{[5-(3-chloropyridin-2-yl)-1H-indazol-3-yl]amino}-1,3-thiazol-5-yl)methyl]piperidin-4-ol

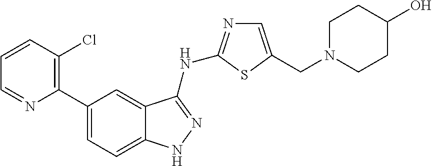

The title compound (54.6 mg, yield 29%) was obtained as colorless crystals from 2-{[5-(3-chloropyridin-2-yl)-1H-indazol-3-yl]amino}-1,3-thiazole-5-carbaldehyde (154 mg) and piperidin-4-ol (53 mg) in the same manner as in Example 33. Melting point 140-142° C.

### Example 53

### 5-(3-chloropyridin-2-yl)-N-{5-[(methylamino)methyl]-1,3-thiazol-2-yl}-1H-indazole-3-amine

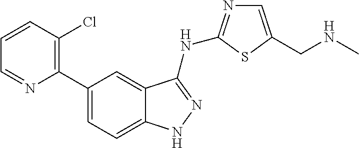

To an ethyl acetate solution (1 mL) of tert-butyl [(2-{[5-(3-chloropyridin-2-yl)-1H-indazol-3-yl]amino}-1,3-thiazol-5-yl)methyl]methylcarbamate (51.1 mg) was added 4N hydrogen chloride-ethyl acetate solution (2 mL), and the mixture was stirred at room temperature for 30 min. The mixture was concentrated under reduced pressure, and the powder was collected by filtration and washed with ethyl acetate. To the powder was added saturated aqueous sodium hydrogen carbonate, and extracted with ethyl acetate. The extract was dried over anhydrous magnesium sulfate, and concentrated under reduced pressure. The obtained crude crystals were recrystallized (tetrahydrofuran-ethyl acetate) to give the title compound (12.2 mg, yield 30%) as colorless crystals. Melting point 200-201° C.

### Example 54

### (2-{[5-(3-chloropyridin-2-yl)-1H-indazol-3-yl]amino}-1,3-thiazol-5-yl)acetonitrile

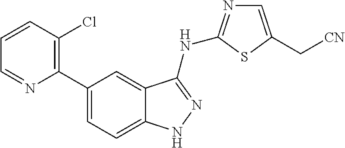

To a tetrahydrofuran solution (2 mL) of tert-butyl 5-(3-chloropyridin-2-yl)-3-{[5-(cyanomethyl)-1,3-thiazol-2-yl]amino}-1H-indazole-1-carboxylate (12.5 mg) was added trifluoroacetic acid (2 mL), and the mixture was stirred for 4 hr at room temperature. The solvent was evaporated under reduced pressure, and diluted with ethyl acetate. The mixture was basified by adding saturated aqueous sodium hydrogen carbonate, and the organic layer was washed with water and saturated brine, dried over anhydrous magnesium sulfate, and concentrated under reduced pressure. The residue was subjected to NH silica gel column chromatography, and the ethyl acetate eluant was concentrated under reduced pressure. The crude crystals were recrystallized (ethyl acetate-diisopropyl ether) to give the title compound (1.3 mg, yield 13%) as colorless crystals. MS: 367 (MH⁺)

### Example 55

### ethyl (2-{[5-(3-chloropyridin-2-yl)-1H-indazol-3-yl]amino}-1,3-thiazol-5-yl)acetate

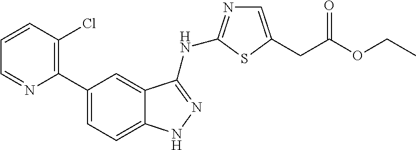

An ethanol solution (30 mL) of 5-(3-chloropyridin-2-yl)-N-1,3-thiazol-2-yl-1H-indazole-3-amine (1.07 g) and 3-bromo-4-oxobutaneacidethyl (0.886 g) was heated overnight under reflux. Saturated aqueous sodium hydrogen carbonate was added, and tetrahydrofuran was extracted with ethyl acetate. The organic layer was washed with water and saturated brine, dried over anhydrous magnesium sulfate, and concentrated under reduced pressure. The precipitated solid was washed with diisopropyl ether to give the title compound (1.076 g, yield 74%) as a colorless solid. Melting point 259-261° C.

### Example 56

### (2-{[5-(3-chloropyridin-2-yl)-1H-indazol-3-yl]amino}-1,3-thiazol-5-yl)acetic acid

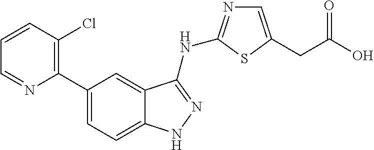

A concentrated hydrochloric acid suspension (20 mL) of ethyl (2-{[5-(3-chloropyridin-2-yl)-1H-indazol-3-yl]amino}-1,3-thiazol-5-yl) acetate (762 mg) was heated under reflux for 2 hr. The mixture was concentrated under reduced pressure, diluted with water, and basified by adding saturated aqueous sodium hydrogen carbonate. The aqueous layer was washed with ethyl acetate, neutralized with 1N hydrochloric acid, and extracted with ethyl acetate. The organic layer was washed with saturated brine, dried over anhydrous magnesium sulfate, and concentrated under reduced pressure. The obtained crude crystals were recrystallized (ethyl acetate) to give the title compound (654 mg, yield 91%) as a colorless solid. MS: 386(MW).

### Example 57

### 2-(2-{[5-(3-chloropyridin-2-yl)-1H-indazol-3-yl]amino}-1,3-thiazol-5-yl)ethanol

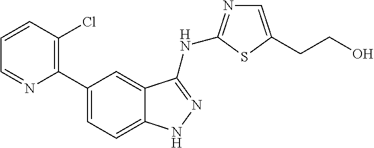

To a tetrahydrofuran solution (7 mL) of ethyl (2-{[5-(3-chloropyridin-2-yl)-1H-indazol-3-yl]amino}-1,3-thiazol-5-yl)acetate (150 mg) was added lithium borohydride (100 mg), and the mixture was stirred overnight at room temperature. 1N hydrochloric acid was added, and the mixture was basified by adding saturated aqueous sodium hydrogen carbonate. The aqueous layer was extracted with ethyl acetate, washed with water and saturated brine, dried over anhydrous magnesium sulfate, and concentrated under reduced pressure. The obtained crude crystals were recrystallized (tetrahydrofuran-ethyl acetate) to give the title compound (76.4 mg, yield 57%) as a colorless solid. Melting point 206-208° C.

### Example 58

### 2-(2-{[5-(3-chloropyridin-2-yl)-1H-indazol-3-yl]amino}-1,3-thiazol-5-yl)acetamide

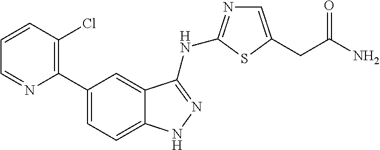

To an N,N-dimethylformamide solution (5 mL) of (2-{[5-(3-chloropyridin-2-yl)-1H-indazol-3-yl]amino}-1,3-thiazol-5-yl)acetic acid (151 mg) were added 1-ethyl-3-(3-dimethylaminopropyl)carbodiimide hydrochloride (90 mg) and 1-hydroxybenzotriazole monohydrate (72 mg), and the mixture was stirred at room temperature for 30 min. Aqueous ammonia (28%, 2 mL) was added, and the mixture was stirred at room temperature for 30 min. The mixture was diluted with ethyl acetate, saturated aqueous sodium hydrogen carbonate, washed with water and saturated brine, dried over anhydrous magnesium sulfate, and concentrated under reduced pressure. The obtained crude crystals were recrystallized (tetrahydrofuran) to give the title compound (40.4 mg, yield 27%) as a colorless solid. Melting point>230° C.

### Example 59

### 5-(3-chloropyridin-2-yl)-N-pyridin-2-yl-1H-indazole-3-amine

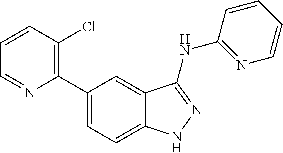

To an N-methyl-2-pyrrolidinone solution (20 ml) of 5-(3-chloropyridin-2-yl)-1H-indazole-3-amine (1.3 g) was added 2-chloropyridine monohydrochloride (3.4 g), and the mixture was stirred at 160° C. for 16 hr. The mixture was cooled to room temperature, diluted with ethyl acetate, and washed with saturated aqueous sodium hydrogen carbonate. The organic layer was washed with saturated brine, dried over anhydrous magnesium sulfate, and concentrated under reduced pressure. The residue was purified by NH-silica gel column chromatography (eluate: ethyl acetate) to give the title compound (146 mg, yield 8.6%) as white crystals. Melting point 164° C.

### Example 60

### 5-(3-chloropyridin-2-yl)-N-pyrazin-2-yl-1H-indazole-3-amine

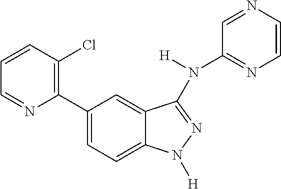

To a solution of 5-(3-chloropyridin-2-yl)-N-pyrazin-2-yl-1-{[2-(trimethylsilyl)ethoxy]methyl}-1H-indazole-3-amine (250 mg) in ethanol (10 mL) was added 3N hydrochloric acid (10 mL), and heated under reflux for 16 hr. The reaction mixture was cooled to room temperature, saturated aqueous sodium hydrogen carbonate was added, and the mixture was extracted with ethyl acetate. The extract was washed with water and saturated brine, dried over anhydrous magnesium sulfate, and concentrated under reduced pressure. The residue was purified by silica gel column chromatography (ethyl acetate:hexane=1:1-1:0), and the obtained crude crystal was recrystallized from ethyl acetate to give the title compound (79 mg, yield 44%) as colorless crystals. Melting point 228-230° C.

### Example 61

### 5-(3-chloropyridin-2-yl)-N-(6-methylpyridazin-3-yl)-1H-indazole-3-amine

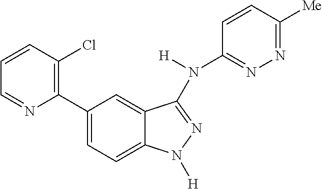

To a solution of 5-(3-chloropyridin-2-yl)-N-(6-methylpyridazin-3-yl)-1-{[2-(trimethylsilyl)ethoxy]methyl}-1H-indazole-3-amine (0.170 g) in ethanol (7 mL) was added 3N hydrochloric acid (7 mL), and the mixture was heated under reflux for 6 hr. The reaction mixture was cooled to room temperature, saturated aqueous sodium hydrogen carbonate was added, and the mixture was extracted with a mixed solvent of ethyl acetate and tetrahydrofuran. The extract was washed with water and saturated brine, dried over anhydrous magnesium sulfate, and concentrated under reduced pressure. The residue was purified by silica gel column chromatography (ethyl acetate:methanol=1:0-7:3), and the obtained crude crystals were recrystallized from diethyl ether to give the title compound (0.060 g, yield 49%) as yellow crystals. MS: 337 (MH⁺).

### Example 62

### 5-(3-chloropyridin-2-yl)-N-1H-pyrazol-3-yl-1H-indazole-3-amine

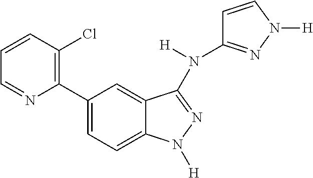

To a solution of 3-[(5-(3-chloropyridin-2-yl)-1-{[2-(trimethylsilyl)ethoxy]methyl}-1H-indazol-3-yl)amino]-N,N-dimethyl-1H-pyrazole-1-sulfonamide (0.285 g) in ethanol (6 mL) was added 3N hydrochloric acid (6 mL), and heated under reflux for 16 hr. The reaction mixture was cooled to room temperature, saturated aqueous sodium hydrogen carbonate was added, and the mixture was extracted with ethyl acetate. The extract was washed with water and saturated brine, dried over anhydrous magnesium sulfate, and concentrated under reduced pressure. The residue was purified by NH-silica gel column chromatography (ethyl acetate:methanol=1:0-9:1), and the obtained crude crystals were recrystallized from hexane-ethyl acetate to give the title compound (0.037 g, yield 23%) as colorless crystals. Melting point 218-220° C.

### Example 63

### 5-(3-chloropyridin-2-yl)-N-(1-methyl-1H-pyrazol-3-yl)-1H-indazole-3-amine

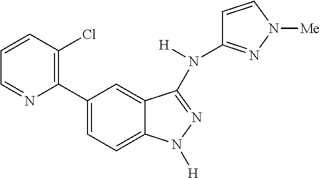

To a solution of tert-butyl 5-(3-chloropyridin-2-yl)-3-[(1-methyl-1H-pyrazol-3-yl)amino]-1H-indazole-1-carboxylate (0.235 g) in ethanol (10 mL) was added 3N hydrochloric acid (10 mL), and heated under reflux for 1 hr. The reaction mixture was cooled to room temperature, saturated aqueous sodium hydrogen carbonate was added, and the mixture was extracted with ethyl acetate. The extract was washed with water and saturated brine, dried over anhydrous magnesium sulfate, and concentrated under reduced pressure. The residue was purified by NH-silica gel column chromatography (ethyl acetate:methanol=1:0-9:1), and the obtained crude crystals were recrystallized (hexane-ethyl acetate) to give the title compound (0.142 g, yield 79%) as colorless crystals. Melting point 190-191° C., MS: 325 (MH⁺), ¹H NMR (300 MHz, CDCl₃) δ ppm 3.82 (3H, s), 6.51 (1H, d, J=2.3 Hz), 6.82 (1H, s), 7.19-7.27 (2H, m), 7.42 (1H, dd, J=8.8, 0.7 Hz), 7.78-7.84 (2H, m), 8.05-8.06 (1H, m), 8.61 (1H, dd, J=4.7, 1.5 Hz), 9.21 (1H, s).

### Example 64

### 7-(benzyloxy)-5-isobutyl-N-1,3-thiazol-2-yl-1H-indazole-3-amine

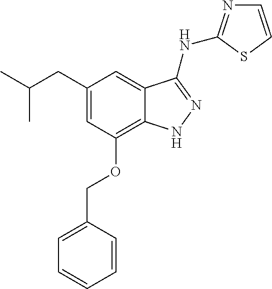

The title compound (71.1 mg, yield 46%) was obtained as colorless crystals from N-[7-(benzyloxy)-5-isobutyl-1H-indazol-3-yl]thiourea (144 mg) in the same manner as in Example 2. Melting point 185-186° C.

### Example 65

### 5-isobutyl-7-(pyridin-2-ylmethoxy)-N-1,3-thiazol-2-yl-1H-indazole-3-amine

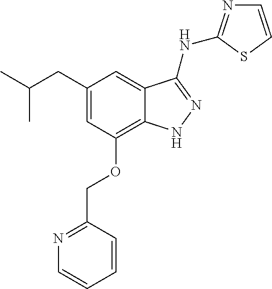

The title compound (115 mg, yield 70%) was obtained as colorless crystals from N-[5-isobutyl-7-(pyridin-2-ylmethoxy)-1H-indazol-3-yl]thiourea (154 mg) in the same manner as in Example 2. Melting point 158-159° C.

### Example 66

### 5-isobutyl-7-(pyridin-3-ylmethoxy)-N-1,3-thiazol-2-yl-1H-indazole-3-amine

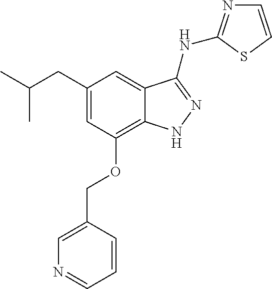

The title compound (58.7 mg, yield 46%) was obtained as colorless crystals from N-[5-isobutyl-7-(pyridin-3-ylmethoxy)-1H-indazol-3-yl]thiourea (120 mg) in the same manner as in Example 2. Melting point 228-229° C.

### Example 67

### 5-isobutyl-7-(pyridin-4-ylmethoxy)-N-1,3-thiazol-2-yl-1H-indazole-3-amine

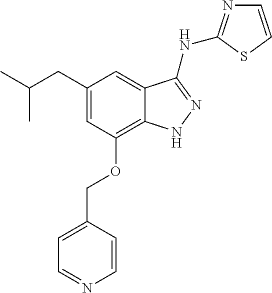

The title compound (114 mg, yield 61%) was obtained as colorless crystals from N-[5-isobutyl-7-(pyridin-4-ylmethoxy)-1H-indazol-3-yl]thiourea (175 mg) in the same manner as in Example 2. Melting point 225-226° C.

### Example 68

### 5-isobutyl-7-[(1-methyl-1H-imidazol-2-yl)methoxy]-N-1,3-thiazol-2-yl-1H-indazole-3-amine

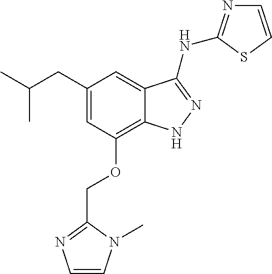

The title compound (52.6 mg, yield 82%) was obtained as colorless crystals from N-{5-isobutyl-7-[(1-methyl-1H-imidazol-2-yl)methoxy]-1H-indazol-3-yl}thiourea (53.7 mg) in the same manner as in Example 2. MS: 359(MH⁺).

### Example 69

### 5-isobutyl-7-{[4-(methylsulfonyl)benzyl]oxy}-N-1,3-thiazol-2-yl-1H-indazole-3-amine

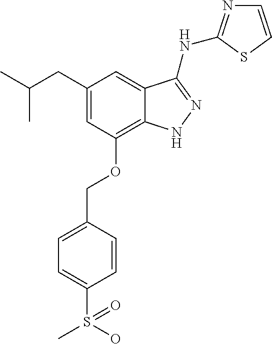

The title compound (111 mg, yield 57%) was obtained as colorless crystals from N-(5-isobutyl-7-{[4-(methylsulfonyl)benzyl]oxy}-1H-indazol-3-yl)thiourea (183 mg) in the same manner as in Example 2. Melting point 222-223° C.

### Example 70

### 7-[(2-fluorobenzyl)oxy]-5-isobutyl-N-1,3-thiazol-2-yl-1H-indazole-3-amine

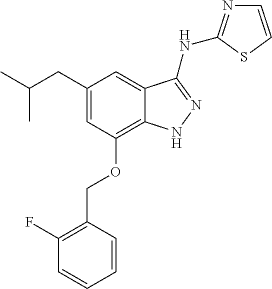

The title compound (57.8 mg, yield 46%) was obtained as colorless crystals from N-{7-[(2-fluorobenzyl)oxy]-5-isobutyl-1H-indazol-3-yl}thiourea (128 mg) in the same manner as in Example 2. Melting point 157-158° C.

### Example 71

### 5-isobutyl-N-1,3-thiazol-2-yl-7-(1,3-thiazol-2-ylmethoxy)-1H-indazole-3-amine

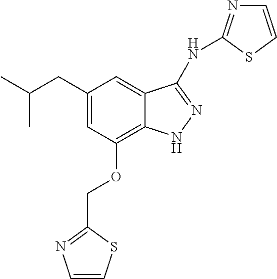

The title compound (52.5 mg, yield 45%) was obtained as colorless crystals from N-[5-isobutyl-7-(1,3-thiazol-2-ylmethoxy)-1H-indazol-3-yl]thiourea (115 mg) in the same manner as in Example 2. Melting point 168-170° C.

### Example 72

### 5-isobutyl-N-1,3-thiazol-2-yl-7-[2-(3-thienyl)ethoxy]-1H-indazole-3-amine

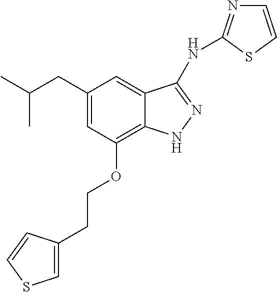

To a solution of N-{5-isobutyl-7-[2-(3-thienyl)ethoxy]-1H-indazol-3-yl}thiourea (236 mg) in ethanol-1N hydrochloric acid (9 mL-3 mL) was added 2-bromo-1,1-diethoxyethane (0.28 mL), and the mixture was stirred overnight at 80° C. The reaction mixture was basified with saturated aqueous sodium hydrogen carbonate, diluted with ethyl acetate-tetrahydrofuran, washed with saturated brine, dried over anhydrous magnesium sulfate and concentrated. The obtained residue was subjected to NH-silica gel column chromatography (eluate: ethyl acetate), and the obtained crude crystals were recrystallized (ethyl acetate-diisopropyl ether) to give the title compound (68.3 mg, yield 28%) as colorless crystals. Melting point 150-151° C.,

MS: 399 (MH⁺), ¹H NMR (300 MHz, DMSO-d₆) δ ppm 0.79-0.95 (6H, m) 1.75-1.97 (1H, m) 2.44-2.55 (2H, m) 3.13 (2H, t, J=6.5 Hz) 4.30 (2H, t, J=6.6 Hz) 6.69 (1H, s) 6.96 (1H, d, J=3.6 Hz) 7.21 (1H, dd, J=4.9, 1.3 Hz) 7.33 (1H, d, J=3.6 Hz) 7.37-7.45 (2H, m) 7.45-7.51 (1H, m) 11.11 (1H, s) 12.39 (1H, s).

### Example 73

### 2-{[5-isobutyl-3-(1,3-thiazol-2-ylamino)-1H-indazole-7-yl]oxy}-N,N-dimethylacetamide

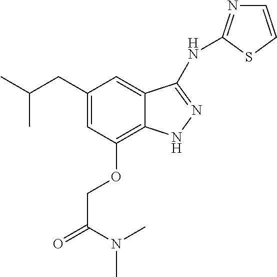

To a solution of 2-({3-[(aminocarbonothioyl)amino]-5-isobutyl-1H-indazole-7-yl}oxy)-N,N-dimethylacetamide (183 mg) in ethanol-1N hydrochloric acid (6 mL-2 mL) was added 2-s bromo-1,1-diethoxyethane (0.24 mL), and the mixture was stirred overnight at 80° C. The reaction mixture was basified with saturated aqueous sodium hydrogen carbonate, diluted with ethyl acetate, washed with saturated brine, dried over anhydrous magnesium sulfate, and concentrated. The obtained residue was subjected to NH-silica gel column chromatography (eluate: ethyl acetate), and the obtained crude crystals were recrystallized (ethyl acetate) to give the title compound (64.4 mg, yield 33%) was obtained as colorless crystals. Melting point 96-98° C.,

MS: 374 (MH⁺), ¹H NMR (300 MHz, DMSO-d₆) δ ppm 0.88 (6H, d, is J=6.6 Hz) 1.73-2.03 (1H, m) 2.40-2.56 (2H, m) 2.85 (3H, s) 3.05 (3H, s) 4.96 (2H, s) 6.59 (1H, s) 6.95 (1H, d, J=3.6 Hz) 7.33 (1H, d, J=3.6 Hz) 7.41 (1H, s) 11.09 (1H, s) 12.40 (1H, s).

### Example 74

### 5-isobutyl-7-(2-pyridin-2-ylethoxy)-N-1,3-thiazol-2-yl-1H-indazole-3-amine

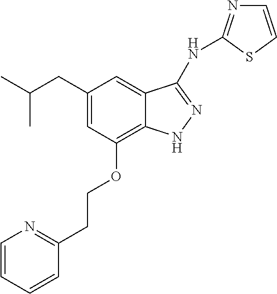

The title compound (13.4 mg, yield 27%) was obtained as colorless crystals from N-[5-isobutyl-7-(2-pyridin-2-ylethoxy)-1H-indazol-3-yl]thiourea (46.0 mg) in the same manner as in Example 2. Melting point 108-109° C.

### Example 75

### 5-isobutyl-N-[5-(morpholin-4-ylmethyl)-1,3-thiazol-2-yl]-7-(pyridin-2-ylmethoxy)-1H-indazole-3-amine

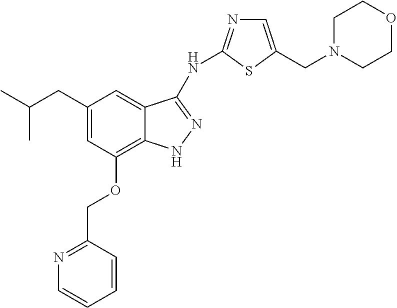

The title compound (87.6 mg, yield 72%) was obtained as colorless crystals from 2-{[5-isobutyl-7-(pyridin-2-ylmethoxy)-1H-indazol-3-yl]amino}-1,3-thiazole-5-carbaldehyde (103 mg) and morpholine (0.027 mL) in the same manner as in Example 33. Melting point 203-206° C.

### Example 76

### N-{5-[(dimethylamino)methyl]-1,3-thiazol-2-yl}-5-isobutyl-7-(pyridin-2-ylmethoxy)-1H-indazole-3-amine

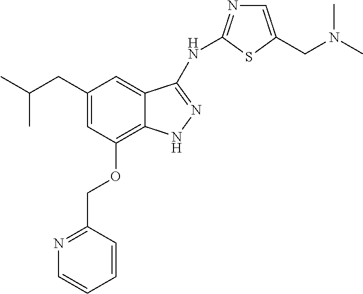

The title compound (81.9 mg, yield 72%) was obtained as colorless crystals from 2-{[5-isobutyl-7-(pyridin-2-ylmethoxy)-1H-indazol-3-yl]amino}-1,3-thiazole-5-carbaldehyde (106 mg) and dimethylamine (2M tetrahydrofuran solution, 0.195 mL) in the same manner as in Example 33. Melting point 177-179° C.

### Example 77

### (2-{[5-isobutyl-7-(pyridin-2-ylmethoxy)-1H-indazol-3-yl]amino}-1,3-thiazol-5-yl)methanol

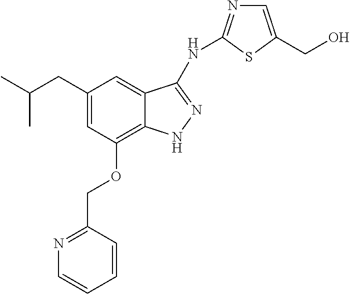

The title compound (46.6 mg, yield 73%) was obtained as colorless crystals from 2-{[5-isobutyl-7-(pyridin-2-ylmethoxy)-1H-indazol-3-yl]amino}-1,3-thiazole-5-carbaldehyde (63.6 mg) in the same manner as in Example 32. Melting point 158-159° C.

### Example 78

### N-{5-[(dimethylamino)methyl]-1,3-thiazol-2-yl}-5-isobutyl-7-[2-(3-thienyl)ethoxy]-1H-indazole-3-amine

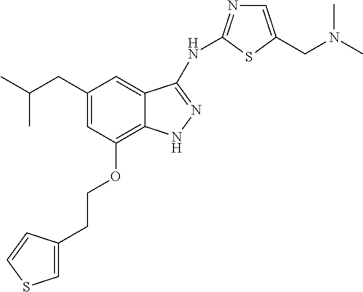

The title compound (37.0 mg, yield 69%) was obtained as colorless crystals from 2-({5-isobutyl-7-[2-(3-thienyl)ethoxy]-1H-indazol-3-yl}amino)-1,3-thiazole-5-carbaldehyde (50 mg) and dimethylamine (2M tetrahydrofuran solution, 0.10 mL) in the same manner as in Example 33. Melting point 172-174° C.

Example 79N-{5-[(dimethylamino)methyl]-1,3-thiazol-2-yl}-5-isobutyl-7-(pyridin-2-ylmethoxy)-1H-indazole-3-amine trihydrochloride

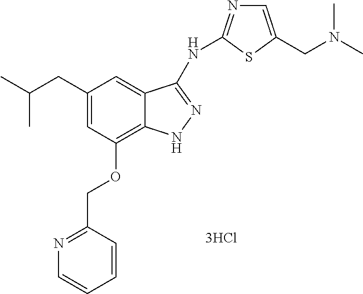

The title compound (45.6 mg, yield 98%) was obtained as pale-yellow crystals from N-{5-[(dimethylamino)methyl]-1,3-thiazol-2-yl}-5-isobutyl-7-(pyridin-2-ylmethoxy)-1H-indazole-3-amine (37.3 mg) in the same manner as in Example 40.

MS: 437 (MH⁺-3HCl)

### Example 80

### 5-isobutyl-N-[5-(morpholin-4-ylmethyl)-1,3-thiazol-2-yl]-7-(pyridin-2-ylmethoxy)-1H-indazole-3-amine trihydrochloride

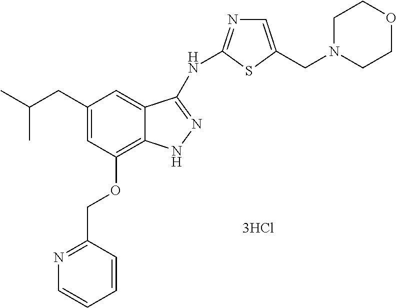

The title compound (46.0 mg, yield 100%) was obtained as pale-yellow crystals from 5-isobutyl-N-[5-(morpholin-4-ylmethyl)-1,3-thiazol-2-yl]-7-(pyridin-2-ylmethoxy)-1H-indazole-3-amine (37.5 mg) in the same manner as in Example 40.

MS: 479 (MH⁺−3HCl).

### Example 81

### 5-(3-chloropyridin-2-yl)-7-(1-methyl-1H-pyrazol-4-yl)-N-1,3-thiazol-2-yl-1H-indazole-3-amine

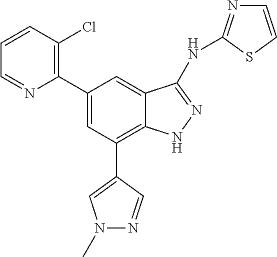

The title compound (121 mg, yield 62%) was obtained as colorless crystals from N-[5-(3-chloropyridin-2-yl)-7-(1-methyl-1H-pyrazol-4-yl)-1H-indazol-3-yl]thiourea (184 mg) in the same manner as in Example 2. Melting point 239-242° C.

### Example 82

### 7-bromo-5-(3-chloropyridin-2-yl)-N-1,3-thiazol-2-yl-1H-indazole-3-amine

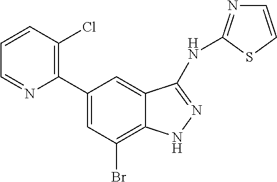

The title compound (94.2 mg, yield 38%) was obtained as colorless crystals from N-[7-bromo-5-(3-chloropyridin-2-yl)-1H-indazol-3-yl]thiourea (234 mg) in the same manner as in Example 2. Melting point 194-196° C.

### Example 83

### 5-(3-chloropyridin-2-yl)-7-methyl-N-1,3-thiazol-2-yl-1H-indazole-3-amine

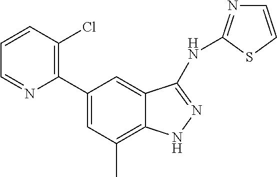

The title compound (269 mg, yield 68%) was obtained as colorless crystals from 5-(3-chloropyridin-2-yl)-7-methyl-1H-indazole-3-amine (299 mg) in the same manner as in Example 15. Melting point 205-207° C.

### Example 84

### 5-(3-chloropyridin-2-yl)-7-(pyridin-2-ylmethoxy)-N-1,3-thiazol-2-yl-1H-indazole-3-amine

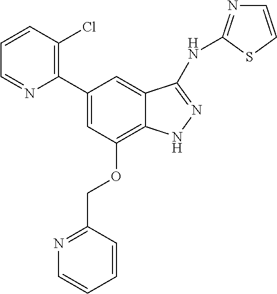

The title compound (37.9 mg, yield 42%) was obtained as colorless crystals from N-[5-(3-chloropyridin-2-yl)-7-(pyridin-2-ylmethoxy)-1H-indazol-3-yl]thiourea (85.7 mg) in the same manner as in Example 2. Melting point 203-204° C.

### Example 85

### 5-(3-chloropyridin-2-yl)-7-(2-pyridin-2-ylethoxy)-N-1,3-thiazol-2-yl-1H-indazole-3-amine

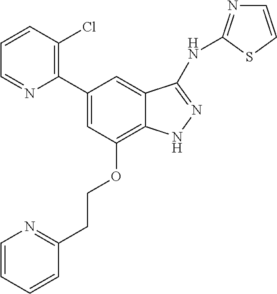

To a tetrahydrofuran solution (2 mL) of 5-(3-chloropyridin-2-yl)-7-(2-pyridin-2-ylethoxy)-1H-indazole-3-s amine (52.0 mg) was added 1,1′-carbonothioyldipyridine-2(1H)-one (37 mg) at 0° C., the mixture was stirred for 30 min, and concentrated aqueous ammonia (1 mL) was added. The reaction mixture was stirred at room temperature for 1 hr, water was added, and the mixture was extracted with ethyl acetate. The ethyl acetate layer was washed with saturated brine, dried over anhydrous magnesium sulfate and concentrated to give yellow crude crystals. A mixture of the obtained crude crystals, 2-bromo-1,1-diethoxyethane (0.65 mL), 1N hydrochloric acid (1 mL) and ethanol (3 mL) was stirred overnight at 80° C. The reaction mixture was neutralized with saturated aqueous sodium hydrogen carbonate, and extracted with ethyl acetate. The ethyl acetate layer was washed with saturated brine, dried over anhydrous magnesium sulfate, and concentrated. The obtained residue was subjected to NH-silica gel column chromatography (eluate: ethyl acetate), and the obtained crude crystals were recrystallized (ethyl acetate-diisopropyl ether) to give the title compound (36.8 mg, yield 58%) as colorless crystals. Melting point 185-187° C., MS: 449 (MH⁺), 1H NMR (300 MHz, DMSO-d₆) δ ppm 3.19-3.38 (2H, m) 4.56 (2H, t, J=6.6 Hz) 7.00 (1H, d, J=3.6 Hz) 7.21 (1H, d, J=1.1 Hz) 7.22-7.29 (1H, m) 7.35 (1H, d, J=3.6 Hz) 7.42 (1H, dd, J=8.1, 4.5 Hz) 7.49 (1H, d, J=7.7 Hz) 7.69-7.80 (1H, m) 8.05 (1H, dd, J=8.1, 1.5 Hz) 8.13 (1H, s) 8.46-8.54 (1H, m) 8.64 (1H, dd, J=4.5, 1.5 Hz) 11.40 (1H, s) 12.69 (1H, s).

### Example 86

### 2-(3-{[5-(3-chloropyridin-2-yl)-3-(1,3-thiazol-2-ylamino)-1H-indazole-7-yl]oxy}propyl)-1H-isoindole-1,3(2H)-dione

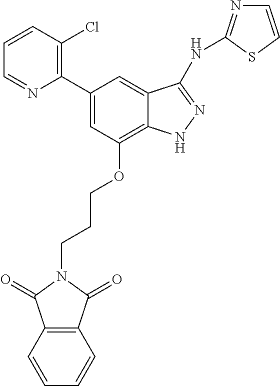

The title compound (229 mg, yield 72%) was obtained as colorless crystals from N-{5-(3-chloropyridin-2-yl)-7-[3-(1,3-dioxo-1,3-dihydro-2H-isoindol-2-yl)propoxy]-1H-indazol-3-yl}thiourea (319 mg) in the same manner as in Example 2. Melting point>250° C.

### Example 87

### (2-{[5-(3-chloropyridin-2-yl)-7-(2-pyridin-2-ylethoxy)-1H-indazol-3-yl]amino}-1,3-thiazol-5-yl)methanol

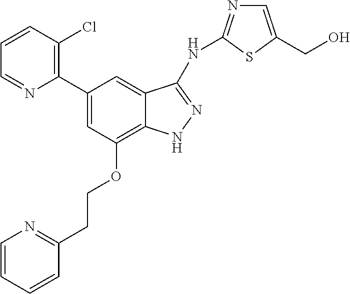

The title compound (46.5 mg, yield 43%) was obtained as colorless crystals from 2-{[5-(3-chloropyridin-2-yl)-7-(2-pyridin-2-ylethoxy)-1H-indazol-3-yl]amino}-1,3-thiazole-5-carbaldehyde (108 mg) in the same manner as in Example 32. Melting point 178-179° C.

### Example 88

### 5-(3-chloropyridin-2-yl)-N-{5-[(dimethylamino)methyl]-1,3-thiazol-2-yl}-7-(2-pyridin-2-ylethoxy)-1H-indazole-3-amine

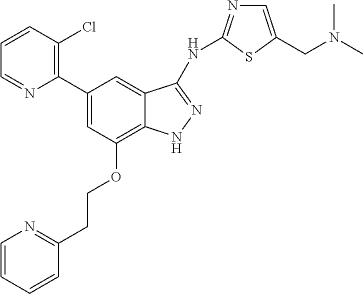

The title compound (131 mg, yield 82%) was obtained as colorless crystals from 2-{[5-(3-chloropyridin-2-yl)-7-(2-pyridin-2-ylethoxy)-1H-indazol-3-yl]amino}-1,3-thiazole-5-carbaldehyde (150 mg) and dimethylamine (2M tetrahydrofuran solution, 0.31 mL) in the same manner as in Example 33. Melting point 170-171° C.

### Example 89

### 5-(3-chloropyridin-2-yl)-N-(5-{[methoxy(methyl)amino]methyl}-1,3-thiazol-2-yl)-7-(2-pyridin-2-ylethoxy)-1H-indazole-3-amine

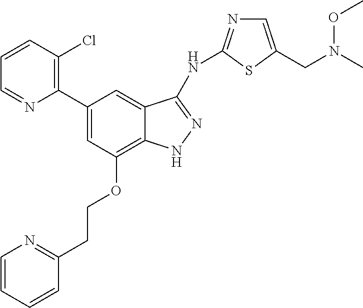

The title compound (129 mg, yield 82%) was obtained as colorless crystals from 2-{[5-(3-chloropyridin-2-yl)-7-(2-pyridin-2-ylethoxy)-1H-indazol-3-yl]amino}-1,3-thiazole-5-carbaldehyde (150 mg), N-methoxymethaneamine hydrochloride (46 mg), and triethylamine (0.088 mL) in the same manner as in Example 33. Melting point 97-98° C.

### Example 90

### 5-(3-chloropyridin-2-yl)-N-[5-(morpholin-4-ylmethyl)-1,3-thiazol-2-yl]-7-(2-pyridin-2-ylethoxy)-1H-indazole-3-amine

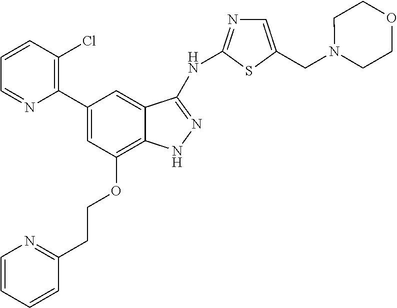

The title compound (143 mg, yield 83%) was obtained as colorless crystals from 2-{[5-(3-chloropyridin-2-yl)-7-(2-pyridin-2-ylethoxy)-1H-indazol-3-yl]amino}-1,3-thiazole-5-carbaldehyde (150 mg) and morpholine (0.033 mL) in the same manner as in Example 33. Melting point 165-167° C.

### Example 91

### 7-(3-aminopropoxy)-5-(3-chloropyridin-2-yl)-N-1,3-thiazol-2-yl-1H-indazole-3-amine trihydrochloride

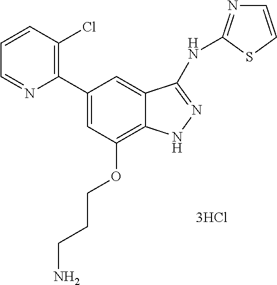

To an ethanol solution (5 mL) of 2-(3-{[5-(3-chloropyridin-2-yl)-3-(1,3-thiazol-2-ylamino)-1H-indazole-7-yl]oxy}propyl)-1H-isoindole-1,3(2H)-dione (212 mg) was added hydrazine monohydrate (0.058 mL), and the mixture was stirred overnight at 60° C. The mixture was diluted with a mixed solvent of ethyl acetate and tetrahydrofuran, saturated aqueous sodium hydrogen carbonate, washed with water and saturated brine, and dried over anhydrous magnesium sulfate. The solvent was concentrated under reduced pressure, and 4N hydrogen chloride -ethyl acetate (1 mL) was added to the residue and concentrated under reduced pressure. The obtained crystal was collected by filtration and washed with ethyl acetate to give the title compound (63.1 mg, yield 31%). Melting point 171-173° C.

### Example 92

### 5-(3-chloropyridin-2-yl)-7-[3-(dimethylamino)propoxy]-N-1,3-thiazol-2-yl-1H-indazole-3-amine

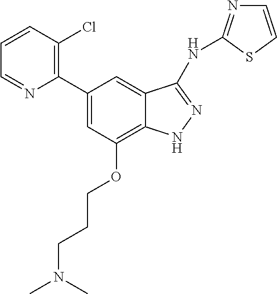

To a methanol solution (2 mL) of 7-(3-aminopropoxy)-5-(3-chloropyridin-2-yl)-N-1,3-thiazol-2-yl-1H-indazole-3-amine trihydrochloride (25 mg) were added formalin (37%) (0.024 mL), triethylamine (0.028 mL) and sodium triacetoxyhydroborate (63 mg), and the mixture was stirred overnight at room temperature. Saturated aqueous sodium hydrogen carbonate and ethyl acetate were added, and the organic layer was washed with water and saturated brine and dried over anhydrous magnesium sulfate. The solvent was evaporated under reduced pressure and subjected to NH-silica gel column chromatography (eluate: ethyl acetate), and the obtained crude crystals were recrystallized (ethyl acetate) to give the title compound (6.5 mg, yield 31%) as a colorless solid. Melting point 215-217° C.

### Example 93

### 5-(3-chloropyridin-2-yl)-N-{5-[(methylamino)methyl]-1,3-thiazol-2-yl}-7-(2-pyridin-2-ylethoxy)-1H-indazole-3-amine

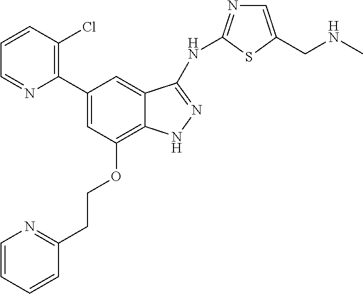

The title compound (14.7 mg, yield 38%) was obtained as colorless crystals from tert-butyl [(2-{[5-(3-chloropyridin-2-yl)-7-(2-pyridin-2-ylethoxy)-1H-indazol-3-yl]amino}-1,3-thiazol-5-yl)methyl]methylcarbamate (46.7 mg) in the same manner as in Example 53. Melting point 180-182° C.

### Example 94

### 2-(2-{[5-(3-chloropyridin-2-yl)-7-(2-pyridin-2-ylethoxy)-1H-indazol-3-yl]amino}-1,3-thiazol-5-yl)ethanol

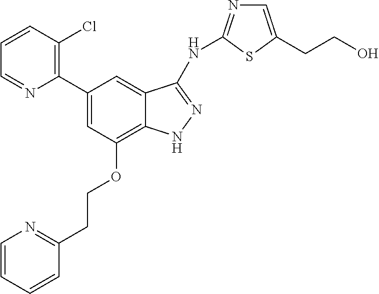

The title compound (11.2 mg, yield 12%) was obtained as colorless crystals from (2-{[5-(3-chloropyridin-2-yl)-7-(2-pyridin-2-ylethoxy)-1H-indazol-3-yl]amino}-1,3-thiazol-5-yl)ethyl acetate (101 mg) in the same manner as in Example 57.

MS: 493 (MH⁺).

### Example 95

### methyl 6-({[5-(3-chloropyridin-2-yl)-3-(1,3-thiazol-2-ylamino)-1H-indazole-7-yl]oxy}methyl)nicotinate

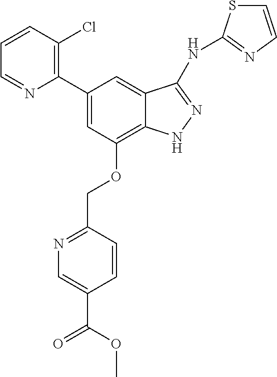

The title compound (610 mg, yield 56%) was obtained as colorless crystals from methyl 6-{[3-[(aminocarbonothioyl)amino]-5-(3-chloropyridin-2-yl)-1H-indazole-7-yl]oxy}methyl)nicotinate (1.13 g) in the same manner as in Example 2. Melting point 210-212° C.

### Example 96

### 6-({[5-(3-chloropyridin-2-yl)-3-(1,3-thiazol-2-ylamino)-1H-indazole-7-yl]oxy}methyl)nicotinic acid

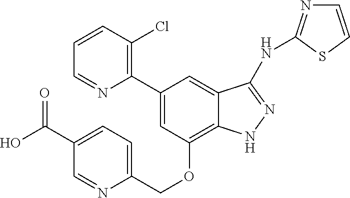

The title compound (16.8 mg, yield 25%) was obtained as colorless crystals from methyl 6-({[5-(3-chloropyridin-2-yl)-3-(1,3-thiazol-2-ylamino)-1H-indazole-7-yl]oxy}methyl)nicotinate (68 mg) in the same manner as in Example 56. MS: 479 (MH⁺).

### Example 97

### 5-(3-chloropyridin-2-yl)-1-methyl-7-(2-pyridin-2-ylethoxy)-N-1,3-thiazol-2-yl-1H-indazole-3-amine

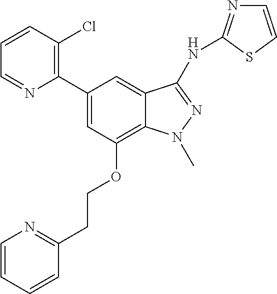

The title compound (2.5 mg, yield 16%) was obtained as colorless crystals from N-[5-(3-chloropyridin-2-yl)-1-methyl-7-(2-pyridin-2-ylethoxy)-1H-indazol-3-yl]thiourea (14.7 mg) in the same manner as in Example 2. MS: 463 (MH⁺).

### Example 98

### 5-(3-chloropyridin-2-yl)-N-pyrazin-2-yl-7-(2-pyridin-2-ylethoxy)-1H-indazole-3-amine

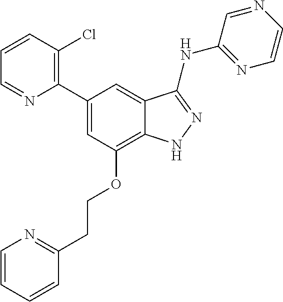

A concentrated hydrochloric acid suspension (5 mL) of 5-(3-chloropyridin-2-yl)-1-(methoxymethyl)-N-pyrazin-2-yl-7-(2-pyridin-2-ylethoxy)-1H-indazole-3-amine (89.6 mg) was stirred at 60° C. for 3 hr, concentrated under reduced pressure, diluted with water, and basified by adding saturated aqueous sodium hydrogen carbonate. The aqueous layer was extracted with a mixed solvent of ethyl acetate and tetrahydrofuran, and the combined organic layer was washed with water and saturated brine, dried over anhydrous magnesium sulfate, and concentrated under reduced pressure. The obtained residue was purified by silica gel column chromatography (hexane:ethyl acetate=1:1-0:1), and the obtained crystals were recrystallized (ethyl acetate-diethyl ether) to give the title compound (46.9 mg, yield 57%) as a colorless solid. Melting point 117-119° C.

### Example 99

### 5-(3-chloropyridin-2-yl)-1-(methoxymethyl)-N-pyrazin-2-yl-7-(2-pyridin-2-ylethoxy)-1H-indazole-3-amine

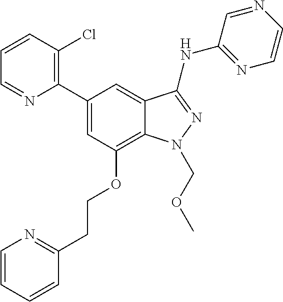

A mixture of 5-(3-chloropyridin-2-yl)-1-(methoxymethyl)-7-(2-pyridin-2-ylethoxy)-1H-indazole-3-amine (137 mg), 2-chloropyrazine (0.036 mL), cesium carbonate (218 mg), tris(dibenzylidene)dipalladium(0) (16 mg), (9,9-dimethyl-9H-xanthen-4,5-diyl)bis(diphenylphosphine) (29 mg) and 1,4-dioxane (3 mL) was stirred overnight at 100° C. under nitrogen atmosphere. The insoluble materials were removed by filtration, and the filtrate was concentrated. The residue was purified by NH-silica gel column chromatography (hexane-ethyl acetate=5:1-0:1) and silica gel column chromatography (ethyl acetate-methanol=1:0-50:1) to give the title compound (100 mg, yield 61%) as pale-yellow crystals. Melting point 120-122° C.

### Example 100

### 5-(3-chloropyridin-2-yl)-N-(1-methyl-1H-pyrazol-3-yl)-7-(2-pyridin-2-ylethoxy)-1H-indazole-3-amine

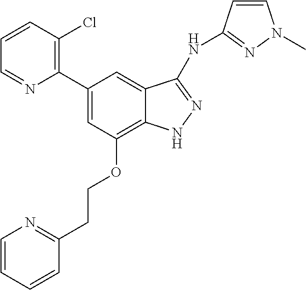

To an ethanol solution (1 mL) of tert-butyl 5-(3-chloropyridin-2-yl)-3-[(1-methyl-1H-pyrazol-3-yl)amino]-7-(2-pyridin-2-ylethoxy)-1H-indazole-1-carboxylate (55.9 mg) was added concentrated hydrochloric acid (2 mL), and the mixture was stirred at room temperature for 1 hr. The reaction solution was basified by adding saturated aqueous sodium hydrogen carbonate, and the aqueous layer was extracted with ethyl acetate-tetrahydrofuran. The organic layer was washed with saturated brine, dried over anhydrous magnesium sulfate, and concentrated under reduced pressure. The obtained residue was purified by silica gel column chromatography (ethyl acetate:methanol=1:0-50:1), and crystallized from dichloromethane-diisopropyl ether to give the title compound (25.2 mg, yield 55%) as colorless crystals. Melting point 89-91° C., MS: 446 (MH⁺), ¹H NMR (300 MHz, DMSO-d₆) δ ppm 3.25-3.34 (2H, m) 3.72 (3H, s) 4.53 (2H, t, J=6.6 Hz) 6.50 (1H, d, J=1.9 Hz) 7.13 (1H, s) 7.24 (1H, dd, J=6.8, 5.3 Hz) 7.39 (1H, dd, J=8.3, 4.5 Hz) 7.45-7.54 (2H, m) 7.68-7.80 (1H, m) 7.97-8.09 (2H, m) 8.51 (1H, d, J=3.8 Hz) 8.62 (1H, dd, J=4.5, 1.5 Hz) 9.27 (1H, s) 12.10 (1H, s).

### Example 101

### N-[5-(3-chloropyridin-2-yl)-1H-indazol-3-yl]-N′-ethylurea

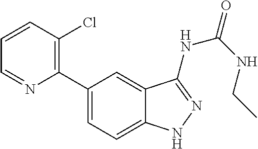

To a toluene-tetrahydrofuran solution (2 mL-2 mL) of tert-butyl 3-amino-5-(3-chloropyridin-2-yl)-1H-indazole-1-carboxylate (160 mg) were added ethylisocyanate (0.055 mL) and triethylamine (0.13 mL), and the mixture was stirred overnight while heating under reflux. The reaction solution was concentrated under reduced pressure, and the residue was purified by silica gel column chromatography (hexane:ethyl acetate=2:1 to 1:1). This was dissolved in dichloromethane (1 mL), 4N hydrogen chloride-ethyl acetate solution (1 mL) was added, and the mixture was stirred at room temperature for 30 min. Saturated aqueous sodium hydrogen carbonate was added to the reaction mixture, and the mixture was extracted with ethyl acetate. The organic layer was washed with water and saturated brine, dried over anhydrous magnesium sulfate, and concentrated under reduced pressure. The obtained crude crystals were recrystallized (ethyl acetate-diisopropyl ether.) to give the title compound (65.2 mg, yield 45%) as colorless crystals. Melting point 178-180° C., MS: 316 (MH⁺), ¹H NMR (300 MHz, DMSO-d₆) δ ppm 1.12 (3H, t, J=7.2 Hz) 3.18-3.29 (2H, m) 7.35-7.51 (2H, m) 7.67 (1H, dd, J=8.9, 1.7 Hz) 7.76 (1H, t, J=5.5 Hz) 8.04 (1H, dd, J=8.0, 1.5 Hz) 8.44 (1H, s) 8.59-8.67 (1H, m) 9.48 (1H, s) 12.46 (1H, s).

### Example 102

### 1-[5-(3-chloropyridin-2-yl)-1H-indazol-3-yl]-3-phenylurea

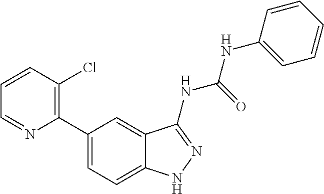

The title compound (110 mg, yield 31%) was obtained as white crystals from tert-butyl 3-amino-5-(3-chloropyridin-2-yl)-1H-indazole-1-carboxylate (345 mg) and phenylisocyanate (240 mg) in the same manner as in Example 101. Melting point 233° C.

### Example 103

### 1-[5-(3-chloropyridin-2-yl)-1H-indazol-3-yl]-3-isopropylurea

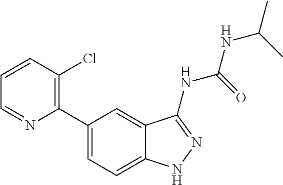

The title compound (36 mg, yield 10%) was obtained as white crystals from tert-butyl 3-amino-5-(3-chloropyridin-2-yl)-1H-indazole-1-carboxylate (345 mg) and 2-isocyanatopropane (170 mg) in the same manner as in Example 101. Melting point 185° C.

### Example 104

### N-{[5-(3-chloropyridin-2-yl)-1H-indazol-3-yl]carbamoyl}glycine

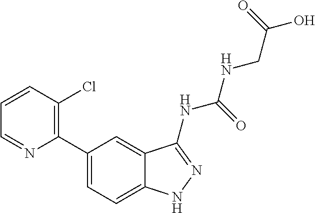

An oily substance obtained from tert-butyl 3-amino-5-(3-chloropyridin-2-yl)-1H-indazole-1-carboxylate (300 mg) and ethyl N-(oxomethylene)glycinate (150 mg) in the same manner as in Example 101 was purified by preparative HPLC. The title compound (160 mg, yield 55%) in which hydrolysis of the ester group proceeded was obtained as white crystals. Melting point 193° C.

### Example 105

### 5-(3-chloropyridin-2-yl)-N-(1-methyl-1H-pyrazol-3-yl)-7-[4-(methylsulfonyl)phenoxy]-1H-indazole-3-amine

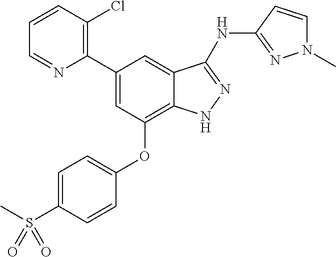

To an ethanol suspension (4 mL) of 5-(3-chloropyridin-2-yl)-1-(methoxymethyl)-N-(1-methyl-1H-pyrazol-3-yl)-7-[4-(methylsulfonyl)phenoxy]-1H-indazole-3-amine (290 mg) was added 6N hydrochloric acid, and the mixture was stirred at 60° C. for 3 hr. The reaction mixture was basified by adding saturated aqueous sodium hydrogen carbonate. The aqueous layer was extracted with a mixed solvent of ethyl acetate and tetrahydrofuran, and the combined organic layer was washed with water and saturated brine, dried over anhydrous magnesium sulfate, and concentrated under reduced pressure. The concentrate was purified by NH-silica gel column chromatography (eluate: ethyl acetate) and preparative HPLC to give the title compound (52.2 mg, yield 20%) as a colorless solid. Melting is point 249-250° C., MS: 495 (MH⁺), ¹H NMR (300 MHz, DMSO-d₆) δ ppm 3.19 (3H, s) 3.74 (3H, s) 6.52 (1H, d, J=2.3 Hz) 7.23 (2H, d, J=8.7 Hz) 7.37 (1H, s) 7.40 (1H, dd, J=8.3, 4.5 Hz) 7.52 (1H, d, J=1.9 Hz) 7.93 (2H, d, J=8.7 Hz) 8.04 (1H, d, J=8.0 Hz) 8.44 (1H, s) 8.61 (1H, d, J=3.4 Hz) 9.53 (1H, s) 12.30 (1H, s).

### Example 106

### 5-(benzyloxy)-7-(2-pyridin-2-ylethoxy)-N-1,3-thiazol-2-yl-1H-indazole-3-amine

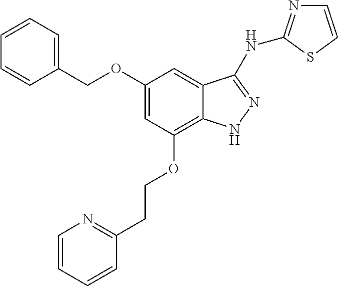

The title compound (338 mg, yield 80%) was obtained as colorless crystals from 5-(benzyloxy)-7-(2-pyridin-2-ylethoxy)-1H-indazole-3-amine (447 mg) in the same manner as in Example 15. Melting point 160-161° C.

### Example 107

### 5-isopropoxy-7-(2-pyridin-2-ylethoxy)-N-1,3-thiazol-2-yl-1H-indazole-3-amine

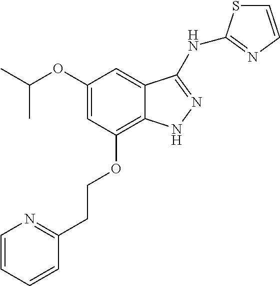

The title compound (58.6 mg, yield 56%) was obtained as colorless crystals from N-[5-isopropoxy-7-(2-pyridin-2-ylethoxy)-1H-indazol-3-yl]thiourea (98 mg) in the same manner as in Example 2. Melting point 105-106° C.

### Example 108

### 5-[(1S)-2-methoxy-1-methylethoxy]-7-(2-pyridin-2-ylethoxy)-N-1,3-thiazol-2-yl-1H-indazole-3-amine dihydrochloride

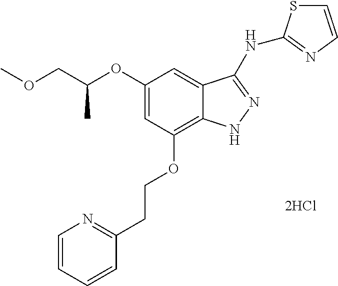

To a solution of N-[5-[(1S)-2-methoxy-1-methylethoxy]-7-(2-pyridin-2-ylethoxy)-1H-indazol-3-yl]thiourea (173 mg) in ethanol-1N hydrochloric acid (6 mL-2 mL) was added 2-bromo-1,1-diethoxyethane (0.20 mL), and the mixture was stirred at 80° C. for 4 hr. The reaction mixture was basified with saturated aqueous sodium hydrogen carbonate, diluted with ethyl acetate, washed with saturated brine, dried over anhydrous magnesium sulfate, and concentrated. The obtained residue was purified by NH-silica gel column chromatography (eluate: ethyl acetate) and silica gel column chromatography (hexane:ethyl acetate=1:1-0:1). The obtained residue was dissolved by adding ethyl acetate (1 mL), and 1N hydrochloric acid (1 mL) was added and stirred. The solvent was concentrated under reduced pressure, and the obtained crystals were washed with ethyl acetate to give the title compound (169 mg, yield 79%) as colorless crystals. Melting point 150-153° C., MS: 426 (MH⁺-2HCl), ¹H NMR (300 MHz, DMSO-d₆) δ ppm 1.27 (3H, d, J=6.2 Hz) 3.31 (3H, s) 3.43-3.55 (2H, m) 3.55-3.68 (2H, m) 4.43-4.64 (3H, m) 6.59 (1H, d, J=1.7 Hz) 7.12 (1H, d, J=4.0 Hz) 7.26 (1H, d, J=1.5 Hz) 7.45 (1H, d, J=4.1 Hz) 7.84-8.04 (1H, m) 8.17 (1 H, d, J=7.9 Hz) 8.43-8.65 (1H, m) 8.86 (1H, d, J=4.9 Hz) 12.78 (2H, br. s.).

### Example 109

### 5-[(1-methyl-1H-imidazol-2-yl)thio]-N-1,3-thiazol-2-yl-1H-indazole-3-amine

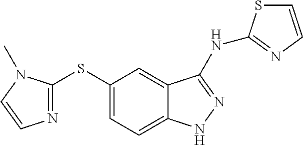

To a solution of 5-[(1-methyl-1H-imidazol-2-yl)thio]-1H-indazole-3-amine (120 mg) in tetrahydrofuran (5 mL) was added 1,1′-carbonothioyldipyridine-2(1H)-one (140 mg) at 0° C., and the mixture was stirred for 30 min. Concentrated aqueous ammonia (1 mL) was added, and the reaction mixture was stirred at room temperature for 1 hr. Water was added, and the mixture was extracted with ethyl acetate. The ethyl acetate layer was washed with saturated brine, dried over anhydrous magnesium sulfate, and concentrated to give yellow crude crystals. A mixture of the obtained crude crystal, 2-bromo-1,1-diethoxyethane (0.15 mL), 1N hydrochloric acid (2 mL) and ethanol (3 mL) was stirred at 80° C. for 2 hr. The reaction mixture was neutralized with saturated aqueous sodium hydrogen carbonate and extracted with ethyl acetate. The ethyl acetate layer was washed with saturated brine, dried over anhydrous magnesium sulfate, and concentrated. The residue was purified by NH-silica gel column chromatography (ethyl acetate) to give the title compound (130 mg, yield 80%) as colorless crystals. Melting point>250° C., MS: 329 (MH⁺), ¹H NMR (300 MHz, DMSO-d₆) δ ppm 3.32 (3H, s) 6.99 (1H, d, J=3.6 Hz) 7.03 (1H, s) 7.25 (1 H, dd, J=8.8, 1.8 Hz) 7.32-7.45 (3H, m) 8.13 (1H, s) 11.36 (1H, s) 12.47 (1H, s).

### Example 110

### 5-(isopropylthio)-N-1,3-thiazol-2-yl-1H-indazole-3-amine

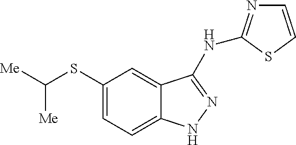

The title compound (0.66 g, yield 48%) was obtained as colorless crystals from N-[5-(isopropylthio)-1H-indazol-3-yl]thiourea (1.29 g) in the same manner as in Example 2. Melting point 222-223° C.

### Example 111

### 5-(isobutylthio)-N-1,3-thiazol-2-yl-1H-indazole-3-amine

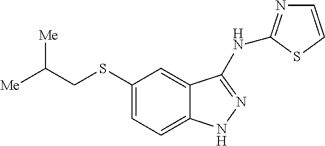

The title compound (300 mg, yield 71%) was obtained as colorless crystals from N-[5-(isobutylthio)-1H-indazol-3-s yl]thiourea (440 mg) in the same manner as in Example 2. Melting point>212° C.

### Example 112

### 5-(cyclopentylthio)-N-1,3-thiazol-2-yl-1H-indazole-3-amine

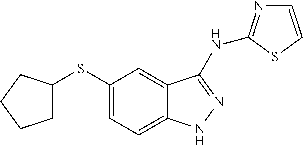

The title compound (230 mg, yield 61%) was obtained as colorless crystals from 5-(cyclopentylthio)-1H-indazole-3-amine (278 mg) in the same manner as in Example 15. Melting point 232-233° C.

### Example 113

### 5-(isopropylsulfonyl)-N-1,3-thiazol-2-yl-1H-indazole-3-amine

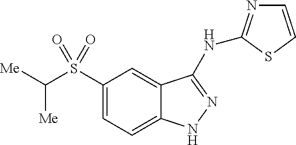

To a suspension of 5-(isopropylthio)-N-1,3-thiazol-2-yl-1H-indazole-3-amine (0.27 g) in tetrahydrofuran (6 mL), ethanol (2 mL) and water (2 mL) was added Oxone (0.86 g) at 0° C., and the mixture was stirred at room temperature for 3 hr. The mixture was diluted with ethyl acetate, washed with water and saturated brine, dried over anhydrous magnesium sulfate, and concentrated under reduced pressure. The residue was subjected to NH-silica gel column chromatography (eluate: ethyl acetate), and concentrated under reduced pressure. The obtained crystal was recrystallized (ethyl acetate-hexane) to give the title compound (0.28 g, 94%) as colorless crystals. Melting point 146-148° C.

### Example 114

### 5-(isopropylsulfinyl)-N-1,3-thiazol-2-yl-1H-indazole-3-amine

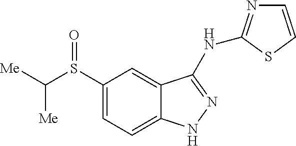

To a solution of 5-(isopropylthio)-N-1,3-thiazol-2-yl-1H-indazole-3-amine (0.26 g) in tetrahydrofuran (6 mL) and water (2 mL) was added sodium periodic acid (0.20 g) at 0° C., and the mixture was stirred overnight at room temperature. The mixture was diluted with ethyl acetate, washed with water and saturated brine, dried over anhydrous magnesium sulfate, and concentrated under reduced pressure. The residue was purified by silica gel column chromatography (eluate:tetrahydrofuran) and concentrated under reduced pressure. The obtained crude crystals were recrystallized (ethyl acetate) to give the title compound (0.13 g, 47%) as colorless crystals. Melting point>240° C.

### Example 115

### 5-(isobutylsulfonyl)-N-1,3-thiazol-2-yl-1H-indazole-3-amine

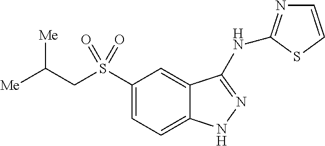

The title compound (210 mg, yield 67%) was obtained as colorless crystals from 5-(isobutylthio)-N-1,3-thiazol-2-yl-1H-indazole-3-amine (280 mg) in the same manner as in Example 113. Melting point 205-206° C.

### Example 116

### 5-(cyclopentylsulfonyl)-N-1,3-thiazol-2-yl-1H-indazole-3-amine

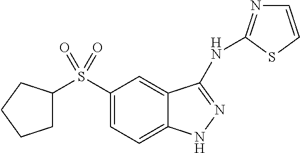

The title compound (160 mg, yield 70%) was obtained as colorless crystals from 5-(cyclopentylthio)-N-1,3-thiazol-2-yl-1H-indazole-3-amine (210 mg) in the same manner as in Example 113. Melting point 224-225° C.

### Example 117

### 5-(isopropylsulfonyl)-7-(2-pyridin-2-ylethoxy)-N-1,3-thiazol-2-yl-1H-indazole-3-amine

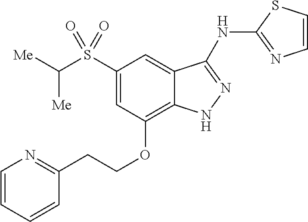

To a solution of 5-(isopropylsulfonyl)-7-(2-pyridin-2-ylethoxy)-1H-indazole-3-amine (0.33 g) in tetrahydrofuran (8 mL) was added 1,1′-carbonothioyldipyridine-2(1H)-one (0.26 g) is at 0° C. for 30 min, and the mixture was stirred. Concentrated aqueous ammonia (0.40 mL) was added, and the reaction mixture was stirred at room temperature for 1 hr, water was added, and the mixture was extracted with ethyl acetate. The ethyl acetate layer was washed with saturated brine, dried over anhydrous magnesium sulfate and concentrated to give yellow crude crystals. The mixture of the obtained crude crystal, 2-bromo-1,1-diethoxyethane (0.39 g), concentrated hydrochloric acid (0.5 mL) and ethanol (10 ml) was heated under reflux overnight. The reaction mixture was neutralized with saturated aqueous sodium hydrogen carbonate and extracted with ethyl acetate. The ethyl acetate layer was washed with saturated brine, dried over anhydrous magnesium sulfate, and concentrated under reduced pressure. The residue was subjected to NH-silica gel column chromatography (eluate: ethyl acetate) to give the title compound (0.25 g, yield 61%) as colorless crystals. Melting point 190-191° C., MS: 444(MH⁺), ¹H NMR (300 MHz, DMSO-d₆) δ ppm 1.17 (6H, t, J=6.9 Hz) 3.31 (2H, t, J=6.6 Hz) 3.43 (1H, septet, J=6.9 Hz) 4.61 (2H, t, J=6.6 Hz) 7.03 (1H, d, J=3.6 Hz) 7.18 (1H, s) 7.22-7.28 (1H, m) 7.37 (1H, d, J=3.6 Hz) 7.49 (1H, d, J=7.8 Hz) 7.74 (1H, dt, J=1.5, 7.8 Hz) 8.40 (1H, s) 8.50-8.54 (1H, m) 11.54 (1H, s) 13.10 (1H, s).

### Example 118

### 5-(isopropylsulfonyl)-7-[3-(methylsulfonyl)propoxy]-N-1,3-thiazol-2-yl-1H-indazole-3-amine

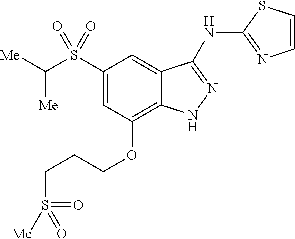

The title compound (193 mg, yield 43%) was obtained as pale-yellow crystals from 5-(isopropylsulfonyl)-7-[3-(methylsulfonyl)propoxy]-1H-indazole-3-amine (368 mg) in the same manner as in Example 117. Melting point 212-213° C.

### Example 119

### 5-(isopropylsulfonyl)-N-[5-(morpholin-4-ylmethyl)-1,3-thiazol-2-yl]-7-(2-pyridin-2-ylethoxy)-1H-indazole-3-amine

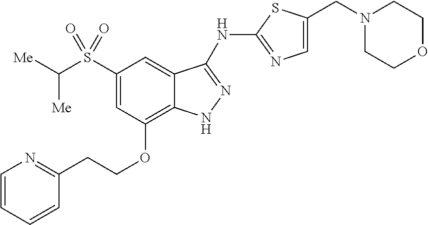

To a mixture of 2-{[5-(isopropylsulfonyl)-7-(2-pyridin-2-ylethoxy)-1H-indazol-3-yl]amino}-1,3-thiazole-5-carbaldehyde (0.15 g), morpholine (56 mg) and tetrahydrofuran (10 mL) was added sodium triacetoxyhydroborate (0.28 g), and the mixture was stirred overnight at room temperature. To the reaction mixture was added saturated aqueous sodium hydrogen carbonate, and the mixture was extracted with ethyl acetate. The ethyl acetate layer was washed with saturated brine, dried over anhydrous magnesium sulfate, and concentrated under reduced pressure. The residue was purified by NH-silica gel column chromatography, and the title compound (94 mg, yield 53%) was obtained from the tetrahydrofuran eluate as pale-yellow crystals. Melting point>190° C.

### Example 120

### 5-(isopropylsulfonyl)-7-{[5-(methylthio)pyridin-2-yl]methoxy}-N-1,3-thiazol-2-yl-1H-indazole-3-amine

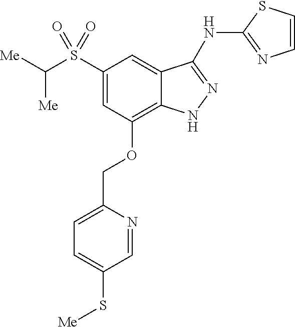

The title compound (0.37 g, yield 65%) was obtained as pale-yellow crystals from 5-(isopropylsulfonyl)-7-{[4-(methylthio)benzyl]oxy}-1H-indazole-3-amine (0.46 g) in the same manner as in Example 117. Melting point 151-152° C.

### Example 121

### 5-(isopropylsulfonyl)-7-{[5-(methylsulfonyl)pyridin-2-yl]methoxy}-N-1,3-thiazol-2-yl-1H-indazole-3-amine

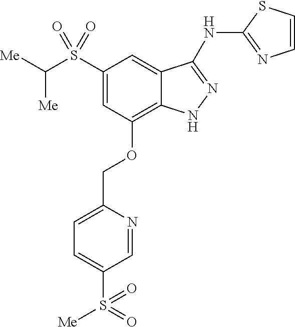

To a mixture of 5-(isopropylsulfonyl)-7-{[5-(methylthio)pyridin-2-yl]methoxy}-N-1,3-thiazol-2-yl-1H-indazole-3-amine (0.28 g), tetrahydrofuran (6 mL), methanol (6 mL) and water (0.5 mL) was added Oxone (0.43 g), and the mixture was stirred at room temperature for 2 hr. The residual Oxone was decomposed with sodium sulfite and concentrated. To the residue was added saturated aqueous sodium hydrogen carbonate, and the precipitated crystals were collected by filtration, washed with water and dried. The obtained crystals were subjected to NH-silica gel column chromatography, and the title compound (0.14 g, yield 47%) was obtained from the tetrahydrofuran eluate as colorless crystals. Melting point 160-161° C.

### Example 122

### 7-(benzyloxy)-5-(isopropylsulfonyl)-N-1,3-thiazol-2-yl-1H-indazole-3-amine

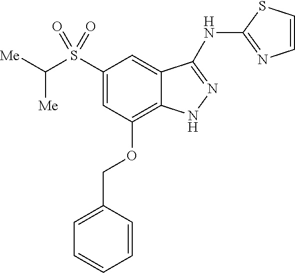

The title compound (0.75 g, yield 86%) was obtained as colorless crystals from 7-(benzyloxy)-5-(isopropylsulfonyl)-1H-indazole-3-amine (0.73 g) in the same manner as in Example 117. Melting point 257-258° C.

### Example 123

### 7-[(2-fluorobenzyl)oxy]-5-(isopropylsulfonyl)-N-1,3-thiazol-2-yl-1H-indazole-3-amine

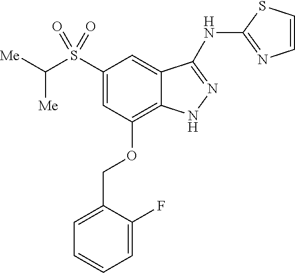

A mixture of 5-(isopropylsulfonyl)-3-(1,3-thiazol-2-ylamino)-1H-indazol-7-ol (0.16 g), 1-(chloromethyl)-2-fluorobenzene (68 mg), potassium carbonate (65 mg) and N,N-dimethylformamide (6 mL) was stirred overnight at room temperature. Water was added to the reaction mixture, and the mixture was extracted with ethyl acetate. The ethyl acetate layer was washed with saturated brine, dried over anhydrous magnesium sulfate, and concentrated. The residue was purified by silica gel column chromatography (ethyl acetate:hexane=4:1) to give the title compound (51 mg, yield 23%) as pale-yellow crystals. Melting point 268-269° C.

### Example 124

### 5-(isopropylsulfonyl)-1-(methoxymethyl)-N-pyrazin-2-yl-7-(2-pyridin-2-ylethoxy)-1H-indazole-3-amine

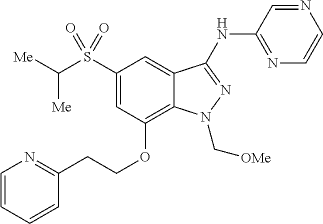

A mixture of 5-(isopropylsulfonyl)-1-(methoxymethyl)-7-(2-pyridin-2-ylethoxy)-1H-indazole-3-amine (0.25 g), 2-chloropyrazine (90 mg), cesium carbonate (0.30 g), tris(dibenzylidene)dipalladium(0) (17 mg), (9,9-dimethyl-9H-xanthen-4,5-diyl)bis(diphenylphosphine) (32 mg) and 1,4-dioxane (6 mL) was stirred overnight at 100° C. under nitrogen atmosphere. The insoluble materials were removed by filtration, and the filtrate was concentrated. The residue was purified by silica gel column chromatography, and the title compound (0.20 g, yield 66%) was obtained from the ethyl acetate eluate as yellow non-crystalline powder. MS: 483 (MH⁺).

### Example 125

### 5-(isopropylsulfonyl)-N-pyrazin-2-yl-7-(2-pyridin-2-ylethoxy)-1H-indazole-3-amine

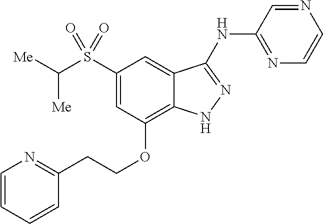

A mixture of 5-(isopropylsulfonyl)-1-(methoxymethyl)-N-pyrazin-2-yl-7-(2-pyridin-2-ylethoxy)-1H-indazole-3-amine (0.20 g) and concentrated hydrochloric acid (4 mL) was stirred at 50° C. for 1 hr. The reaction mixture was neutralized by adding saturated aqueous sodium hydrogen carbonate, and extracted with ethyl acetate. The ethyl acetate layer was washed with saturated brine, dried over anhydrous magnesium sulfate, and concentrated under reduced pressure. The residue was purified by NH-silica gel column chromatography, and the title compound (116 mg, yield 65%) was obtained from the tetrahydrofuran eluate as pale-yellow crystals. Melting point 195-196° C.

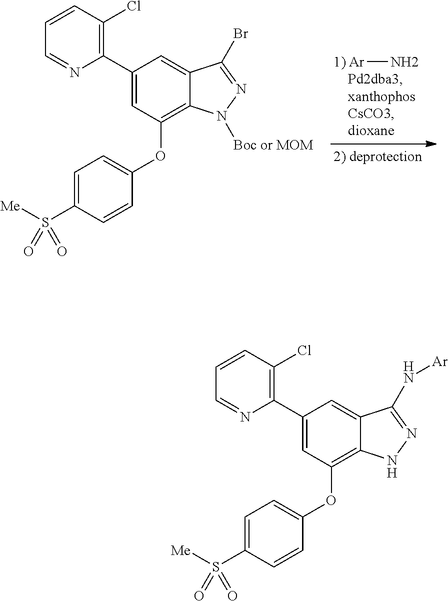

(Wherein Ar is an optionally substituted 5- to 6-membered nitrogen-containing heteroaromatic group.)

### Example 126

### 5-(3-chloropyridin-2-yl)-7-(4-(methylsulfonyl)phenoxy)-N-(pyrazin-2-yl)-1H-indazol-3-amine

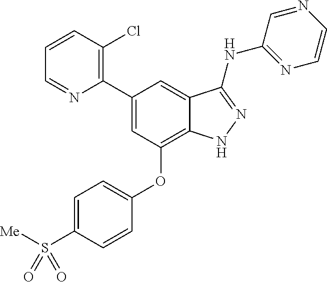

5-(3-Chloropyridin-2-yl)-1-(methoxymethyl)-7-(4-(methylsulfonyl)phenoxy)-N-(pyrazin-2-yl)-1H-indazol-3-amine (91.9 mg, 0.171 mmol) was dissolved in ethanol (2 ml) and conc. hydrochloric acid (2 ml). The mixture was stirred at 60° C. for 2 h, and concentrated in vacuo. Ethyl acetate and saturated aqueous NaHCO₃ were added to the residue. The organic layer was separated, washed with water and saturated brine, dried over magnesium sulfate, filtered, and concentrated in vacuo. The residue was recrystallized from ethyl acetate-ether to give 14.9 mg of the title compound (18%) and 15.2 mg as a second crop (18%) as a light yellow solid. ¹H NMR (400 MHz, DMSO-d₆) δ ppm 3.21 (s, 3H) 7.29 (d, J=8.84 Hz, 2H) 7.37-7.49 (m, 2H) 7.96 (d, J=9.09 Hz, 2H) 8.05 (dd, J=8.08, 1.52 Hz, 1H) 8.09 (d, J=2.27 Hz, 1H) 8.23 (dd, J=2.53, 1.52 Hz, 1H) 8.41 (d, J=1.01 Hz, 1H) 8.61 (dd, J=4.67, 1.39 Hz, 1H) 9.17 (d, J=1.52 Hz, 1H) 10.26 (s, 1H) 12.97 (s, 1H). [M+H] calc'd for C₂₃H₁₇ClN₆O₃S, 493; found, 493.

### Example 127

### 5-(3-chloropyridin-2-yl)-7-(4-(methylsulfonyl)phenoxy)-N-(pyrimidin-2-yl)-1H-indazol-3-amine

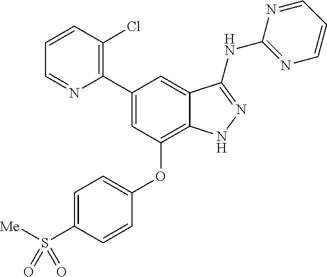

3-Bromo-5-(3-chloropyridin-2-yl)-1-(methoxymethyl)-7-(4-(methylsulfonyl)phenoxy)-1H-indazole (196 mg, 0.41 mmol, 1 eq), 2-amino-pyrimidine (47 mg, 0.49 mmol, 1.2 eq), tris(dibenzylidene-acetone)dipalladium(0) (19 mg, 0.02 mmol, 0.05 eq), 4,5-bis(diphenylphosphino)-9,9-dimethylxanthene (36 mg, 0.06 mmol, 0.15 eq), and cesium carbonate (267 mg, 0.82 mmol, 2 eq) were suspended in degassed anhydrous 1,4-dioxane (4 ml). The mixture was heated under N₂ at 100° C. for overnight. After cooling to room temperature, the insoluble material was filtered and washed with ethyl acetate, and the filtrate was concentrated in vacuo. The residue was purified with NH-silica gel column chromatography using 33-100% ethyl acetate in hexane as an eluent to give 79 mg of the product, which was dissolved in ethanol (2 ml) and concentrated hydrochloric acid (2 ml). The mixture was stirred at room temperature for 2 h, and concentrated in vacuo. Ethyl acetate and saturated aqueous NaHCO₃ were added to the residue and the organic layer was separated, washed with water, and brine, dried over magnesium sulfate, filtered, and concentrated in vacuo. The residue was recrystallized from tetrahydrofuran-ethyl acetate to give 17.0 mg of the title compound (8.4% in 2 steps) as a colorless solid. ¹H NMR (400 MHz, DMSO-d₆) δ ppm 3.21 (s, 3H) 6.83 (t, J=4.80 Hz, 1H) 7.30 (d, J=9.09 Hz, 2H) 7.36-7.43 (m, 2H) 7.91 (s, 1H) 7.97 (d, J=9.09 Hz, 2H) 8.01 (dd, J=8.21, 1.39 Hz, 1H) 8.41 (d, J=4.80 Hz, 2H) 8.58 (dd, J=4.55, 1.52 Hz, 1H) 9.83 (s, 1H) 13.13 (s, 1H). [M+H] calc'd for C₂₃H₁₇ClN₆O₃S, 493; found, 493.

### Example 128

### N-(5-(3-chloropyridin-2-yl)-7-(4-(methylsulfonyl)phenoxy)-1H-indazol-3-yl)isoxazol-3-amine

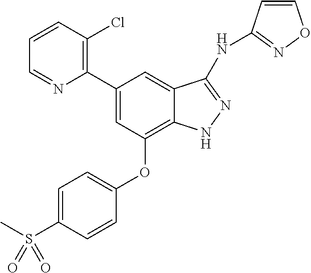

The title compound was prepared according to the procedure outlined in Example 127, using 3-bromo-5-(3-chloropyridin-2-yl)-1-(methoxymethyl)-7-(4-(methylsulfonyl)phenoxy)-1H-indazole (301 mg, 0.576 mmol) and 3-amino-isoxazole (59 mg, 0.691 mmol) to give 30.6 mg of the title compound (48%) as a colorless solid. ¹H NMR (400 MHz, DMSO-d₆) δ ppm 3.20 (s, 3H) 6.91 (d, J=1.52 Hz, 1H) 7.26 (d, J=8.84 Hz, 2H) 7.38-7.47 (m, 2H) 7.95 (d, J=8.84 Hz, 2H) 8.05 (dd, J=8.08, 1.26 Hz, 1H) 8.40 (s, 1H) 8.62 (dd, J=4.55, 1.26 Hz, 1H) 8.70 (d, J=1.52 Hz, 1H) 10.17 (s, 1H) 12.73 (s, 1H). [M+H] calc'd for C₂₂H₁₆ClN₅O₄S, 482; found, 482.

### Example 129

### 2-(3-(5-(3-chloropyridin-2-yl)-7-(4-(methylsulfonyl)phenoxy)-1H-indazol-3-ylamino)-1H-pyrazol-1-yl)ethanol

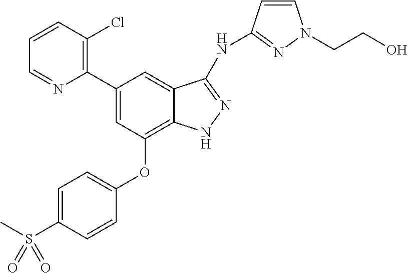

tert-Butyl 3-bromo-5-(3-chloropyridin-2-yl)-7-(4-(methylsulfonyl)phenoxy)-1H-indazole-1-carboxylate (205 mg, 0.354 mmol, 1 eq), 1-(2-(tert-butyldimethylsilyloxy)ethyl)-1H-pyrazol-3-amine (103 mg, 0.425 mmol, 1.2 eq), tris(dibenzylidene-acetone)dipalladium(0) (17 mg, 0.018 mmol, 0.05 eq), 4,5-bis(diphenylphosphino)-9,9-dimethylxanthene (31 mg, 0.05 mmol, 0.15 eq), and cesium carbonate (230 mg, 0.708% mmol, 2 eq) were suspended in degassed anhydrous 1,4-dioxane (4 ml). The mixture was heated under N₂ at 100° C. for overnight. After cooling to room temperature, the insoluble material was filtered and washed with ethyl acetate, and the filtrate was concentrated in vacuo. The residue was purified with NH-silica gel column chromatography using 33-100% ethyl acetate in hexane as an eluent to give 59 mg of the product, which was dissolved in ethanol (2 ml) and concentrated hydrochloric acid (2 ml). The mixture was stirred at room temperature for 3 h, and concentrated in vacuo. Ethyl acetate and saturated aqueous NaHCO₃ were added to the residue and the organic layer was separated, washed with water, and brine, dried over magnesium sulfate, filtered, and concentrated in vacuo. The residue was recrystallized from tetrahydrofuran-ethyl acetate to give 13.9 mg of the title compound (7.5% in 2 steps) as a colorless solid.

¹H NMR (400 MHz, DMSO-d₆) δ ppm 3.20 (s, 3H) 3.73 (q, J=5.73 Hz, 2H) 4.03 (t, J=5.68 Hz, 2H) 4.87 (t, J=5.31 Hz, 1H) 6.53 (d, J=2.02 Hz, 1H) 7.24 (d, J=9.09 Hz, 2H) 7.37 (d, J=1.26 Hz, 1H) 7.40 (dd, J=8.08, 4.80 Hz, 1H) 7.55 (d, J=2.02 Hz, 1H) 7.94 (d, J=8.84 Hz, 2H) 8.04 (dd, J=8.08, 1.52 Hz, 1H) 8.45 (s, 1H) 8.61 (dd, J=4.67, 1.39 Hz, 1H) 9.59 (s, 1H) 12.31 (s, 1H). [M+H] calc'd for C₂₄H₂₁ClN₆O₄S, 525; found, 525.

### Example 130

### 2-(3-(5-(3-chloropyridin-2-yl)-7-(4-(methylsulfonyl)phenoxy)-1H-indazol-3-ylamino)-1H-pyrazol-1-yl)acetic acid

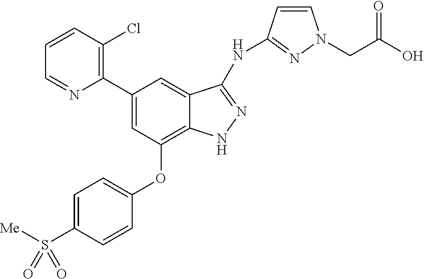

3-Bromo-5-(3-chloropyridin-2-yl)-1-(methoxymethyl)-7-(4-(methylsulfonyl)phenoxy)-1H-indazole (582 mg, 1.11 mmol, 1 eq), methyl 2-(3-amino-1H-pyrazol-1-yl)acetate (260 mg, 1.67 mmol, 1.2 eq), tris(dibenzylidene-acetone)dipalladium(0) (51 mg, 0.055 mmol, 0.05 eq), 4,5-bis(diphenylphosphino)-9,9-dimethylxanthene (97 mg, 0.167 mmol, 0.15 eq), and cesium carbonate (724 mg, 2.22 mmol, 2 eq) were suspended in degassed anhydrous 1,4-dioxane (10 ml). The mixture was heated under N₂ at 100° C. for overnight. After cooling to room temperature, the insoluble material was filtered and washed with ethyl acetate, and the filtrate was concentrated in vacuo. The residue was purified with NH-silica gel column chromatography using 33-100% ethyl acetate in hexane as an eluent to give 50 mg of the product, which was dissolved in methanol (2 ml) and concentrated HCl at room temperature. The mixture was stirred at room temperature for 1 h, and concentrated in vacuo. Ethyl acetate and saturated aqueous NaHCO₃ were added to the mixture. The organic layer was separated, washed with water, dried over magnesium sulfate, filtered, and concentrated in vacuo. The residue was purified with NH-silica gel column chromatography to give 8.9 mg of the product. To a solution of the product in MeOH (2 ml) was added 1N NaOH (0.03 ml, 0.03 mmol) at room temperature. The mixture was stirred at room temperature for 3 h, and neutralized with 1 N HCl. The mixture was extracted with ethyl acetate. The combined organic layer was washed with saturated brine, dried over magnesium sulfate, filtered, and concentrated in vacuo. The residue was purified with preparative HPLC to give trifluoroacetic acid salt of the title compound, which was diluted with ethyl acetate and neutralized with saturated aqueous NaHCO₃. The organic layer was washed is with saturated brine, dried over magnesium sulfate, filtered, and concentrated in vacuo to give the 2.0 mg of the title compound (0.3% in 3 steps) as a light yellow solid. ¹H NMR (400 MHz, DMSO-d₆) δ ppm 3.20 (s, 3H) 4.82 (s, 2H) 6.58 (d, J=2.27 Hz, 1H) 7.24 (d, J=8.84 Hz, 2H) 7.37 (d, J=1.26 Hz, 1H) 7.40 (dd, J=8.08, 4.55 Hz, 1H) 7.59 (d, J=2.27 Hz, 1H) 7.94 (d, J=8.84 Hz, 2H) 8.04 (dd, J=8.08, 1.52 Hz, 1H) 8.44 (d, J=1.26 Hz, 1H) 8.61 (dd, J=4.67, 1.39 Hz, 1H) 9.63 (s, 1 H) 12.35 (br. s., 1H) 12.98 (br. s., 1H). [M+H] calc'd for C₂₄H₉ClN₆O₅S, 539; found, 539.

### Example 131

### 3-(3-(5-(3-chloropyridin-2-yl)-7-(4-(methylsulfonyl)phenoxy)-1H-indazol-3-ylamino)-1H-pyrazol-1-yl)propane-1,2-diol

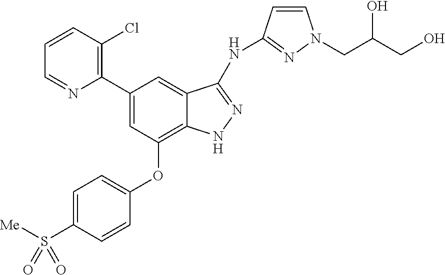

tert-Butyl 3-bromo-5-(3-chloropyridin-2-yl)-7-(4-(methylsulfonyl)phenoxy)-1H-indazole-1-carboxylate (300 mg, 0.52 mmol, 1 eq), 1-((2,2-dimethyl-1,3-dioxolan-4-yl)methyl)-1H-pyrazol-3-amine (123 mg, 0.62 mmol, 1.2 eq), tris(dibenzylidene-acetone)dipalladium(0) (24 mg, 0.026 mmol, 0.05 eq), 4,5-bis(diphenylphosphino)-9,9-dimethylxanthene (46 mg, 0.078 mmol, 0.15 eq), and cesium carbonate (339 mg, 1.04 mmol, 2 eq) were suspended in degassed anhydrous 1,4-dioxane (5 ml). The mixture was heated under N₂ at 100° C. for overnight. After cooling to room temperature, the insoluble material was filtered and washed with ethyl acetate, and the filtrate was concentrated in vacuo. The residue was purified with NH-silica gel column chromatography using 33-100% ethyl acetate in hexane as an eluent to give a light yellow oil, which was dissolved in is ethanol (2 ml) and concentrated hydrochloric acid (2 ml). The mixture was stirred at room temperature for 2 h, and concentrated in vacuo. Ethyl acetate and saturated aqueous NaHCO₃ were added to the residue and the organic layer was separated, washed with water, and saturated brine, dried over magnesium sulfate, filtered, and concentrated in vacuo. The residue was purified with preparative HPLC to give trifluoroacetic acid salt of the title compound, which was diluted with ethyl acetate and neutralized with saturated aqueous NaHCO₃. The organic layer was washed with saturated brine, dried over magnesium sulfate, filtered, and concentrated in vacuo to give the 87.1 mg of the title compound (30% in 2 steps) as a colorless solid. ¹H NMR (400 MHz, DMSO-d₆) δ ppm 3.20 (s, 3H) 3.33 (s, 2H) 3.78-3.93 (m, 2H) 4.09 (dd, J=13.14, 3.79 Hz, 1H) 4.70 (t, J=5.56 Hz, 1H) 4.95 (d, J=5.31 Hz, 1H) 6.53 (d, J=2.02 Hz, 1H) 7.24 (d, J=8.84 Hz, 2H) 7.37 (d, J=1.26 Hz, 1H) 7.40 (dd, J=8.08, 4.55 Hz, 1H) 7.52 (d, J=2.02 Hz, 1H) 7.94 (d, J=8.84 Hz, 2H) 8.04 (dd, J=8.08, 1.52 Hz, 1H) 8.45 (s, 1H) 8.61 (dd, J=4.55, 1.52 Hz, 1H) 9.59 (s, 1H) 12.32 (s, 1H). [M+H] calc'd for C₂₅H₂₃ClN₆O₅S, 555; found, 555.

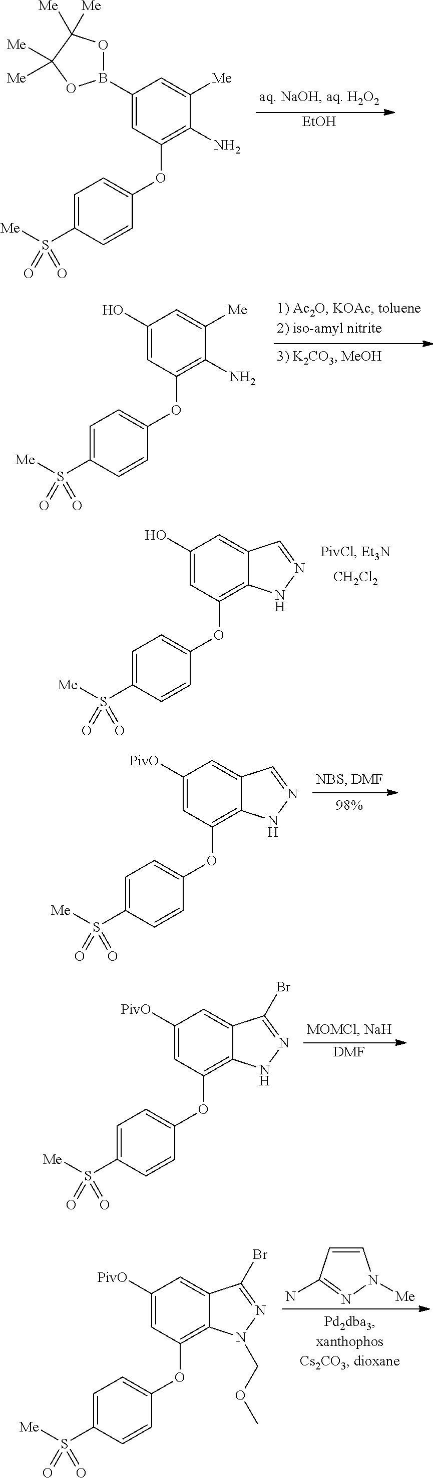

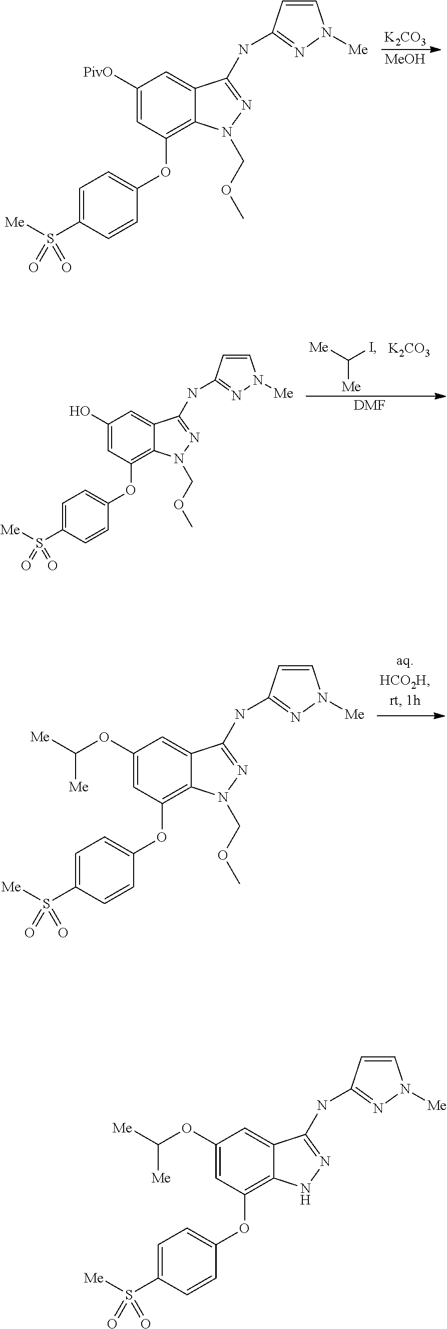

### Example 132

### 5-isopropoxy-1-(methoxymethyl)-N-(1-methyl-1H-pyrazol-3-yl)-7-(4-(methylsulfonyl)phenoxy)-1H-indazol-3-amine

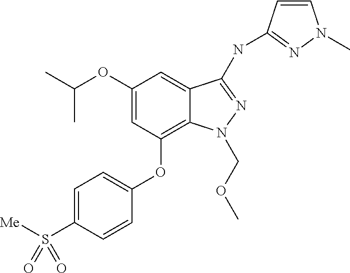

To a stirred solution of 1-(methoxymethyl)-3-(1-methyl-1H-pyrazol-3-ylamino)-7-(4-(methylsulfonyl)phenoxy)-1H-indazol-5-ol (96.3 mg, 0.217 mmol) in DMF (3 ml) were added potassium carbonate (36 mg, 0.26 mmol, 1.2 eq) and 2-propyl iodide (0.024 ml, 0.239 mmol, 1.1 eq) at room temperature. The mixture was stirred at 50° C. for overnight. After dilution with ethyl acetate, the mixture was washed with water and saturated brine, dried over magnesium sulfate, filtered, and concentrated in vacuo. The residue was purified with NH-silica gel column chromatography using 20-66% ethyl acetate in hexane as an eluent to give 71.7 mg of the title compound (67%) as a colorless solid. ¹H NMR (400 MHz, DMSO-d₆) 8 ppm 1.30 (s, 3H) 1.31 (s, 3H) 3.08-3.11 (m, 3H) 3.21 (s, 3H) 3.75 (s, 3H) 4.53 (quin, J=6.06 Hz, 1H) 5.36 (s, 2H) 6.56 (d, J=2.27 Hz, 1H) 6.68 (d, J=2.02 Hz, 1H) 7.24 (d, J=8.84 Hz, 2H) 7.53 (d, J=2.02 Hz, 1H) 7.59 (d, J=2.02 Hz, 1H) 7.94 (d, J=8.84 Hz, 2 H) 9.40 (s, 1H). [M+H] calc'd for C₂₃H₂₇N₅O₅S, 486; found, 486.

### Example 133

### 5-isopropoxy-N-(1-methyl-1H-pyrazol-3-yl)-7-(4-(methylsulfonyl)phenoxy)-1H-indazol-3-amine

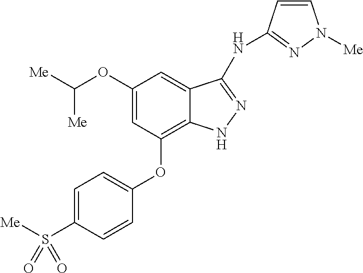

A solution of 5-isopropoxy-1-(methoxymethyl)-N-(1-methyl-1H-pyrazol-3-yl)-7-(4-(methylsulfonyl)phenoxy)-1H-indazol-3-amine (48.5 mg, 0.10 mmol) in formic acid (2 ml) was stirred at 0° C. for 1 h, and concentrated in vacuo. The residue was diluted with ethyl acetate and saturated aqueous NaHCO₃. The organic layer was washed with water and saturated brine, dried over magnesium sulfate, filtered, and concentrated in vacuo. The residue was purified with preparative HPLC to give trifluoroacetic acid salt of the title compound, which was diluted with ethyl acetate and neutralized with saturated aqueous NaHCO₃. The organic layer was washed with saturated brine, dried over magnesium sulfate, filtered, and concentrated in vacuo to give 2.0 mg of the title compound (4.5%) as a brown solid. ¹H NMR (400 MHz, DMSO-d₆) δ ppm 1.29 (s, 3H) 1.31 (s, 3H) 3.19 (s, 3H) 3.73 (s, 3H) 4.50 (quin, J=6.00 Hz, 1H) 6.46 (d, J=2.27 Hz, 1H) 6.65 (d, J=2.02 Hz, 1H) 7.12-7.24 (m, 2 H) 7.50 (dd, J=7.58, 2.02 Hz, 2H) 7.84-7.99 (m, 2H) 9.16 (s, 1H) 11.84 (s, 1H). [M+H] calc'd for C₂₁H₂₃N₅O₄S, 442; found, 442.

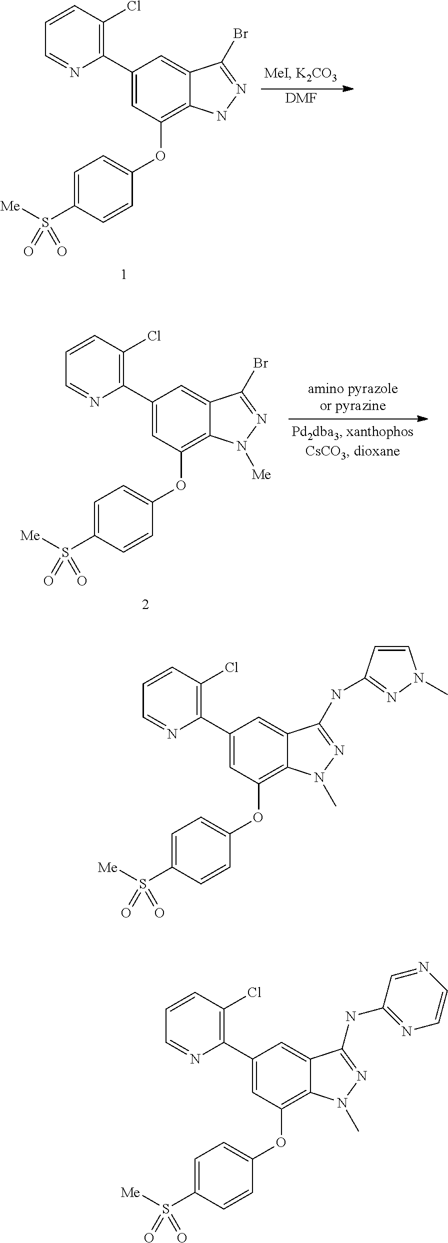

### Example 134

### 5-(3-chloropyridin-2-yl)-1-methyl-N-(1-methyl-1H-pyrazol-3-yl)-7-(4-(methylsulfonyl)phenoxy)-1H-indazol-3-amine

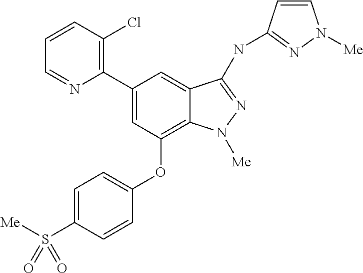

3-Bromo-5-(3-chloropyridin-2-yl)-1-methyl-7-(4-(methylsulfonyl)phenoxy)-1H-indazole (127 mg, 0.258 mmol, 1 eq), 3-amino-1-methylpyrazole (31 mg, 0.31 mmol, 1.2 eq), tris(dibenzylidene-acetone)dipalladium(0) (12 mg, 0.013 mmol, 0.05 eq), 4,5-bis(diphenylphosphino)-9,9-dimethylxanthene (23 mg, 0.039 mmol, 0.15 eq), and cesium carbonate (169 mg, 0.516 mmol, 2 eq) were suspended in degassed anhydrous 1,4-dioxane (4 ml). The mixture was heated under N₂ at 100° C. for overnight. After cooling to room temperature, the insoluble material was filtered and washed with ethyl acetate, and the filtrate was concentrated in vacuo. The residue was purified with NH-silica gel column chromatography using 33-100% ethyl acetate in hexane as an eluent to give 28.7 mg of the title compound (22%) as a colorless solid. ¹H NMR (400 MHz, DMSO-d₆) δ ppm 3.21 (s, 3H) 3.74 (s, 3H) 3.85 (s, 3H). 6.55 (d, J=1.77 Hz, 1H) 7.27 (d, J=8.59 Hz, 2H) 7.35-7.46 (m, 2H) 7.53 (d, J=1.52 Hz, 1H) 7.96 (d, J=8.59 Hz, 2H) 8.04 (d, J=7.33 Hz, 1H) 8.46 (s, 1H) 8.61 (d, J=3.54 Hz, 1H) 9.62 (s, 1H). [M+H] calc'd for C₂₄H₂₁ClN₆O₃S, 509; found, 509.

### Example 135

### 5-(3-chloropyridin-2-yl)-1-methyl-7-(4-(methylsulfonyl)phenoxy)-N-(pyrazin-2-yl)-1H-indazol-3-amine

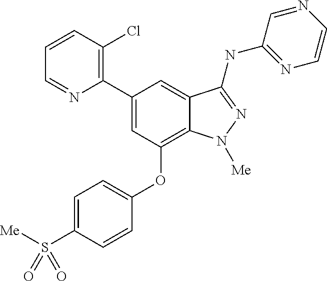

3-Bromo-5-(3-chloropyridin-2-yl)-1-methyl-7-(4-(methylsulfonyl)phenoxy)-1H-indazole (143 mg, 0.29 mmol, 1 eq), aminopyrazine (34 mg, 0.35 mmol, 1.2 eq), tris(dibenzylidene-acetone)dipalladium(0) (13 mg, 0.0145 mmol, 0.05 eq), 4,5-bis(diphenylphosphino)-9,9-dimethylxanthene (26 mg, 0.0435 mmol, 0.15 eq), and cesium carbonate (190 mg, 0.58 mmol, 2 eq) were suspended in degassed anhydrous 1,4-dioxane (2 ml). The mixture was heated under N₂ at 100° C. for overnight. After cooling to room temperature, the insoluble material was filtered and washed with ethyl acetate, and the filtrate was concentrated in vacuo. The residue was purified with NH-silica gel column chromatography using 33-100% ethyl acetate in hexane as an eluent to give 25.7 mg of the title compound (17%) as a colorless solid. ¹H NMR (400 MHz, DMSO-d₆) δ ppm 3.22 (s, 3H) 4.00 (s, 3H) 7.29-7.35 (m, 2H) 7.39-7.47 (m, 2H) 7.94-8.01 (m, 2H) 8.05 (dd, J=8.21, 1.39 Hz, 1H) 8.11 (d, J=2.53 Hz, 1H) 8.24 (dd, J=2.53, 1.52 Hz, 1H) 8.43 (d, J=1.52 Hz, 1H) 8.61 (dd, J=4.55, 1.52 Hz, 1H) 9.18 (d, J=1.26 Hz, 1H) 10.30 (s, 1H). [M+H] calc'd for C₂₄H₁₉ClN₆O₃S, 507; found, 507.

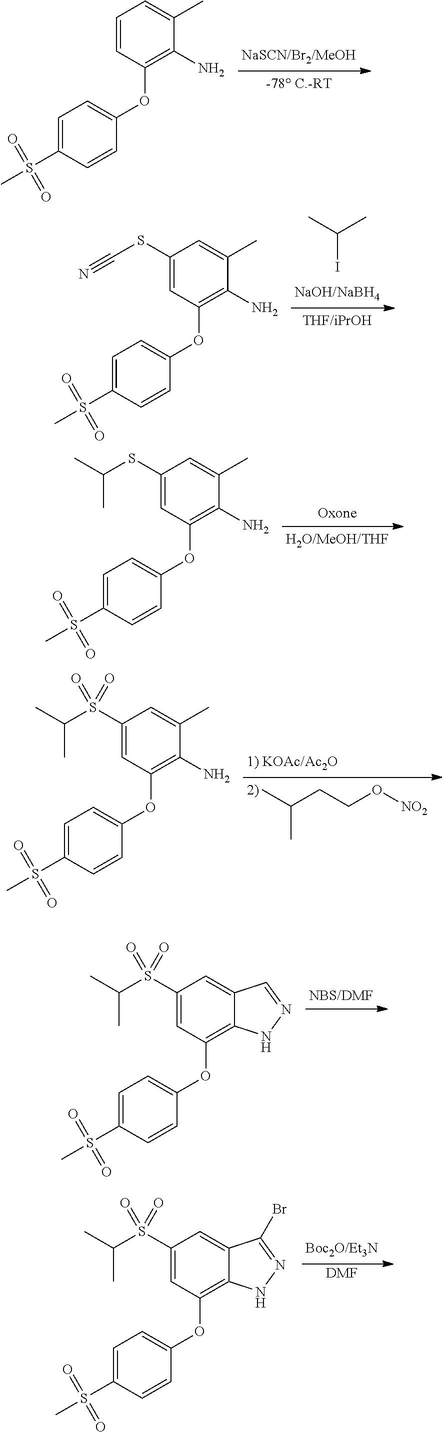

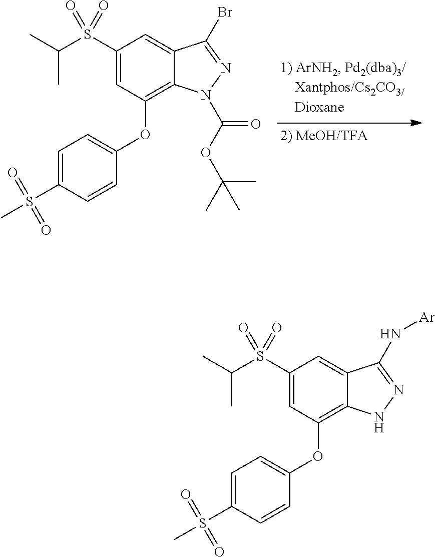

(Wherein Ar is an optionally substituted 5- to 6-membered nitrogen-containing heteroaromatic group.)

### Example 136

### 5-(isopropylsulfonyl)-N-(1-methyl-1H-pyrazol-3-yl)-7-(4-(methylsulfonyl)phenoxy)-1H-indazol-3-amine trifluoroacetic acid salt

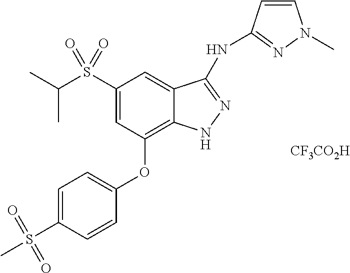

tert-Butyl 3-bromo-5-(isopropylsulfonyl)-7-(4-(methylsulfonyl)phenoxy)-1H-indazole-1-carboxylate (500 mg, 0.87 mmol, 1 eq), 3-amino-1-methylpyrazole (101 mg, 1.04 mmol, 1.3 eq), tris(dibenzylidene-acetone)dipalladium(0) (39 mg, 0.043 mmol, 0.05 eq), 4,5-bis(diphenylphosphino)-9,9-dimethylxanthene (76 mg, 0.13 mmol, 0.15 eq), and cesium carbonate (569 mg, 1.74 mmol, 2 eq) were suspended in degassed anhydrous 1,4-dioxane (10 ml). The mixture was heated under N₂ at 100° C. for overnight. The suspension was filtered and the solid was washed with dichloromethane. The filtrate was concentrated and then re-dissolved in methanol (10 ml) to which trifluoroacetic acid (3 ml) was added. After stirring at room temperature for 1 h, the solvent was removed in vacuo. The residue was purified with preparative HPLC to give 102 mg of the title compound (24%) as a white solid. ¹H NMR (400 MHz, CHLOROFORM-d) δ ppm 1.30 (d, J=6.82 Hz, 6H) 3.11 (s, 3H) 3.19 (quin, J=6.88 Hz, 1H) 3.73 (s, 3H) 6.46 (d, J=2.27 Hz, 1H) 7.21 (m, J=8.84 Hz, 2H) 7.33 (t, J=1.89 Hz, 2H) 7.97 (m, J=9.09 Hz, 2H) 8.06 (d, J=1.26 Hz, 1H). [M+H] calc'd for C₂₁H₂₃N₅O₅S₂, 490; found, 490.

### Example 137

### 5-(isopropylsulfonyl)-7-(4-(methylsulfonyl)phenoxy)-N-(pyrazin-2-yl)-1H-indazol-3-amine trifluoroacetic acid salt

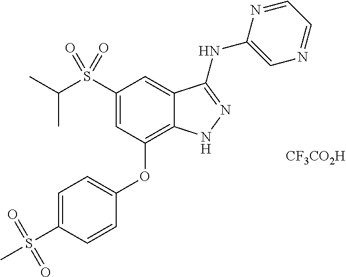

The title compound was prepared according to the procedure outlined in Example 136, using tert-butyl 3-bromo-5-(isopropylsulfonyl)-7-(4-(methylsulfonyl)phenoxy)-1H-indazole-1-carboxylate and aminopyrazine. ¹H NMR (400 MHz, MeOD) δ ppm 1.25 (d, J=6.82 Hz, 6H) 3.11-3.17 (m, 3H) 3.33-3.39 (m, 1 H) 7.36 (m, 2H) 7.42 (d, J=1.26 Hz, 1H) 8.03 (m, 2H) 8.07 (d, J=1.52 Hz, 1H) 8.26-8.32 (m, 1H) 8.40 (d, J=2.53 Hz, 1H) 9.13 (s, 1H). [M+H] calc'd for C₂₁H₂₁N₅O₅S₂, 488; found, 488.

### Example 138

### 5-(3-chloropyridin-2-yl)-N-(1-(2-(methylsulfonyl)ethyl)-1H-pyrazol-3-yl)-7-(4-(methylsulfonyl)phenoxy)-1H-indazol-3-amine trifluoroacetic acid salt

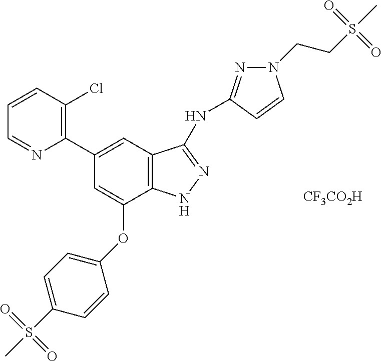

The title compound was prepared according to the procedure outlined in Example 136, using tert-butyl 3-bromo-5-(3-chloropyridin-2-yl)-7-(4-(methylsulfonyl)phenoxy)-1H-indazole-1-carboxylate and 1-(2-(methylsulfonyl)ethyl)-1H-1s pyrazol-3-amine. ¹H NMR (400 MHz, CHLOROFORM-d) δ ppm 2.95 (s, 3H) 3.04 (s, 3H) 3.08 (s, 2H) 3.66 (t, J=6.69 Hz, 2H) 5.06 (t, J=6.95 Hz, 2H) 7.29-7.38 (m, 2H) 7.39-7.44 (m, 1H) 7.83 (d, J=1.26 Hz, 1H) 7.92-8.03 (m, 3H) 8.09 (s, 1H) 8.69 (d, J=3.54 Hz, 1H). [M+H] calc'd for C₂₅H₂₃ClN₆O₅S₂, 587; found, 587.

### Example 139

### N-(1-(2-aminoethyl)-1H-pyrazol-3-yl)-5-(3-chloropyridin-2-yl)-7-(4-(methylsulfonyl)phenoxy)-1H-indazol-3-amine trifluoroacetic acid salt

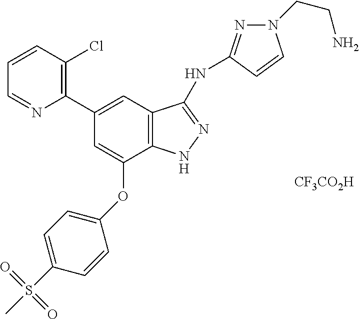

The title compound was prepared according to the procedure outlined in Example 136, using tert-butyl 3-bromo-5-(3-chloropyridin-2-yl)-7-(4-(methylsulfonyl)phenoxy)-1H-indazole-1-carboxylate and tert-butyl 2-(3-amino-1H-pyrazol-1-yl)ethylcarbamate. [M+H] calc'd for C₂₄H₂₂ClN₇O₃S, 524; found, 524.

### Example 140

### 1-(3-(5-(3-chloropyridin-2-yl)-7-(4-(methylsulfonyl)phenoxy)-1H-indazol-3-ylamino)-1H-pyrazol-1-yl)-3-methoxypropan-2-ol

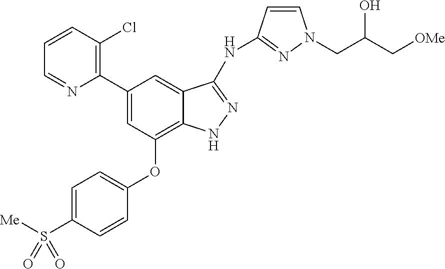

tert-Butyl 3-bromo-5-(3-chloropyridin-2-yl)-7-(4-(methylsulfonyl)phenoxy)-1H-indazole-1-carboxylate (221 mg, 0.382 mmol, 1 eq), 1-(2-(tert-butyldimethylsilyloxy)-3-methoxypropyl)-1H-pyrazol-3-amine (131 mg, 0.46 mmol, 1.2 eq), tris(dibenzylidene-acetone)dipalladium(0) (18 mg, 0.019 mmol, 0.05 eq), 4,5-bis(diphenylphosphino)-9,9-dimethylxanthene (34 mg, 0.057 mmol, 0.15 eq), and cesium carbonate (249 mg, 0.764 mmol, 2 eq) were suspended in degassed anhydrous 1,4-dioxane (5 ml). The mixture was heated under N₂ at 100° C. for overnight. After cooling to room temperature, the insoluble material was filtered and washed with ethyl acetate, and the filtrate was concentrated in vacuo. The residue was purified with NH-silica gel column chromatography using 10-50% ethyl acetate in hexane as an eluent to give a light yellow oil, which was dissolved in ethanol (2 ml) and concentrated hydrochloric acid (2 ml). The mixture was stirred at room temperature for 2 h, and concentrated in vacuo. Ethyl acetate and saturated aqueous NaHCO₃ were added to the residue and the organic layer was separated, washed with water, and saturated brine, dried over magnesium sulfate, filtered, and concentrated in vacuo. The residue was purified with preparative HPLC to give trifluoroacetic acid salt of the title compound, which was diluted with ethyl acetate and neutralized with saturated aqueous NaHCO₃. The organic layer was washed with saturated brine, dried over magnesium sulfate, filtered, and concentrated in vacuo to give 34.1 mg of the title compound (16% in 2 steps) as a colorless solid. ¹H NMR (400 MHz, DMSO-d₆) δ ppm 3.20 (s, 3H) 3.26 (s, 4H) 3.73-4.17 (m, 4H) 5.12 (d, J=5.31 Hz, 1H) 6.53 (d, J=1.52 Hz, 1H) 7.24 (d, J=8.59 Hz, 2H) 7.32-7.45 (m, 2H) 7.52 (s, 1H) 7.94 (d, J=8.59 Hz, 2H) 8.04 (d, J=7.33 Hz, 1H) 8.45 (s, 1H) 8.61 (d, J=3.79 Hz, 1H) 9.59 (s, 1H) 12.32 (s, 1H). [M+H] calc'd for C₂₆H₂₅ClN₆O₅S, 569; found, 569.

### Example 141

### 3-(3-(5-(3-chloropyridin-2-yl)-1-methyl-7-(4-(methylsulfonyl)phenoxy)-1H-indazol-3-ylamino)-1H-pyrazol-1-yl)propane-1,2-diol

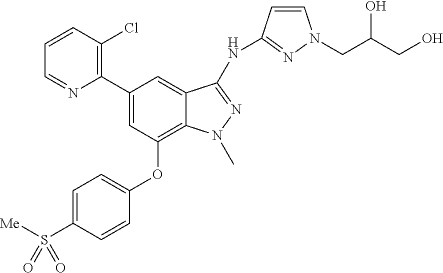

The title compound was prepared according to the procedure outlined in Example 131, using 3-bromo-5-(3-chloropyridin-2-yl)-1-methyl-7-(4-(methylsulfonyl)phenoxy)-1H-indazole and 1-((2,2-dimethyl-1,3-dioxolan-4-yl)methyl)-1H-pyrazol-3-amine. ¹H NMR (400 MHz, DMSO-d₆) δ ppm 3.21 (s, 3H) 3.26-3.43 (m, 2H) 3.74-3.96 (m, 5H) 4.10 (dd, J=13.14, 3.79 Hz, 1H) 4.71 (t, J=5.68 Hz, 1H) 4.95 (d, J=5.05 Hz, 1H) 6.55 (d, J=2.02 Hz, 1H) 7.27 (d, J=8.84 Hz, 2H) 7.36-7.45 (m, 2H) 7.53 (d, J=2.27 Hz, 1H) 7.96 (d, J=8.84 Hz, 2H) 8.04 (dd, J=8.08, 1.52 Hz, 1H) 8.46 (d, J=1.26 Hz, 1H) 8.61 (dd, J=4.55, 1.52 Hz, 1H) 9.65 (s, 1H). [M+H] calc'd for C₂₆H₂₅ClN₆O₅S, 569; found, 569.

### Example 142

### 1-(3-(5-(3-chloropyridin-2-yl)-7-(4-(methylsulfonyl)phenoxy)-1H-indazol-3-ylamino)-1H-pyrazol-1-yl)-3-methylbutane-2,3-diol

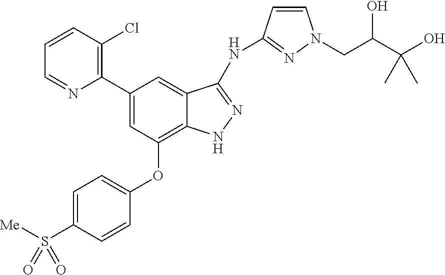

The title compound was prepared according to the procedure outlined in Example 131, using tert-butyl 3-bromo-5-(3-chloropyridin-2-yl)-7-(4-(methylsulfonyl)phenoxy)-1H-indazole-1-carboxylate and 1-((2,2,5,5-tetramethyl-1,3-dioxolan-4-yl)methyl)-1H-pyrazol-3-amine. ¹H NMR (400 MHz, DMSO-d₆) δ ppm 1.08 (s, 3H) 1.12 (s, 3H) 3.20 (s, 3H) 3.56 (ddd, J=9.54, 6.00, 1.89 Hz, 1H) 3.77 (dd, J=13.77, 9.47 Hz, 1 H) 4.25 (d, J=1.52 Hz, 1H) 4.43 (s, 1H) 4.93 (d, J=6.06 Hz, 1 H) 6.51 (d, J=2.27 Hz, 1H) 7.24 (d, J=9.09 Hz, 2H) 7.37 (d, J=1.26 Hz, 1H) 7.40 (dd, J=8.08, 4.55 Hz, 1H) 7.53 (d, J=2.02 Hz, 1H) 7.94 (d, J=8.84 Hz, 2H) 8.04 (dd, J=8.08, 1.52 Hz, 1H) 8.44 (s, 1H) 8.61 (dd, J=4.67, 1.39 Hz, 1H) 9.58 (s, 1H) 12.31 (s, 1H). [M+H] calc'd for C₂₇H₂₇ClN₆O₅S, 583; found, 583.

### Example 143

### 2-(3-(5-(3-chloropyridin-2-yl)-1-methyl-7-(4-(methylsulfonyl)phenoxy)-1H-indazol-3-ylamino)-1H-pyrazol-1-yl)ethanol

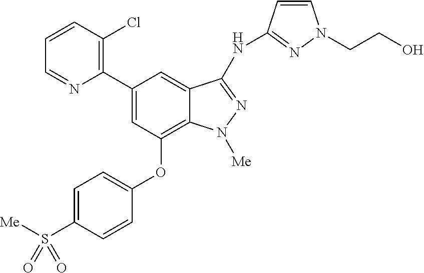

The title compound was prepared according to the procedure outlined in Example 129, using 3-bromo-5-(3-chloropyridin-2-yl)-1-methyl-7-(4-(methylsulfonyl)phenoxy)-1H-indazole and 1-(2-(tert-butyldimethylsilyloxy)ethyl)-1H-pyrazol-3-amine. ¹H NMR (400 MHz, DMSO-d₆) δ ppm 3.21 (s, 3H) 3.74 (q, J=5.73 Hz, 2H) 3.86 (s, 3H) 4.03 (t, J=5.68 Hz, 2H) 4.88 (t, J=5.31 Hz, 1H) 6.55 (d, J=2.27 Hz, 1H) 7.27 (d, J=9.09 Hz, 2H) 7.36-7.45 (m, 2H) 7.55 (d, J=2.27 Hz, 1H) is 7.96 (d, J=8.84 Hz, 2H) 8.04 (dd, J=8.08, 1.52 Hz, 1H) 8.47 (d, J=1.26 Hz, 1H) 8.61 (dd, J=4.67, 1.39 Hz, 1H) 9.66 (s, 1 H). [M+H] calc'd for C₂₅H₂₃ClN₆O₄S, 539; found, 539.

### Example 144

### 1-(3-(5-(3-chloropyridin-2-yl)-1-methyl-7-(4-(methylsulfonyl)phenoxy)-1H-indazol-3-ylamino)-1H-pyrazol-1-yl)-3-methylbutane-2,3-diol

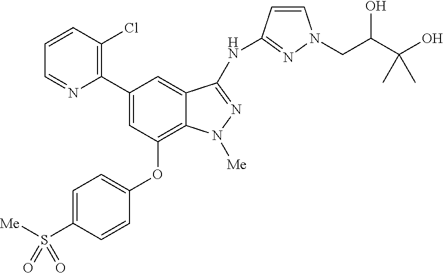

The title compound was prepared according to the procedure outlined in Example 129, using 3-bromo-5-(3-chloropyridin-2-yl)-1-methyl-7-(4-(methylsulfonyl)phenoxy)-1H-indazole and 1-((2,2,5,5-tetramethyl-1,3-dioxolan-4-yl)methyl)-1H-pyrazol-3-amine. ¹H NMR (400 MHz, DMSO-d₆) 5 ppm 1.08 (s, 3 H) 1.12 (s, 3H) 3.21 (s, 3H) 3.51-3.60 (m, 1H) 3.71-3.83 (m, 1H) 3.86 (s, 3H) 4.21-4.32 (m, 1H) 4.44 (s, 1H) 4.93 (d, J=5.81 Hz, 1H) 6.53 (d, J=2.02 Hz, 1H) 7.27 (d, J=8.84 Hz, 2H) 7.35-7.45 (m, 2H) 7.54 (d, J=2.27 Hz, 1H) 7.96 (d, J=8.84 Hz, 2H) 8.04 (dd, J=8.08, 1.52 Hz, 1H) 8.46 (d, J=1.52 Hz, 1H) 8.61 (dd, J=4.55, 1.52 Hz, 1H) 9.66 (s, 1H). [M+H] calc'd for C₂₈H₂₉ClN₆O₅S, 597; found, 597.

### Experimental Example 1

### Determination of GK Activity Value (Fluorescence Assay)

GK enzyme reactions were performed in 50 mmol/L HEPES pH 7.4, 200 mmol/L KCl, 5 mmol/L MgCl₂, 2 mmol/L DTT, containing 50 μmol/L 2′-(or-3′)-O-(N-methylanthraniloyl)adenosine 5′-triphosphate (Mant-ATP) (Jena Bioscience GmbH), 5 mmol/L D-glucose, 5% DMSO and 6 μg/mL GST-hLGK1 obtained in Reference Example 2A in a total volume 50 μL. The reactions were performed in 384 well black plates (Nalge Nunc International K.K.). Prior to the reaction, the enzyme and test compound were incubated for 10 min at 37° C., and 25 mM D-glucose solution (10 μL) was added to start the reaction. The final concentration of the test compound is 10 μmol/L. After the incubation for 60 min at 37° C., the reaction was quenched by adding 25 μL of a quenching solution (containing 200 mM HEPES (pH 7.4), 20 mM MgCl₂, 200 mM EDTA, 0.03% Triton-X 100, 0.3% Coating 3 reagent (Caliper Life Sciences, Inc.)).

Mant-ATP (substrate, 2′-(or-3′)—O—(N-methylanthraniloyl)adenosine 5′-triphosphate) and Mant-ADP (reaction resultant product) were separated from each well after the reaction by a microchip type capillary electrophoresis apparatus 250 HTS (Caliper Life Sciences, Inc.). The reaction rate [(reaction resultant product peak height)/(reaction resultant product peak height+substrate peak height)×100(%)] was calculated from the ratio of the substrate peak height and reaction resultant product peak height obtained by fluorescence detection (excitation wavelength 355 nm, measurement wavelength 460 nm) and used as the index of GK activity.

As a control group, the reaction rate was calculated in the same manner as above without the test compounds.

The percentage obtained by dividing the reaction rate of the well added with the test compound (test compound addition group) by the reaction rate of the control group was taken as the GK activity value (Emax) of the test compound. The results are shown in Table 1.

TABLE 1

| Example No. | Emax (%) |
|---|---|
| 20 | 156 |
| 28 | 231 |
| 30 | 129 |
| 34 | 186 |
| 63 | 175 |
| 72 | 194 |
| 73 | 111 |
| 85 | 231 |
| 100 | 200 |
| 101 | 202 |
| 105 | 140 |
| 108 | 199 |
| 109 | 218 |
| 117 | 211 |

### Experimental Example 2

### Determination of GK Activity Value (Luminescence Assay)

The activation properties of compounds for GK may be determined using a black 384-well-plate format under the following reaction conditions: 25 mM Hepes pH 7.2, 25 mM NaCl, 10 mM MgCl₂, 0.01% Brij35, 1 mM DTT, 5 μM ATP, 5 mM Glucose 2% DMSO. The amount of ATP consumed may be determined quantitatively by addition of equal volume of luciferase reagent (luciferase+beetle luciferin—KinaseGlo Luminescent Kinase Assay kit from Promega). The luminescence intensity may be measured by using the Analyst HT from LJL Biosystems.

The assay reaction may be initiated as follows: 4 μl of substrate mixture (12.5 μM ATP and 12.5 mM Glucose) was added to each well of the plate, followed by the addition of 2 μl of activator (2 fold serial dilutions for 11 data points for each activator) containing 10% DMSO. 4 μL of 1.25 nM GK solution obtained in Reference Example 3A may be added to initiate the reaction. The reaction mixture may then be incubated at room temperature for 60 min, and quenched and developed by addition of 10 μL of luciferase reagent. Luminescence intensities of the resulting reaction mixtures may be measured after a 10 min incubation at room temperature. The luminescence intensity may be measured by using the Analyst HT from LJL Biosystems.

% ACTmax values may be calculated by non-linear curve fitting of the compound concentrations and luminescence intensities to a standard inhibition/activation equation. % ACTmax represents the calculated maximal gain in GK enzyme activity at a saturating concentration of the compound. % ACTmax values for select compounds of the present invention are given in Table 2.

TABLE 2

| Example No. | % ACTmax |
|---|---|
| 131 | 147.8 |
| 133 | 146.3 |
| 134 | 24.1 |
| 136 | 110.9 |
| 140 | 146.5 |

### Formulation Example 1

### Production of Capsule

| | |
|---|---|
| 1) compound of Example 1 | 30 mg |
| 2) finely divided powder cellulose | 10 mg |
| 3) lactose | 19 mg |
| 4) magnesium .stearate | 1 mg |
| total | 60 mg |

1), 2), 3) and 4) are mixed and filled in a gelatin capsule.

### Formulation Example 2

### Production of Tablet

| | |
|---|---|
| 1) compound of Example 1 | 30 g |
| 2) lactose | 50 g |
| 3) cornstarch | 15 g |
| 4) calcium carboxymethylcellulose | 44 g |
| 5) magnesium stearate | 1 g |
| 1000 tablets total | 140 g |

The total amount of 1), 2) and 3), and 30 g of 4) are m kneaded with water, vacuum dried and sized. The sized powder is mixed with 14 g of 4) and 1 g of 5), and the mixture is punched by a tabletting machine. In this way, 1000 tablets containing 30 mg of the compound of Example 1 per tablet are obtained.

### INDUSTRIAL APPLICABILITY

The glucokinase activator of the present invention has a superior activity and is useful as a pharmaceutical agent such as an agent for the prophylaxis or treatment of diabetes, obesity and the like, and the like.

This application is based on U.S. provisional application No. 60/929,240 filed in United States, the contents of which are incorporated in full herein by this reference.

1. A compound represented by the formula (I): 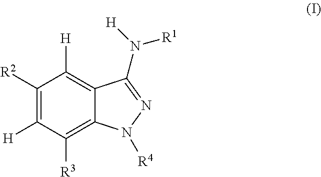
wherein
R¹ is
an optionally substituted 4 to 7-membered nitrogen-containing heterocyclic group,
optionally substituted carbamoyl, or
optionally substituted sulfamoyl;
R² is
optionally substituted alkyl,
optionally substituted alkoxy,
an optionally substituted 3 to 7-membered cyclic group,
—SR′, —SOR′, or —SO₂R′ (R′ is a substituent);
R³ is
hydrogen,
halogen,
optionally substituted alkyl,
optionally substituted alkenyl,
optionally substituted alkoxy,
—O-Cy (Cy is an optionally substituted 3 to 7-membered cyclic group which may be condensed with benzene),
—SR″, —SOR″, or —SO₂R″ (R″ is a substituent), or
an optionally substituted 3 to 7-membered cyclic group which may be condensed with benzene;
R⁴ is
hydrogen, or
optionally substituted alkyl;
provided that
when R³ is hydrogen, halogen, or methoxy,
then R² is not optionally substituted alkyl, or optionally substituted alkoxy; further provided that 5-[5-{[(2S)-2-amino-3-phenylpropyl]oxy}-2-(3-furyl)pyridin-3-yl]-N-pyridin-4-yl-1H-indazol-3-amine and 5-[5-{[(2S)-2-amino-3-phenylpropyl]oxy}-2-(3-furyl)pyridin-3-yl]-1-(4-methoxybenzyl)-N-pyridin-4-yl-1H-indazol-3-amine are excluded;
or a salt thereof.

2. The compound according to claim 1,
wherein
R¹ is
an optionally substituted 4 to 7-membered nitrogen-containing heterocyclic group, or optionally substituted sulfamoyl.

3. The compound according to claim 1,
wherein
R² is
an optionally substituted 3 to 7-membered cyclic group,
—SR′, or —SOR′, or —SO₂R′ (R′ is a substituent).

4. The compound according to claim 1,
wherein
R¹ is
(i) a 4 to 7-membered nitrogen-containing heterocyclic group optionally substituted by C₁₋₆ alkyl optionally substituted by one or more of the same or different substituents selected from
hydroxy,
cyano,
optionally substituted amino,
optionally substituted 5 to 6-membered cyclic amino,
carboxy,
C₁₋₆ alkoxycarbonyl, and
optionally substituted carbamoyl, or
(ii) optionally substituted carbamoyl.

5. The compound according to claim 1,
wherein
R² is
(i) C₁₋₆ alkyl,
(ii) C₁₋₆ alkoxy optionally substituted by one or more of the same or different substituents selected from C₆₋₁₀ aryl and C₁₋₆ alkoxy,
(iii) —SR′, —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), or
(iv) an optionally substituted 3 to 7-membered cyclic group.

6. The compound according to claim 1,
wherein
R³ is
(i) hydrogen,
(ii) halogen,
(iii) C₁₋₆ alkyl,
(iv) C₂₋₆ alkenyl optionally substituted by 5 to 6-membered heterocyclic group,
(v) C₁₋₆ alkoxy optionally substituted by one or more of the same or different substituents selected from
(a) optionally substituted amino,
(b) phenyl optionally substituted by one or more of the same or different substituents selected from
halogen, and
C₁₋₆ alkylsulfonyl,
(c) 5 to 6-membered heterocyclic ring which may be substituted by one or more of the same or different substituents selected from
C₁₋₆ alkyl,
C₁₋₆ alkylthio,
C₁₋₆ alkylsulfonyl,
carboxy,
C₁₋₆ alkoxycarbonyl and
oxo,
and which may be condensed with benzene,
(d) carbamoyl optionally substituted by C₁₋₆ alkyl, and
(e) C₁₋₆ alkylsulfonyl,
(vi) phenoxy or 5 to 6-membered heteroaryloxy, each of which may be substituted by one or more of the same or different substituents selected from
halogen,
C₁₋₆ alkylsulfonyl, and
optionally substituted carbamoyl, or
(vii) 5 to 6-membered heterocyclic ring which may be substituted by C₁₋₆ alkyl, and which may be condensed with benzene.

7. The compound according to claim 1,
wherein
R⁴ is
(i) hydrogen, or
(ii) C₁₋₆ alkyl optionally substituted by one or more of the same or different substituents selected from C₆₋₁₀ aryl and C₁₋₆ alkoxy.

8. The compound according to claim 1,
wherein
R¹ is
(i) a 4 to 7-membered nitrogen-containing heterocyclic group optionally substituted by C₁₋₆ alkyl optionally substituted by one or more of the same or different substituents selected from
hydroxy,
cyano,
optionally substituted amino,
optionally substituted 5 to 6-membered cyclic amino,
carboxy,
C₁₋₆ alkoxycarbonyl, and
optionally substituted carbamoyl, or
(ii) optionally substituted carbamoyl;
R² is
(i) C₁₋₆ alkyl,
(ii) C₁₋₆ alkoxy optionally substituted by one or more of the same or different substituents selected from C₆₋₁₀ aryl and C₁₋₆ alkoxy,
(iii)-SR′, —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), or
(iv) an optionally substituted 3 to 7-membered cyclic group;
R³ is
(i) hydrogen,
(ii) halogen,
(iii) C₁₋₆ alkyl,
(iv) C₂₋₆ alkenyl optionally substituted by 5 to 6-membered heterocyclic group,
(v) C₁₋₆ alkoxy optionally substituted by one or more of the same or different substituents selected from
(a) optionally substituted amino,
(b) phenyl optionally substituted by one or more of the same or different substituents selected from
halogen, and
C₁₋₆ alkylsulfonyl,
(c) 5 to 6-membered heterocyclic ring which may be substituted by one or more of the same or different substituents selected from
C₁₋₆ alkyl,
C₁₋₆ alkylthio,
C₁₋₆ alkylsulfonyl,
carboxy,
C₁₋₆ alkoxycarbonyl and
oxo,
and which may be condensed with benzene,
(d) carbamoyl optionally substituted by C₁₋₆ alkyl, and
(e) C₁₋₆ alkylsulfonyl,
(vi) phenoxy or 5 to 6-membered heteroaryloxy optionally substituted by one or more of the same or different substituents selected from
halogen,
C₁₋₆ alkylsulfonyl, and
optionally substituted carbamoyl, or
(vii) 5 to 6-membered heterocyclic ring which may be substituted by C₁₋₆ alkyl, and which may be condensed with benzene; and
R⁴ is
(i) hydrogen, or
(ii) C₁₋₆ alkyl optionally substituted by one or more of the same or different substituents selected from C₆₋₁₀ aryl and C₁₋₆ alkoxy.

9. The compound according to claim 1,
wherein
R³ is
optionally substituted alkyl,
optionally substituted alkenyl,
C₂₋₆ alkoxy, or substituted C₁₋₆ alkoxy,
—O-Cy (Cy is an optionally substituted 3 to 7-membered cyclic group which may be condensed with benzene),
—SR″, —SOR″, or —SO₂R″ (R″ is a substituent), or
an optionally substituted 3 to 7-membered cyclic group which may be condensed with benzene.

10. The compound according to claim 9,
wherein
R¹ is
(i) a 4 to 7-membered nitrogen-containing heterocyclic group optionally substituted by C₁₋₆ alkyl optionally substituted by one or more of the same or different substituents selected from
hydroxy,
cyano,
optionally substituted amino,
optionally substituted alkoxy,
—SR″″, —SOR″″, or —SO₂R″″ (R″″ is a substituent),
optionally substituted 5 to 6-membered cyclic amino,
carboxy,
C₁₋₆ alkoxycarbonyl, and
optionally substituted carbamoyl, or
(ii) optionally substituted carbamoyl.

11. The compound according to claim 9,
wherein
R² is
(i) C₁₋₆ alkyl,
(ii) C₁₋₆ alkoxy optionally substituted by one or more of the same or different substituents selected from C₆₋₁₀ aryl and C₁₋₆ alkoxy,
(iii) —SR′, —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), or
(iv) an optionally substituted 3 to 7-membered cyclic group.

12. The compound according to claim 9,
wherein
R³ is
(i) C₁₋₆ alkyl,
(ii) C₂₋₆ alkenyl optionally substituted by 5 to 6-membered heterocyclic group,
(iii) C₂₋₆ alkoxy, or C₁₋₆ alkoxy substituted by one or more of the same or different substituents selected from
(a) optionally substituted amino,
(b) phenyl optionally substituted by one or more of the same or different substituents selected from
halogen, and
₁₋₆ alkylsulfonyl,
(c) 5 to 6-membered heterocyclic group which may be substituted by one or more of the same or different substituents selected from
C₁₋₆ alkyl,
C₁₋₆ alkylthio,
C₁₋₆ alkylsulfonyl,
carboxy,
C₁₋₆ alkoxycarbonyl and
oxo,
and which may be condensed with benzene,
(d) carbamoyl optionally substituted by C₁₋₆ alkyl, and
(e) C₁₋₆ alkylsulfonyl,
(iv) phenoxy or 5 to 6-membered heteroaryloxy, each of which may be substituted by one or more of the same or different substituents selected from
halogen,
C₁₋₆ alkylsulfonyl, and
optionally substituted carbamoyl, or
(v) 5 to 6-membered heterocyclic group which may be substituted by C₁₋₆ alkyl, and which may be condensed with benzene.

13. The compound according to claim 9,
wherein
R⁴ is
(i) hydrogen, or
(ii) C₁₋₆ alkyl optionally substituted by one or more of the same or different substituents selected from C₆₋₁₀ aryl and C₁₋₆ alkoxy.

14. The compound according to claim 9,
wherein
R¹ is
(i) a 4 to 7-membered nitrogen-containing heterocyclic group optionally substituted by C₁₋₆ alkyl optionally substituted by one or more of the same or different substituents selected from
hydroxy,
cyano,
optionally substituted amino,
optionally substituted alkoxy,
—SR″″, —SOR″″, or —SO₂R″″ (R″″ is a substituent),
optionally substituted 5 to 6-membered cyclic amino,
carboxy,
C₁₋₆ alkoxycarbonyl, and
optionally substituted carbamoyl, or
(ii) optionally substituted carbamoyl;
R² is
C₁₋₆ alkyl,
(ii) C₁₋₆ alkoxy optionally substituted by one or more of the same or different substituents selected from C₆₋₁₀ aryl and C₁₋₆ alkoxy,
(iii) —SR′, —SOR′, or —SO₂R′ (R′ is C₁₋₆ alkyl, C₃₋₇ cycloalkyl, or 4 to 7-membered nitrogen-containing heterocyclic group optionally substituted by C₁₋₆ alkyl), or
(iv) an optionally substituted 3 to 7-membered cyclic group;
R³ is
C₁₋₆ alkyl,
(ii) C₂₋₆ alkenyl optionally substituted by 5 to 6-membered heterocyclic group,
(iii) C₂₋₆ alkoxy, or C₁₋₆ alkoxy substituted by one or more of the same or different substituents selected from
(a) optionally substituted amino,
(b) phenyl optionally substituted by one or more of the same or different substituents selected from
halogen, and
C₁₋₆ alkylsulfonyl,
(c) 5 to 6-membered heterocyclic group which may be substituted by one or more of the same or different substituents selected from
C₁₋₆ alkyl,
C₁₋₆ alkylthio,
C₁₋₆ alkylsulfonyl,
carboxy,
C₁₋₆ alkoxycarbonyl and
oxo,
and which may be condensed with benzene,
(d) carbamoyl optionally substituted by C₁₋₆ alkyl, and
(e) C₁₋₆ alkylsulfonyl,
(iv) phenoxy or 5 to 6-membered heteroaryloxy, each of which may be substituted by one or more of the same or different substituents selected from
halogen,
C₁₋₆ alkylsulfonyl, and
optionally substituted carbamoyl, or
(v) 5 to 6-membered heterocyclic group which may be substituted by C₁₋₆ alkyl, and which may be condensed with benzene;
R⁴ is
(i) hydrogen, or
(ii) C₁₋₆ alkyl optionally substituted by one or more of the same or different substituents selected from C₆₋₁₀ aryl and C₁₋₆ alkoxy.

15. 1-(3-(5-(3-chloropyridin-2-yl)-7-(4-(methylsulfonyl)phenoxy)-1H-indazol-3-ylamino)-1H-pyrazol-1-yl)-3-methoxypropan-2-ol and a salt thereof.

16. 5-(isopropylsulfonyl)-N-(1-methyl-1H-pyrazol-3-yl)-7-(4-(methylsulfonyl)phenoxy)-1H-indazol-3-amine and a salt thereof.

17. 5-isopropoxy-N-(1-methyl-1H-pyrazol-3-yl)-7-(4-(methylsulfonyl)phenoxy)-1H-indazol-3-amine and a salt thereof.

18. 5-(3-chloropyridin-2-yl)-1-methyl-N-(1-methyl-1H-pyrazol-3-yl)-7-(4-(methylsulfonyl)phenoxy)-1H-indazol-3-amine and a salt thereof.

19. 3-(3-(5-(3-chloropyridin-2-yl)-7-(4-(methylsulfonyl)phenoxy)-1H-indazol-3-ylamino)-1H-pyrazol-1-yl)propane-1,2-diol and a salt thereof.

20. A prodrug of the compound according to claim 1.

21. A pharmaceutical composition which comprises the compound according to claim 1 or a prodrug thereof.

22. The pharmaceutical composition according to claim 21 which is an agent for activating glucokinase.

23. The pharmaceutical composition according to claim 21 which is an agent for preventing or treating diabetes or obesity.

24. A method of activating glucokinase which comprises administering to a subject according to claim 1.

25. A method of preventing or treating diabetes or obesity which comprises administering to a subject a compound according to claim 1.

26-27. (canceled)


SEQUENCE LISTING (4 sequences)

SEQ ID NO: 1 - DNA, 27 bp, Artificial sequence
  Synthetic construct; primer for cloning glucokinase
cagctctccatccaagcagccgttgct

SEQ ID NO: 2 - DNA, 21 bp, Artificial sequence
  Synthetic construct; primer for cloning glucokinase
ggcggcctgggtcctgacaag

SEQ ID NO: 3 - DNA, 69 bp, Artificial sequence
  Synthetic construct; primer for cloning glucokinase
ggatccatgcccagaccaagatcccaactcccacaacccaactcccaggtagagcagatcctggcagag

SEQ ID NO: 4 - DNA, 24 bp, Artificial sequence
  Synthetic construct; primer for cloning glucokinase
gaattcctggcccagcatacaggc